(19) United States
(12) Patent Application Publication
(10) Pub. No.: US 2025/0011344 A1
(43) Pub. Date: Jan. 9, 2025

(54) PRODRUGS AND DERIVATIVES OF PSILOCIN AND USES THEREOF

(71) Applicant: Terran Biosciences Inc., New York, NY (US)

(72) Inventors: Sam CLARK, Miami, FL (US); Matthew Alexander James DUNCTON, Las Vegas, NV (US)

(21) Appl. No.: 18/684,970

(22) PCT Filed: Aug. 19, 2022
(86) PCT No.: PCT/US2022/040922

§ 371 Date: Feb. 20, 2024

Related U.S. Application Data
(60) Provisional application No. 63/289,025, filed on Dec. 13, 2021.
(60) Provisional application No. 63/235,543, filed on Aug. 20, 2021.

(51) Int. Cl. C07F 7/08; C07D 209/16; C07D 401/14; C07D 403/12; C07D 405/12; C07D 413/12; C07D 491/107; C07F 9/572

(52) U.S. Cl. CPC C07F 7/0812; C07D 209/16; C07D 401/14; C07D 403/12; C07D 405/12; C07D 413/12; C07D 491/107; C07F 9/5728

(57) ABSTRACT

Described herein are compounds that are derivatives (e.g., prodrugs) of psilocin. Also described herein are uses of the compounds provided herein for treating or preventing a disease, disorder, or condition in which an increased level of psilocin is beneficial.

### CROSS REFERENCE TO RELATED APPLICATIONS

This application claims the benefit of U.S. Provisional Patent Application No. 63/235,543, filed Aug. 20, 2021, and U.S. Provisional Patent Application No. 63/289,025, filed on Dec. 13, 2021, the content of each of which is incorporated by reference herein in its entirety.

### BACKGROUND OF THE INVENTION

Nearly 1 in 5 adults in the United States suffer from mental illness, and over 50% of Americans will be diagnosed with a psychiatric disorder at some point in their lifetime. 1 in 25 Americans is afflicted with severe mental illness, such as major depression, schizophrenia, or bipolar disorder.

### SUMMARY OF THE INVENTION

In one aspect, provided herein are compounds of Formula (I), or a pharmaceutically acceptable salt, solvate, or isotopolog thereof:

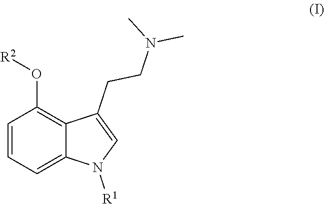

wherein:
R¹ is hydrogen, —OH, unsubstituted or substituted alkyl, OR, or C(O)OR; wherein R is unsubstituted alkyl;
R² is —C(O)R³, —C(O)OR³, —CH(R⁴)OC(O)R⁵, —CH(R⁴)OC(O)OR⁵, —C(O)NR⁶R⁷, —CH(R⁴)OC(O)NR⁶R⁷, —S(O)₂NR⁶R⁷, —S(O)₂OR⁵, —P(O)OR⁸(NR⁹R¹⁰), —P(O)(OR¹¹)(OR¹²), —CH(R⁴)OP(O)(OR¹¹)(OR¹²), or —Si(R³)(R⁴)(R⁵);
each of R³, R⁴, R⁵, and R⁸ is independently hydrogen, alkyl, heteroalkyl, cycloalkyl, heterocyclylalkyl, aryl, or heteroaryl, wherein alkyl, heteroalkyl, cycloalkyl, heterocyclylalkyl, aryl, or heteroaryl is unsubstituted or substituted with one or more R^{A};
each of R⁶ and R⁷ is independently hydrogen, alkyl, heteroalkyl, cycloalkyl, heterocyclylalkyl, aryl, or heteroaryl, wherein alkyl, heteroalkyl, cycloalkyl, heterocyclylalkyl, aryl, or heteroaryl is unsubstituted or substituted with one or more R^{A}; or R⁶ and R⁷ together with the atom to which they are attached form a heterocyclylalkyl ring or a heteroaryl ring that is unsubstituted or substituted with one or more R^{A};
each of R⁹ and R¹⁰ is independently hydrogen, alkyl, heteroalkyl, cycloalkyl, heterocyclylalkyl, aryl, heteroaryl, or hydrogen, wherein alkyl, heteroalkyl, cycloalkyl, heterocyclylalkyl, aryl, or heteroaryl is unsubstituted or substituted with one or more R^{A}, or R⁹ and R¹⁰ together with the atom to which they are attached form a heterocyclylalkyl ring or a heteroaryl ring that is unsubstituted or substituted with one or more R^{A};
each of R¹¹ and R¹² is independently hydrogen, alkyl, heteroalkyl, cycloalkyl, heterocyclylalkyl, aryl, heteroaryl, or hydrogen, wherein alkyl, heteroalkyl, cycloalkyl, heterocyclylalkyl, aryl, or heteroaryl is unsubstituted or substituted with one or more R^{A};
each R^{A} is independently alkyl, heteroalkyl, cycloalkyl, heterocyclylalkyl, aryl, heteroaryl, an amino acid side chain, —OR¹³, —N(R¹⁸)R¹⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹], —C(O)N(R¹⁸)R¹⁹, —OC(O)N(R¹⁸)R¹⁹, or —OP(O)OR²⁰(OR²¹), wherein alkyl, heteroalkyl, cycloalkyl, heterocyclylalkyl, aryl, or heteroaryl is unsubstituted or substituted with one or more alkyl, aryl, halogen, —S—R¹³, —OR¹³, —NR(R¹⁸)R¹⁹, —C(O)R¹⁴, —OC(O)R¹⁵, —OC(O)OR¹⁶, or —OC(O)N(R¹⁸)R¹⁹;
each of R¹³, R¹⁴, R¹⁵, R¹⁶, or R¹⁷ is independently hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, and heteroaryl is unsubstituted or substituted with one or more R^{B};
each of R¹⁸ and R¹⁹ is independently hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, or heteroaryl is unsubstituted or substituted with one or more R^{B}; or R¹⁸ and R¹⁹ together with the atom to which they are attached form a heterocyclylalkyl ring or heteroaryl ring, each of which is unsubstituted or substituted with one or more R^{B};
each R^{B} is independently halogen, amino, cyano, hydroxyl, alkyl, alkenyl, heteroalkyl, cycloalkyl, heterocyclylalkyl, aryl, heteroaryl, arylalkyl, —OC(O)R¹⁸, —C(O)R¹⁸, —C(O)OR¹⁹, NHC(O)OR¹⁸, or heteroarylalkyl, wherein cycloalkyl, heterocyclylalkyl, aryl, or heteroaryl is unsubstituted or substituted with one or more halogen, amino, cyano, hydroxyl, alkyl, acetyl, or benzoyl.

In certain embodiments, the compound of Formula (I) has the structure of Formula (Ia):

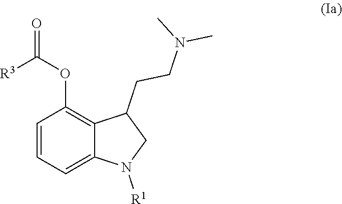

In certain embodiments, R³ is unsubstituted or substituted alkyl, heteroalkyl, cycloalkyl, or heterocyclylalkyl. In certain embodiments, R³ is unsubstituted or substituted alkyl. In certain embodiments, R³ is alkyl substituted with one or more substituent R^{A}, and wherein each R^{A} is independently selected from alkyl, heteroalkyl, cycloalkyl, heterocyclylalkyl, aryl, heteroaryl, an amino acid side chain, —OR¹³, —N(R¹⁸)R¹⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹], —C(O)N(R¹⁸)R¹⁹, —OC(O)N(R¹⁸)R¹⁹, or —OP(O)OR²⁰(OR²¹). In certain embodiments, R³ is unsubstituted alkyl. In certain embodiments, R³ is methyl, ethyl, n-propyl, isopropyl, n-butyl, tert-butyl, n-pentyl, 3-methyl-1-butyl, or —C₁₀H₂₁. In certain embodiments, R³ is alkyl substituted with —N(R¹⁸)R¹⁹, wherein each of R¹⁸ and R¹⁹ is independently hydrogen or methyl.

In certain embodiments, R³ is alkyl substituted with —C(O)OR¹³. In certain embodiments, R¹³ is hydrogen or alkyl. In certain embodiments, R¹³ is hydrogen, methyl, ethyl, or tert-butyl.

In certain embodiments, R³ is alkyl substituted with —N(R¹³)C(O)R¹⁴, wherein each of R¹³ and R¹⁴ is independently hydrogen or methyl.

In certain embodiments, R³ is alkyl substituted with —N(R¹³)C(O)R¹⁴, wherein each of R¹³ is hydrogen or methyl, and R¹⁴ is hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, and heteroaryl is unsubstituted or further substituted with one or more halogen, amino, cyano, hydroxyl, alkyl, acetyl, benzoyl, phenyl, or NH-Boc.

In certain embodiments, R³ is alkyl substituted with —N(R¹³)C(O)OR¹⁴, wherein each of R¹³ and R¹⁴ is independently hydrogen, methyl, ethyl, isopropyl, or tert-butyl.

In certain embodiments, R³ is heterocyclylalkyl.

In certain embodiments, R³ is

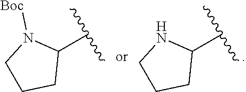 [SMILES: *C1CCCN1C(=O)OC(C)(C)C ; *C1CCCN1]

In certain embodiments, R³ is

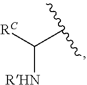

wherein R^{C} is a natural amino acid side chain, and R′ is hydrogen or -Boc.

In certain embodiments, R³ is

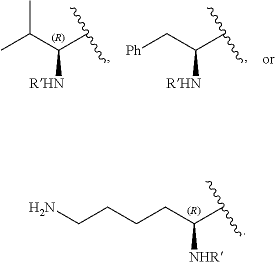

In certain embodiments, R³ is selected from aziridinyl, oxetanyl, azetidinyl, tetrahydrofuranyl, pyrrolidinyl, piperidinyl, piperazinyl, morpholinyl, thiomorpholinyl, thiomorpholinyl dioxide, diazinanyl,

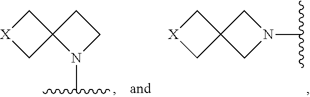

wherein X is —CH₂—, —O—, —S—, —SO₂, —NH—, or —NMe.

In certain embodiments, R³ is

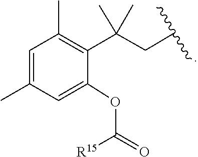

In certain embodiments, R³ is alkyl substituted with —OC(O)R¹⁵, wherein R¹⁵ is hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, and heteroaryl is unsubstituted or further substituted with one or more halogen, amino, cyano, hydroxyl, alkyl, acetyl, or benzoyl.

In certain embodiments, R³ is heteroalkyl.

In certain embodiments, is unsubstituted or substituted aryl (e.g., phenyl).

In certain embodiments, is substituted phenyl.

In certain embodiments, R³ is phenyl substituted with —OC(O)R¹⁸, wherein R¹⁸ is hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl.

In certain embodiments, the compound of Formula (I) has the structure of Formula (Ib):

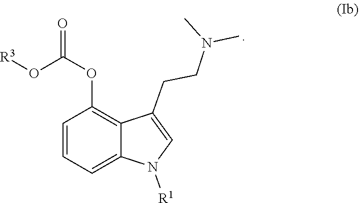

In certain embodiments, R³ is unsubstituted or substituted alkyl, heteroalkyl, cycloalkyl, or heterocyclylalkyl.

In certain embodiments, R³ is unsubstituted or substituted alkyl.

In certain embodiments, R³ is alkyl substituted with one or more substituent R^{A}, and wherein each R^{A} is independently selected from alkyl, heteroalkyl, cycloalkyl, heterocyclylalkyl, aryl, heteroaryl, an amino acid side chain, —OR¹³, —N(R¹⁸)R¹⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹], —C(O)N(R¹⁸)R¹⁹, —OC(O)N(R¹⁸)R¹⁹, or —OP(O)OR²⁰(OR²¹).

In certain embodiments, R³ is alkyl substituted with heterocyclylalkyl.

In certain embodiments, wherein R³ is alkyl substituted with aziridinyl, oxetanyl, azetidinyl, tetrahydrofuranyl, pyrrolidinyl, piperidinyl, piperazinyl, morpholinyl, thiomorpholinyl, thiomorpholinyl dioxide, diazinanyl,

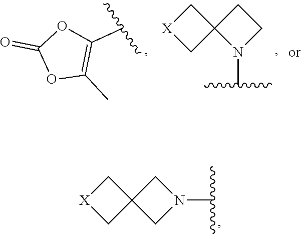

wherein X is —CH₂—, —O—, —S—, —SO₂, —NH—, or —NMe.

In certain embodiments, R³ is unsubstituted alkyl.

In certain embodiments, R³ is methyl, ethyl, n-propyl, isopropyl, n-butyl, tert-butyl, n-pentyl, 3-methyl-1-butyl, or —C₁₀H₂₁.

In certain embodiments, R³ is heteroalkyl.

In certain embodiments, R³ is heterocyclylalkyl.

In certain embodiments, R³ is selected from aziridinyl, oxetanyl, azetidinyl, tetrahydrofuranyl, pyrrolidinyl, piperidinyl, piperazinyl, morpholinyl, thiomorpholinyl, thiomorpholinyl dioxide, diazinanyl,

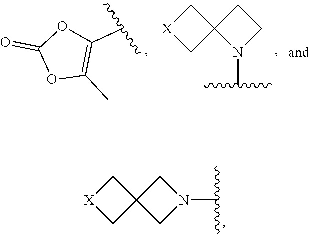

wherein X is —CH₂—, —O—, —SO₂, —NH—, or —NMe.

In certain embodiments, R³ is alkyl substituted with one or more —OC(O)R¹⁵.

In certain embodiments, R³ is isopropyl substituted with two —OC(O)R¹⁵ wherein each R¹⁵ is alkyl.

In certain embodiments, R³ is alkyl substituted with —N(R¹³)C(O)OR¹⁴, wherein each of R¹³ and R¹⁴ is independently hydrogen, methyl, or ethyl.

In certain embodiments, R³ is alkyl substituted with —OC(O)R¹⁵, wherein R¹⁵ is hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, and heteroaryl is unsubstituted or further substituted with one or more halogen, amino, cyano, hydroxyl, alkyl, acetyl, or benzoyl.

In certain embodiments, R³ is oxetanyl.

In certain embodiments, the compound of Formula (I) has the structure of Formula (Ic):

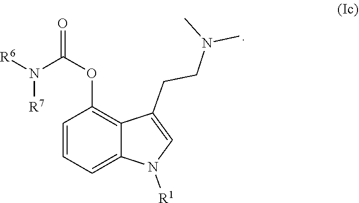

In certain embodiments, each of R⁶ and R⁷ is independently hydrogen, alkyl, heteroalkyl, cycloalkyl, heterocyclylalkyl, aryl, or heteroaryl, wherein alkyl, heteroalkyl, cycloalkyl, heterocyclylalkyl, aryl, or heteroaryl is unsubstituted or substituted with one or more R^{A}.

In certain embodiments, R⁶ and R⁷ together with the atom to which they are attached form a heterocyclylalkyl ring or a heteroaryl ring that is unsubstituted or substituted with one or more R^{A}.

In certain embodiments, R⁶ and R⁷ together with the atom to which they are attached form aziridinyl, oxetanyl, azetidinyl, tetrahydrofuranyl, pyrrolidinyl, piperidinyl, piperazinyl, morpholinyl, thiomorpholinyl, thiomorpholinyl dioxide, diazinanyl,

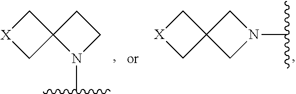

wherein —CH₂—, —O—, —S—, —SO₂, —NH—, or —NMe.

In certain embodiments, R⁶ and R⁷ together with the atom to which they are attached form

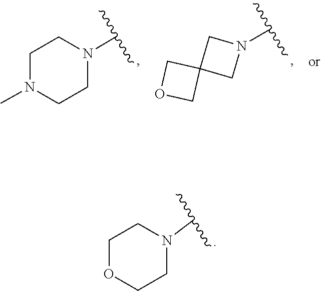

In certain embodiments, R⁶ is methyl.

In certain embodiments, R⁷ is alkyl substituted with —C(O)OR¹³, wherein R¹³ is hydrogen or alkyl.

In certain embodiments, R⁷ is alkyl substituted with —C(O)OR¹³, wherein R¹³ is hydrogen, methyl, ethyl, or tert-butyl.

In certain embodiments, the compound of Formula (I) has the structure of Formula (Id):

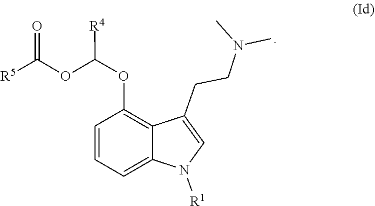

In certain embodiments, R⁴ is hydrogen, unsubstituted or substituted alkyl, heteroalkyl, cycloalkyl, or heterocyclylalkyl.

In certain embodiments, R⁴ is hydrogen or unsubstituted or substituted alkyl.

In certain embodiments, R⁴ is hydrogen, methyl, ethyl, n-propyl, isopropyl, n-butyl, tert-butyl, n-pentyl, 3-methyl-1-butyl, or —C₁₀H₂₁.

In certain embodiments, R⁴ is hydrogen.

In certain embodiments, R⁵ is unsubstituted or substituted alkyl, heteroalkyl, cycloalkyl, or heterocyclylalkyl.

In certain embodiments, R⁵ is unsubstituted or substituted alkyl.

In certain embodiments, R⁵ is alkyl substituted with one or more substituent R^{A}, and wherein each R^{A} is independently selected from alkyl, heteroalkyl, cycloalkyl, heterocyclylalkyl, aryl, heteroaryl, an amino acid side chain, —OR¹³, —N(R¹⁸)R¹⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹], —C(O)N(R¹⁸)R¹⁹, —OC(O)N(R¹⁸)R¹⁹, or —OP(O)OR²⁰(OR²¹).

In certain embodiments, R⁵ is unsubstituted alkyl.

In certain embodiments, R⁵ is methyl, ethyl, n-propyl, isopropyl, n-butyl, tert-butyl, n-pentyl, 3-methyl-1-butyl, or —C₁₀H₂₁.

In certain embodiments, R⁵ is alkyl substituted with C(O)OR¹³.

In certain embodiments, R¹³ is hydrogen or alkyl.

In certain embodiments, R¹³ is hydrogen, methyl, ethyl, or tert-butyl.

In certain embodiments, R⁵ is alkyl substituted with —N(R¹⁸)R¹⁹, wherein each of R¹⁸ and R¹⁹ is independently hydrogen or methyl.

In certain embodiments, R⁵ is alkyl substituted with —N(R¹³)C(O)R¹⁴, wherein each of R¹³ and R¹⁴ is independently hydrogen or methyl.

In certain embodiments, R⁵ is alkyl substituted with —N(R¹³)C(O)R¹⁴, wherein each of R¹³ is hydrogen or methyl, and R¹⁴ is hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, and heteroaryl is unsubstituted or further substituted with one or more halogen, amino, cyano, hydroxyl, alkyl, acetyl, or benzoyl.

In certain embodiments, R⁵ is alkyl substituted with —N(R¹³)C(O)OR¹⁴, wherein each of R¹³ and R¹⁴ is independently hydrogen, methyl, or ethyl.

The compound or pharmaceutically acceptable salt, solvate, or isotopolog of any one of claims 36 to 39, wherein R⁵ is alkyl substituted with —N(R¹³)C(O)OR¹⁴, wherein R¹³ is hydrogen or methyl, and R¹⁴ is hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, and heteroaryl is unsubstituted or further substituted with one or more halogen, amino, cyano, hydroxyl, alkyl, acetyl, or benzoyl.

In certain embodiments, R⁵ is heterocyclylalkyl.

In certain embodiments, R⁵ is selected from aziridinyl, oxetanyl, azetidinyl, tetrahydrofuranyl, pyrrolidinyl, piperidinyl, piperazinyl, morpholinyl, thiomorpholinyl, thiomorpholinyl dioxide, diazinanyl,

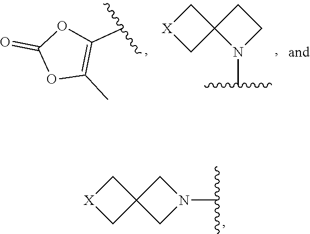

wherein —CH₂—, —O—, —S—, —SO₂, —NH—, or —NMe.

In certain embodiments, R⁵ is optionally substituted piperidinyl.

In certain embodiments, R⁵ is

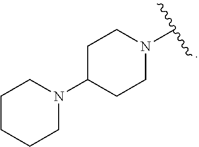 [SMILES: *N1CCC(N2CCCCC2)CC1]

In certain embodiments, R³ is alkyl substituted with —OC(O)R⁵, wherein R¹⁵ is hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, and heteroaryl is unsubstituted or further substituted with one or more halogen, amino, cyano, hydroxyl, alkyl, acetyl, or benzoyl.

In certain embodiments, R⁵ is heteroalkyl.

In certain embodiments, R⁵ is unsubstituted or substituted aryl (e.g., phenyl).

In certain embodiments, R⁵ is t-butyl, —CH(NH₂)CH(CH₃)₂, —CH₂N(CH₃)₂, —CH₂CH₂OCH₃, —CH₂CH₂NH(CH₃)₂, —CH₂CH₂C(CH₃)₂OC(O)CH₃, —CH₂CH₂C(CH₃)₂NHC(O)CH₃, or —CH₂CH₂C(CH₃)₂NHC(O)OCH₂CH₃.

In certain embodiments, the compound of Formula (I) has the structure of Formula (Ie):

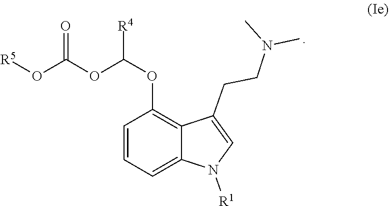

In certain embodiments, R⁴ is hydrogen, unsubstituted or substituted alkyl, heteroalkyl, cycloalkyl, or heterocyclylalkyl.

In certain embodiments, R⁴ is unsubstituted or substituted alkyl.

In certain embodiments, R⁴ is methyl, ethyl, n-propyl, isopropyl, n-butyl, tert-butyl, n-pentyl, 3-methyl-1-butyl, or —C₁₀H₂₁.

In certain embodiments, R⁴ is hydrogen.

In certain embodiments, R⁵ is unsubstituted or substituted alkyl, heteroalkyl, cycloalkyl, or heterocyclylalkyl.

In certain embodiments, R⁵ is unsubstituted or substituted alkyl.

In certain embodiments, R⁵ is alkyl substituted with one or more substituent R^{A}, and wherein each R^{A} is independently selected from alkyl, heteroalkyl, cycloalkyl, heterocyclylalkyl, aryl, heteroaryl, an amino acid side chain, —OR¹³, —N(R¹⁸)R¹⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹], —C(O)N(R¹⁸)R¹⁹, —OC(O)N(R¹⁸)R¹⁹, or —OP(O)OR²⁰(OR²¹).

In certain embodiments, R⁵ is unsubstituted alkyl.

In certain embodiments, R⁵ is methyl, ethyl, n-propyl, isopropyl, n-butyl, tert-butyl, n-pentyl, 3-methyl-1-butyl, or —C₁₀H₂₁.

In certain embodiments, R⁵ is alkyl substituted with —N(R¹⁸)R¹⁹, wherein each of R¹⁸ and R¹⁹ is independently hydrogen or methyl.

In certain embodiments, R⁵ is alkyl substituted with —N(R¹³)C(O)R¹⁴, wherein each of R¹³ and R¹⁴ is independently hydrogen or methyl.

In certain embodiments, R⁵ is alkyl substituted with —N(R¹³)C(O)R¹⁴, wherein each of R¹³ is hydrogen or methyl, and R¹⁴ is hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, and heteroaryl is unsubstituted or further substituted with one or more halogen, amino, cyano, hydroxyl, alkyl, acetyl, or benzoyl.

In certain embodiments, R⁵ is alkyl substituted with —N(R¹³)C(O)OR¹⁴, wherein each of R¹³ and R¹⁴ is independently hydrogen, methyl, or ethyl.

In certain embodiments, R⁵ is heterocyclylalkyl.

In certain embodiments, R⁵ is selected from aziridinyl, oxetanyl, azetidinyl, tetrahydrofuranyl, pyrrolidinyl, piperidinyl, piperazinyl, morpholinyl, thiomorpholinyl, thiomorpholinyl dioxide, diazinanyl,

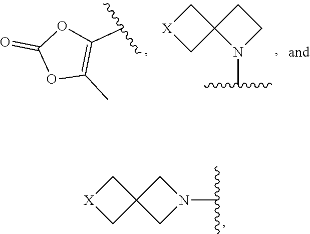

and wherein X is —CH₂—, —O—, —S—, —SO₂, —NH—, or —NMe.

In certain embodiments, R⁵ is morpholinyl, isopropyl, or ethyl.

In certain embodiments, R³ is alkyl substituted with —OC(O)R¹⁵, wherein R¹⁵ is hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, and heteroaryl is unsubstituted or further substituted with one or more halogen, amino, cyano, hydroxyl, alkyl, acetyl, or benzoyl.

In certain embodiments, R⁵ is heteroalkyl.

In certain embodiments, R⁵ is unsubstituted or substituted aryl (e.g., phenyl).

In certain embodiments, R⁵ is

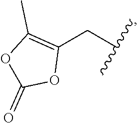 [SMILES: *Cc1oc(=O)oc1C]

—CH₂CH₂NHCH₃, —CH₂CH₂NHCOCH₃, or —CH₂CH₂NHCO(O)CH₂CH₃.

In certain embodiments, the compound of Formula (I) has the structure of Formula (If):

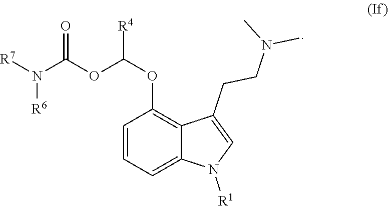

In certain embodiments, R⁴ is hydrogen, unsubstituted or substituted alkyl, heteroalkyl, cycloalkyl, or heterocyclylalkyl.

In certain embodiments, R⁴ is unsubstituted or substituted alkyl.

In certain embodiments, R⁴ is methyl, ethyl, n-propyl, isopropyl, n-butyl, tert-butyl, n-pentyl, 3-methyl-1-butyl, or —C₁₀H₂₁.

In certain embodiments, R⁴ is hydrogen.

In certain embodiments, each of R⁶ and R⁷ is independently alkyl, heteroalkyl, cycloalkyl, heterocyclylalkyl, aryl, or heteroaryl, wherein alkyl, heteroalkyl, cycloalkyl, heterocyclylalkyl, aryl, or heteroaryl is unsubstituted or substituted with one or more R^{A}.

In certain embodiments, R⁶ is hydrogen or methyl, and R⁷ is hydrogen alkyl, heteroalkyl, cycloalkyl, heterocyclylalkyl, aryl, or heteroaryl, wherein alkyl, heteroalkyl, cycloalkyl, heterocyclylalkyl, aryl, or heteroaryl is unsubstituted or substituted with one or more R^{A}.

In certain embodiments, R⁶ and R⁷ together with the atom to which they are attached form a heterocyclylalkyl ring or a heteroaryl ring that is unsubstituted or substituted with one or more R^{A}.

In certain embodiments, R⁶ and R⁷ together with the atom to which they are attached form aziridinyl, oxetanyl, azetidinyl, tetrahydrofuranyl, pyrrolidinyl, piperidinyl, piperazinyl, morpholinyl, thiomorpholinyl, thiomorpholinyl dioxide, diazinanyl,

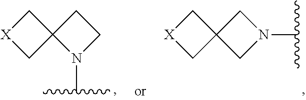

wherein X is —CH₂—, —O—, —S—, —SO₂, —NH—, or —NMe.

In certain embodiments, R⁶ and R⁷ together with the atom to which they are attached form optionally substituted piperidinyl.

In certain embodiments, R⁶ and R⁷ together with the atom to which they are attached form

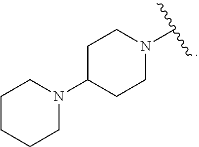 [SMILES: *N1CCC(N2CCCCC2)CC1]

In certain embodiments, the compound of Formula (I) has the structure of Formula (Ig):

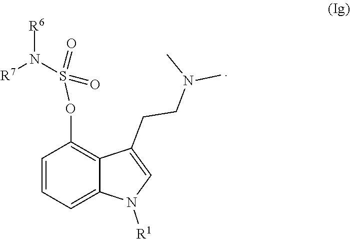

In certain embodiments, each of R⁶ and R⁷ is independently hydrogen, alkyl, heteroalkyl, cycloalkyl, heterocyclylalkyl, aryl, or heteroaryl, wherein alkyl, heteroalkyl, cycloalkyl, heterocyclylalkyl, aryl, or heteroaryl is unsubstituted or substituted with one or more R^{A}.

In certain embodiments, each of R⁶ and R⁷ are each independently hydrogen or alkyl.

In certain embodiments, each of R⁶ and R⁷ are each independently hydrogen or methyl.

In certain embodiments, R⁶ and R⁷ are each hydrogen.

In certain embodiments, R⁶ and R⁷ together with the atom to which they are attached form a heterocyclylalkyl ring or a heteroaryl ring that is unsubstituted or substituted with one or more R^{A}.

In certain embodiments, R⁶ and R⁷ together with the atom to which they are attached form aziridinyl, oxetanyl, azetidinyl, tetrahydrofuranyl, pyrrolidinyl, piperidinyl, piperazinyl, morpholinyl, thiomorpholinyl, thiomorpholinyl dioxide, diazinanyl,

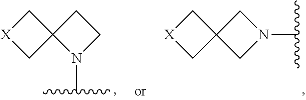

wherein X is —CH₂—, —O—, —S—, —SO₂, —NH—, or —NMe.

In certain embodiments, the compound of Formula (I) has the structure of Formula (Ih):

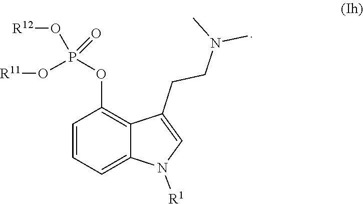

In certain embodiments, each of R¹¹ and R¹² is hydrogen, independently unsubstituted or substituted alkyl, heteroalkyl, cycloalkyl, or heterocyclylalkyl.

In certain embodiments, each of R¹¹ and R¹² is independently hydrogen, or unsubstituted or substituted alkyl.

In certain embodiments, each of R¹¹ and R¹² is independently alkyl substituted with one or more substituent R^{A}, and wherein each R^{A} is independently selected from alkyl, heteroalkyl, cycloalkyl, heterocyclylalkyl, aryl, heteroaryl, an amino acid side chain, —OR¹³, —N(R¹⁹)R¹⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹], —C(O)N(R¹⁸)R¹⁹, —OC(O)N(R¹⁸)R¹⁹, or —OP(O)OR²⁰(OR²¹).

In certain embodiments, each of R¹¹ and R¹² is independently unsubstituted alkyl.

In certain embodiments, each of R¹¹ and R¹² is independently methyl, ethyl, n-propyl, isopropyl, n-butyl, tert-butyl, n-pentyl, 3-methyl-1-butyl, or —C₁₀H₂₁.

In certain embodiments, each of R¹¹ and R¹² is independently alkyl substituted with —OC(O)R^{5A}, wherein R^{5A} is hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, and heteroaryl is unsubstituted or further substituted with one or more halogen, amino, cyano, hydroxyl, alkyl, acetyl, or benzoyl.

In certain embodiments, each of R¹¹ and R¹² is independently alkyl substituted with —OC(O)OR¹⁶, wherein R¹⁶ is hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, and heteroaryl is unsubstituted or further substituted with one or more halogen, amino, cyano, hydroxyl, alkyl, acetyl, or benzoyl.

In certain embodiments, R¹⁶ is hydrogen or alkyl.

In certain embodiments, R¹⁶ is hydrogen, methyl, ethyl, isopropyl or tert-butyl.

In certain embodiments, each of R¹¹ and R¹² is independently heteroalkyl.

In certain embodiments, each of R¹¹ and R¹² is independently unsubstituted or substituted aryl (e.g., phenyl).

In certain embodiments, the compound of Formula (I) has the structure of Formula (Ih′):

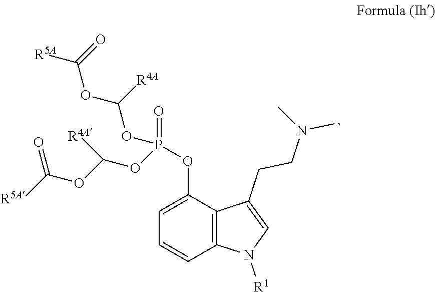

wherein
R^{4A} and R^{4A}′ are each independently hydrogen or alkyl, and R^{5A} and R^{5A}′ are each independently hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, and heteroaryl is unsubstituted or further substituted with one or more halogen, amino, cyano, hydroxyl, alkyl, acetyl, or benzoyl.

In certain embodiments, R^{4A} and R^{4A}′ are each hydrogen.

In certain embodiments, R^{5A} and R^{5A}′ are each methyl, ethyl, n-propyl, isopropyl, n-butyl, tert-butyl, n-pentyl, 3-methyl-1-butyl, or —C₁₀H₂₁.

In certain embodiments, R^{5A} and R^{5A}′ are each isopropyl or tert-butyl.

In certain embodiments, the compound has the structure of Formula (Ib′):

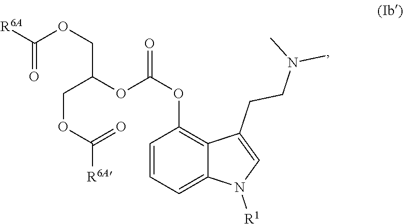

wherein R^{6A} and R^{6A}′ are each independently hydrogen or alkyl.

In certain embodiments, R^{6A} and R^{6A}′ are each independently —CH₃, —C₂H₅, C₃H₇, C₄H₉, C₅H₁₁, C₆H₁₃, C₇H₁₅, C₈H₁₇, C₉H₁₉, C₁₀H₂₁, C₁₁H₂₃, C₁₂H₂₅, C₁₃H₂₇, C₁₄H₂₉, C₁₅H₃₁, C₁₆H₃₃, or C₁₇H₃₅.

In certain embodiments, R^{6A} and R^{6A}′ are the same.

In certain embodiments, the compound has the structure of Formula (Ib″):

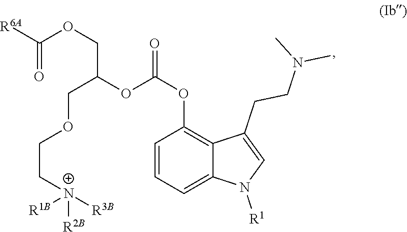

wherein each of R^{6A}, R^{1B}, R^{2B}, and R^{3B} are independently hydrogen or alkyl.

In certain embodiments, R^{6A} is —CH₃, —C₂H₅, C₃H₇, C₄H₉, C₅H₁₁, C₆H₁₃, C₇H₁₅, C₈H₁₇, C₉H₁₉, C₁₀H₂₁, CH₂₃, C₁₂H₂₅, C₁₃H₂₇, C₁₄H₂₉, C₁₅H₃₁, C₁₆H₃₃, or C₁₇H₃₅.

In certain embodiments, R^{1B}, R^{2B}, and R^{3B} are each independently alkyl.

In certain embodiments, each of R^{1B}, R^{2B}, and R^{3B} are independently methyl, ethyl, n-propyl, isopropyl, n-butyl, tert-butyl, n-pentyl, 3-methyl-1-butyl, or —C₁₀H₂₁.

In certain embodiments, R^{1B}, R^{2B}, and R^{3B} are each methyl.

In certain embodiments, R¹ is hydrogen.

In certain embodiments, the compound of Formula (I) has the structure of Formula (Ii):

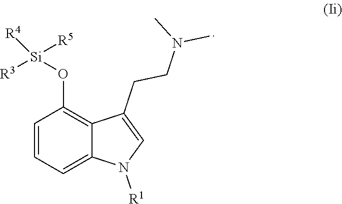

In certain embodiments, each of R³, R⁴ and R⁵ is independently hydrogen, unsubstituted or substituted alkyl, heteroalkyl, cycloalkyl, or heterocyclylalkyl.

In certain embodiments, each of R³, R⁴ and R⁵ is unsubstituted or substituted alkyl.

In certain embodiments, each of R³, R⁴ and R⁵ is independently alkyl substituted with one or more substituent R^{A}, and wherein each R^{A} is independently selected from alkyl, heteroalkyl, cycloalkyl, heterocyclylalkyl, aryl, heteroaryl, an amino acid side chain, —OR¹³, —N(R¹⁸)R¹⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹], —C(O)N(R¹⁸)R¹⁹, —OC(O)N(R¹⁸)R¹⁹, or —OP(O)OR²⁰(OR²¹).

In certain embodiments, each of R³, R⁴ and R⁵ is independently unsubstituted alkyl.

In certain embodiments, each of R³, R⁴ and R⁵ is independently methyl, ethyl, n-propyl, isopropyl, n-butyl, tert-butyl, n-pentyl, 3-methyl-1-butyl, or —C₁₀H₂₁.

In certain embodiments, R³, R⁴ and R⁵ are the same unsubstituted alkyl.

In certain embodiments, R³ and R⁴ are methyl, ethyl or isopropyl.

In certain embodiments, R⁵ is ethyl, isopropyl, or tert-butyl.

In certain embodiments, (i) R³ and R⁴ are methyl, R⁵ is ethyl; (ii) R³, R⁴ and R⁵ are isopropyl; or (iii) R³, R⁴ and R⁵ are ethyl.

In certain embodiments, each of R³, R⁴ and R⁵ is independently heteroalkyl.

In certain embodiments, each of R³, R⁴ and R⁵ is independently unsubstituted or substituted aryl (e.g., phenyl).

In certain embodiments, the compound of Formula (I) has the structure of Formula (Ij):

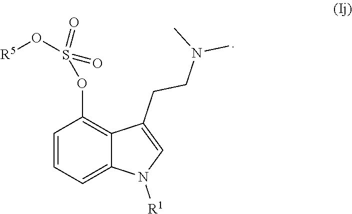

In certain embodiments, R⁵ is unsubstituted or substituted alkyl, heteroalkyl, cycloalkyl, or heterocyclylalkyl.

In certain embodiments, R⁵ is unsubstituted or substituted alkyl.

In certain embodiments, R⁵ is alkyl substituted with one or more substituent R^{A}, and wherein each R^{A} is independently selected from alkyl, heteroalkyl, cycloalkyl, heterocyclylalkyl, aryl, heteroaryl, an amino acid side chain, —OR¹³, —N(R¹⁸)R¹⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹], —C(O)N(R¹⁸)R¹⁹, —OC(O)N(R¹⁸)R¹⁹, or —OP(O)OR²⁰(OR²¹).

In certain embodiments, R⁵ is unsubstituted alkyl.

In certain embodiments, R⁵ is methyl, ethyl, n-propyl, isopropyl, n-butyl, tert-butyl, n-pentyl, 3-methyl-1-butyl, or —C₁₁H₂₁.

In certain embodiments, R⁵ is alkyl substituted with —C(O)OR¹³, wherein R¹³ is hydrogen or alkyl.

In certain embodiments, R⁵ is hydrogen, methyl, ethyl, isopropyl, or tert-butyl.

In certain embodiments, R⁵ is alkyl substituted with —N(R¹⁸)R¹⁹, wherein each of R¹⁸ and R¹⁹ is independently hydrogen or methyl.

In certain embodiments, R⁵ is alkyl substituted with —N(R¹³)C(O)R¹⁴, wherein each of R¹³ and R¹⁴ is independently hydrogen or methyl.

In certain embodiments, R⁵ is alkyl substituted with —N(R¹³)C(O)R¹⁴, wherein each of R¹³ is hydrogen or methyl, and R¹⁴ is hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, and heteroaryl is unsubstituted or further substituted with one or more halogen, amino, cyano, hydroxyl, alkyl, acetyl, or benzoyl.

In certain embodiments, R⁵ is alkyl substituted with —N(R¹³)C(O)OR¹⁴, wherein each of R¹³ and R¹⁴ is independently hydrogen, methyl, or ethyl.

In certain embodiments, R⁵ is heterocyclylalkyl.

In certain embodiments, R⁵ is selected from aziridinyl, oxetanyl, azetidinyl, tetrahydrofuranyl, pyrrolidinyl, piperidinyl, piperazinyl, morpholinyl, thiomorpholinyl, thiomorpholinyl dioxide, diazinanyl,

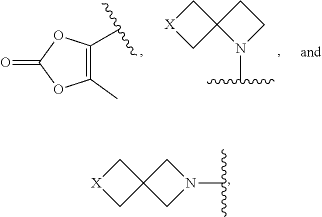

wherein X is —CH₂—, —O—, —S—, —SO₂, —NH—, or —NMe.

In certain embodiments, the compound of Formula (I) is selected from the group consisting of

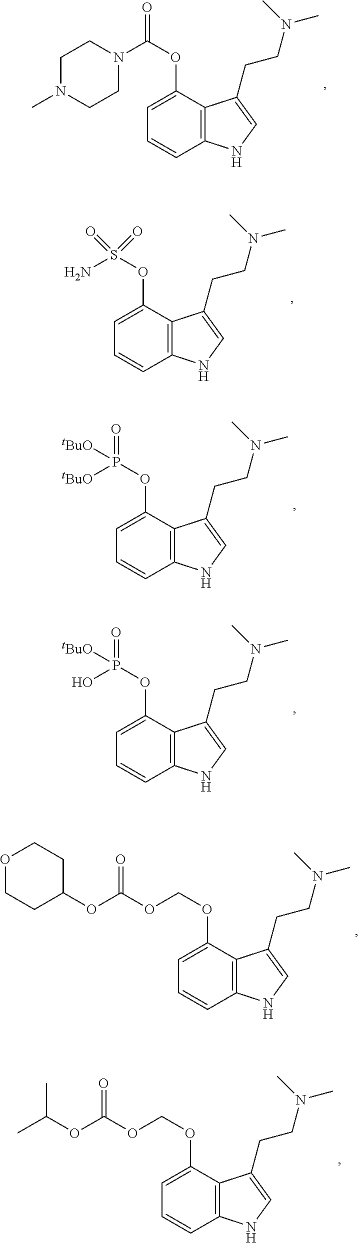

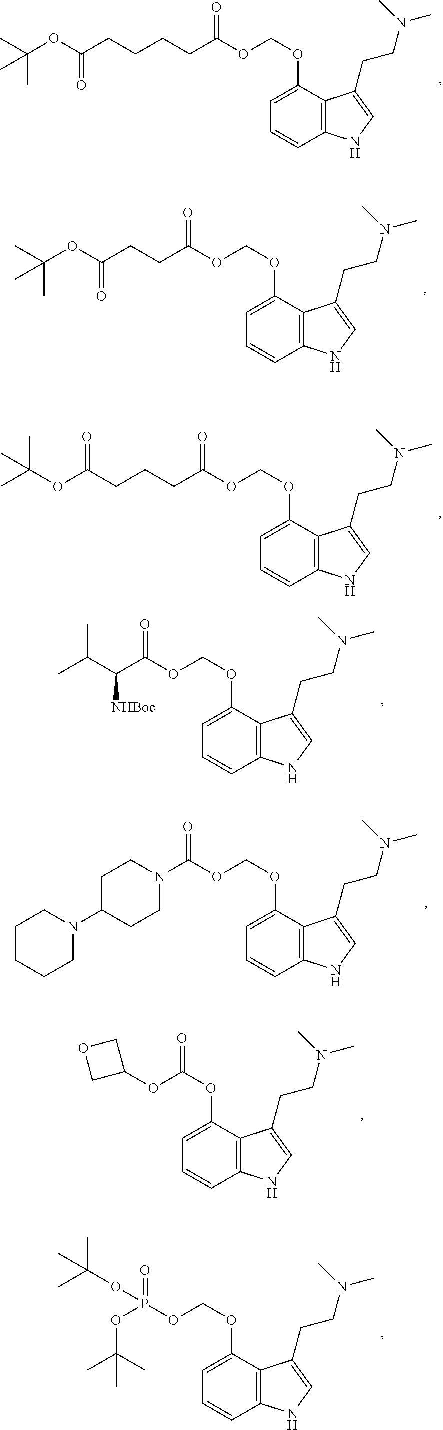

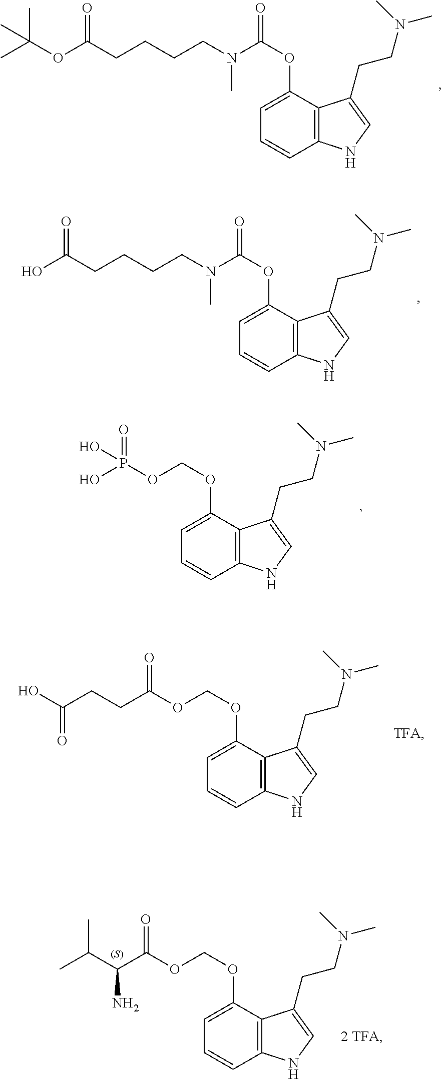

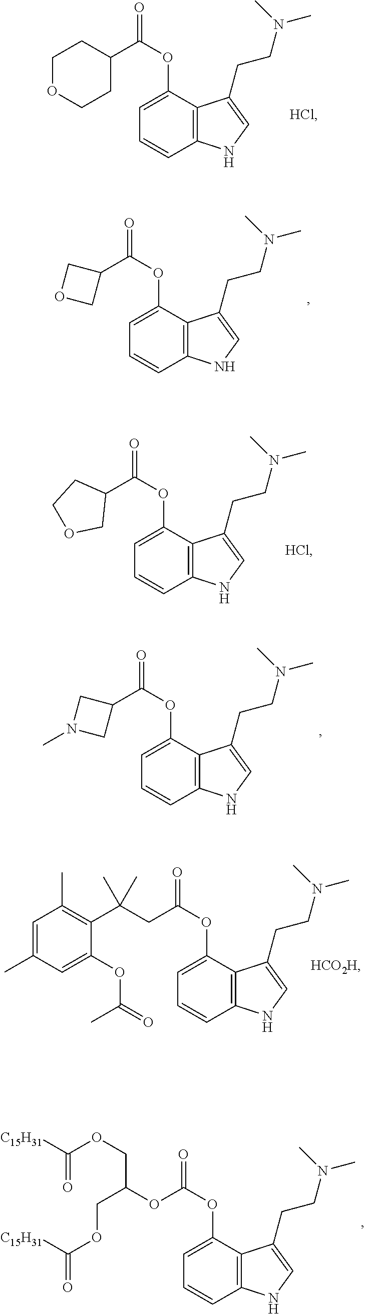

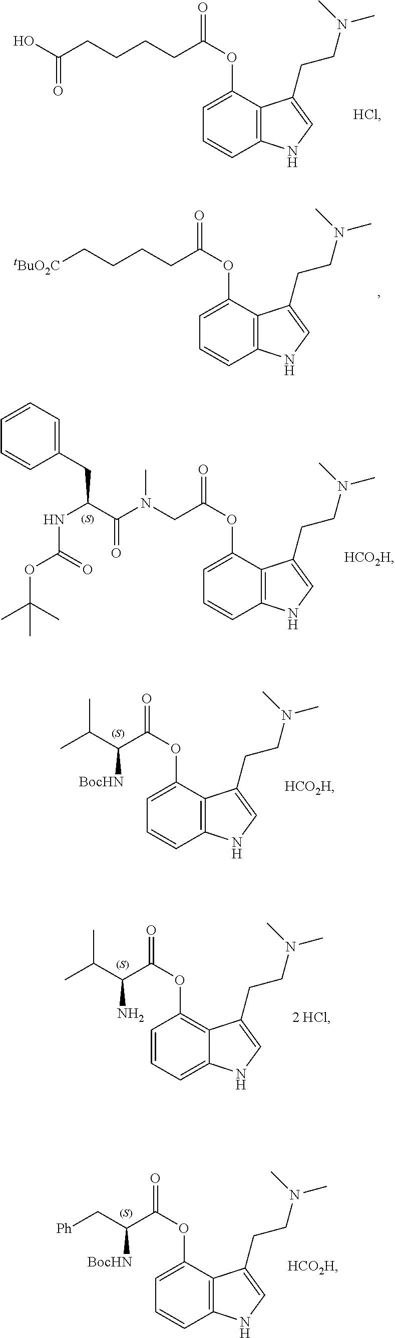

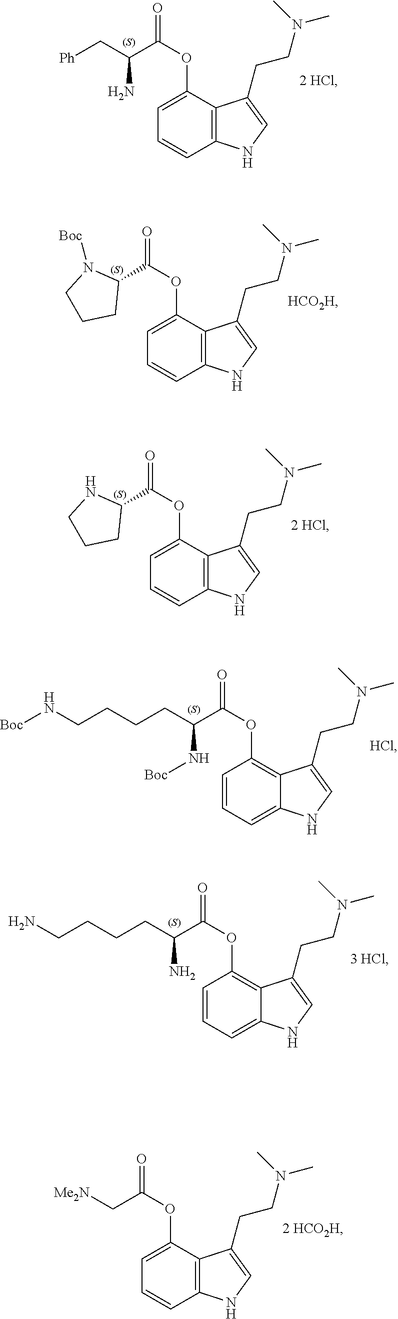

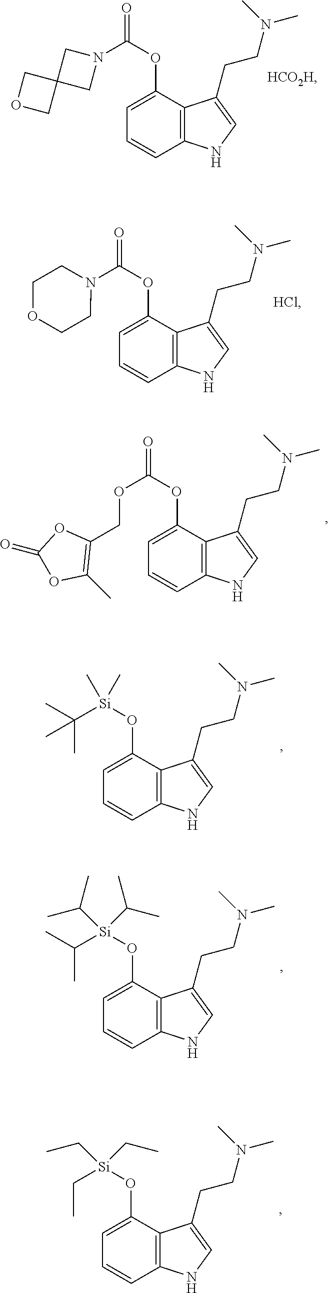

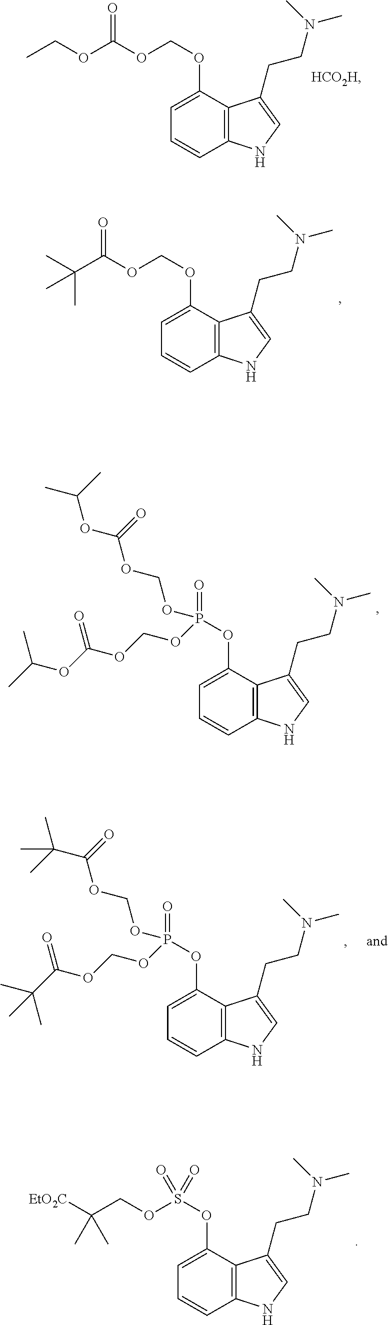

In another aspect, provided herein are compounds of Formula (II), or a pharmaceutically acceptable salt, solvate, or isotopolog thereof:

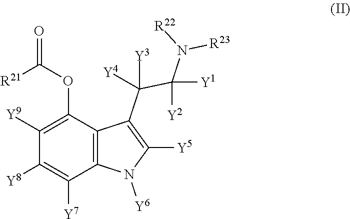

wherein:
R²¹ is CH₃, CH₂D, CHD₂, or CD₃;
each of R²² and R²³ is independently hydrogen or alkyl, wherein one or more of the hydrogens in the alkyl is optionally substituted with deuterium;
each of Y¹, Y², Y³, Y⁴, Y⁵, Y⁶, Y⁷, Y⁸, and Y⁹ is independently hydrogen or deuterium; and
wherein when R²¹ is CH₃, and R²² and R²³ do not comprise deuterium, at least one of Y¹, Y², Y³, Y⁴, Y⁵, Y⁶, Y⁷, Y⁸, and Y⁹ is deuterium.

In certain embodiments, R²¹ is —CH₃.

In certain embodiments, R²¹ is —CD₃.

In certain embodiments, R²² and R²³ are each independently —CH₃, —CH₂D, —CHD₂, or —CD₃.

In certain embodiments, at least one of R²² and R²³ comprises deuterium.

In certain embodiments, one of R²² and R²³ is —CD₃.

In certain embodiments, both R²² and R²³ are —CD₃.

In certain embodiments, Y¹ is D.

In certain embodiments, Y³ is D.

In certain embodiments, Y¹ and Y² are each D.

In certain embodiments, Y³ and Y⁴ are each D.

In certain embodiments, Y¹, Y², Y³, and Y⁴ are each D.

In certain embodiments, Y⁶ is H.

In certain embodiments, the compound of Formula (II) is selected from the group consisting of:

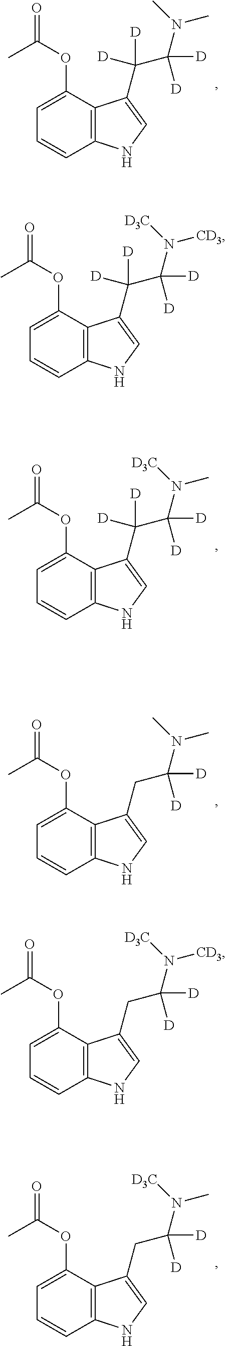

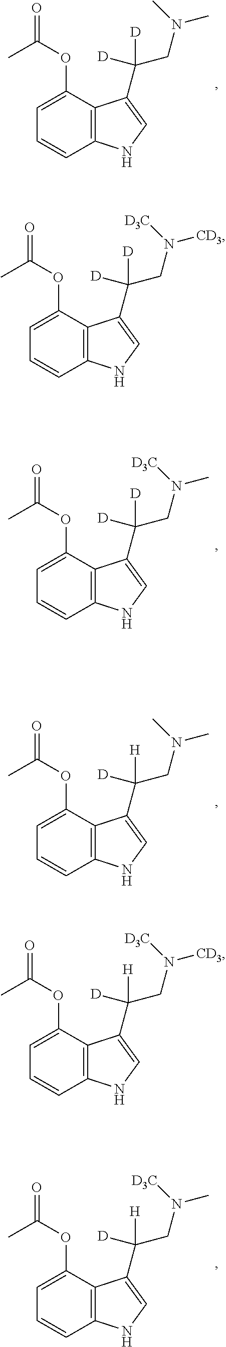

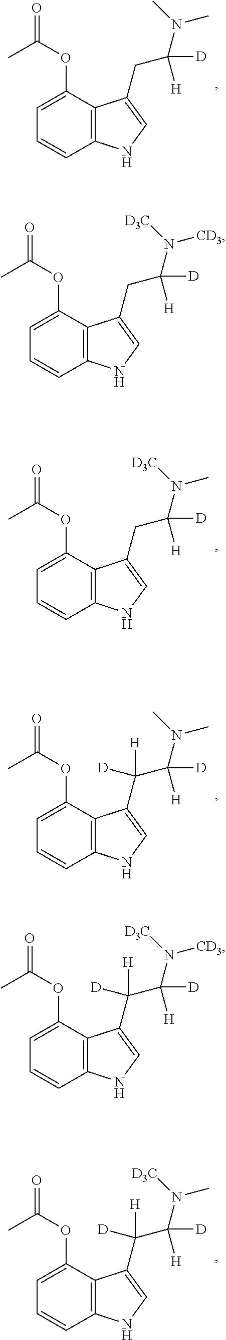

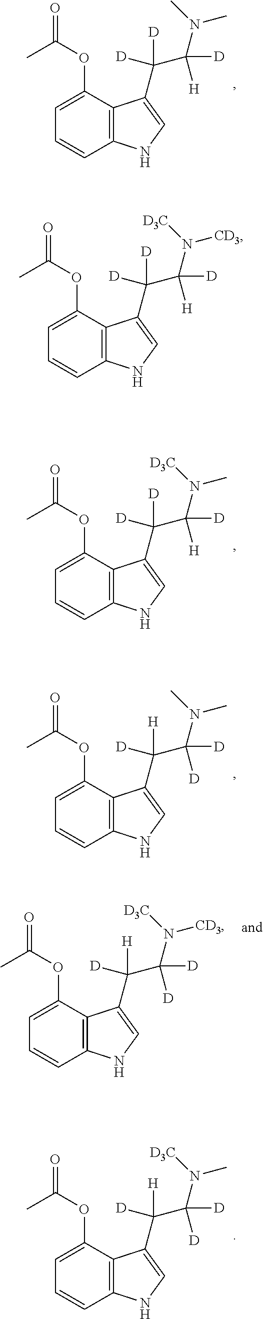

In certain embodiments, the compound of Formula (I) is a compound described in Table 1.

In yet another aspect, provided herein are pharmaceutically compositions comprising a compound described herein, or a pharmaceutically acceptable salt thereof, and a pharmaceutically acceptable excipient or carrier.

In yet another aspect provided herein are methods of treating or preventing a disease, disorder, or condition in which an increased level of psilocin is beneficial, comprising administering to a subject in need thereof an effective amount of the compound or pharmaceutically acceptable salt, solvate, or isotopolog described herein or the pharmaceutical composition described herein.

In certain embodiments, the disease, disorder, or condition is selected from post-traumatic stress disorder, major depression, schizophrenia, Alzheimer's disease, frontotemporal dementia, Parkinson's disease, Parkinson's dementia, dementia, Lewy body dementia, multiple system atrophy, and substance abuse.

### BRIEF DESCRIPTION OF THE DRAWINGS

FIG. 1 shows the mean Concentration-Time Profiles of psilocin following IV dosing of psilocin (1 mg/kg) to male Sprague Dawley (SD) rats.

FIG. 2 shows the mean Concentration-Time Profiles of psilocin following Oral (PO) dosing of psilocin (2 mg/kg) to male Sprague Dawley (SD) rats.

FIG. 3 shows the mean Concentration-Time Profiles of metabolite psilocin following oral dosing of psilocybin (2 mg/kg) to male Sprague Dawley (SD) rats.

FIG. 4 shows the mean Concentration-Time Profiles of metabolite psilocin following oral dosing of the O-TBDMS ether prodrug of psilocin (2 mg/Kg) to male Sprague Dawley (SD) rats.

FIG. 5 shows the mean Concentration-Time Profiles of metabolite psilocin following oral dosing of the O-TIPS ether prodrug of psilocin (2 mg/Kg) to male Sprague Dawley (SD) rats.

FIG. 6 shows the mean Concentration-Time Profiles of metabolite psilocin following oral dosing of the O-adipate ester hydrochloride prodrug of psilocin (2 mg/Kg) to male Sprague Dawley (SD) rats.

FIG. 7 shows the mean Concentration-Time Profiles of metabolite psilocin following oral dosing of the tetrahydrofuran-3-ester hydrochloride prodrug of psilocin (2 mg/Kg) to male Sprague Dawley (SD) rats.

FIG. 8 shows the mean Concentration-Time Profiles of metabolite psilocin following oral dosing of the trimethyl lock formate prodrug of psilocin (2 mg/Kg) to male Sprague Dawley (SD) rats.

FIG. 9 shows the mean Concentration-Time Profiles of metabolite psilocin following oral dosing of the 2-oxa-6-azaspiro[3.3]heptane carboxalate formate prodrug of psilocin (2 mg/Kg) to male Sprague Dawley (SD) rats.

FIG. 10 shows the mean Concentration-Time Profiles of metabolite psilocin following oral dosing of the O-TES ether prodrug of psilocin (2 mg/Kg) to male Sprague Dawley (SD) rats.

FIG. 11 shows the mean Concentration-Time Profiles of metabolite psilocin following oral dosing of the lysine trihydrochloride prodrug of psilocin (2 mg/Kg) to male Sprague Dawley (SD) rats.

FIG. 12 shows the mean Concentration-Time Profiles of metabolite psilocin following oral dosing of the oxane hydrochloride prodrug of psilocin (2 mg/Kg) to male Sprague Dawley (SD) rats.

FIG. 13 shows the mean Concentration-Time Profiles of metabolite psilocin following oral dosing of the morpholine carbamate hydrochloride prodrug of psilocin (2 mg/Kg) to male Sprague Dawley (SD) rats.

FIG. 14 shows the mean Concentration-Time Profiles of metabolite psilocin following oral dosing of the O-methyl ethyl carbonate formate prodrug of psilocin (2 mg/Kg) to male Sprague Dawley (SD) rats.

FIG. 15 shows the mean Concentration-Time Profiles of metabolite psilocin following oral dosing of the di-tert-butyl phosphonate hydrochloride prodrug of psilocin (2 mg/Kg) to male Sprague Dawley (SD) rats.

FIG. 16 shows the mean Concentration-Time Profiles of metabolite psilocin following oral dosing of the Boc-valine formate prodrug of psilocin (2 mg/Kg) to male Sprague Dawley (SD) rats.

FIG. 17 shows the mean Concentration-Time Profiles of metabolite psilocin following oral dosing of the Boc-proline formate prodrug of psilocin (2 mg/Kg) to male Sprague Dawley (SD) rats.

FIG. 18 shows the mean Concentration-Time Profiles of metabolite psilocin following oral dosing of the phenylalanine dihydrochloride prodrug of psilocin (2 mg/Kg) to male Sprague Dawley (SD) rats.

FIG. 19 shows the mean Concentration-Time Profiles of metabolite psilocin following oral dosing of the Boc-phenylalanine formate prodrug of psilocin (2 mg/Kg) to male Sprague Dawley (SD) rats.

FIG. 20 shows the mean Concentration-Time Profiles of metabolite psilocin following oral dosing of the pivaloyloxymethyl (POM) prodrug of psilocin (2 mg/Kg) to male Sprague Dawley (SD) rats.

FIG. 21 shows the mean Concentration-Time Profiles of metabolite psilocin following oral dosing of the O-proline ester dihydrochloride prodrug of psilocin (2 mg/Kg) to male Sprague Dawley (SD) rats.

FIG. 22 shows the mean Concentration-Time Profiles of metabolite psilocin following oral dosing of the N-POM ether prodrug of psilocin (2 mg/Kg) to male Sprague Dawley (SD) rats.

FIG. 23 shows the mean Concentration-Time Profiles of metabolite psilocin following oral dosing of the N-POM ether O-pivaloyl prodrug of psilocin (2 mg/Kg) to male Sprague Dawley (SD) rats.

FIG. 24 shows the mean Concentration-Time Profiles of metabolite psilocin following oral dosing of the O-methyl glutarate ether t-butyl ester prodrug of psilocin (2 mg/Kg) to male Sprague Dawley (SD) rats.

FIG. 25 shows the mean Concentration-Time Profiles of metabolite psilocin following oral dosing of the O-methyl succinate ether t-butyl ester prodrug of psilocin (2 mg/Kg) to male Sprague Dawley (SD) rats.

FIG. 26 shows the mean Concentration-Time Profiles of metabolite psilocin following oral dosing of the O-methyl adipate ether t-butyl ester prodrug of psilocin (2 mg/Kg) to male Sprague Dawley (SD) rats.

FIG. 27 shows the mean Concentration-Time Profiles of metabolite psilocin following oral dosing of the valine dihydrochloride prodrug of psilocin (2 mg/Kg) to male Sprague Dawley (SD) rats.

FIG. 28 shows the mean Concentration-Time Profiles of metabolite psilocin following oral dosing of the N-Boc-L-phenylalanine-sarcosine ester formate prodrug of psilocin (2 mg/Kg) to male Sprague Dawley (SD) rats.

FIG. 29 shows the mean Concentration-Time Profiles of metabolite psilocin following oral dosing of the dimethylglycine ester diformate prodrug of psilocin (2 mg/Kg) to male Sprague Dawley (SD) rats.

### DETAILED DESCRIPTION

Described herein are compounds analogs including prodrugs and deuterated analogs of psilocin. The prodrug analogs of psilocin can be metabolically converted to psilocin or its derivatives upon administration to a subject. Compound disclosed herein can be useful for the treatment of a neurological disease, such as a psychiatric disorder, a substance abuse disorder, or a condition where increasing neuronal plasticity would be beneficial.

### Definitions

Compounds herein can include all stereoisomers, enantiomers, diastereomers, mixtures, racemates, atropisomers, and tautomers thereof.

Unless otherwise specified, any compound disclosed herein can be substituted. Non-limiting examples of optional substituents include hydroxyl groups, sulfhydryl groups, halogens, amino groups, nitro groups, nitroso groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocyclylalkyl groups, heteroaryl groups, cycloalkyl groups, acyl groups, acyloxy groups, carbamate groups, amide groups, ureido groups, epoxy groups, and ester groups.

Non-limiting examples of alkyl groups include straight, branched, and cyclic alkyl and alkylene groups. An alkyl group can be, for example, a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted.

Alkyl groups can include branched and unbranched alkyl groups. Non-limiting examples of straight alkyl groups include methyl, ethyl, propyl, butyl, pentyl, hexyl, heptyl, octyl, nonyl, and decyl.

Branched alkyl groups include any straight alkyl group substituted with any number of alkyl groups. Non-limiting examples of branched alkyl groups include isopropyl, isobutyl, sec-butyl, and t-butyl.

Non-limiting examples of substituted alkyl groups includes hydroxymethyl, chloromethyl, trifluoromethyl, aminomethyl, 1-chloroethyl, 2-hydroxyethyl, 1,2-difluoroethyl, and 3-carboxypropyl.

Non-limiting examples of cycloalkyl groups include cyclopropyl, cyclobutyl, cyclopentyl, cyclohexyl, cycloheptyl, and cyclooctyl groups. Cycloalkyl groups also include fused-, bridged-, and spiro-bicycles and higher fused-, bridged-, and spiro-systems. A cycloalkyl group can be substituted with any number of straight, branched, or cyclic alkyl groups. Non-limiting examples of cyclic alkyl groups include cyclopropyl, 2-methyl-cycloprop-1-yl, cycloprop-2-en-1-yl, cyclobutyl, 2,3-dihydroxycyclobut-1-yl, cyclobut-2-en-1-yl, cyclopentyl, cyclopent-2-en-1-yl, cyclopenta-2,4-dien-1-yl, cyclohexyl, cyclohex-2-en-1-yl, cycloheptyl, cyclooctanyl, 2,5-dimethylcyclopent-1-yl, 3,5-dichlorocyclohex-1-yl, 4-hydroxycyclohex-1-yl, 3,3,5-trimethylcyclohex-1-yl, octahydropentalenyl, octahydro-TH-indenyl, 3a,4,5,6,7,7a-hexahydro-3H-inden-4-yl, decahydroazulenyl, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

Non-limiting examples of alkenyl groups include straight, branched, and cyclic alkenyl groups. The olefin or olefins of an alkenyl group can be, for example, E, Z, cis, trans, terminal, or exo-methylene. An alkenyl group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of alkenyl and alkenylene groups include ethenyl, prop-1-en-1-yl, isopropenyl, but-1-en-4-yl; 2-chloroethenyl, 4-hydroxybuten-1-yl, 7-hydroxy-7-methyloct-4-en-2-yl, and 7-hydroxy-7-methyloct-3,5-dien-2-yl.

Non-limiting examples of alkynyl groups include straight, branched, and cyclic alkynyl groups. The triple bond of an alkynyl group can be internal or terminal. An alkynyl or alkynylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of alkynyl groups include ethynyl, prop-2-yn-1-yl, prop-1-yn-1-yl, and 2-methyl-hex-4-yn-1-yl; 5-hydroxy-5-methylhex-3-yn-1-yl, 6-hydroxy-6-methylhept-3-yn-2-yl, and 5-hydroxy-5-ethylhept-3-yn-1-yl.

A halo-alkyl group can be any alkyl group substituted with any number of halogen atoms, for example, fluorine, chlorine, bromine, and iodine atoms. A halo-alkenyl group can be any alkenyl group substituted with any number of halogen atoms. A halo-alkynyl group can be any alkynyl group substituted with any number of halogen atoms.

An alkoxy group can be, for example, an oxygen atom substituted with any alkyl, alkenyl, or alkynyl group. An ether or an ether group comprises an alkoxy group. Non-limiting examples of alkoxy groups include methoxy, ethoxy, propoxy, isopropoxy, and isobutoxy.

A heterocycle can be any ring containing a ring atom that is not carbon, for example, N, O, S, P, Si, B, or any other heteroatom. A heterocycle can be substituted with any number of substituents, for example, alkyl groups and halogen atoms. A heterocycle can be aromatic (heteroaryl) or non-aromatic. Non-limiting examples of heterocycles include pyrrole, pyrrolidine, pyridine, piperidine, succinimide, maleimide, morpholine, imidazole, thiophene, furan, tetrahydrofuran, pyran, and tetrahydropyran.

Non-limiting examples of heterocycles include: heterocyclic units having a single ring containing one or more heteroatoms, non-limiting examples of which include, diazirinyl, aziridinyl, azetidinyl, pyrazolidinyl, imidazolidinyl, oxazolidinyl, isoxazolinyl, thiazolidinyl, isothiazolinyl, oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, piperidin-2-onyl, 2,3,4,5-tetrahydro-1H-azepinyl, 2,3-dihydro-1H-indole, and 1,2,3,4-tetrahydroquinoline; and ii) heterocyclic units having 2 or more rings one of which is a heterocyclic ring, non-limiting examples of which include hexahydro-1H-pyrrolizinyl, 3a,4,5,6,7,7a-hexahydro-1H-benzo[d]imidazolyl, 3a,4,5,6,7,7a-hexahydro-1H-indolyl, 1,2,3,4-tetrahydroquinolinyl, and decahydro-1H-cycloocta[b]pyrrolyl.

Non-limiting examples of heteroaryl include: i) heteroaryl rings containing a single ring, non-limiting examples of which include, 1,2,3,4-tetrazolyl, [1,2,3]triazolyl, [1,2,4]triazolyl, triazinyl, thiazolyl, 1H-imidazolyl, oxazolyl, isoxazolyl, isothiazolyl, furanyl, thiophenyl, pyrimidinyl, 2-phenylpyrimidinyl, pyridinyl, 3-methylpyridinyl, and 4-dimethylaminopyridinyl; and ii) heteroaryl rings containing 2 or more fused rings one of which is a heteroaryl ring, non-limiting examples of which include: 7H-purinyl, 9H-purinyl, 6-amino-9H-purinyl, 5H-pyrrolo[3,2-d]pyrimidinyl, 7H-pyrrolo[2,3-d]pyrimidinyl, pyrido[2,3-d]pyrimidinyl, 4,5,6,7-tetrahydro-1-H-indolyl, quinoxalinyl, quinazolinyl, quinolinyl, 8-hydroxy-quinolinyl, and isoquinolinyl.

“Alkyl” refers to an optionally substituted straight-chain, or optionally substituted branched-chain saturated hydrocarbon having from one to about ten carbon atoms, or from one to six carbon atoms, wherein an sp³-hybridized carbon of the alkyl residue is attached to the rest of the molecule by a single bond. Examples include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl, and hexyl, and longer alkyl groups, such as heptyl, octyl, and the like. Whenever it appears herein, a numerical range such as “C₁-C₆ alkyl” means that the alkyl group consists of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁-C₁₀ alkyl, a C₁-C₉ alkyl, a C₁-C₈ alkyl, a C₁-C₇ alkyl, a C₁-C₆ alkyl, a C₁-C₅ alkyl, a C₁-C₄ alkyl, a C₁-C₃ alkyl, a C₁-C₂ alkyl, or a C₁ alkyl. Unless stated otherwise specifically in the specification, an alkyl group is optionally substituted, for example, with oxo, halogen, amino, nitrile, nitro, hydroxyl, haloalkyl, alkoxy, aryl, cycloalkyl, heterocyclylalkyl, heteroaryl, and the like. In some embodiments, the alkyl is optionally substituted with oxo, halogen, —CN, —CF₃, —OH, —OMe, —NH₂, or —NO₂. In some embodiments, the alkyl is optionally substituted with oxo, halogen, —CN, —CF₃, —OH, or —OMe. In some embodiments, the alkyl is optionally substituted with halogen.

“Alkenyl” refers to an optionally substituted straight-chain, or optionally substituted branched-chain hydrocarbon having one or more carbon-carbon double-bonds and having from two to about ten carbon atoms, more preferably two to about six carbon atoms, wherein an sp²-hybridized carbon of the alkenyl residue is attached to the rest of the molecule by a single bond. The group may be in either the cis or trans conformation about the double bond(s), and should be understood to include both isomers. Examples include, but are not limited to, ethenyl (—CH═CH₂), 1-propenyl (—CH₂CH═CH₂), isopropenyl [—C(CH₃)═CH₂], butenyl, 1,3-butadienyl, and the like. Whenever it appears herein, a numerical range such as “C₂-C₆ alkenyl” means that the alkenyl group may consist of 2 carbon atoms, 3 carbon atoms, 4 carbon atoms, 5 carbon atoms, or 6 carbon atoms, although the present definition also covers the occurrence of the term “alkenyl” where no numerical range is designated. In some embodiments, the alkenyl is a C₂-C₁₀ alkenyl, a C₂-C₉ alkenyl, a C₂-C₈ alkenyl, a C₂-C₇ alkenyl, a C₂-C₆ alkenyl, a C₂-C₅ alkenyl, a C₂-C₄ alkenyl, a C₂-C₃ alkenyl, or a C₂ alkenyl. Unless stated otherwise specifically in the specification, an alkenyl group is optionally substituted, for example, with oxo, halogen, amino, nitrile, nitro, hydroxyl, haloalkyl, alkoxy, aryl, cycloalkyl, heterocyclylalkyl, heteroaryl, and the like. In some embodiments, an alkenyl is optionally substituted with oxo, halogen, —CN, —CF₃, —OH, —OMe, —NH₂, or —NO₂. In some embodiments, an alkenyl is optionally substituted with oxo, halogen, —CN, —CF₃, —OH, or —OMe. In some embodiments, the alkenyl is optionally substituted with halogen.

“Alkynyl” refers to an optionally substituted straight-chain or optionally substituted branched-chain hydrocarbon having one or more carbon-carbon triple-bonds and having from two to about ten carbon atoms, more preferably from two to about six carbon atoms. Examples include, but are not limited to, ethynyl, 2-propynyl, 2-butynyl, 1,3-butadiynyl, and the like. Whenever it appears herein, a numerical range such as “C₂-C₆ alkynyl” means that the alkynyl group may consist of 2 carbon atoms, 3 carbon atoms, 4 carbon atoms, 5 carbon atoms, or 6 carbon atoms, although the present definition also covers the occurrence of the term “alkynyl” where no numerical range is designated. In some embodiments, the alkynyl is a C₂-C₁₀ alkynyl, a C₂-C₉ alkynyl, a C₂-C₈ alkynyl, a C₂-C₇ alkynyl, a C₂-C₆ alkynyl, a C₂-C₅ alkynyl, a C₂-C₄ alkynyl, a C₂-C₃ alkynyl, or a C₂ alkynyl. Unless stated otherwise specifically in the specification, an alkynyl group is optionally substituted, for example, with oxo, halogen, amino, nitrile, nitro, hydroxyl, haloalkyl, alkoxy, aryl, cycloalkyl, heterocyclylalkyl, heteroaryl, and the like. In some embodiments, an alkynyl is optionally substituted with oxo, halogen, —CN, —CF₃, —OH, —OMe, —NH₂, or —NO₂. In some embodiments, an alkynyl is optionally substituted with oxo, halogen, —CN, —CF₃, —OH, or —OMe. In some embodiments, the alkynyl is optionally substituted with halogen.

“Alkoxy” refers to a radical of the formula —ORₐ where Rₐ is an alkyl radical as defined. Unless stated otherwise specifically in the specification, an alkoxy group may be optionally substituted, for example, with oxo, halogen, amino, nitrile, nitro, hydroxyl, haloalkyl, alkoxy, aryl, cycloalkyl, heterocyclylalkyl, heteroaryl, and the like. In some embodiments, an alkoxy is optionally substituted with oxo, halogen, —CN, —CF₃, —OH, —OMe, —NH₂, or —NO₂. In some embodiments, an alkoxy is optionally substituted with oxo, halogen, —CN, —CF₃, —OH, or —OMe. In some embodiments, the alkoxy is optionally substituted with halogen.

“Aminoalkyl” refers to an alkyl radical, as defined above, that is substituted by one or more amines. In some embodiments, the alkyl is substituted with one amine. In some embodiments, the alkyl is substituted with one, two, or three amines. Hydroxyalkyl include, for example, aminomethyl, aminoethyl, aminopropyl, aminobutyl, or aminopentyl. In some embodiments, the hydroxyalkyl is aminomethyl.

“Aryl” refers to a radical derived from a hydrocarbon ring system comprising hydrogen, 6 to 30 carbon atoms, and at least one aromatic ring. The aryl radical may be a monocyclic, bicyclic, tricyclic, or tetracyclic ring system, which may include fused (when fused with a cycloalkyl or heterocyclylalkyl ring, the aryl is bonded through an aromatic ring atom) or bridged ring systems. In some embodiments, the aryl is a 6- to 10-membered aryl. In some embodiments, the aryl is a 6-membered aryl. Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. In some embodiments, the aryl is phenyl. Unless stated otherwise specifically in the specification, an aryl may be optionally substituted, for example, with halogen, amino, nitrile, nitro, hydroxyl, alkyl, alkenyl, alkynyl, haloalkyl, alkoxy, aryl, cycloalkyl, heterocyclylalkyl, heteroaryl, and the like. In some embodiments, an aryl is optionally substituted with halogen, methyl, ethyl, —CN, —CF₃, —OH, —OMe, —NH₂, or —NO₂. In some embodiments, an aryl is optionally substituted with halogen, methyl, ethyl, —CN, —CF₃, —OH, or —OMe. In some embodiments, the aryl is optionally substituted with halogen.

“Cycloalkyl” refers to a stable, partially or fully saturated, monocyclic or polycyclic carbocyclic ring, which may include fused (when fused with an aryl or a heteroaryl ring, the cycloalkyl is bonded through a non-aromatic ring atom), bridged, or spiro ring systems. Representative cycloalkyls include, but are not limited to, cycloalkyls having from three to fifteen carbon atoms (C₃-C₁₅ cycloalkyl), from three to ten carbon atoms (C₃-C₁₀ cycloalkyl), from three to eight carbon atoms (C₃-C₈ cycloalkyl), from three to six carbon atoms (C₃-C₆ cycloalkyl), from three to five carbon atoms (C₃-C₅ cycloalkyl), or three to four carbon atoms (C₃-C₄ cycloalkyl). In some embodiments, the cycloalkyl is a 3- to 6-membered cycloalkyl. In some embodiments, the cycloalkyl is a 5- to 6-membered cycloalkyl. Monocyclic cycloalkyls include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example, cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocyclylalkyl, heteroaryl, and the like. In some embodiments, a cycloalkyl is optionally substituted with oxo, halogen, methyl, ethyl, —CN, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Deuteroalkyl” refers to an alkyl radical, as defined above, that is substituted by one or more deuteriums. In some embodiments, the alkyl is substituted with one deuterium. In some embodiments, the alkyl is substituted with one, two, or three deuteriums. In some embodiments, the alkyl is substituted with one, two, three, four, five, or six deuteriums. Deuteroalkyl include, for example, CD₃, CH₂D, CHD₂, CH₂CD₃, CD₂CD₃, CHDCD₃, CH₂CH₂D, or CH₂CHD₂. In some embodiments, the deuteroalkyl is CD₃.

“Haloalkyl” refers to an alkyl radical, as defined above, that is substituted by one or more halogens. In some embodiments, the alkyl is substituted with one, two, or three halogens. In some embodiments, the alkyl is substituted with one, two, three, four, five, or six halogens. Haloalkyl include, for example, trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. In some embodiments, the haloalkyl is trifluoromethyl.

“Halo” or “halogen” refers to bromo, chloro, fluoro, or iodo. In some embodiments, halogen is fluoro or chloro. In some embodiments, halogen is fluoro.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, or —CH(CH₃)OCH₃. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocyclylalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Heterocyclylalkyl” refers to a stable 3- to 24-membered partially or fully saturated ring radical comprising 2 to 23 carbon atoms and from one to 8 heteroatoms selected from the group consisting of nitrogen, oxygen, phosphorous, and sulfur. Unless stated otherwise specifically in the specification, the heterocyclylalkyl radical may be a monocyclic, bicyclic, tricyclic, or tetracyclic ring system, which may include fused (when fused with an aryl or a heteroaryl ring, the heterocyclylalkyl is bonded through a non-aromatic ring atom) or bridged ring systems; and the nitrogen, carbon, or sulfur atoms in the heterocyclylalkyl radical may be optionally oxidized; the nitrogen atom may be optionally quaternized.

Representative heterocyclylalkyls include, but are not limited to, heterocyclylalkyls having from two to fifteen carbon atoms (C₂-C₁₅ heterocyclylalkyl), from two to ten carbon atoms (C₂-C₁₀ heterocyclylalkyl), from two to eight carbon atoms (C₂-C₈ heterocyclylalkyl), from two to six carbon atoms (C₂-C₆ heterocyclylalkyl), from two to five carbon atoms (C₂-C₅ heterocyclylalkyl), or two to four carbon atoms (C₂-C₄ heterocyclylalkyl). In some embodiments, the heterocyclylalkyl is a 3- to 6-membered heterocyclylalkyl. In some embodiments, the cycloalkyl is a 5- to 6-membered heterocyclylalkyl. Examples of such heterocyclylalkyl radicals include, but are not limited to, aziridinyl, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocyclylalkyl also includes all ring forms of the carbohydrates, including but not limited to, the monosaccharides, the disaccharides, and the oligosaccharides. It is understood that when referring to the number of carbon atoms in a heterocyclylalkyl, the number of carbon atoms in the heterocyclylalkyl is not the same as the total number of atoms (including the heteroatoms) that make up the heterocyclylalkyl (i.e. skeletal atoms of the heterocyclylalkyl ring). Unless stated otherwise specifically in the specification, a heterocyclylalkyl is optionally substituted, for example, with oxo, halogen, amino, nitrile, nitro, hydroxyl, alkyl, alkenyl, alkynyl, haloalkyl, alkoxy, aryl, cycloalkyl, heterocyclylalkyl, heteroaryl, and the like. In some embodiments, a heterocyclylalkyl is optionally substituted with oxo, halogen, methyl, ethyl, —CN, —CF₃, —OH, —OMe, —NH₂, or —NO₂. In some embodiments, a heterocyclylalkyl is optionally substituted with oxo, halogen, methyl, ethyl, —CN, —CF₃, —OH, or —OMe. In some embodiments, the heterocyclylalkyl is optionally substituted with halogen. In one embodiment, heterocyclylalkyl is

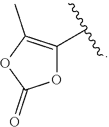 [SMILES: *c1oc(=O)oc1C]

“Heteroaryl” refers to a 5- to 14-membered ring system radical comprising hydrogen atoms, one to thirteen carbon atoms, one to six heteroatoms selected from the group consisting of nitrogen, oxygen, phosphorous, and sulfur, and at least one aromatic ring. The heteroaryl radical may be a monocyclic, bicyclic, tricyclic, or tetracyclic ring system, which may include fused (when fused with a cycloalkyl or heterocyclylalkyl ring, the heteroaryl is bonded through an aromatic ring atom) or bridged ring systems; and the nitrogen, carbon, or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-TH-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl is optionally substituted, for example, with halogen, amino, nitrile, nitro, hydroxyl, alkyl, alkenyl, alkynyl, haloalkyl, alkoxy, aryl, cycloalkyl, heterocyclylalkyl, heteroaryl, and the like. In some embodiments, a heteroaryl is optionally substituted with halogen, methyl, ethyl, —CN, —CF₃, —OH, —OMe, —NH₂, or —NO₂. In some embodiments, a heteroaryl is optionally substituted with halogen, methyl, ethyl, —CN, —CF₃, —OH, or —OMe. In some embodiments, the heteroaryl is optionally substituted with halogen.

Any compound herein can be purified. A compound herein can be least 1% pure, at least 2% pure, at least 3% pure, at least 4% pure, at least 5% pure, at least 6% pure, at least 7% pure, at least 8% pure, at least 9% pure, at least 10% pure, at least 11% pure, at least 12% pure, at least 13% pure, at least 14% pure, at least 15% pure, at least 16% pure, at least 17% pure, at least 18% pure, at least 19% pure, at least 20% pure, at least 21% pure, at least 22% pure, at least 23% pure, at least 24% pure, at least 25% pure, at least 26% pure, at least 27% pure, at least 28% pure, at least 29% pure, at least 30% pure, at least 31% pure, at least 32% pure, at least 33% pure, at least 34% pure, at least 35% pure, at least 36% pure, at least 37% pure, at least 38% pure, at least 39% pure, at least 40% pure, at least 41% pure, at least 42% pure, at least 43% pure, at least 44% pure, at least 45% pure, at least 46% pure, at least 47% pure, at least 48% pure, at least 49% pure, at least 50% pure, at least 51% pure, at least 52% pure, at least 53% pure, at least 54% pure, at least 55% pure, at least 56% pure, at least 57% pure, at least 58% pure, at least 59% pure, at least 60% pure, at least 61% pure, at least 62% pure, at least 63% pure, at least 64% pure, at least 65% pure, at least 66% pure, at least 67% pure, at least 68% pure, at least 69% pure, at least 70% pure, at least 71% pure, at least 72% pure, at least 73% pure, at least 74% pure, at least 75% pure, at least 76% pure, at least 77% pure, at least 78% pure, at least 79% pure, at least 80% pure, at least 81% pure, at least 82% pure, at least 83% pure, at least 84% pure, at least 85% pure, at least 86% pure, at least 87% pure, at least 88% pure, at least 89% pure, at least 90% pure, at least 91% pure, at least 92% pure, at least 93% pure, at least 94% pure, at least 95% pure, at least 96% pure, at least 97% pure, at least 98% pure, at least 99% pure, at least 99.1% pure, at least 99.2% pure, at least 99.3% pure, at least 99.4% pure, at least 99.5% pure, at least 99.6% pure, at least 99.7% pure, at least 99.8% pure, or at least 99.9% pure.

### Pharmaceutically Acceptable Salts.

The present disclosure provides the use of pharmaceutically-acceptable salts of any compound described herein. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt. In some embodiments, a pharmaceutically-acceptable salt is an ammonium salt.

Metal salts can arise from the addition of an inorganic base to a compound of the present disclosure. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound of the present disclosure. In some embodiments, the organic amine is trimethyl amine, triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrazole, pyrazolidine, pyrazoline, pyridazine, pyrimidine, imidazole, or pyrazine.

In some embodiments, an ammonium salt is a triethyl amine salt, trimethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrazole salt, a pyridazine salt, a pyrimidine salt, an imidazole salt, or a pyrazine salt.

Acid addition salts can arise from the addition of an acid to a compound of the present disclosure. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisic acid, gluconic acid, glucuronic acid, saccharic acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisate salt, a gluconate salt, a glucuronate salt, a saccharate salt, a formate salt, a benzoate salt, a glutamate salt, a pantothenate salt, an acetate salt, a propionate salt, a butyrate salt, a fumarate salt, a succinate salt, a methanesulfonate salt, an ethanesulfonate salt, a benzenesulfonate salt, a p-toluenesulfonate salt, a citrate salt, an oxalate salt, or a maleate salt. Pharmaceutical compositions.

According to another embodiment, the present disclosure provides a composition comprising a compound of the present disclosure and a pharmaceutically acceptable carrier, adjuvant, or vehicle. The amount of compound in the composition is an amount effective to treat the relevant disease, disorder, or condition in a patient in need thereof (an “effective amount”). In some embodiments, a composition of the present disclosure is formulated for oral administration to a patient.

The term “pharmaceutically acceptable carrier, adjuvant, or vehicle” refers to a non-toxic carrier, adjuvant, or vehicle that does not destroy the pharmacological activity of the agent with which it is formulated. Pharmaceutically acceptable carriers, adjuvants or vehicles that may be used in the disclosed compositions include, but are not limited to, ion exchangers, alumina, stearates such as aluminum stearate, lecithin, serum proteins such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present disclosure may be administered orally, parenterally, enterally, intracistemally, intraperitoneally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the composition is administered orally, intraperitoneally, or intravenously. In some embodiments, the composition is a transmucosal formulation. Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

To aid in delivery of the composition, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, may also be added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Alternatively, pharmaceutically acceptable compositions may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

In some embodiments, the pharmaceutically acceptable composition is formulated for oral administration. Such formulations may be administered with or without food. In some embodiments, the pharmaceutically acceptable composition is administered without food. In other embodiments, the pharmaceutically acceptable composition is administered with food.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present disclosure, it can be desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing a compound of this disclosure with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinyl pyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

Therapeutic agents can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this disclosure include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

### Compounds

In one aspect, disclosed herein are psilocin analogs.

Provided herein are compounds of Formula (I), or a pharmaceutically acceptable salt, solvate, or isotopolog thereof:

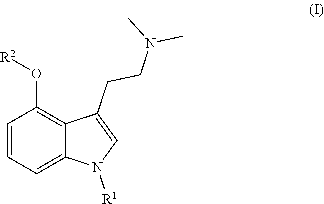

wherein:
R¹ is hydrogen, —OH, unsubstituted or substituted alkyl, —OR, or C(O)OR; wherein R is unsubstituted alkyl;
R² is —C(O)R³, —C(O)OR³, —CH(R⁴)OC(O)R⁵, —CH(R⁴)OC(O)OR⁵, —C(O)NR⁶R⁷, —CH(R⁴)OC(O)NR⁶R⁷, —S(O)₂NR⁶R⁷, —S(O)₂OR⁵, —P(O)OR⁸(NR⁹R¹⁰), —P(O)(OR¹¹)(OR¹²), —CH(R⁴)OP(O)(OR¹¹)(OR¹²), or —Si(R³)(R⁴)(R⁵);
each of R³, R⁴, R⁵, and R⁸ is independently hydrogen, alkyl, heteroalkyl, cycloalkyl, heterocyclylalkyl, aryl, or heteroaryl, wherein alkyl, heteroalkyl, cycloalkyl, heterocyclylalkyl, aryl, or heteroaryl is unsubstituted or substituted with one or more R^{A};
each of R⁶ and R⁷ is independently hydrogen, alkyl, heteroalkyl, cycloalkyl, heterocyclylalkyl, aryl, or heteroaryl, wherein alkyl, heteroalkyl, cycloalkyl, heterocyclylalkyl, aryl, or heteroaryl is unsubstituted or substituted with one or more R^{A}; or R⁶ and R⁷ together with the atom to which they are attached form a heterocyclylalkyl ring or a heteroaryl ring that is unsubstituted or substituted with one or more R^{A};
each of R⁹ and R¹⁰ is independently hydrogen, alkyl, heteroalkyl, cycloalkyl, heterocyclylalkyl, aryl, heteroaryl, or hydrogen, wherein alkyl, heteroalkyl, cycloalkyl, heterocyclylalkyl, aryl, or heteroaryl is unsubstituted or substituted with one or more R^{A}; or R⁹ and R¹⁰ together with the atom to which they are attached form a heterocyclylalkyl ring or a heteroaryl ring that is unsubstituted or substituted with one or more R^{A}.
each of R¹¹ and R¹² is independently hydrogen, alkyl, heteroalkyl, cycloalkyl, heterocyclylalkyl, aryl, heteroaryl, or hydrogen, wherein alkyl, heteroalkyl, cycloalkyl, heterocyclylalkyl, aryl, or heteroaryl is unsubstituted or substituted with one or more R^{A};
each R^{A} is independently alkyl, heteroalkyl, cycloalkyl, heterocyclylalkyl, aryl, heteroaryl, an amino acid side chain, —OR¹³, —N(R¹⁸)R¹⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹], —C(O)N(R¹⁸)R¹⁹, —OC(O)N(R¹⁸)R¹⁹, or —OP(O)OR²⁰(OR²¹), wherein alkyl, heteroalkyl, cycloalkyl, heterocyclylalkyl, aryl, or heteroaryl is unsubstituted or substituted with one or more alkyl, aryl, halogen, —S—R¹³, —OR¹³, —NR(R¹⁸)R¹⁹, —C(O)R¹⁴, —OC(O)R¹⁵, —OC(O)OR¹⁶, or —OC(O)N(R¹⁸)R¹⁹;
each of R¹³, R¹⁴, R¹⁵, R¹⁶, or R¹⁷ is independently hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, and heteroaryl is unsubstituted or substituted with one or more R^{B};
each of R¹⁸ and R¹⁹ is independently hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, or heteroaryl is unsubstituted or substituted with one or more R^{B}; or R¹⁸ and R¹⁹ together with the atom to which they are attached form a heterocyclylalkyl ring or heteroaryl ring, each of which is unsubstituted or substituted with one or more R^{B};
each R^{B} is independently halogen, amino, cyano, hydroxyl, alkyl, alkenyl, heteroalkyl, cycloalkyl, heterocyclylalkyl, aryl, heteroaryl, arylalkyl, —OC(O)R¹⁸, —C(O)R¹⁸, —C(O)OR¹⁸, NHC(O)OR¹⁸, or heteroarylalkyl, wherein cycloalkyl, heterocyclylalkyl, aryl, or heteroaryl is unsubstituted or substituted with one or more halogen, amino, cyano, hydroxyl, alkyl, acetyl, or benzoyl.

In some embodiments, R¹ is hydrogen, —OH, unsubstituted or substituted alkyl, OR, or C(O)OR; wherein R is unsubstituted alkyl. In some embodiments, R¹ is hydrogen or unsubstituted or substituted alkyl. In some embodiments, R¹ is hydrogen. In some embodiments, R¹ is unsubstituted or substituted alkyl. In some embodiments, R¹ is unsubstituted alkyl. In some embodiments, R¹ is methyl, ethyl, n-propyl, isopropyl, n-butyl, tert-butyl, n-pentyl, 3-methyl-1-butyl, or —C₁₀H₂₁. In some embodiments, R¹ is methyl.

In some embodiments, R is methyl, ethyl, n-propyl, isopropyl, n-butyl, tert-butyl, n-pentyl, 3-methyl-1-butyl, or —C₁₀H₂₁. In some embodiments, R is methyl.

In some embodiments, R² is —C(O)R³, —C(O)OR³, —CH(R⁴)OC(O)R⁵, —CH(R⁴)OC(O)OR⁵, —C(O)NR⁶R⁷, —CH(R⁴)OC(O)NR⁶R⁷, —S(O)₂NR⁶R⁷, —P(O)OR⁸(NR⁹R¹⁰), or —P(O)(OR¹¹)(OR¹²).

In some embodiments, R² is —C(O)R³, —C(O)OR³, —CH(R⁴)OC(O)R⁵, —CH(R⁴)OC(O)OR⁵, —C(O)NR⁶R⁷, —CH(R⁴)OC(O)NR⁶R⁷, —P(O)OR⁸(NR⁹R¹⁰), or —P(O)(OR¹¹)(OR¹²).

In some embodiments, R² is —C(O)R³, —C(O)OR³, —CH(R⁴)OC(O)R⁵, —CH(R⁴)OC(O)OR⁵, —C(O)NR⁶R⁷, —CH(R⁴)OC(O)NR⁶R⁷, or —S(O)₂NR⁶R⁷.

In some embodiments, R² is —C(O)R³, —C(O)OR³, —CH(R⁴)OC(O)R⁵, —CH(R⁴)OC(O)OR⁵, —C(O)NR⁶R⁷, —CH(R⁴)OC(O)NR⁶R⁷, —S(O)₂NR⁶R⁷, or —P(O)(OR¹¹)(OR¹²).

In some embodiments, R² is —C(O)R³, —C(O)OR³, —CH(R⁴)OC(O)R⁵, —CH(R⁴)OC(O)OR⁵, —C(O)NR⁶R⁷, —CH(R⁴)OC(O)NR⁶R⁷, —S(O)₂NR⁶R⁷, or —P(O)OR⁸(NR⁹R¹⁰).

In some embodiments, R² is —C(O)R³, —C(O)OR³, —CH(R⁴)OC(O)R⁵, —CH(R⁴)OC(O)OR⁵, —C(O)NR⁶R⁷, or —CH(R⁴)OC(O)NR⁶R⁷. In some embodiments, R² is —C(O)OR³, —CH(R⁴)OC(O)R⁵, —CH(R⁴)OC(O)OR⁵, —C(O)NR⁶R⁷, or —CH(R⁴)OC(O)NR⁶R⁷. In some embodiments, R² is —C(O)R³, —CH(R⁴)OC(O)R⁵, —CH(R⁴)OC(O)OR⁵, —C(O)NR⁶R⁷, or —CH(R⁴)OC(O)NR⁶R⁷. In some embodiments, R² is —C(O)R³, —C(O)OR³, —CH(R⁴)OC(O)OR⁵, —C(O)NR⁶R⁷, or —CH(R⁴)OC(O)NR⁶R⁷. In some embodiments, R² is —C(O)R³, —C(O)OR³, —CH(R⁴)OC(O)R⁵, —C(O)NR⁶R⁷, or —CH(R⁴)OC(O)NR⁶R⁷. In some embodiments, R² is —C(O)R³, —C(O)OR³, —CH(R⁴)OC(O)R⁵, —CH(R⁴)OC(O)OR⁵, or —CH(R⁴)OC(O)NR⁶R⁷. In some embodiments, R² is —C(O)R³, —C(O)OR³, —CH(R⁴)OC(O)R⁵, —CH(R⁴)OC(O)OR⁵, or —C(O)NR⁶R⁷.

In some embodiments, R² is —C(O)R³, —C(O)OR³, or —C(O)NR⁶R⁷. In some embodiments, R² is —C(O)OR³ or —C(O)NR⁶R⁷. In some embodiments, R² is —C(O)R³ or —C(O)NR⁶R⁷. In some embodiments, R² is —C(O)R³ or —C(O)OR³. In some embodiments, R² is —CH(R⁴)OC(O)R⁵, —CH(R⁴)OC(O)OR⁵, or —CH(R⁴)OC(O)NR⁶R⁷. In some embodiments, R² is —CH(R⁴)OC(O)OR⁵ or —CH(R⁴)OC(O)NR⁶R⁷. In some embodiments, R² is —CH(R⁴)OC(O)R⁵ or —CH(R⁴)OC(O)NR⁶R⁷. In some embodiments, R² is —CH(R⁴)OC(O)R⁵ or —CH(R⁴)OC(O)OR⁵.

In some embodiments, R² is —C(O)R³. In some embodiments, R² is —C(O)OR³. In some embodiments, R² is —CH(R⁴)OC(O)R⁵. In some embodiments, R² is —CH(R⁴)OC(O)OR⁵. In some embodiments, R² is —C(O)NR⁶R⁷. In some embodiments, R² is —CH(R⁴)OC(O)NR⁶R⁷.

In some embodiments, R² is —S(O)₂NR⁶R⁷, —P(O)OR⁸(NR⁹R¹⁰), or —P(O)(OR¹¹)(OR¹²).

In some embodiments, R² is —S(O)₂NR⁶R⁷.

In some embodiments, R² is —P(O)OR⁸(NR⁹R¹⁰) or —P(O)(OR¹¹)(OR¹²).

In some embodiments, R² is —P(O)OR⁸(NR⁹R¹⁰).

In some embodiments, R² is or —P(O)(OR¹¹)(OR¹²).

### Ester Prodrugs

In some embodiments of compound of Formula (I), the compound has the structure of Formula (Ia):

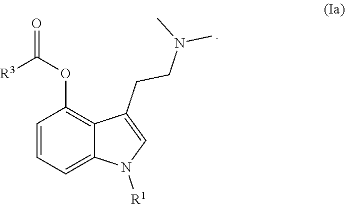

In some embodiments, R³ is unsubstituted or substituted alkyl, heteroalkyl, cycloalkyl, or heterocyclylalkyl.

In some embodiments, R³ is unsubstituted or substituted alkyl. In some embodiments, R³ is alkyl substituted with one or more substituent R^{A}, and wherein each R^{A} is independently selected from alkyl, heteroalkyl, cycloalkyl, heterocyclylalkyl, aryl, heteroaryl, an amino acid side chain, —OR¹³, —N(R¹⁸)R¹⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹], —C(O)N(R¹⁸)R¹⁹, —OC(O)N(R¹⁸)R¹⁹, or —OP(O)OR²⁰(OR²¹).

In some embodiments, R³ is unsubstituted alkyl. In some embodiments, R³ is methyl, ethyl, n-propyl, isopropyl, n-butyl, tert-butyl, n-pentyl, 3-methyl-1-butyl, or —C₁₀H₂₁.

In some embodiments, R³ is alkyl substituted with —C(O)OR¹³. In some embodiments, R¹³ is hydrogen or alkyl. In some embodiments, R¹³ is hydrogen, methyl, ethyl, or tert-butyl.

In some embodiments, R³ is

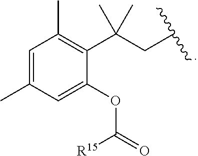

In some embodiments, R³ is alkyl substituted with —N(R¹⁸)R¹⁹, wherein each of R¹⁸ and R¹⁹ is independently hydrogen or methyl.

In some embodiments, R³ is alkyl substituted with —N(R¹³)C(O)R¹⁴, wherein each of R¹³ and R¹⁴ is independently hydrogen or methyl.

In some embodiments, R³ is alkyl substituted with —N(R¹³)C(O)R¹⁴, wherein each of R¹³ is hydrogen or methyl, and R¹⁴ is hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, and heteroaryl is unsubstituted or further substituted with one or more halogen, amino, cyano, hydroxyl, alkyl, acetyl, benzoyl, phenyl, or NH-Boc.

In some embodiments, R³ is alkyl substituted with —N(R¹³)C(O)OR¹⁴, wherein each of R¹³ and R¹⁴ is independently hydrogen, methyl, ethyl, isopropyl, or tert-butyl.

In some embodiments, R³ is alkyl substituted with —OC(O)R¹⁵, wherein R¹⁵ is hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, and heteroaryl is unsubstituted or further substituted with one or more halogen, amino, cyano, hydroxyl, alkyl, acetyl, or benzoyl.

In some embodiments, R³ is heterocyclylalkyl.

In some embodiments, R³ is selected from aziridinyl, oxetanyl, azetidinyl, tetrahydrofuranyl, pyrrolidinyl, piperidinyl, piperazinyl, morpholinyl, thiomorpholinyl, thiomorpholinyl dioxide, diazinanyl,

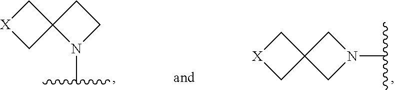

wherein X is —CH₂—, —O—, —S—, —SO₂, —NH—, or —NMe-.

In some embodiments, R³ is heterocyclylalkyl. In some embodiments, R³ is selected from aziridinyl, oxetanyl, azetidinyl, tetrahydrofuranyl, pyrrolidinyl, piperidinyl, piperazinyl, morpholinyl, thiomorpholinyl, thiomorpholinyl dioxide, diazinanyl,

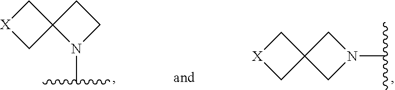

wherein X is —CH₂—, —O—, —S—, —SO₂, —NH—, or —NMe. In some embodiments, R³ is

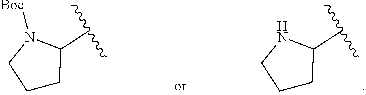 [SMILES: *C1CCCN1C(=O)OC(C)(C)C ; *C1CCCN1]

In some embodiments, R³ is oxetanyl.

In some embodiments, R³ is heteroalkyl.

In some embodiments, R³ is unsubstituted or substituted aryl (e.g., phenyl). In some embodiments, R³ is substituted phenyl. In some embodiments, R³ is phenyl substituted with —OC(O)R¹⁸, wherein R¹⁸ is hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl.

In some embodiments, R³ is

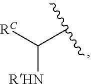

wherein R^{C} is a natural amino acid side chain, and R′ is hydrogen or -Boc. In some embodiments, R³ is

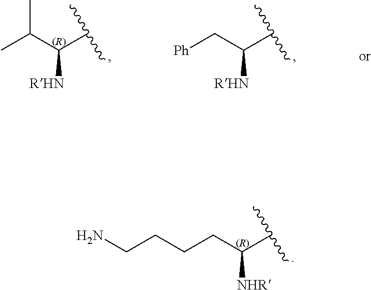

In some embodiments, R³ is selected from alkyl, cycloalkyl, substituted alkyl, substituted cycloalkyl, vinyl, aryl, heteroaryl, substituted aryl, and substituted heteroaryl. In some embodiments, R³ is selected from hydrogen, —CD₃, Et, n-Pr, iPr, tBu, n-pentyl, iso-amyl, n-hexyl, n-heptyl, n-octyl, n-nonyl, cyclopropyl, cyclobutyl, cyclopentyl, cyclohexyl, cycloheptyl, cyclooctyl, CH₂CF₃, —CH₂-cyclopropyl, Ph, 2-pyridyl, 3-pyridyl, 4-pyridyl, 2-pyrimidyl, 4-pyrimidyl, 5-pyrimidyl, and 6-pyrimidyl.

In some embodiments, R³ is alkyl or heteroalkyl. In some embodiments, R³ is unsubstituted alkyl or unsubstituted heteroalkyl. In some embodiments, R³ is alkyl. In some embodiments, R³ is unsubstituted alkyl. In some embodiments, R¹ is methoxy, and R³ is alkyl. In some embodiments, R¹ is methoxy, and R³ is unsubstituted alkyl. In some embodiments, R¹ is hydrogen, and R³ is alkyl. In some embodiments, R¹ is hydrogen, and R³ is unsubstituted alkyl.

In some embodiments, R³ is heteroalkyl. In some embodiments, R³ is unsubstituted heteroalkyl. In some embodiments, R³ is methyl, ethyl, n-propyl, isopropyl, n-butyl, tert-butyl, n-pentyl, or 3-methyl-1-butyl. In some embodiments, R³ is aryl. In some embodiments, R³ is phenyl. In some embodiments, R³ is heterocyclylalkyl. In some embodiments, R³ is 2-pyridyl, 3-pyridyl, 4-pyridyl, 2-pyrimidyl, 4-pyrimidyl, 5-pyrimidyl, or 6-pyrimidyl. In some embodiments, R³ is ethyl. In some embodiments, R¹ is hydrogen, and R³ is ethyl. In some embodiments, R¹ is methoxy, and R³ is ethyl. In some embodiments, R³ alkyl substituted with heteroaryl. In some embodiments, R³ is

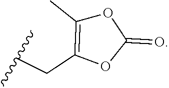 [SMILES: *Cc1oc(=O)oc1C]

In some embodiments, R¹ is methoxy and R³ is

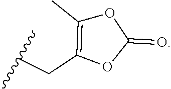 [SMILES: *Cc1oc(=O)oc1C]

In some embodiments, R¹ is hydrogen and R³ is

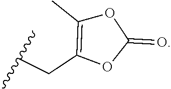 [SMILES: *Cc1oc(=O)oc1C]

In some embodiments, the compound of Formula (Ia) has a formula selected from:

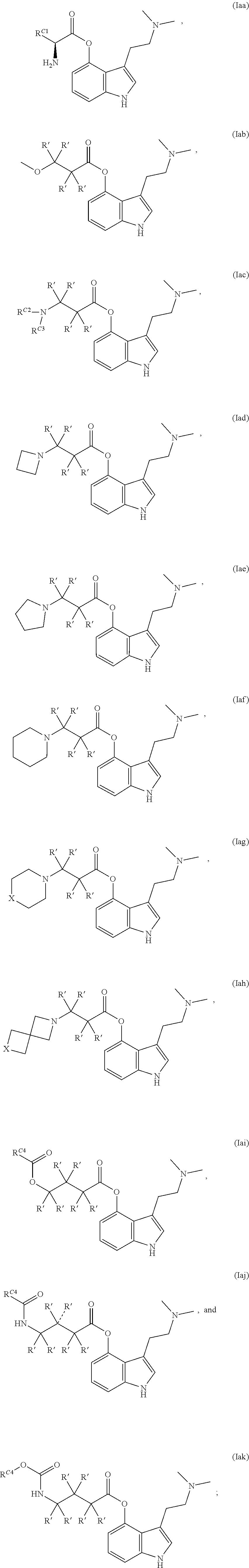

wherein each incidence of R′ is independently hydrogen or methyl; each incidence of X is —CH₂—, —O—, —S—, —SO₂—, —NH—, or —NMe-; R^{C1} is H, Me, CH₂Ph, CH₂CH(CH₃)₂, CH(CH₃)CH₂CH₃, or CH₂CH₂SCH₃; R^{C2} and R^{C3} are each H, CH₃, or CH₂CH₃; and each incidence of R^{C4} is hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, and heteroaryl. In certain embodiments of Formula (Iaa), R^{C1} is H, Me, CH₂Ph, CH₂CH(Me)₂, CH(CH₃)CH₂CH₃, or CH₂CH₂SCH₃. In certain embodiments of Formula (Iac), R^{C2} and R^{C3} are each H, CH₃, or CH₂CH₃. In certain embodiments of Formula (lac), R^{C2} and R^{C3} are each CH₃. In certain embodiments, R^{C4} is selected from hydrogen, —CD₃, Et, n-Pr, iPr, tBu, n-pentyl, iso-amyl, n-hexyl, n-heptyl, n-octyl, n-nonyl, cyclopropyl, cyclobutyl, cyclopentyl, cyclohexyl, cycloheptyl, cyclooctyl, CH₂CF₃, —CH₂-cyclopropyl, Ph, 2-pyridyl, 3-pyridyl, 4-pyridyl, 2-pyrimidyl, 4-pyrimidyl, 5-pyrimidyl, and 6-pyrimidyl.

In some embodiments, the compound of Formula (Ia) is selected from the group consisting of:

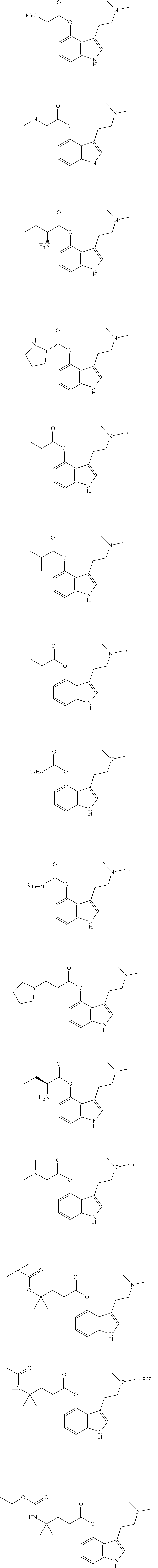

### Trimethyllock Prodrugs

In some embodiments, the compound of Formula (Ia) has the structure of Formula (Ial):

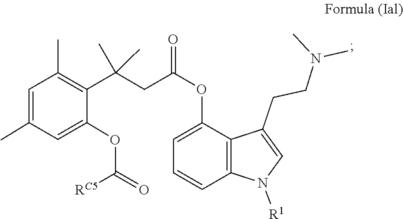

wherein R^{C5} is selected from hydrogen, —CD₃, Et, n-Pr, iPr, tBu, n-pentyl, iso-amyl, n-hexyl, n-heptyl, n-octyl, n-nonyl, cyclopropyl, cyclobutyl, cyclopentyl, cyclohexyl, cycloheptyl, cyclooctyl, CH₂CF₃, —CH₂-cyclopropyl, Ph, 2-pyridyl, 3-pyridyl, 4-pyridyl, 2-pyrimidyl, 4-pyrimidyl, 5-pyrimidyl, and 6-pyrimidyl.

In some embodiments, the compound of Formula (Ial) is selected from the group consisting of:

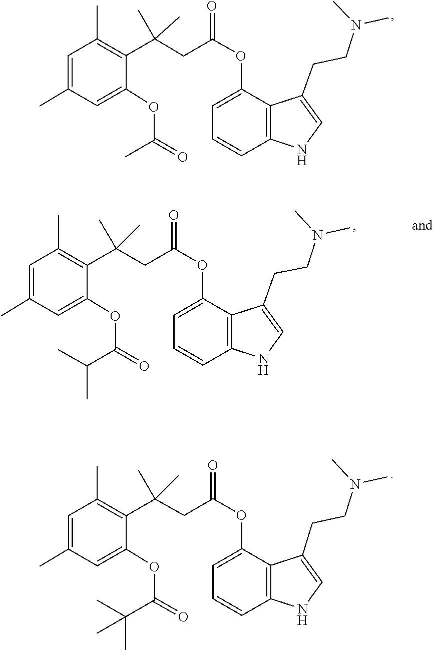

### Carbamate Prodrugs

In some embodiments, the compound of Formula (I) has the structure of Formula (Ic):

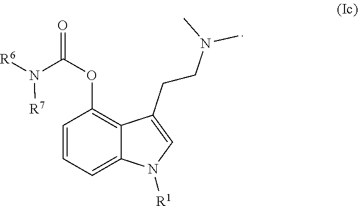

In some embodiments, R⁶ and R⁷ together with the atom to which they are attached form a heterocyclylalkyl ring or a heteroaryl ring that is unsubstituted or substituted with one or more R^{A}.

In some embodiments, R⁶ and R⁷ together with the atom to which they are attached form a heterocyclylalkyl that is unsubstituted. In some embodiments, R⁶ and R⁷ together with the atom to which they are attached form a

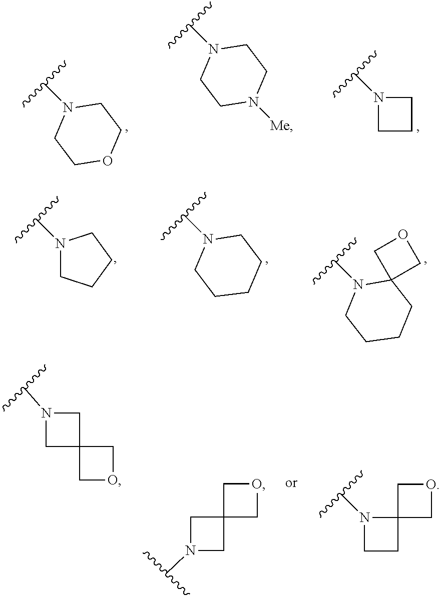

In some embodiments, R⁶ and R⁷ together with the atom to which they are attached form aziridinyl, oxetanyl, azetidinyl, tetrahydrofuranyl, pyrrolidinyl, piperidinyl, piperazinyl, morpholinyl, thiomorpholinyl, thiomorpholinyl dioxide, diazinanyl,

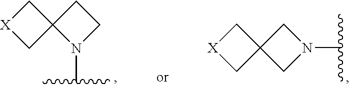

wherein X is —CH₂—, —O—, —S—, —SO₂, —NH—, or —NMe. In some embodiments, R⁶ and R⁷ together with the atom to which they are attached form

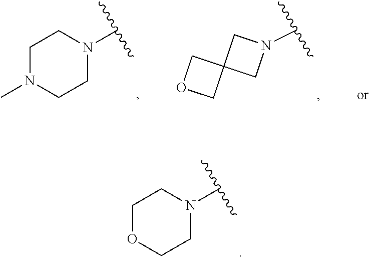

In some embodiments, R⁶ and R⁷ together with the atom to which they are attached form unsubstituted or substituted piperidinyl. In some embodiments, R⁶ and R⁷ together with the atom to which they are attached form unsubstituted or substituted 1-piperidinyl.

In some embodiments, the compound of Formula (Ic) has a formula selected from:

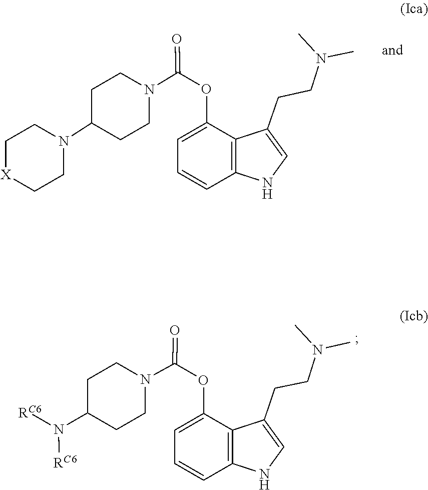

wherein X is —CH₂—, —O—, —S—, —SO₂, —NH—, or —NMe, and each R^{C6} is independently hydrogen, —CH₃, —CD₃, or —CH₂CH₃.

In some embodiments, the compound of Formula (Ic) is selected from the group consisting of:

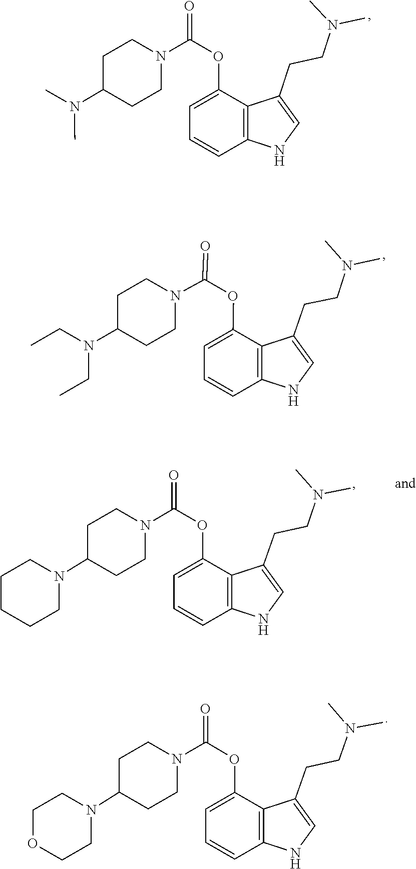

In some embodiments, R⁷ is alkyl substituted with —C(O)OR¹³, wherein R¹³ is hydrogen or alkyl. In some embodiments, R⁷ is alkyl substituted with —C(O)OR¹³, wherein R¹³ is hydrogen, methyl, ethyl, or tert-butyl.

In some embodiments, each of R⁶ and R⁷ is independently hydrogen, alkyl, heteroalkyl, cycloalkyl, heterocyclylalkyl, aryl, or heteroaryl, wherein alkyl, heteroalkyl, cycloalkyl, heterocyclylalkyl, aryl, or heteroaryl is unsubstituted or substituted with one or more R^{A}.

In some embodiments, R⁶ is hydrogen, and R⁷ is hydrogen, alkyl, heteroalkyl, cycloalkyl, heterocyclylalkyl, aryl, or heteroaryl, wherein alkyl, heteroalkyl, cycloalkyl, heterocyclylalkyl, aryl, or heteroaryl is unsubstituted or substituted with one or more R^{A}. In some embodiments, R⁶ is hydrogen, and R⁷ is unsubstituted or substituted alkyl, heteroalkyl, cycloalkyl, or heterocyclylalkyl. In some embodiments, R⁶ is hydrogen, and R⁷ is unsubstituted or substituted alkyl. In some embodiments, R⁶ is hydrogen, and R⁷ is unsubstituted alkyl. In some embodiments, R⁶ is hydrogen, and R⁷ is methyl, ethyl, n-propyl, isopropyl, n-butyl, tert-butyl, n-pentyl, 3-methyl-1-butyl, or —C₁₀H₂₁. In some embodiments, R⁶ is hydrogen, and R⁷ is alkyl substituted with heterocyclylalkyl. In some embodiments, R⁶ is hydrogen, and R⁷ is alkyl substituted with aziridinyl, oxetanyl, azetidinyl, tetrahydrofuranyl, pyrrolidinyl, piperidinyl, piperazinyl, morpholinyl, thiomorpholinyl, thiomorpholinyl dioxide, diazinanyl,

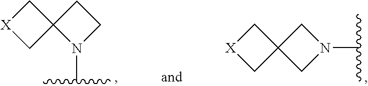

wherein X is —CH₂—, —O—, —S—, —SO₂, —NH—, or NMe.

In some embodiments, R⁶ is methyl.

In some embodiments, the compound of Formula (Ic) has a structure of Formula (Icc):

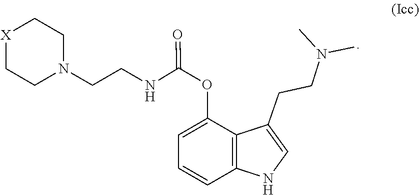

In some embodiments, the compound of Formula (Ic) is selected from the group consisting of:

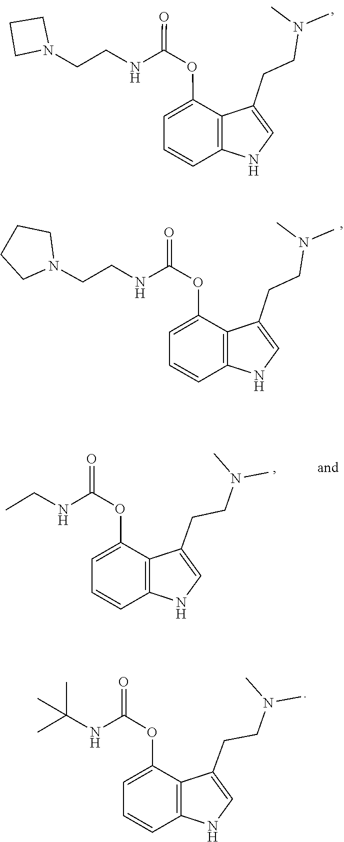

### Carbonate Prodrugs

In some embodiments, the compound of Formula (I) has the structure of Formula (Ib):

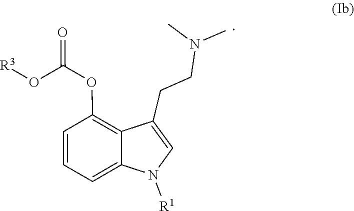

In some embodiments, R³ is hydrogen, alkyl, heteroalkyl, cycloalkyl, heterocyclylalkyl, aryl, or heteroaryl, wherein alkyl, heteroalkyl, cycloalkyl, heterocyclylalkyl, aryl, or heteroaryl is unsubstituted or substituted with one or more R^{A}.

In some embodiments, R³ is unsubstituted or substituted alkyl, heteroalkyl, cycloalkyl, or heterocyclylalkyl.

In some embodiments, R³ is unsubstituted or substituted alkyl. In some embodiments, R³ is alkyl substituted with one or more substituent R^{A}, and wherein each R^{A} is independently selected from alkyl, heteroalkyl, cycloalkyl, heterocyclylalkyl, aryl, heteroaryl, an amino acid side chain, —OR¹³, —N(R¹⁸)R¹⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹], —C(O)N(R¹⁸)R¹⁹, —OC(O)N(R¹⁸)R¹⁹, or —OP(O)OR²⁰(OR²¹).

In some embodiments, R³ is unsubstituted alkyl. In some embodiments, R³ is methyl, ethyl, n-propyl, isopropyl, n-butyl, tert-butyl, n-pentyl, 3-methyl-1-butyl, or —C₁₀H₂₁.

In some embodiments, R³ is alkyl substituted with —N(R¹⁸)R¹⁹, wherein each of R¹⁸ and R¹⁹ is independently hydrogen or methyl.

In some embodiments, R³ is alkyl substituted with —N(R¹³)C(O)R¹⁴, wherein each of R¹³ and R¹⁴ is independently hydrogen or methyl.

In some embodiments, R³ is alkyl substituted with —N(R¹³)C(O)R¹⁴, wherein each of R¹³ is hydrogen or methyl, and R¹⁴ is hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, and heteroaryl is unsubstituted or further substituted with one or more halogen, amino, cyano, hydroxyl, alkyl, acetyl, or benzoyl.

In some embodiments, R³ is alkyl substituted with —N(R¹³)C(O)OR¹⁴, wherein each of R¹³ and R¹⁴ is independently hydrogen, methyl, or ethyl.

In some embodiments, R³ is alkyl substituted with one or more —OC(O)R¹⁵. In some embodiments, R³ is isopropyl substituted with two —OC(O)R¹⁵ wherein each R¹⁵ is alkyl. In some embodiments, R³ is alkyl substituted with —OC(O)R¹⁵, wherein R¹⁵ is hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, and heteroaryl is unsubstituted or further substituted with one or more halogen, amino, cyano, hydroxyl, alkyl, acetyl, or benzoyl.

In some embodiments, R³ is heterocyclylalkyl.

In some embodiments, R³ is selected from aziridinyl, oxetanyl, azetidinyl, tetrahydrofuranyl, pyrrolidinyl, piperidinyl, piperazinyl, morpholinyl, thiomorpholinyl, thiomorpholinyl dioxide, diazinanyl,

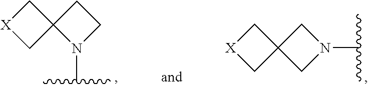

wherein X is —CH₂—, —O—, —S—, —SO₂, —NH—, or —NMe.

In some embodiments, R³ is alkyl substituted with heterocyclylalkyl. In some embodiments, R³ is alkyl substituted with aziridinyl, oxetanyl, azetidinyl, tetrahydrofuranyl, pyrrolidinyl, piperidinyl, piperazinyl, morpholinyl, thiomorpholinyl, thiomorpholinyl dioxide, diazinanyl,

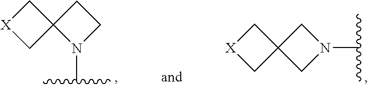

wherein X is —CH₂—, —O—, —S—, —SO₂, —NH—, or —NMe.

In some embodiments, R³ is heteroalkyl.

In some embodiments, R³ is unsubstituted or substituted aryl (e.g., phenyl). In some embodiments, R³ is substituted phenyl. In some embodiments, R³ is phenyl substituted with —OC(O)R¹⁸, wherein R¹⁸ is hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl.

In some embodiments, R³ is selected from alkyl, cycloalkyl, substituted alkyl, substituted cycloalkyl, vinyl, aryl, heteroaryl, substituted aryl, and substituted heteroaryl. In some embodiments, R³ is selected from hydrogen, —CD₃, Et, n-Pr, iPr, tBu, n-pentyl, iso-amyl, n-hexyl, n-heptyl, n-octyl, n-nonyl, cyclopropyl, cyclobutyl, cyclopentyl, cyclohexyl, cycloheptyl, cyclooctyl, CH₂CF₃, —CH₂-cyclopropyl, Ph, 2-pyridyl, 3-pyridyl, 4-pyridyl, 2-pyrimidyl, 4-pyrimidyl, 5-pyrimidyl, and 6-pyrimidyl.

In some embodiments, R³ is alkyl or heteroalkyl. In some embodiments, R³ is unsubstituted alkyl or unsubstituted heteroalkyl. In some embodiments, R³ is alkyl. In some embodiments, R³ is unsubstituted alkyl. In some embodiments, R¹ is methoxy, and R³ is alkyl. In some embodiments, R¹ is methoxy, and R³ is unsubstituted alkyl. In some embodiments, R¹ is hydrogen, and R³ is alkyl. In some embodiments, R¹ is hydrogen, and R³ is unsubstituted alkyl.

In some embodiments, R³ is heteroalkyl. In some embodiments, R³ is unsubstituted heteroalkyl. In some embodiments, R³ is methyl, ethyl, n-propyl, isopropyl, n-butyl, tert-butyl, n-pentyl, or 3-methyl-1-butyl. In some embodiments, R³ is aryl. In some embodiments, R³ is phenyl. In some embodiments, R³ is heterocyclylalkyl. In some embodiments, R³ is 2-pyridyl, 3-pyridyl, 4-pyridyl, 2-pyrimidyl, 4-pyrimidyl, 5-pyrimidyl, or 6-pyrimidyl. In some embodiments, R³ is ethyl. In some embodiments, R¹ is hydrogen, and R³ is ethyl. In some embodiments, R¹ is methoxy, and R³ is ethyl. In some embodiments, R³ alkyl substituted with heteroaryl. In some embodiments, R³ is

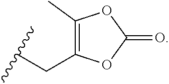 [SMILES: *Cc1oc(=O)oc1C]

In some embodiments, R¹ is methoxy and R³ is

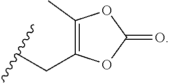 [SMILES: *Cc1oc(=O)oc1C]

In some embodiments, R¹ is hydrogen and R³ is

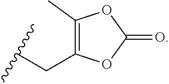 [SMILES: *Cc1oc(=O)oc1C]

In some embodiments, the compound of Formula (Ib) has a structure of the formula selected from:

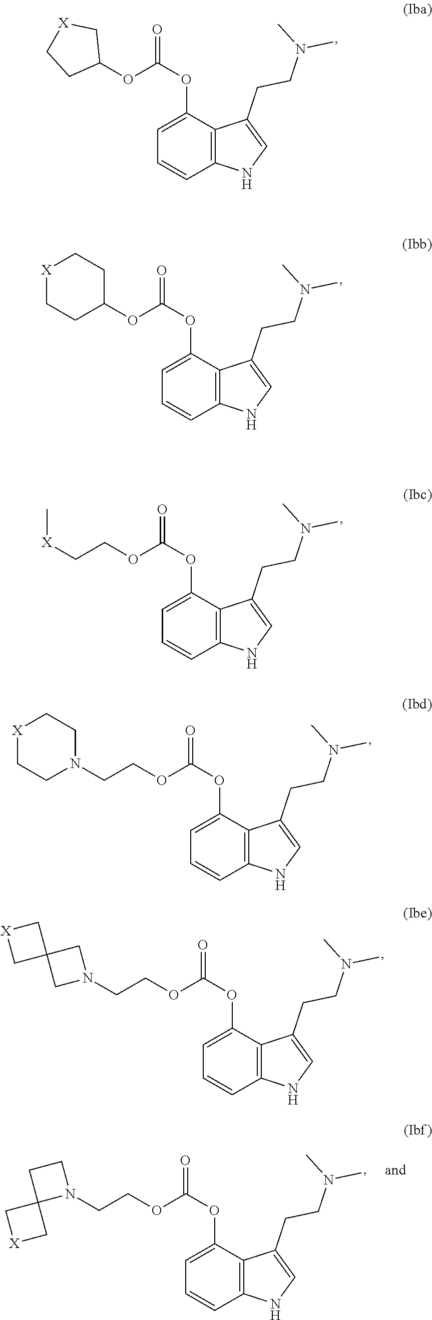

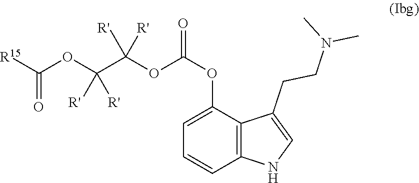

wherein X is —CH₂—, —O—, —S—, —SO₂, —NH—, or —NMe, each R′ is independently hydrogen or —CH₃; and R¹⁵ is defined herein above.

In some embodiments, the compound of Formula (Ib) is selected from the group consisting of:

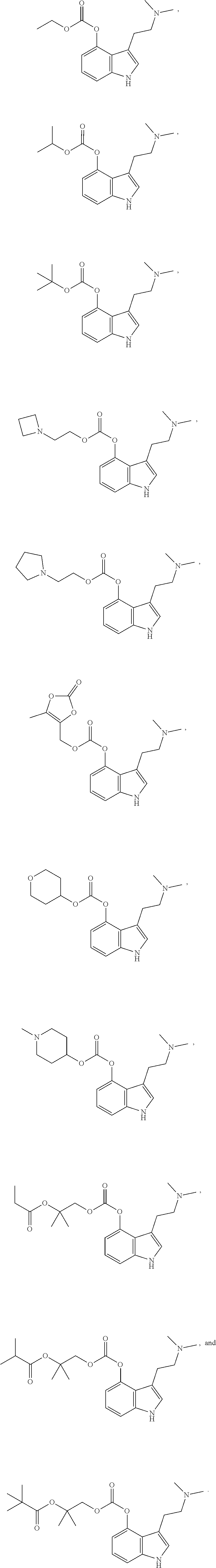

### Acyloxymethyl Prodrugs

In some embodiments, the compound of Formula (I) has the structure of Formula (Id):

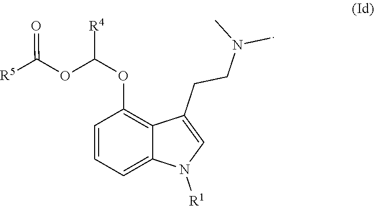

In some embodiments, R⁴ is hydrogen, unsubstituted or substituted alkyl, heteroalkyl, cycloalkyl, or heterocyclylalkyl. In some embodiments, R⁴ is unsubstituted or substituted alkyl. In some embodiments, R⁴ is methyl, ethyl, n-propyl, isopropyl, n-butyl, tert-butyl, n-pentyl, 3-methyl-1-butyl, or —C₁₀H₂₁. In some embodiments, R⁴ is hydrogen, methyl, or isopropyl. In some embodiments, R⁴ is hydrogen. In some embodiments, R⁴ is methyl. In some embodiments, R⁴ is isopropyl.

In some embodiments, R⁵ is unsubstituted or substituted alkyl, heteroalkyl, cycloalkyl, or heterocyclylalkyl.

In some embodiments, R⁵ is unsubstituted or substituted alkyl. In some embodiments, R⁵ is alkyl substituted with one or more substituent R^{A}, and wherein each R^{A} is independently selected from alkyl, heteroalkyl, cycloalkyl, heterocyclylalkyl, aryl, heteroaryl, an amino acid side chain, —OR¹³, —N(R¹⁸)R¹⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹], —C(O)N(R¹⁸)R¹⁹, —OC(O)N(R¹⁸)R¹⁹, or —OP(O)OR²⁰(OR²¹).

In some embodiments, R⁵ is unsubstituted alkyl. In some embodiments, R⁵ is methyl, ethyl, n-propyl, isopropyl, n-butyl, tert-butyl, n-pentyl, 3-methyl-1-butyl, or —C₁₀H₂₁.

In some embodiments, R⁵ is alkyl substituted with —N(R¹⁸)R¹⁹, wherein each of R¹⁸ and R¹⁹ is independently hydrogen or methyl.

In some embodiments, R⁵ is alkyl substituted with —N(R¹³)C(O)R¹⁴, wherein each of R¹³ and R¹⁴ is independently hydrogen or methyl.

In some embodiments, R⁵ is alkyl substituted with —N(R¹³)C(O)R¹⁴, wherein each of R¹³ is hydrogen or methyl, and R¹⁴ is hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, and heteroaryl is unsubstituted or further substituted with one or more halogen, amino, cyano, hydroxyl, alkyl, acetyl, or benzoyl.

In some embodiments, R⁵ is alkyl substituted with —N(R¹³)C(O)OR¹⁴, wherein each of R¹³ and R¹⁴ is independently hydrogen, methyl, or ethyl.

In some embodiments, R⁵ is alkyl substituted with —OC(O)R¹⁵, wherein R¹⁵ is hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, and heteroaryl is unsubstituted or further substituted with one or more halogen, amino, cyano, hydroxyl, alkyl, acetyl, or benzoyl.

In some embodiments, R⁵ is alkyl substituted with —C(O)OR¹⁵. In some embodiments, R⁵ is alkyl substituted with —C(O)OR¹⁵, wherein R¹⁵ is hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, and heteroaryl is unsubstituted or further substituted with one or more halogen, amino, cyano, hydroxyl, alkyl, acetyl, or benzoyl. In some embodiments, R¹³ is hydrogen or alkyl. In some embodiments, R¹³ is hydrogen, methyl, ethyl, or tert-butyl.

In some embodiments, R⁵ is heterocyclylalkyl.

In some embodiments, R⁵ is selected from aziridinyl, oxetanyl, azetidinyl, tetrahydrofuranyl, pyrrolidinyl, piperidinyl, piperazinyl, morpholinyl, thiomorpholinyl, thiomorpholinyl dioxide, diazinanyl,

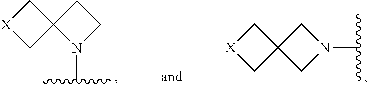

wherein X is —CH₂—, —O—, —S—, —SO₂, —NH—, or —NMe.

In some embodiments, R⁵ is optionally substituted piperidinyl. In some embodiments, R⁵ is

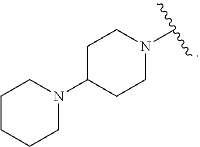 [SMILES: *N1CCC(N2CCCCC2)CC1]

In some embodiments, R⁵ is heterocyclylalkyl. In some embodiments, R⁵ is alkyl substituted with aziridinyl, oxetanyl, azetidinyl, tetrahydrofuranyl, pyrrolidinyl, piperidinyl, piperazinyl, morpholinyl, thiomorpholinyl, thiomorpholinyl dioxide, diazinanyl,

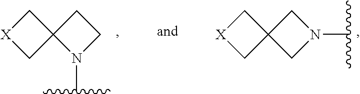

wherein X is —CH₂—, —O—, —S—, —SO₂, —NH—, or —NMe.

In some embodiments, R⁵ is heteroalkyl.

In some embodiments, R⁵ is unsubstituted or substituted aryl (e.g., phenyl). In some embodiments, R⁵ is substituted phenyl. In some embodiments, R⁵ is phenyl substituted with —OC(O)R¹⁸, wherein R¹⁸ is hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl.

In some embodiments, R⁵ is selected from alkyl, cycloalkyl, substituted alkyl, substituted cycloalkyl, vinyl, aryl, heteroaryl, substituted aryl, and substituted heteroaryl. In some embodiments, R⁵ is selected from hydrogen, —CD₃, Et, n-Pr, iPr, tBu, n-pentyl, iso-amyl, n-hexyl, n-heptyl, n-octyl, n-nonyl, cyclopropyl, cyclobutyl, cyclopentyl, cyclohexyl, cycloheptyl, cyclooctyl, CH₂CF₃, —CH₂-cyclopropyl, Ph, 2-pyridyl, 3-pyridyl, 4-pyridyl, 2-pyrimidyl, 4-pyrimidyl, 5-pyrimidyl, and 6-pyrimidyl.

In some embodiments, R⁵ is alkyl or heteroalkyl. In some embodiments, R⁵ is unsubstituted alkyl or unsubstituted heteroalkyl. In some embodiments, R⁵ is alkyl. In some embodiments, R⁵ is unsubstituted alkyl. In some embodiments, R¹ is methoxy, and R⁵ is alkyl. In some embodiments, R¹ is methoxy, and R⁵ is unsubstituted alkyl. In some embodiments, R¹ is hydrogen, and R⁵ is alkyl. In some embodiments, R¹ is hydrogen, and R⁵ is unsubstituted alkyl.

In some embodiments, R⁵ is heteroalkyl. In some embodiments, R⁵ is unsubstituted heteroalkyl. In some embodiments, R⁵ is methyl, ethyl, n-propyl, isopropyl, n-butyl, tert-butyl, n-pentyl, or 3-methyl-1-butyl. In some embodiments, R⁵ is aryl. In some embodiments, R⁵ is phenyl. In some embodiments, R⁵ is heterocyclylalkyl. In some embodiments, R⁵ is 2-pyridyl, 3-pyridyl, 4-pyridyl, 2-pyrimidyl, 4-pyrimidyl, 5-pyrimidyl, or 6-pyrimidyl. In some embodiments, R⁵ is ethyl. In some embodiments, R¹ is hydrogen, and R⁵ is ethyl. In some embodiments, R¹ is methoxy, and R⁵ is ethyl. In some embodiments, R⁵ alkyl substituted with heteroaryl. In some embodiments, R⁵ is

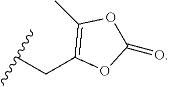 [SMILES: *Cc1oc(=O)oc1C]

In some embodiments, R¹ is methoxy and R⁵ is

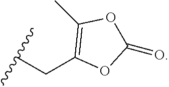 [SMILES: *Cc1oc(=O)oc1C]

In some embodiments, R¹ is hydrogen and R⁵ is

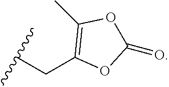 [SMILES: *Cc1oc(=O)oc1C]

In some embodiments, the compound of Formula (Id) has a structure of the formula selected from:

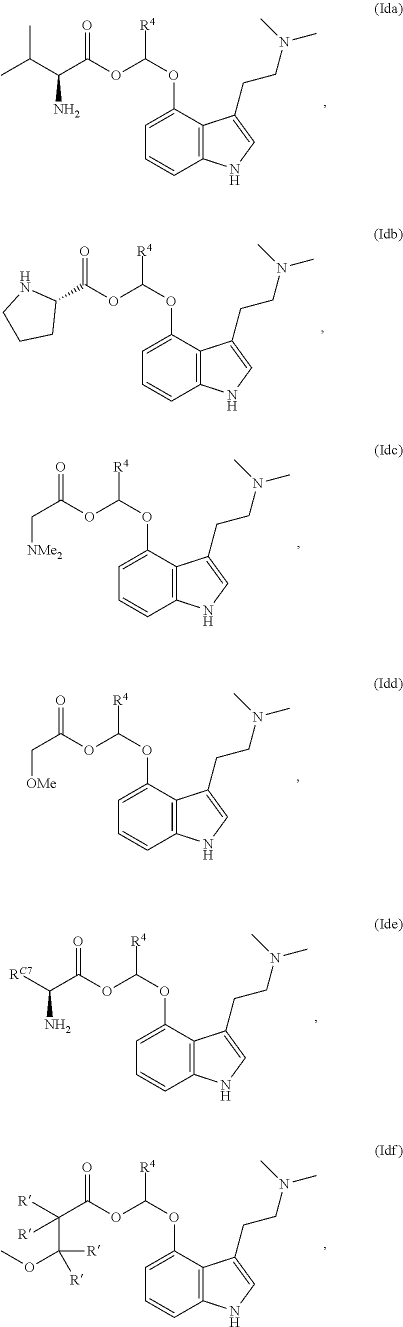

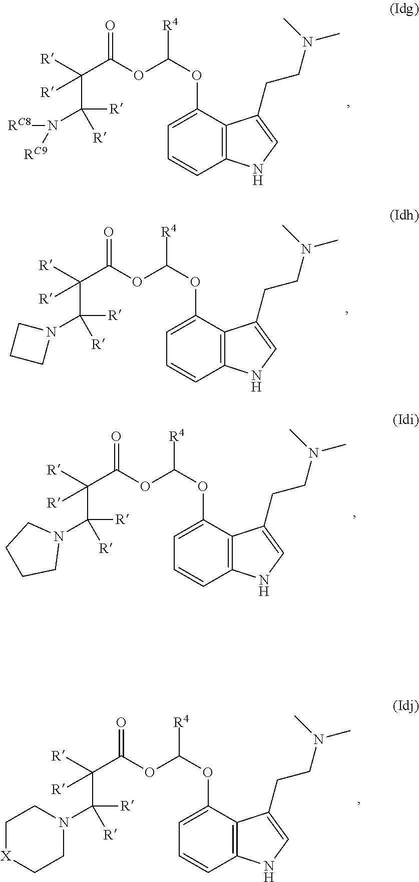

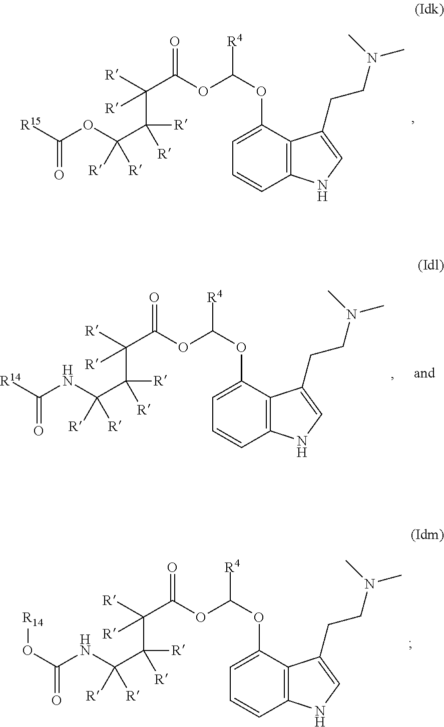

wherein X is —CH₂—, —O—, —S—, —SO₂, —NH—, or —NMe, each R′ is independently hydrogen or —CH₃; RC7 is H, Me, CH₂Ph, CH₂CH(Me)₂, CH(CH₃)CH₂CH₃, or CH₂CH₂SCH₃, R^{C8} and R^{C9} are each H, CH₃, or CH₂CH₃, and R¹⁴ and R¹⁵ are defined herein above.

In some embodiments, the compound of Formula (Id) has a structure of Formula (Ida). In some embodiments, the compound of Formula (Id) has a structure of Formula (Idb). In some embodiments, the compound of Formula (Id) has a structure of Formula (Idc). In some embodiments, the compound of Formula (Id) has a structure of Formula (Idd). In some embodiments, the compound of Formula (Id) has a structure of Formula (Ide). In some embodiments, the compound of Formula (Id) has a structure of Formula (Idf). In some embodiments, the compound of Formula (Id) has a structure of Formula (Idg). In some embodiments, the compound of Formula (Id) has a structure of Formula (Idh). In some embodiments, the compound of Formula (Id) has a structure of Formula (Idi). In some embodiments, the compound of Formula (Id) has a structure of Formula (Idj).

In some embodiments, the compound of Formula (Id) has a structure of Formula (Idk):

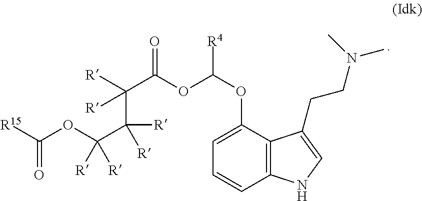

In some embodiments, the compound of Formula (Id) has a structure of Formula (Idl):

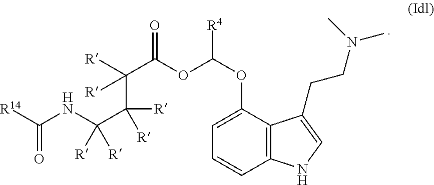

In some embodiments, the compound of Formula (Id) has a structure of Formula (Idm):

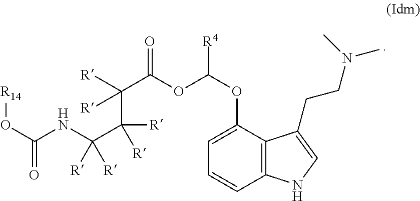

In some embodiments, the compound of Formula (Id) is selected from the group consisting of:

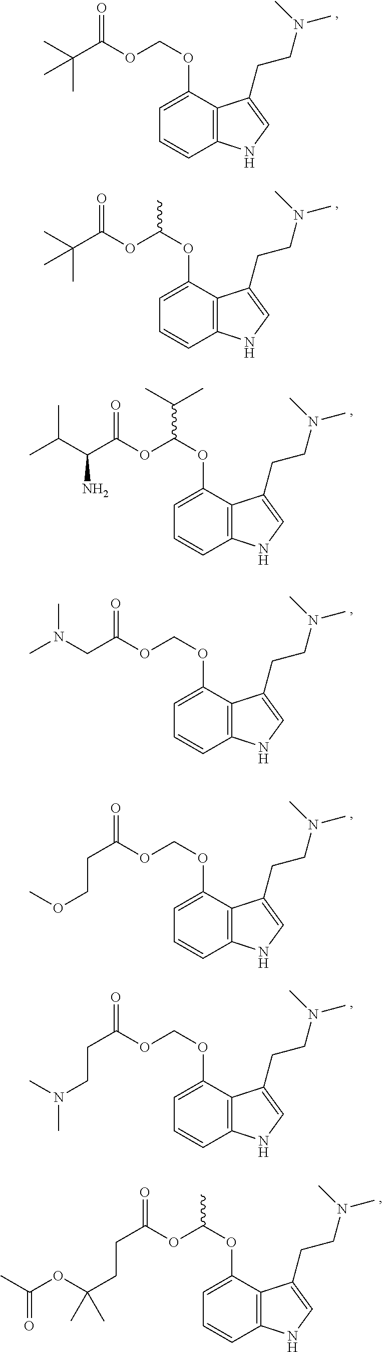

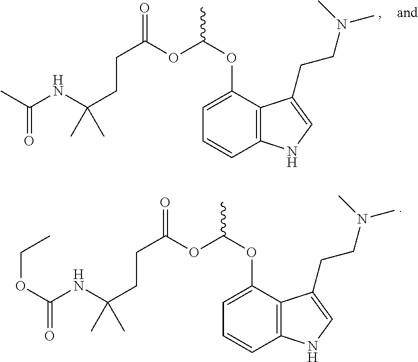

### Alkoxycarbonyloxymethyl Prodrugs

In some embodiments, the compound of Formula (I) has the structure of Formula (Ie):

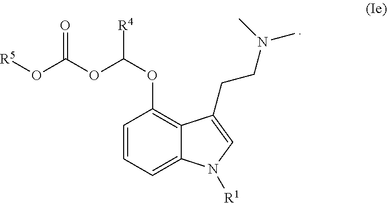

In some embodiments, R⁴ is hydrogen, unsubstituted or substituted hydrogen, alkyl, heteroalkyl, cycloalkyl, or heterocyclylalkyl. In some embodiments, R⁴ is unsubstituted or substituted alkyl. In some embodiments, R⁴ is methyl, ethyl, n-propyl, isopropyl, n-butyl, tert-butyl, n-pentyl, 3-methyl-1-butyl, or —C₁₀H₂₁. In some embodiments, R⁴ is hydrogen, methyl, or isopropyl. In some embodiments, R⁴ is hydrogen. In some embodiments, R⁴ is methyl. In some embodiments, R⁴ is isopropyl.

In some embodiments, R⁵ is unsubstituted or substituted alkyl, heteroalkyl, cycloalkyl, or heterocyclylalkyl.

In some embodiments, R⁵ is unsubstituted or substituted alkyl. In some embodiments, R⁵ is alkyl substituted with one or more substituent R^{A}, and wherein each R^{A} is independently selected from alkyl, heteroalkyl, cycloalkyl, heterocyclylalkyl, aryl, heteroaryl, an amino acid side chain, —OR¹³, —N(R¹⁸)R¹⁹, —C(O)OR³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹], —C(O)N(R¹⁸)R¹⁹, —OC(O)N(R¹⁸)R¹⁹, or —OP(O)OR²⁰(OR²¹).

In some embodiments, R⁵ is unsubstituted alkyl. In some embodiments, R⁵ is methyl, ethyl, n-propyl, isopropyl, n-butyl, tert-butyl, n-pentyl, 3-methyl-1-butyl, or —C₁₀H₂₁.

In some embodiments, R⁵ is alkyl substituted with —N(R¹⁸)R¹⁹, wherein each of R¹⁸ and R¹⁹ is independently hydrogen or methyl.

In some embodiments, R⁵ is alkyl substituted with —N(R¹³)C(O)R¹⁴, wherein each of R¹³ and R¹⁴ is independently hydrogen or methyl.

In some embodiments, R⁵ is alkyl substituted with —N(R¹³)C(O)R¹⁴, wherein each of R¹³ is hydrogen or methyl, and R¹⁴ is hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, and heteroaryl is unsubstituted or further substituted with one or more halogen, amino, cyano, hydroxyl, alkyl, acetyl, or benzoyl.

In some embodiments, R⁵ is alkyl substituted with —N(R¹³)C(O)OR¹⁴, wherein each of R¹³ and R¹⁴ is independently hydrogen, methyl, or ethyl.

In some embodiments, R⁵ is alkyl substituted with —OC(O)R¹⁵, wherein R¹⁵ is hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, and heteroaryl is unsubstituted or further substituted with one or more halogen, amino, cyano, hydroxyl, alkyl, acetyl, or benzoyl.

In some embodiments, R⁵ is heterocyclylalkyl.

In some embodiments, R⁵ is selected from aziridinyl, oxetanyl, azetidinyl, tetrahydrofuranyl, pyrrolidinyl, piperidinyl, piperazinyl, morpholinyl, thiomorpholinyl, thiomorpholinyl dioxide, diazinanyl,

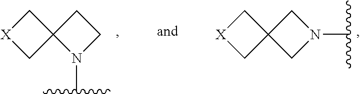

wherein X is —CH₂—, —O—, —S—, —SO₂, —NH—, or —NMe.

In some embodiments, R⁵ is heterocyclylalkyl. In some embodiments, R⁵ is selected from aziridinyl, oxetanyl, azetidinyl, tetrahydrofuranyl, pyrrolidinyl, piperidinyl, piperazinyl, morpholinyl, thiomorpholinyl, thiomorpholinyl dioxide, diazinanyl,

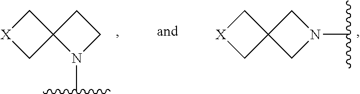

wherein X is —CH₂—, —O—, —S—, —SO₂, —NH—, or —NMe.

In some embodiments, R⁵ is heteroalkyl.

In some embodiments, R⁵ is unsubstituted or substituted aryl (e.g., phenyl). In some embodiments, R⁵ is substituted phenyl. In some embodiments, R⁵ is phenyl substituted with —OC(O)R¹⁸, wherein R¹⁸ is hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl.

In some embodiments, R⁵ is selected from alkyl, cycloalkyl, substituted alkyl, substituted cycloalkyl, vinyl, aryl, heteroaryl, substituted aryl, and substituted heteroaryl. In some embodiments, R⁵ is selected from hydrogen, —CD₃, Et, n-Pr, iPr, tBu, n-pentyl, iso-amyl, n-hexyl, n-heptyl, n-octyl, n-nonyl, cyclopropyl, cyclobutyl, cyclopentyl, cyclohexyl, cycloheptyl, cyclooctyl, CH₂CF₃, —CH₂-cyclopropyl, Ph, 2-pyridyl, 3-pyridyl, 4-pyridyl, 2-pyrimidyl, 4-pyrimidyl, 5-pyrimidyl, and 6-pyrimidyl.

In some embodiments, R⁵ is alkyl or heteroalkyl. In some embodiments, R⁵ is unsubstituted alkyl or unsubstituted heteroalkyl. In some embodiments, R⁵ is alkyl. In some embodiments, R⁵ is unsubstituted alkyl. In some embodiments, R¹ is methoxy, and R⁵ is alkyl. In some embodiments, R¹ is methoxy, and R⁵ is unsubstituted alkyl. In some embodiments, R¹ is hydrogen, and R⁵ is alkyl. In some embodiments, R¹ is hydrogen, and R⁵ is unsubstituted alkyl.

In some embodiments, R⁵ is heteroalkyl. In some embodiments, R⁵ is unsubstituted heteroalkyl. In some embodiments, R⁵ is methyl, ethyl, n-propyl, isopropyl, n-butyl, tert-butyl, n-pentyl, or 3-methyl-1-butyl. In some embodiments, R⁵ is aryl. In some embodiments, R⁵ is phenyl. In some embodiments, R⁵ is heterocyclylalkyl. In some embodiments, R⁵ is 2-pyridyl, 3-pyridyl, 4-pyridyl, 2-pyrimidyl, 4-pyrimidyl, 5-pyrimidyl, or 6-pyrimidyl. In some embodiments, R⁵ is ethyl. In some embodiments, R¹ is hydrogen, and R⁵ is ethyl. In some embodiments, R¹ is methoxy, and R⁵ is ethyl. In some embodiments, R⁵ alkyl substituted with heteroaryl. In some embodiments, R⁵ is

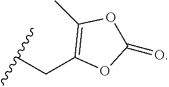 [SMILES: *Cc1oc(=O)oc1C]

In some embodiments, R¹ is methoxy and R⁵ is

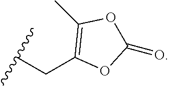 [SMILES: *Cc1oc(=O)oc1C]

In some embodiments, R¹ is hydrogen and R⁵ is

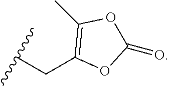 [SMILES: *Cc1oc(=O)oc1C]

In some embodiments, R⁵ is morpholinyl, isopropyl, or ethyl.

In some embodiments, the compound of Formula (Ie) has a structure of the formula selected from:

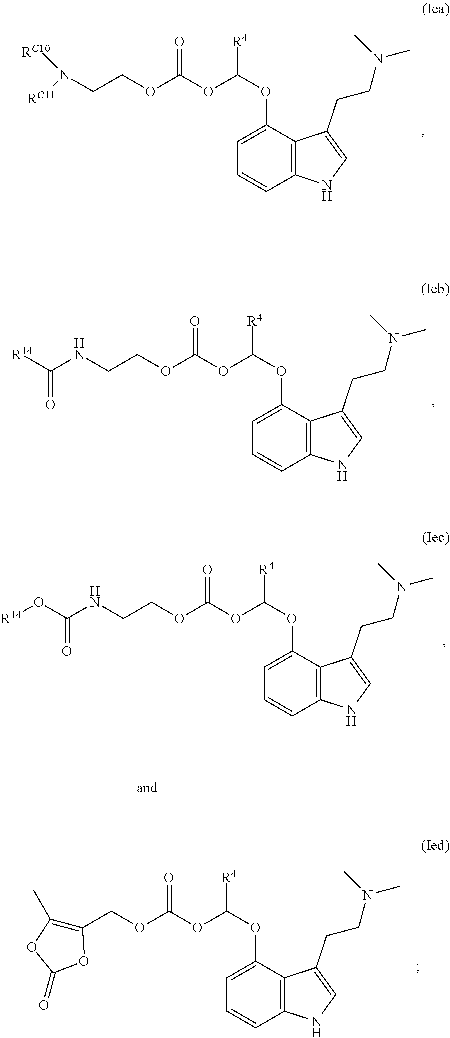

wherein R^{C10} and R^{C11} are each H, CH₃, or CH₂CH₃; and R⁴ and R¹⁴ are defined herein above.

In some preferred embodiments, the compound of Formula (Ie) is selected from the group consisting of:

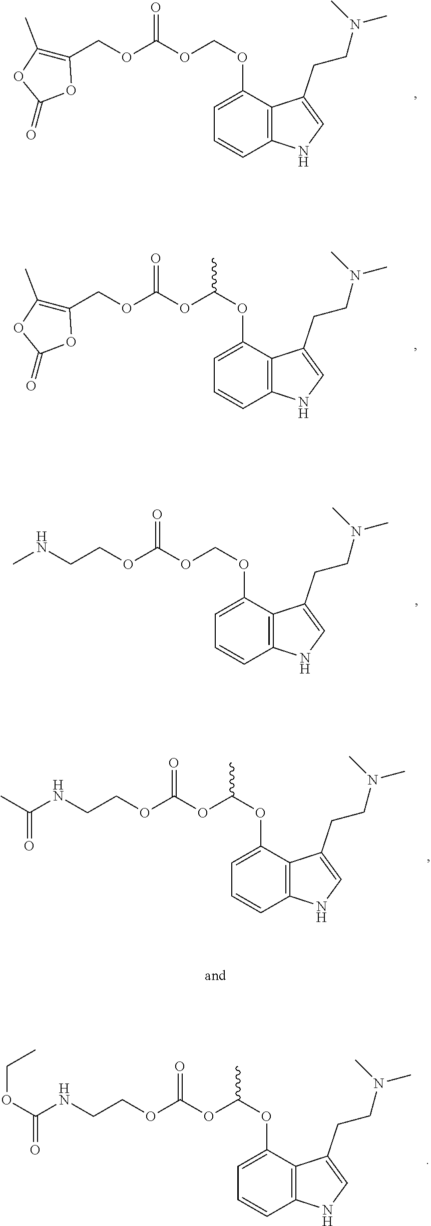

### Aminocarbonyloxymethyl Prodrugs

In some embodiments, the compound of Formula (I) has the structure of Formula (If):

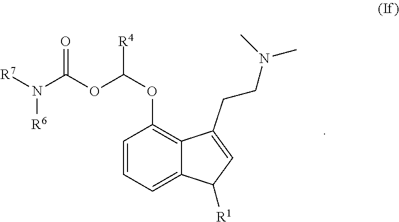

In some embodiments, R⁴ is hydrogen, unsubstituted or substituted alkyl, heteroalkyl, cycloalkyl, or heterocyclylalkyl. In some embodiments, R⁴ is unsubstituted or substituted alkyl. In some embodiments, R⁴ is methyl, ethyl, n-propyl, isopropyl, n-butyl, tert-butyl, n-pentyl, 3-methyl-1-butyl, or —C₁₀H₂₁. In some embodiments, R⁴ is hydrogen, methyl, or isopropyl. In some embodiments, R⁴ is hydrogen. In some embodiments, R⁴ is methyl. In some embodiments, R⁴ is isopropyl.

In some embodiments, R⁶ and R⁷ together with the atom to which they are attached form a heterocyclylalkyl ring or a heteroaryl ring that is unsubstituted or substituted with one or more R^{A}.

In some embodiments, R⁶ and R⁷ together with the atom to which they are attached form a heterocyclylalkyl that is unsubstituted. In some embodiments, R⁶ and R⁷ together with the atom to which they are attached form a

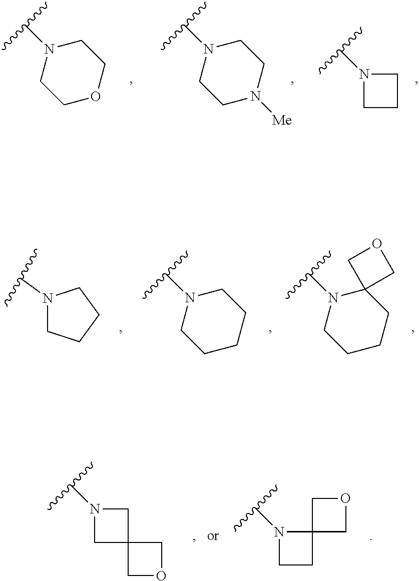

In some embodiments, R⁶ and R⁷ together with the atom to which they are attached form aziridinyl, oxetanyl, azetidinyl, tetrahydrofuranyl, pyrrolidinyl, piperidinyl, piperazinyl, morpholinyl, thiomorpholinyl, thiomorpholinyl dioxide, diazinanyl,

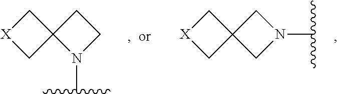

wherein X is —CH₂—, —O—, —S—, —SO₂, —NH—, or —NMe.

In some embodiments, R⁶ and R⁷ together with the atom to which they are attached form unsubstituted or substituted piperidinyl. In some embodiments, R⁶ and R⁷ together with the atom to which they are attached form unsubstituted or substituted 1-piperidinyl. In some embodiments, R⁶ and R⁷ together with the atom to which they are attached form

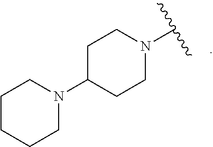 [SMILES: *N1CCC(N2CCCCC2)CC1]

In some embodiments, the compound of Formula (If) has a structure of Formula (Ifa) or Formula (Ifb):

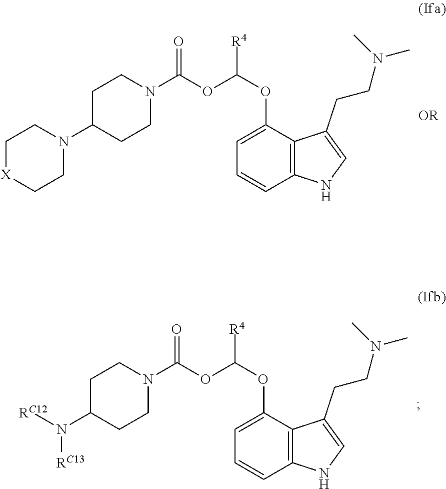

wherein X is —CH₂—, —O—, —S—, —SO₂, —NH—, or —NMe; R^{C12} and R^{C13} are each H, CH₃, CD₃, or CH₂CH₃; and R⁴ is defined herein above.

In some embodiments, the compound of Formula (If) is selected from the group consisting of:

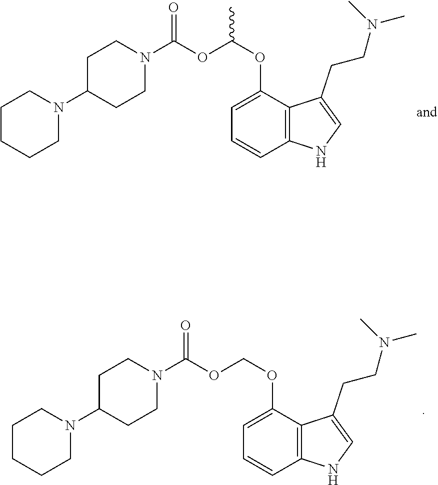 [SMILES: CC(OC(=O)N1CCC(N2CCCCC2)CC1)Oc1cccc2[nH]cc(CCN(C)C)c12 ; CN(C)CCc1c[nH]c2cccc(OCOC(=O)N3CCC(N4CCCCC4)CC3)c12]

In some embodiments, each of R⁶ and R⁷ is independently hydrogen, alkyl, heteroalkyl, cycloalkyl, heterocyclylalkyl, aryl, or heteroaryl, wherein alkyl, heteroalkyl, cycloalkyl, heterocyclylalkyl, aryl, or heteroaryl is unsubstituted or substituted with one or more R^{A}.

In some embodiments, R⁶ is hydrogen, and R⁷ is hydrogen, alkyl, heteroalkyl, cycloalkyl, heterocyclylalkyl, aryl, or heteroaryl, wherein alkyl, heteroalkyl, cycloalkyl, heterocyclylalkyl, aryl, or heteroaryl is unsubstituted or substituted with one or more R^{A}. In some embodiments, R⁶ is hydrogen, and R⁷ is unsubstituted or substituted alkyl, heteroalkyl, cycloalkyl, or heterocyclylalkyl. In some embodiments, R⁶ is hydrogen, and R⁷ is unsubstituted or substituted alkyl. In some embodiments, R⁶ is hydrogen, and R⁷ is unsubstituted alkyl. In some embodiments, R⁶ is hydrogen, and R⁷ is methyl, ethyl, n-propyl, isopropyl, n-butyl, tert-butyl, n-pentyl, 3-methyl-1-butyl, or —C₁₀H₂₁. In some embodiments, R⁶ is hydrogen, and R⁷ is alkyl substituted with heterocyclylalkyl. In some embodiments, R⁶ is hydrogen, and R⁷ is alkyl substituted with aziridinyl, oxetanyl, azetidinyl, tetrahydrofuranyl, pyrrolidinyl, piperidinyl, piperazinyl, morpholinyl, thiomorpholinyl, thiomorpholinyl dioxide, diazinanyl,

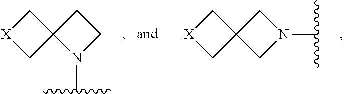

wherein X is —CH₂—, —O—, —S—, —SO₂, —NH—, or NMe.

In some embodiments, the compound of Formula (If) is a compound of Formula (If′):

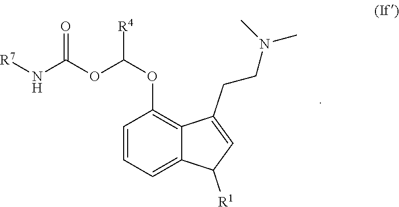

In some embodiments, the compound of Formula (If) is selected from the group consisting of:

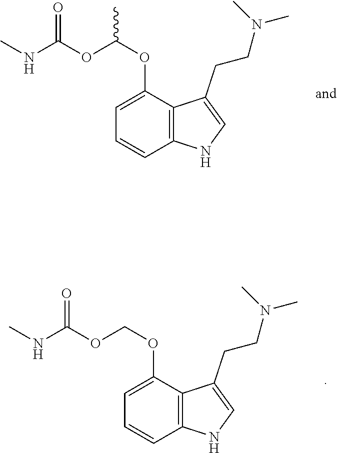

### Phosphonate Prodrugs

In some embodiments, the compound of Formula (I) has the structure of Formula (Ih):

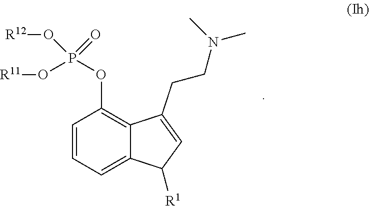

In some embodiments, R¹¹ and R¹² is independently unsubstituted or substituted alkyl, heteroalkyl, cycloalkyl, or heterocyclylalkyl. In some embodiments, R¹¹ and R¹² is independently hydrogen, unsubstituted or substituted alkyl, heteroalkyl, cycloalkyl, or heterocyclylalkyl. In some embodiments, each of R¹¹ and R¹² is independently hydrogen or unsubstituted or substituted alkyl. In some embodiments, each of R¹¹ and R¹² is independently unsubstituted or substituted alkyl.

In some embodiments, each of R¹¹ and R¹² is independently alkyl substituted with one or more substituent R^{A}, and wherein each R^{A} is independently selected from alkyl, heteroalkyl, cycloalkyl, heterocyclylalkyl, aryl, heteroaryl, an amino acid side chain, —OR¹³, —N(R¹⁸)R¹⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹], —C(O)N(R¹⁸)R¹⁹, —OC(O)N(R¹⁸)R¹⁹, or —OP(O)OR²⁰(OR²¹).

In some embodiments, each of R¹¹ and R¹² is independently unsubstituted alkyl.

In some embodiments, each of R¹¹ and R¹² is independently methyl, ethyl, n-propyl, isopropyl, n-butyl, tert-butyl, n-pentyl, 3-methyl-1-butyl, or —C₁₀H₂₁.

In some embodiments, each of R¹¹ and R¹² is independently alkyl substituted with —OC(O)R^{5A}, wherein R^{5A} is hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, and heteroaryl is unsubstituted or further substituted with one or more halogen, amino, cyano, hydroxyl, alkyl, acetyl, or benzoyl.

In some embodiments, R¹¹ and R¹² is independently alkyl substituted with —OC(O)OR¹⁶, wherein R¹⁶ is hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, and heteroaryl is unsubstituted or further substituted with one or more halogen, amino, cyano, hydroxyl, alkyl, acetyl, or benzoyl. In some embodiments, R¹⁶ is hydrogen or alkyl. In some embodiments, R¹⁶ is hydrogen, methyl, ethyl, isopropyl or tert-butyl.

In some embodiments, each of R¹¹ and R¹² is independently heteroalkyl.

In some embodiments, each of R¹¹ and R¹² is independently unsubstituted or substituted aryl (e.g., phenyl).

In some embodiments, the compound of Formula (Ih) has structure of Formula (Ih′):

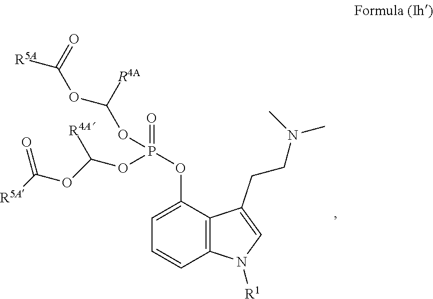

wherein R^{4A} and R^{4A}′ are each independently hydrogen or alkyl, and R^{5A} and R^{5A}′ are each independently hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, and heteroaryl is unsubstituted or further substituted with one or more halogen, amino, cyano, hydroxyl, alkyl, acetyl, or benzoyl.

In some embodiments, the compound of Formula (Ih) is a compound selected from the group consisting of:

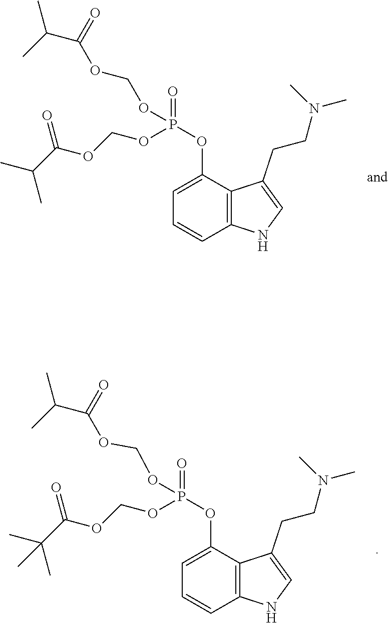

### Sulfamate Prodrugs

In some embodiments, the compound of Formula (I) has the structure of Formula (Ig):

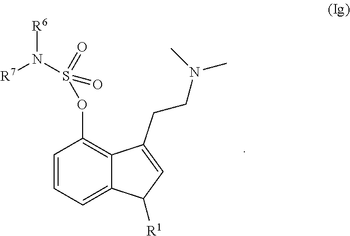

In some embodiments, each of R⁶ and R⁷ is independently hydrogen, alkyl, heteroalkyl, cycloalkyl, heterocyclylalkyl, aryl, or heteroaryl, wherein alkyl, heteroalkyl, cycloalkyl, heterocyclylalkyl, aryl, or heteroaryl is unsubstituted or substituted with one or more R^{A}.

In some embodiments, R⁶ and R⁷ are each independently hydrogen or alkyl. In some embodiments, R⁶ and R⁷ are each independently hydrogen or methyl.

In some embodiments, R⁶ and R⁷ are each hydrogen.

In some embodiments, R⁶ and R⁷ together with the atom to which they are attached form a heterocyclylalkyl ring or a heteroaryl ring that is unsubstituted or substituted with one or more R^{A}.

In some embodiments, R⁶ and R⁷ together with the atom to which they are attached form aziridinyl, oxetanyl, azetidinyl, tetrahydrofuranyl, pyrrolidinyl, piperidinyl, piperazinyl, morpholinyl, thiomorpholinyl, thiomorpholinyl dioxide, diazinanyl,

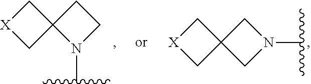

wherein X is —O—, —S—, —SO₂, —NH—, or —NMe.

In some embodiments, the compound of Formula (Ig) is:

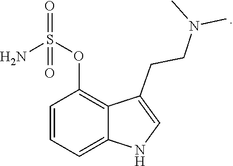 [SMILES: CN(C)CCc1c[nH]c2cccc(OS(N)(=O)=O)c12]

### Glyceride Prodrugs

In some embodiments, the compound of Formula (I) has the structure of Formula (Ib′):

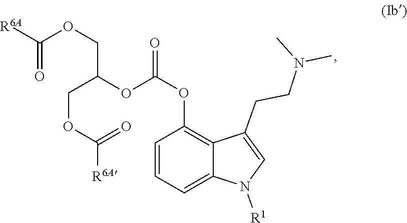

wherein R^{6A} and R^{6A}′ are each independently hydrogen or alkyl.

In some embodiments, R^{6A} and R^{6A}′ are each independently —CH₃, —C₂H₅, C₃H₇, C₄H₉, C₅H₁₁, C₆H₁₃, C₇H₁₅, C₈H₁₇, C₉H₁₉, C₁₀H₂₁, C₁₁H₂₃, C₁₂H₂₅, C₁₃H₂₇, C₁₄H₂₉, C₁₅H₃₁, C₁₆H₃₃, or C₁₇H₃₅. In some embodiments, R^{6A} and R^{6A}′ are the same. In some embodiments, R^{6A} and R^{6A}′ are each C₁₅H₃₁ or C₁₇H₃₅. In some embodiments, R^{6A} and R^{6A}′ are each C₁₅H₃₁. In some embodiments, R^{6A} and R^{6A}′ are each C₁₇H₃₅.

In some embodiments, the compound of Formula (Ib′) is:

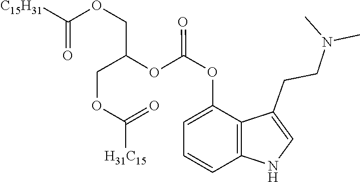

In some embodiments, the compound of Formula (I) has the structure of Formula (Ib″):

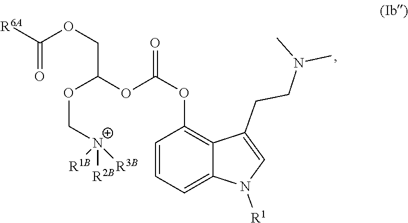

wherein each of R^{6A}, R^{1B}, R^{2B}, and R^{3B} are independently hydrogen or alkyl.

In some embodiments, R^{6A} is —CH₃, —C₂H₅, C₃H₇, C₄H₉, C₅H₁₁, C₆H₁₃, C₇H₁₅, C₈H₁₇, C₉H₁₉, C₁₀H₂₁, C₁₁H₂₃, C₁₂H₂₅, C₁₃H₂₇, C₁₄H₂₉, C₁₅H₃₁, C₁₆H₃₃, or C₁₇H₃₅. In some embodiments, R^{6A} is C₁₅H₃₁ or C₁₇H₃₅. In some embodiments, R^{6A} is C₁₅H₃₁. In some embodiments, R^{6A} is C₁₇H₃₅.

In some embodiments, R^{1B}, R^{2B}, and R^{3B} are each independently alkyl. In some embodiments, each of R^{1B}, R^{2B}, and R^{3B} is independently methyl, ethyl, n-propyl, isopropyl, n-butyl, tert-butyl, n-pentyl, 3-methyl-1-butyl, or —C₁₀H₂₁. In some embodiments, R^{1B}, R^{2B}, and R^{3B} are each methyl.

In some embodiments, the compound of Formula (Ib″) is:

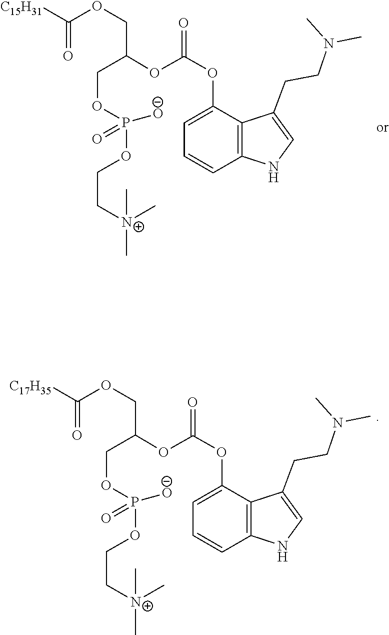

In some embodiments, the compound of Formula (I) has the structure of Formula (Ia′):

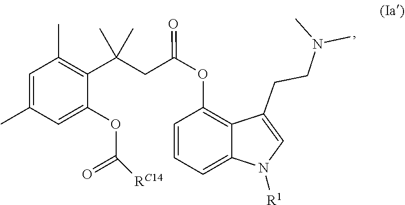

wherein R^{C14} is hydrogen or alkyl.

In some embodiments, R^{C14} is methyl, ethyl, n-propyl, isopropyl, n-butyl, tert-butyl, n-pentyl, 3-methyl-1-butyl, or —C₁₀H₂₁.

In some embodiments, the compound of Formula (Ia) has the structure of Formula (Ia-1):

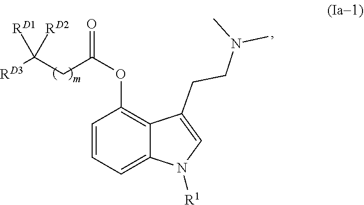

wherein R^{D1} and R^{D2} together with the atom to which they are attached form a cycloalkyl ring or heterocyclylalkyl ring that is unsubstituted or substituted with one or more R^{A}; R^{D3} is independently hydrogen, alkyl, heteroalkyl, cycloalkyl, heterocyclylalkyl, aryl, heteroaryl, —OC(O)R¹⁵, or —C(O)OR¹³, wherein alkyl, heteroalkyl, cycloalkyl, heterocyclylalkyl, aryl, or heteroaryl is unsubstituted or substituted with one or more alkyl, heteroalkyl, cycloalkyl, heterocyclylalkyl, aryl, heteroaryl, an amino acid side chain, —OR¹³, —N(R¹⁸)R¹⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹], —C(O)N(R¹⁸)R¹⁹, —OC(O)N(R¹⁸)R¹⁹, or —OP(O)OR²⁰(OR²¹); m is 0 to 10.

In some embodiments, R^{D1} and R^{D2} together with the atom to which they are attached form an unsubstituted or substituted cycloalkyl ring. In some embodiments, R^{D1} and R^{D2} together with the atom to which they are attached form an unsubstituted cycloalkyl ring. In some embodiments, R^{D1} and R^{D2} together with the atom to which they are attached form a cyclopropyl, cyclobutyl, cyclopentyl, cyclohexyl, or cycloheptyl ring. In one embodiment, R^{D1} and R^{D2} together with the atom to which they are attached form a cyclohexyl.

In some embodiments, m is 0 to 8. In some embodiments, m is 0 to 6. In some embodiments, m is 0 to 4. In some embodiments, m is 1 to 4. In some embodiments, m is 1 to 3. In some embodiments, m is 1 to 2. In some embodiments, m is 0. In some embodiments, m is 1. In some embodiments, m is 2. In some embodiments, m is 3.

In some embodiments, R^{D3} is alkyl substituted with one or more substituent R^{A}, and wherein each R^{A} is independently selected from alkyl, heteroalkyl, cycloalkyl, heterocyclylalkyl, aryl, heteroaryl, an amino acid side chain, —OR¹³, —N(R¹⁸)R¹⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹], —C(O)N(R¹⁸)R¹⁹, —OC(O)N(R¹⁸)R¹⁹, or —OP(O)OR²⁰(OR²¹). In some embodiments, R^{D3} is alkyl substituted —C(O)OR¹³. In some embodiments, R^{D3} is alkyl substituted —C(O)OR¹³, wherein R¹³ is methyl, ethyl, n-propyl, isopropyl, n-butyl, tert-butyl, n-pentyl, 3-methyl-1-butyl, or —C₁₀H₂₁. In some embodiments, R¹³ is methyl, ethyl, isopropyl, or tert-butyl. In some embodiments, R^{D3} is —CH₂OC(O)R¹³, wherein R¹³ is methyl, ethyl, isopropyl, or tert-butyl.

In some embodiments, the compound of Formula (Ib) has the structure of Formula (Ib-1).

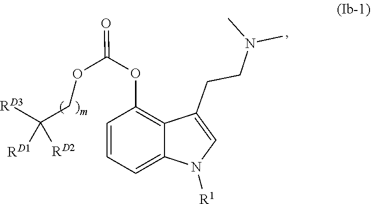

wherein R^{D1} and R^{D2} together with the atom to which they are attached form a cycloalkyl ring or heterocyclylalkyl ring that is unsubstituted or substituted with one or more R^{A}; R^{D3} is independently hydrogen, alkyl, heteroalkyl, cycloalkyl, heterocyclylalkyl, aryl, heteroaryl, —OC(O)R¹⁵, or —C(O)OR¹³, wherein alkyl, heteroalkyl, cycloalkyl, heterocyclylalkyl, aryl, or heteroaryl is unsubstituted or substituted with one or more alkyl, heteroalkyl, cycloalkyl, heterocyclylalkyl, aryl, heteroaryl, an amino acid side chain, —OR¹³, —N(R¹⁸)R¹⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹], —C(O)N(R¹⁸)R¹⁹, —OC(O)N(R¹⁸)R¹⁹, or —OP(O)OR²⁰(OR²¹); m is 0 to 10.

In some embodiments, R^{D1} and R^{D2} together with the atom to which they are attached form an unsubstituted or substituted cycloalkyl ring. In some embodiments, R^{D1} and R^{D2} together with the atom to which they are attached form an unsubstituted cycloalkyl ring. In some embodiments, R^{D1} and R^{D2} together with the atom to which they are attached form a cyclopropyl, cyclobutyl, cyclopentyl, cyclohexyl, or cycloheptyl ring. In one embodiment, R^{D1} and R^{D2} together with the atom to which they are attached form a cyclohexyl.

In some embodiments, m is 0 to 8. In some embodiments, m is 0 to 6. In some embodiments, m is 0 to 4. In some embodiments, m is 1 to 4. In some embodiments, m is 1 to 3. In some embodiments, m is 1 to 2. In some embodiments, m is 0. In some embodiments, m is 1. In some embodiments, m is 2. In some embodiments, m is 3.

In some embodiments, R^{D3} is —C(O)OR¹³. In some embodiments, R¹³ is methyl, ethyl, n-propyl, isopropyl, n-butyl, tert-butyl, n-pentyl, 3-methyl-1-butyl, or —C₁₀H₂₁. In some embodiments, R¹³ is methyl, ethyl, isopropyl, or tert-butyl.

In some embodiments, the compound of Formula (Ic) has the structure of Formula (Ic-1):

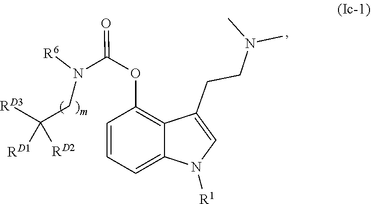

wherein R⁶ is defined herein above; R^{D1} and R^{D2} together with the atom to which they are attached form a cycloalkyl ring or heterocyclylalkyl ring that is unsubstituted or substituted with one or more R^{A}; R^{D3} is independently hydrogen, alkyl, heteroalkyl, cycloalkyl, heterocyclylalkyl, aryl, heteroaryl, —OC(O)R¹⁵, or —C(O)OR¹³, wherein alkyl, heteroalkyl, cycloalkyl, heterocyclylalkyl, aryl, or heteroaryl is unsubstituted or substituted with one or more alkyl, heteroalkyl, cycloalkyl, heterocyclylalkyl, aryl, heteroaryl, an amino acid side chain, —OR¹³, —N(R¹⁸)R¹⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹], —C(O)N(R¹⁸)R¹⁹, —OC(O)N(R¹⁸)R¹⁹, or —OP(O)OR²⁰(OR²¹); m is 0 to 10.

In some embodiments, R^{D1} and R^{D2} together with the atom to which they are attached form an unsubstituted or substituted cycloalkyl ring. In some embodiments, R^{D1} and R^{D2} together with the atom to which they are attached form an unsubstituted cycloalkyl ring. In some embodiments, R^{D1} and R^{D2} together with the atom to which they are attached form a cyclopropyl, cyclobutyl, cyclopentyl, cyclohexyl, or cycloheptyl ring. In one embodiment, R^{D1} and R^{D2} together with the atom to which they are attached form a cyclohexyl.

In some embodiments, m is 0 to 8. In some embodiments, m is 0 to 6. In some embodiments, m is 0 to 4. In some embodiments, m is 1 to 4. In some embodiments, m is 1 to 3. In some embodiments, m is 1 to 2. In some embodiments, m is 0. In some embodiments, m is 1. In some embodiments, m is 2. In some embodiments, m is 3.

In some embodiments, R^{D3} is —C(O)OR¹³. In some embodiments, R¹³ is methyl, ethyl, n-propyl, isopropyl, n-butyl, tert-butyl, n-pentyl, 3-methyl-1-butyl, or —C₁₀H₂₁. In some embodiments, R¹³ is methyl, ethyl, isopropyl, or tert-butyl.

In some embodiments, R⁶ is hydrogen or alkyl. In some embodiment, R⁶ is hydrogen. In some embodiments, R⁶ is alkyl. In some embodiment, R⁶ is methyl, ethyl, n-propyl, isopropyl, n-butyl, tert-butyl, n-pentyl, 3-methyl-1-butyl, or —C₁₀H₂₁.

### Silyl Prodrugs

In some embodiments, the compound of Formula (I) has the structure of Formula (Ii):

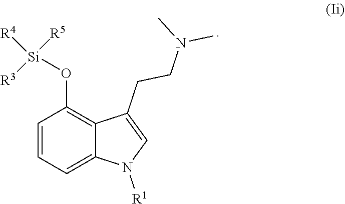

In some embodiments, each of R³, R⁴ and R⁵ is independently hydrogen, unsubstituted or substituted alkyl, heteroalkyl, cycloalkyl, or heterocyclylalkyl.

In some embodiments, each of R³, R⁴ and R⁵ is unsubstituted or substituted alkyl. In some embodiments, each of R³, R⁴ and R⁵ is independently alkyl substituted with one or more substituent R^{A}, and wherein each R^{A} is independently selected from alkyl, heteroalkyl, cycloalkyl, heterocyclylalkyl, aryl, heteroaryl, an amino acid side chain, —OR¹³, —N(R¹⁸)R¹⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹], —C(O)N(R¹⁸)R¹⁹, —OC(O)N(R¹⁸)R¹⁹, or —OP(O)OR²⁰(OR²¹).

In some embodiments, each of R³, R⁴ and R⁵ is independently unsubstituted alkyl. In some embodiments, each of R³, R⁴ and R⁵ is independently methyl, ethyl, n-propyl, isopropyl, n-butyl, tert-butyl, n-pentyl, 3-methyl-1-butyl, or —C₁₀H₂₁.

In some embodiments, R³, R⁴ and R⁵ are the same unsubstituted alkyl. In some embodiments, R³ and R⁴ are methyl, ethyl or isopropyl.

In some embodiments, R⁵ is ethyl, isopropyl, or tert-butyl.

In some embodiments, (i) R³ and R⁴ are methyl, R⁵ is ethyl; (ii) R³, R⁴ and R⁵ are isopropyl; or (iii) R³, R⁴ and R⁵ are ethyl.

In some embodiments, each of R³, R⁴ and R⁵ is independently heteroalkyl.

In some embodiments, each of R³, R⁴ and R⁵ is independently unsubstituted or substituted aryl (e.g., phenyl).

### Sulfate Prodrugs

In some embodiments, the compound of Formula (I) has the structure of Formula (Ij):

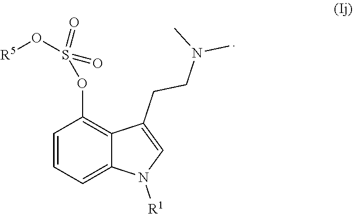

In some embodiments, R⁵ is unsubstituted or substituted alkyl, heteroalkyl, cycloalkyl, or heterocyclylalkyl.

In some embodiments, R⁵ is unsubstituted or substituted alkyl. In some embodiments, R⁵ is alkyl substituted with one or more substituent R^{A}, and wherein each R^{A} is independently selected from alkyl, heteroalkyl, cycloalkyl, heterocyclylalkyl, aryl, heteroaryl, an amino acid side chain, —OR¹³, —N(R¹⁸)R¹⁹, —C(O)OR³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹], —C(O)N(R¹⁸)R¹⁹, —OC(O)N(R¹⁸)R¹⁹, or —OP(O)OR²⁰(OR²¹).

In some embodiments, R⁵ is unsubstituted alkyl. In some embodiments, R⁵ is methyl, ethyl, n-propyl, isopropyl, n-butyl, tert-butyl, n-pentyl, 3-methyl-1-butyl, or —C₁₀H₂₁.

In some embodiments, R⁵ is alkyl substituted with —C(O)OR¹³, wherein R¹³ is hydrogen or alkyl.

In some embodiments, R⁵ is hydrogen, methyl, ethyl, isopropyl, or tert-butyl.

In some embodiments, R⁵ is alkyl substituted with —N(R¹⁸)R¹⁹, wherein each of R¹⁸ and R¹⁹ is independently hydrogen or methyl.

In some embodiments, R⁵ is alkyl substituted with —N(R¹³)C(O)R¹⁴, wherein each of R¹³ and R¹⁴ is independently hydrogen or methyl. In some embodiments, R⁵ is alkyl substituted with —N(R¹³)C(O)R¹⁴, wherein each of R¹³ is hydrogen or methyl, and R¹⁴ is hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, and heteroaryl is unsubstituted or further substituted with one or more halogen, amino, cyano, hydroxyl, alkyl, acetyl, or benzoyl.

In some embodiments, R⁵ is alkyl substituted with —N(R¹³)C(O)OR¹⁴, wherein each of R¹³ and R¹⁴ is independently hydrogen, methyl, or ethyl.

In some embodiments, R⁵ is heterocyclylalkyl. In some embodiments, R⁵ is selected from aziridinyl, oxetanyl, azetidinyl, tetrahydrofuranyl, pyrrolidinyl, piperidinyl, piperazinyl, morpholinyl, thiomorpholinyl, thiomorpholinyl dioxide, diazinanyl,

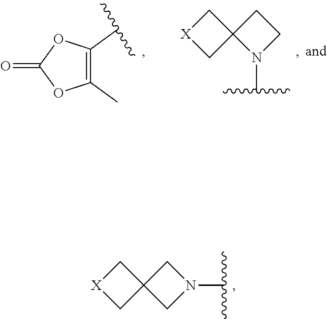

wherein X is —CH₂—, —O—, —S—, —SO₂, —NH—, or —NMe.

### Selected Compounds of the Disclosure

In some embodiments, the compound of Formula (I) is selected from:

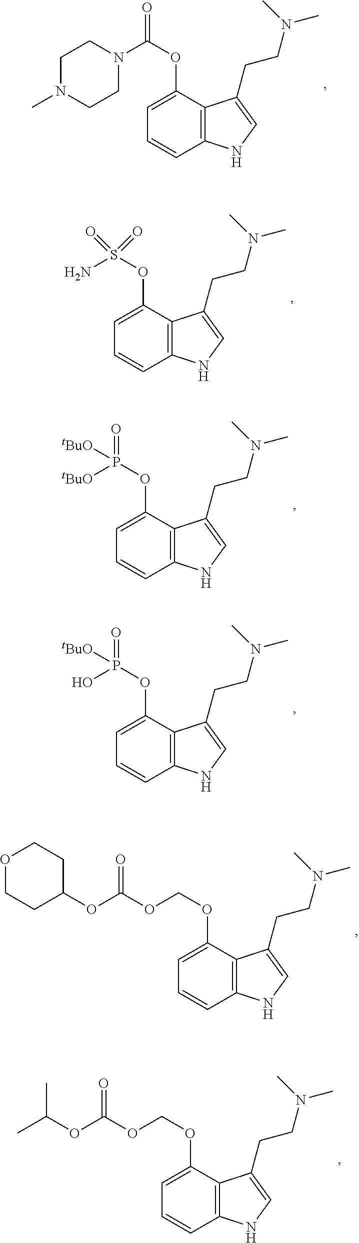

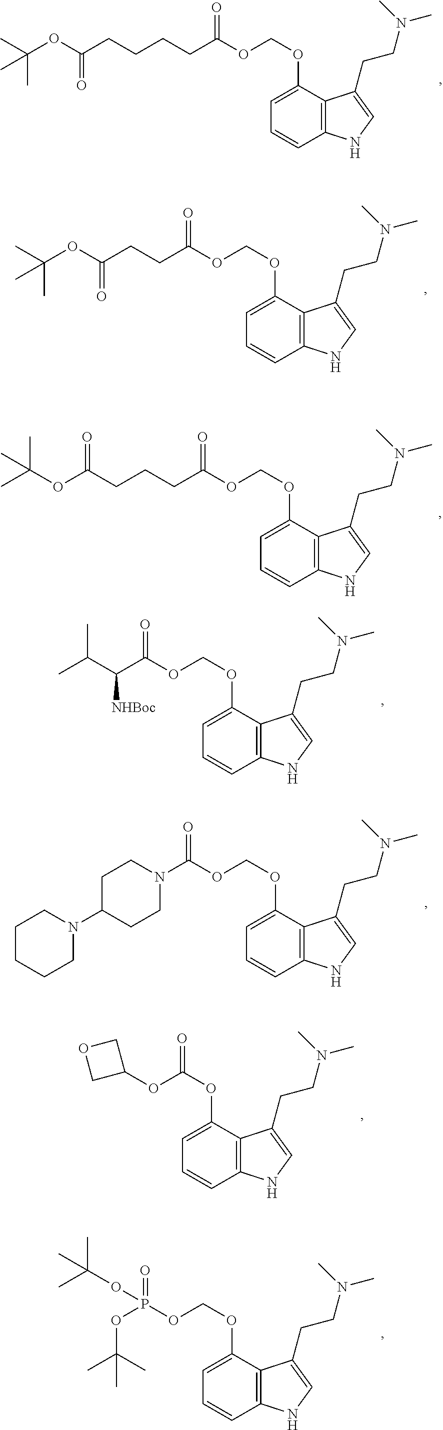

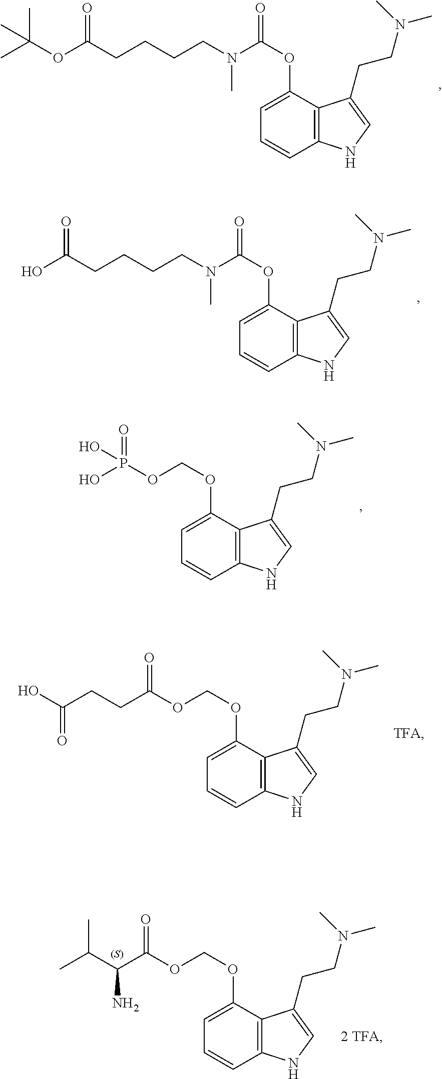

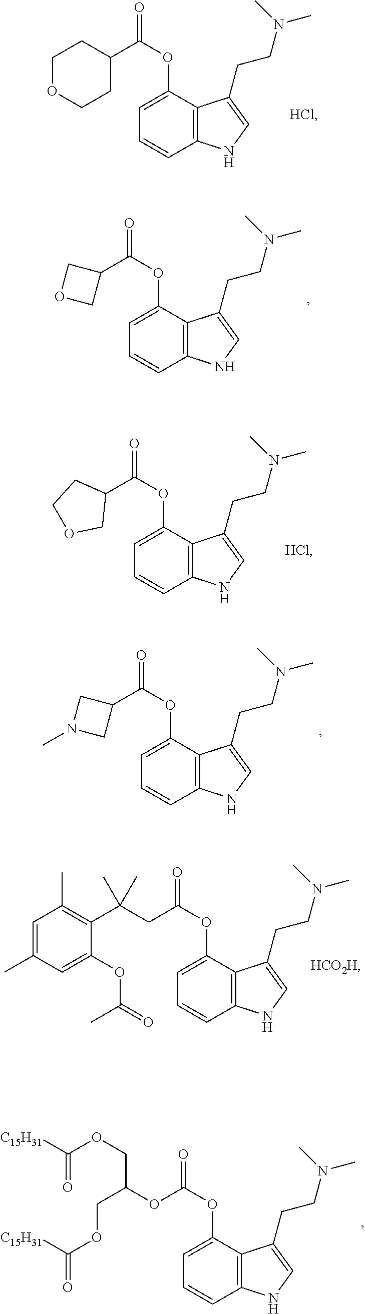

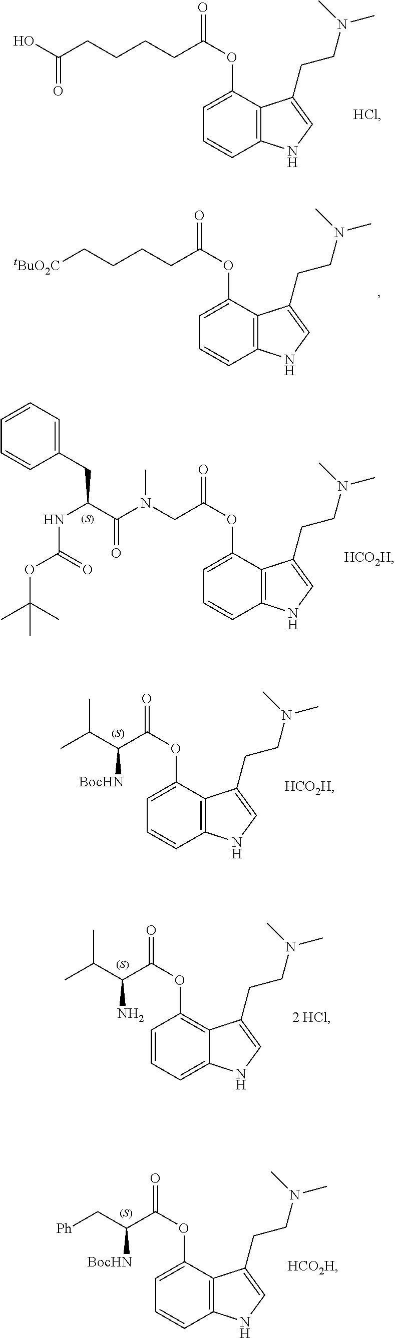

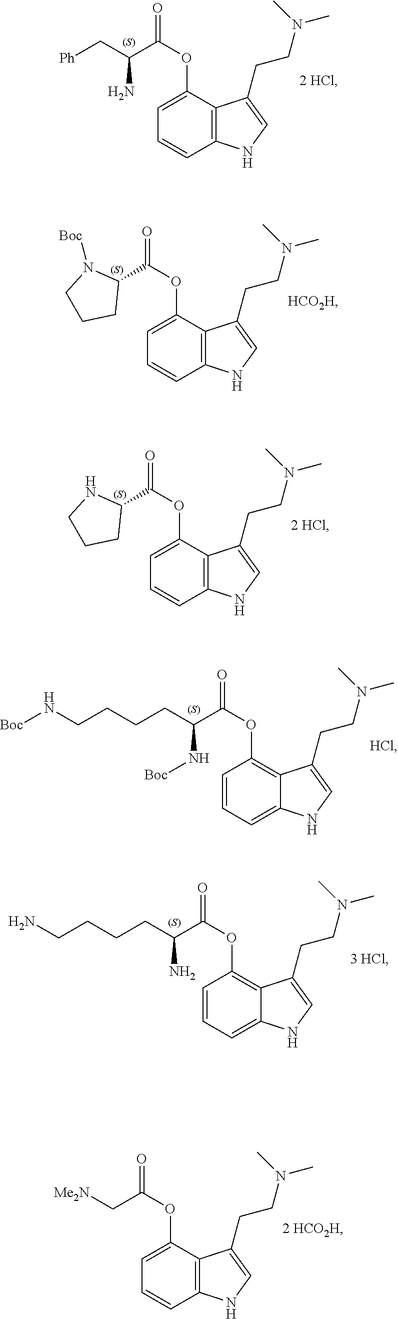

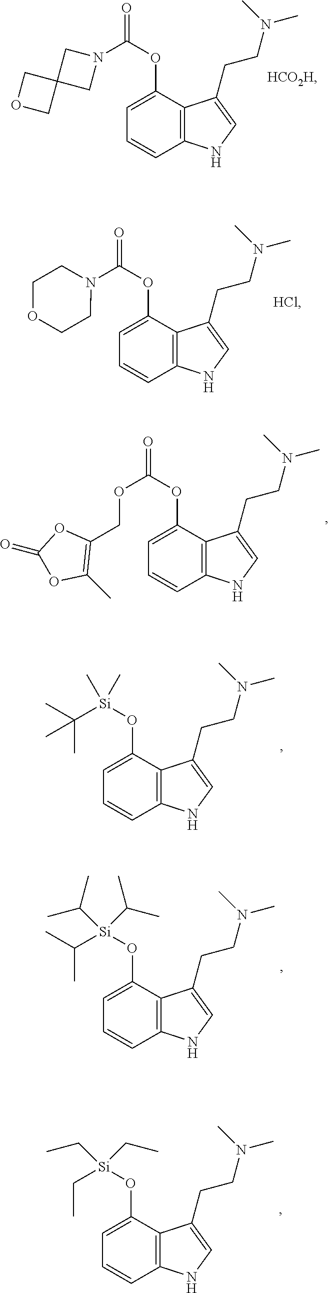

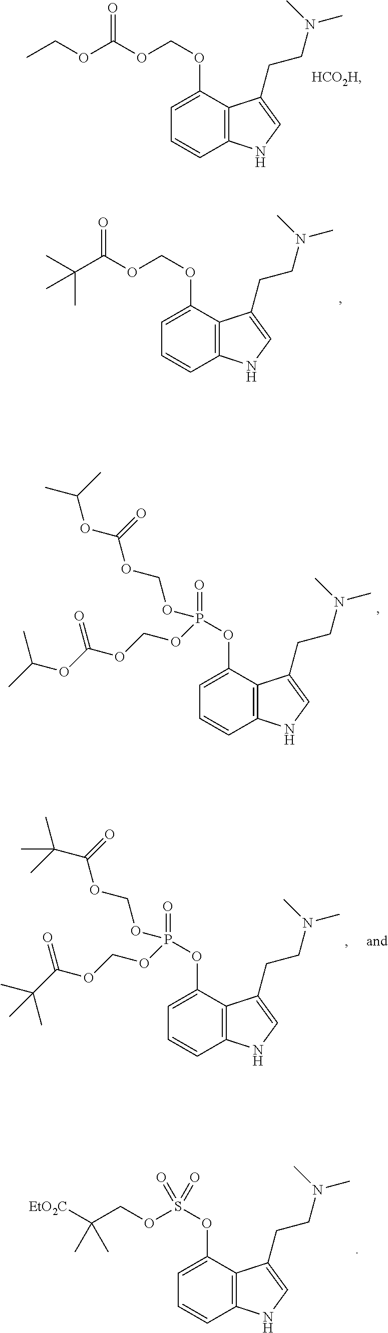

In some embodiments, one or more of the hydrogens in the compound of Formula (I) are replaced with a deuterium.

Selected compounds of the disclosure with corresponding simplified molecular-input line-entry system (SMILES) strings are provided in TABLE 1.

TABLE 1

| | Structure |
|---|---|
| Cpd | SMILES |
| 1 | 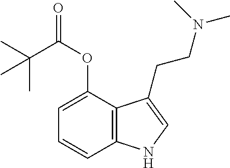 [SMILES: CN(C)CCc1c[nH]c2cccc(OC(=O)C(C)(C)C)c12] |
| | O═C(C(C)(C)C)OC1═CC═CC2═C1C(CN(C)C)═CN2 |
| 2 | 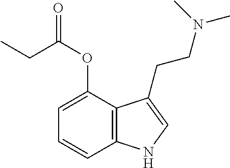 [SMILES: CCC(=O)Oc1cccc2[nH]cc(CCN(C)C)c12] |
| | O═C(CC)OC1═CC═CC2═C1C(CCN(C)C)═CN2 |
| 3 | 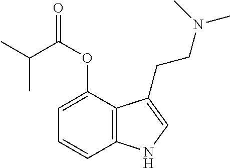 [SMILES: CC(C)C(=O)Oc1cccc2[nH]cc(CCN(C)C)c12] |
| | O═C(C(C)C)OC1═CC═CC2═C1C(CCN(C)C)═CN2 |
| 4 | 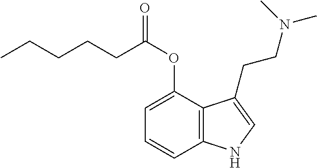 [SMILES: CCCCCC(=O)Oc1cccc2[nH]cc(CCN(C)C)c12] |
| | O═C(CCCCC)OC1═CC═CC2═C1C(CCN(C)C)═CN2 |
| 5 | 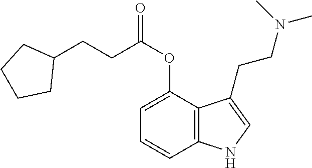 [SMILES: CN(C)CCc1c[nH]c2cccc(OC(=O)CCC3CCCC3)c12] |
| | O═C(CC1CCCC1)OC2═CC═CC3═C2C(CCN(C)C)═CN3 |
| 6 | 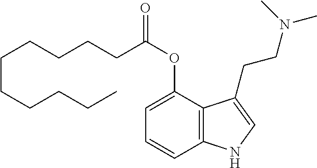 [SMILES: CCCCCCCCCCC(=O)Oc1cccc2[nH]cc(CCN(C)C)c12] |
| | O═C(CCCCCCCCCC)OC1═CC═CC2═C1C(CCN(C)C)═CN2 |
| 7 | 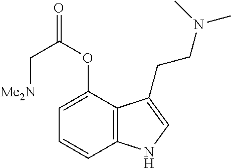 [SMILES: CN(C)CCc1c[nH]c2cccc(OC(=O)CN(C)C)c12] |
| | O═C(CN(C)C)OC1═CC═CC2═C1C(CCN(C)C)═CN2 |
| 8 | 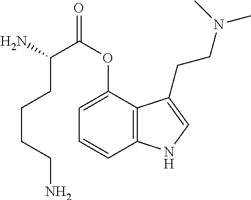 [SMILES: CN(C)CCc1c[nH]c2cccc(OC(=O)[C@@H](N)CCCCN)c12] |
| | O═C([C@@H](N)CCCCN)OC1═CC═CC2═C1C(CCN(C)C)═CN2 |
| 9 | 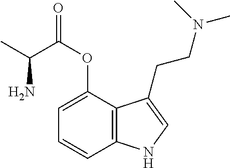 [SMILES: C[C@H](N)C(=O)Oc1cccc2[nH]cc(CCN(C)C)c12] |
| | O═([C@H](C)N)OC1═CC═CC2═C1C(CCN(C)C)═CN2 |
| 10 | 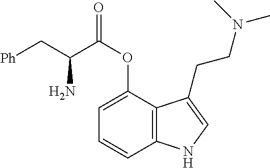 [SMILES: CN(C)CCc1c[nH]c2cccc(OC(=O)[C@@H](N)Cc3ccccc3)c12] |
| | O═C([C@H](CC1═CC═CC═C1)N) |
| | OC2═CC═CC3═C2C(CCN(C)C)═CN3 |
| 11 | 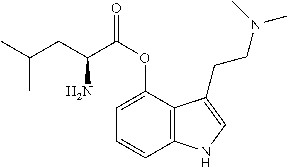 [SMILES: CC(C)C[C@H](N)C(=O)Oc1cccc2[nH]cc(CCN(C)C)c12] |
| | O═C([C@H](CC(C)C)N)OC1═CC═CC2═C1C(CCN(C)C)═CN2 |
| 12 | 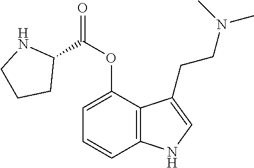 [SMILES: CN(C)CCc1c[nH]c2cccc(OC(=O)[C@@H]3CCCN3)c12] |
| | O═C([C@H]1NCCC1)OC2═CC═CC3═C2C(CCN(C)C)═CN3 |
| 13 | 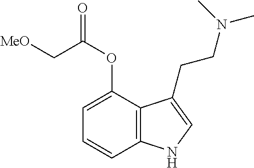 [SMILES: COCC(=O)Oc1cccc2[nH]cc(CCN(C)C)c12] |
| | O═C(COC)OC1═CC═CC2═C1C(CCN(C)C)═CN2 |
| 14 | 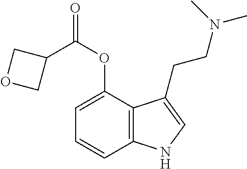 [SMILES: CN(C)CCc1c[nH]c2cccc(OC(=O)C3COC3)c12] |
| | O═C(C1COC1)OC2═CC═CC3═C2C(CCN(C)C)═CN3 |
| 15 | 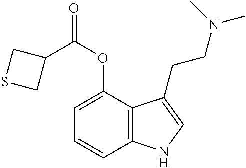 [SMILES: CN(C)CCc1c[nH]c2cccc(OC(=O)C3CSC3)c12] |
| | O═C(C1CSC1)OC2═CC═CC3═C2C(CCN(C)C)═CN3 |
| 16 | 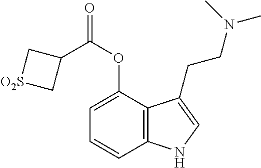 [SMILES: CN(C)CCc1c[nH]c2cccc(OC(=O)C3CS(=O)(=O)C3)c12] |
| | O═C(C1CS(C1)(═O)═O)OC2═CC═CC3═C2C(CCN(C)C)═CN3 |
| 17 | 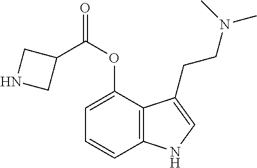 [SMILES: CN(C)CCc1c[nH]c2cccc(OC(=O)C3CNC3)c12] |
| | O═C(C1CNC1)OC2═CC═CC3═C2C(CCN(C)C)═CN3 |
| 18 | 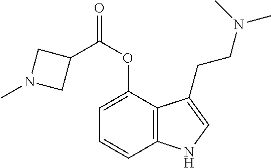 |
| | O═C(C1CN(C)C1)OC2═CC═CC3═C2C(CCN(C)C)═CN3 |
| 19 | 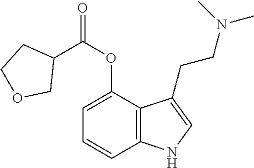 [SMILES: CN(C)CCc1c[nH]c2cccc(OC(=O)C3CCOC3)c12] |
| | O═C(C1CCOC1)OC2═CC═CC3═C2C(CCN(C)C)═CN3 |
| 20 | 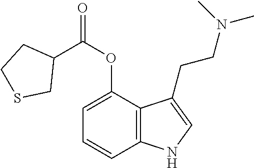 [SMILES: CN(C)CCc1c[nH]c2cccc(OC(=O)C3CCSC3)c12] |
| | O═C(C1CCSC1)OC2═CC═CC3═C2C(CCN(C)C)═CN3 |
| 21 | 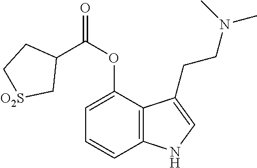 [SMILES: CN(C)CCc1c[nH]c2cccc(OC(=O)C3CCS(=O)(=O)C3)c12] |
| | O═C(C1CCS(C1)(═O)═O)OC2═CC═CC3═C2C(CCN(C)C)═CN3 |
| 22 | 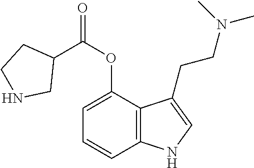 [SMILES: CN(C)CCc1c[nH]c2cccc(OC(=O)C3CCNC3)c12] |
| | O═C(C1CCNC1)OC2═CC═CC3═C2C(CCN(C)C)═CN3 |
| 23 | 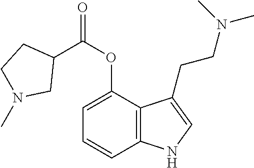 [SMILES: CN(C)CCc1c[nH]c2cccc(OC(=O)C3CCN(C)C3)c12] |
| | O═C(C1CCN(C)C1)OC2═CC═CC3═C2C(CCN(C)C)═CN3 |
| 24 | 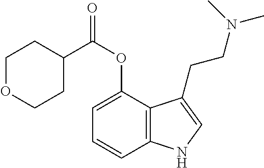 [SMILES: CN(C)CCc1c[nH]c2cccc(OC(=O)C3CCOCC3)c12] |
| | O═C(C1CCOCC1)OC2═CC═CC3═C2C(CCN(C)C)═CN3 |
| 25 | 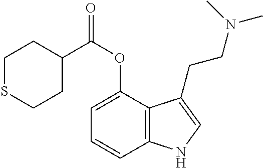 [SMILES: CN(C)CCc1c[nH]c2cccc(OC(=O)C3CCSCC3)c12] |
| | O═C(C1CCSCC1)OC2═CC═CC3═C2C(CCN(C)C)═CN3 |
| 26 | 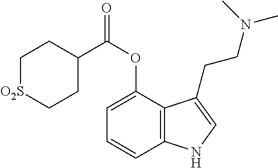 [SMILES: CN(C)CCc1c[nH]c2cccc(OC(=O)C3CCS(=O)(=O)CC3)c12] |
| | O═C(C1CCS(CC1)(═O)═O)OC2═CC═CC3═C2C(CCN(C)C)═CN3 |
| 27 | 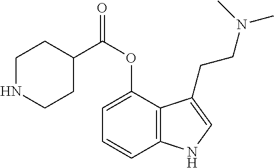 [SMILES: CN(C)CCc1c[nH]c2cccc(OC(=O)C3CCNCC3)c12] |
| | O═C(C1CCNCC1)OC2═CC═CC3═C2C(CCN(C)C)═CN3 |
| 28 | 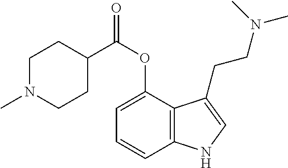 |
| | O═C(C1CCN(C)CC1)OC2═CC═CC3═C2C(CCN(C)C)═CN3 |
| 29 | 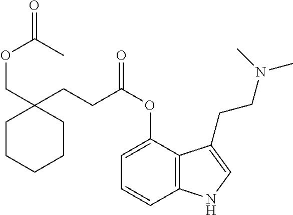 [SMILES: CC(=O)OCC1(CCC(=O)Oc2cccc3[nH]cc(CCN(C)C)c23)CCCCC1] |
| | O═C(CC1(CCCCC1)COC(C)═O)OC2═CC═CC3═C2C(CCN(C)C)═CN3 |
| 30 | 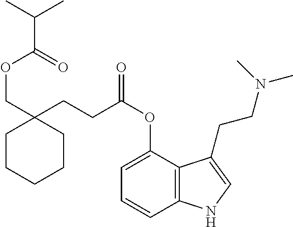 [SMILES: CC(C)C(=O)OCC1(CCC(=O)Oc2cccc3[nH]cc(CCN(C)C)c23)CCCCC1] |
| | O═C(CCC1(CCCCC1)COC(C(C)C)═O) |
| | OC2═CC═CC3═C2C(CCN(C)C)═CN3 |
| 31 | 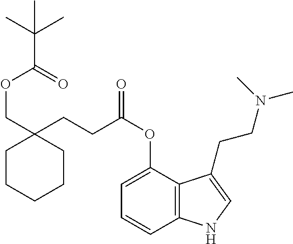 [SMILES: CN(C)CCc1c[nH]c2cccc(OC(=O)CCC3(COC(=O)C(C)(C)C)CCCCC3)c12] |
| | O═C(CCC1(CCCCC1)COC(C(C)(C)C)═O)3 |
| | OC2═CC═CC3═C2C(CCN(C)C)═CN |
| 32 | 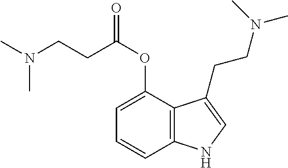 |
| | O═C(CCN(C)C)OC1═CC═CC2═C1C(CCN(C)C)═CN2 |
| 33 | 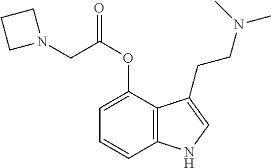 [SMILES: CN(C)CCc1c[nH]c2cccc(OC(=O)CN3CCC3)c12] |
| | O═C(CN1CCC1)OC2═CC═CC3═C2C(CCN(C)C)═CN3 |
| 34 | 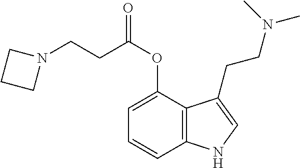 [SMILES: CN(C)CCc1c[nH]c2cccc(OC(=O)CCN3CCC3)c12] |
| | O═C(CCN1CCC1)OC2═CC═CC3═C2C(CCN(C)C)═CN3 |
| 35 | 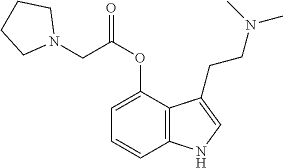 [SMILES: CN(C)CCc1c[nH]c2cccc(OC(=O)CN3CCCC3)c12] |
| | O═C(CN1CCCC1)OC2═CC═CC3═C2C(CCN(C)C)═CN3 |
| 36 | 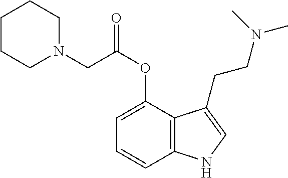 [SMILES: CN(C)CCc1c[nH]c2cccc(OC(=O)CN3CCCCC3)c12] |
| | O═C(CN1CCCCC1)OC2═CC═CC3═C2C(CCN(C)C)═CN3 |
| 37 | 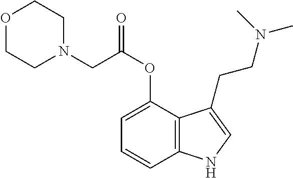 [SMILES: CN(C)CCc1c[nH]c2cccc(OC(=O)CN3CCOCC3)c12] |
| | O═C(CN1CCOCC1)OC2═CC═CC3═C2C(CCN(C)C)═CN3 |
| 38 | 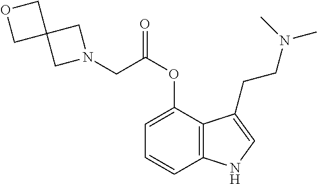 [SMILES: CN(C)CCc1c[nH]c2cccc(OC(=O)CN3CC4(COC4)C3)c12] |
| | O═C(CN1CC2(COC2)C1)OC3═CC═CC4═C3C(CCN(C)C)═CN4 |
| 39 | 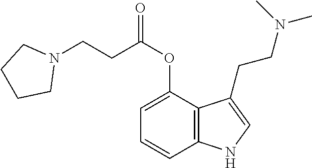 [SMILES: CN(C)CCc1c[nH]c2cccc(OC(=O)CCN3CCCC3)c12] |
| | O═C(CCN1CCCC1)OC2═CC═CC3═C2C(CCN(C)C)═CN3 |
| 40 | 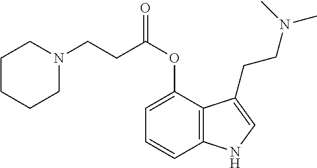 [SMILES: CN(C)CCc1c[nH]c2cccc(OC(=O)CCN3CCCCC3)c12] |
| | O═C(CCN1CCCCC1)OC2═CC═CC3═C2C(CCN(C)C)═CN3 |
| 41 | 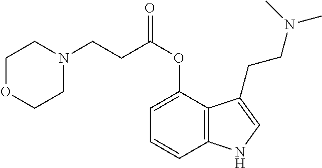 [SMILES: CN(C)CCc1c[nH]c2cccc(OC(=O)CCN3CCOCC3)c12] |
| | O═C(CCN1CCOCC1)OC2═CC═CC3═C2C(CCN(C)C)═CN3 |
| 42 | 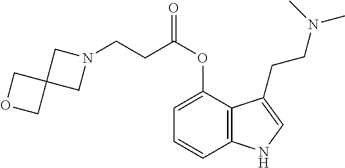 [SMILES: CN(C)CCc1c[nH]c2cccc(OC(=O)CCN3CC4(COC4)C3)c12] |
| | O═C(CCN1CC2(COC2)C1)OC3═CC═CC4═C3C(CCN(C)C)═CN4 |
| 43 | 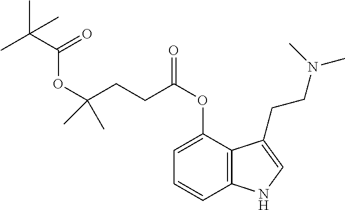 [SMILES: CN(C)CCc1c[nH]c2cccc(OC(=O)CCC(C)(C)OC(=O)C(C)(C)C)c12] |
| | O═C(CCC(C)OC(C(C)(C)C)═O)OC1═CC═CC2═C1C(CCN(C)C)═CN2 |
| 44 | 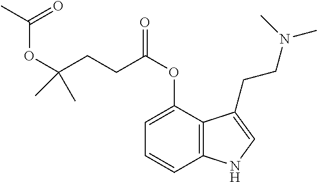 [SMILES: CC(=O)OC(C)(C)CCC(=O)Oc1cccc2[nH]cc(CCN(C)C)c12] |
| | O═C(CCC(C)(C)OC(C)═O)OC1═CC═CC2═C1C(CCN(C)C)═CN2 |
| 45 | 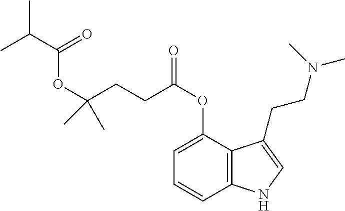 [SMILES: CC(C)C(=O)OC(C)(C)CCC(=O)Oc1cccc2[nH]cc(CCN(C)C)c12] |
| | O═C(CCC(C)(C)OC(C(C)C)═O)OC1═CC═CC2═C1C(CCN(C)C)═NC2 |
| 46 | 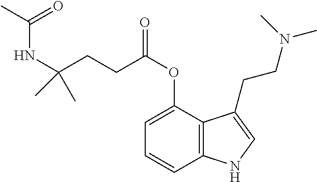 [SMILES: CC(=O)NC(C)(C)CCC(=O)Oc1cccc2[nH]cc(CCN(C)C)c12] |
| | O═C(CCC(C)(C)NC(C)═O)OC1═CC═CC2═C1C(CCN(C)C)═CN2 |
| 47 | 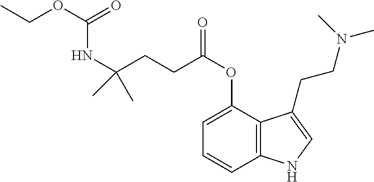 [SMILES: CCOC(=O)NC(C)(C)CCC(=O)Oc1cccc2[nH]cc(CCN(C)C)c12] |
| | O═C(CCC(C)(C)NC(OCC)═O)OC1═CC═CC2═C1C(CCN(C)C)═CN2 |
| 48 | 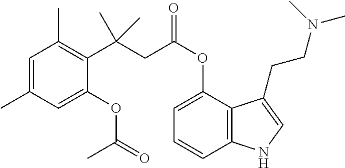 |
| | O═C(CC(C)(C)C1═C(C)C═C(C)C═C1OC(C)═O) |
| | OC2═CC═CC3═C2C(CCN(C)C)═CN3 |
| 49 | 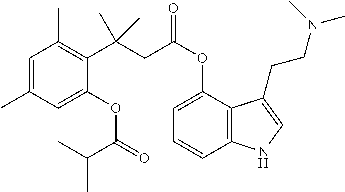 |
| | O═C(CC(C)(C)C1═C(C)C═C(C)C═C1OC(C(C)C)═O) |
| | OC2═CC═CC3═C2C(CCN(C)C)═CN3 |
| 50 | 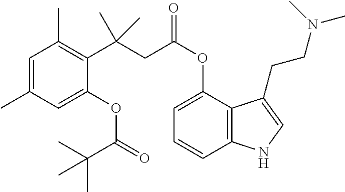 |
| | O═C(CC(C)(C)C1═C(C)C═C(C)C═C1OC(C(C)(C)C)═O) |
| | OC2═CC═CC3═C2C(CCN(C)C)═CN3 |
| 51 | 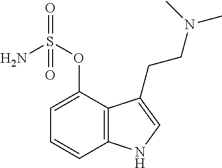 [SMILES: CN(C)CCc1c[nH]c2cccc(OS(N)(=O)=O)c12] |
| | NS(OC1═CC═CC2═C1C(CCN(C)C)═CN2)(═O)═O |
| 52 | 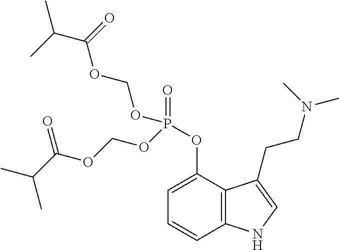 [SMILES: CC(C)C(=O)OCOP(=O)(OCOC(=O)C(C)C)Oc1cccc2[nH]cc(CCN(C)C)c12] |
| | CN(C)CCC1═CNC2═C1C(OP(OCOC(C(C)C)═O)(OCOC(C(C) |
| | C)═O)═O)═C═C2 |
| 53 | 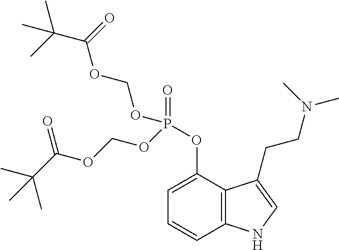 [SMILES: CN(C)CCc1c[nH]c2cccc(OP(=O)(OCOC(=O)C(C)(C)C)OCOC(=O)C(C)(C)C)c12] |
| | CN(C)CCC1═CNC2═C1C(OP(OC(C)OC(C(C)C)═O) |
| | (OC(C)OC(C(C)C)═O)═O)═CC═C2 |
| 54 | 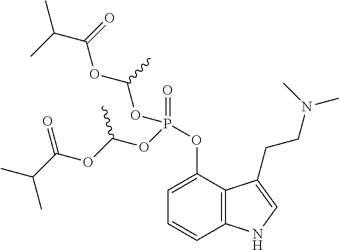 |
| | CN(C)CCC1═CNC2═C1C(OP(C)OC(C(C)C)═O)(OCOC(C(C) |
| | C)═O)═O)CC═C2 |
| 55 | 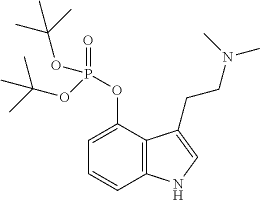 [SMILES: CN(C)CCc1c[nH]c2cccc(OP(=O)(OC(C)(C)C)OC(C)(C)C)c12] |
| | CN(C)CCC1═CNC2═C1C(OP(OC(C)(C)C)(OC(C) |
| | (C)C)═O)═CC═C2 |
| 56 | 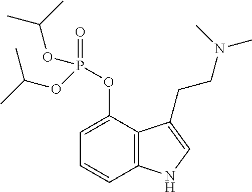 [SMILES: CC(C)OP(=O)(Oc1cccc2[nH]cc(CCN(C)C)c12)OC(C)C] |
| | CN(C)CCC1═CNC2═C1C(OP(OC(C)C)(OC(C)C)═O)═CC═C2 |
| 57 | 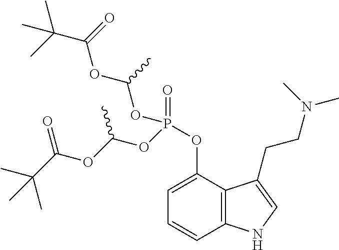 |
| | CN(C)CCC1═CNC2═C1C(OP(C)OC(C(C)(C)C)═O)(OC(C) |
| | OC(C(C)(C)C)═O)═O)═CC═C2 |
| 58 | 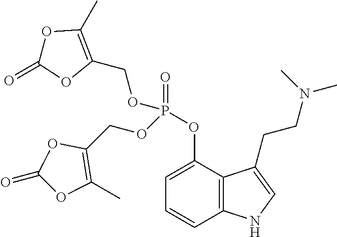 [SMILES: Cc1oc(=O)oc1COP(=O)(OCc1oc(=O)oc1C)Oc1cccc2[nH]cc(CCN(C)C)c12] |
| | CN(C)CCC1═CNC2═C1C(OP(OCC3═C(C)OC(O3)═O) |
| | (OCC(O4)═C(C)OC4═O)═O)═CC═C2 |
| 59 | 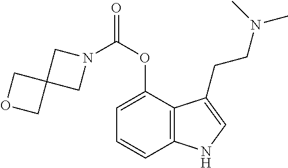 [SMILES: CN(C)CCc1c[nH]c2cccc(OC(=O)N3CC4(COC4)C3)c12] |
| | O═C(N1CC2(COC2)C1)OC3═CC═CC4═C3C(CCN(C)C)═CN4 |
| 60 | 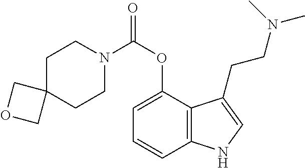 [SMILES: CN(C)CCc1c[nH]c2cccc(OC(=O)N3CCC4(CC3)COC4)c12] |
| | O═C(N1CCC2(COC2)CC1)OC3═CC═CC4═C3C(CCN(C)C)═CN4 |
| 61 | 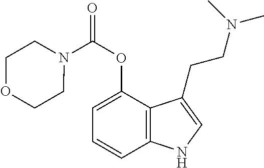 [SMILES: CN(C)CCc1c[nH]c2cccc(OC(=O)N3CCOCC3)c12] |
| | O═C(N1CCOCC1)OC2═CC═CC3═C2C(CCN(C)C)═CN3 |
| 62 | 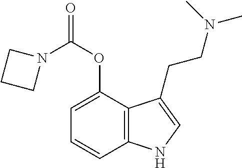 [SMILES: CN(C)CCc1c[nH]c2cccc(OC(=O)N3CCC3)c12] |
| | O═C(N1CCC1)OC2═CC═CC3═C2C(CCN(C)C)═CN3 |
| 63 | 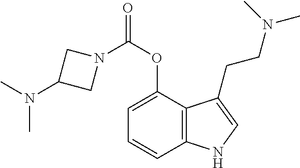 |
| | O═C(N1CC(N(C)C)C1)OC2═CC═CC3═C2C(CCN(C)C)═CN3 |
| 64 | 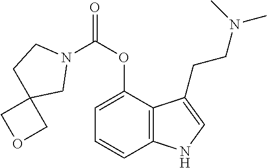 [SMILES: CN(C)CCc1c[nH]c2cccc(OC(=O)N3CCC4(COC4)C3)c12] |
| | O═C(N1CCC2(COC2)C1)OC3═CC═CC4═C3C(CCN(C)C)═CN4 |
| 65 | 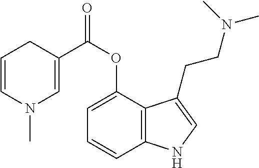 [SMILES: CN1C=CCC(C(=O)Oc2cccc3[nH]cc(CCN(C)C)c23)=C1] |
| | O═C(C1═CN(C)C═CC1)OC2═CC═CC3═C2C(CCN(C)C)═CN3 |
| 66 | 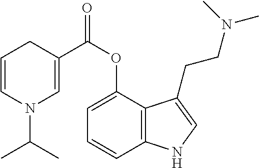 [SMILES: CC(C)N1C=CCC(C(=O)Oc2cccc3[nH]cc(CCN(C)C)c23)=C1] |
| | O═C(C1═CNC(C)C)C═CC1)OC2═CC═CC3═C2C(CCN(C)C)═CN3 |
| 67 | 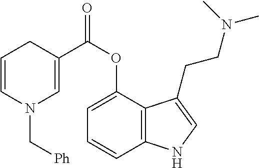 [SMILES: CN(C)CCc1c[nH]c2cccc(OC(=O)C3=CN(Cc4ccccc4)C=CC3)c12] |
| | O═C(C1═CN(CC2═CC═CC═CC═C2)C═CC1) |
| | OC3═CC═CC4═C3C(CCN(C)C)═CN4 |
| 68 | 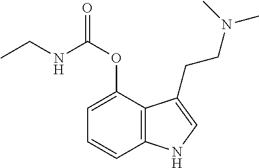 [SMILES: CCNC(=O)Oc1cccc2[nH]cc(CCN(C)C)c12] |
| | O═C(NCC)OC1═CC═CC2═C1C(CCN(C)C)═CN2 |
| 69 | 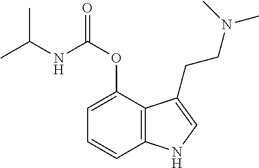 [SMILES: CC(C)NC(=O)Oc1cccc2[nH]cc(CCN(C)C)c12] |
| | O═C(NC(C)C)OC1═CC═CC2═C1C(CCN(C)C)═CN2 |
| 70 | 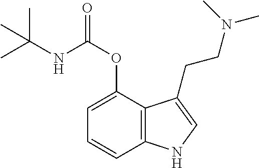 [SMILES: CN(C)CCc1c[nH]c2cccc(OC(=O)NC(C)(C)C)c12] |
| | O═C(NC(C)(C)C)OC1═CC═CC2═C1C(CCN(C)C)═CN2 |
| 71 | 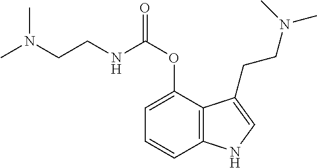 |
| | O═C(NCCN(C)C)OC1═CC═CC2═C1C(CCN(C)C)═CN2 |
| 72 | 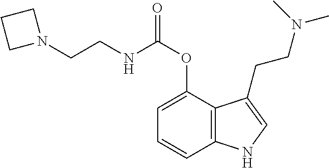 [SMILES: CN(C)CCc1c[nH]c2cccc(OC(=O)NCCN3CCC3)c12] |
| | O═C(NCCN1CCC1)OC2═CC═CC3═C2C(CCN(C)C)═CN3 |
| 73 | 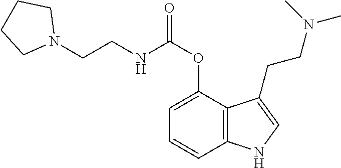 [SMILES: CN(C)CCc1c[nH]c2cccc(OC(=O)NCCN3CCCC3)c12] |
| | O═C(NCCN1CCCC1)OC2═CC═CC3═C2C(CCN(C)C)═CN3 |
| 74 | 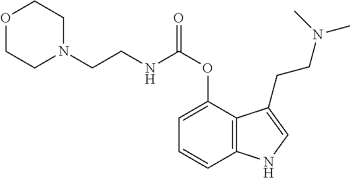 [SMILES: CN(C)CCc1c[nH]c2cccc(OC(=O)NCCN3CCOCC3)c12] |
| | O═C(NCCN1CCOCC1)OC2═CC═CC3═C2C(CCN(C)C)═CN3 |
| 75 | 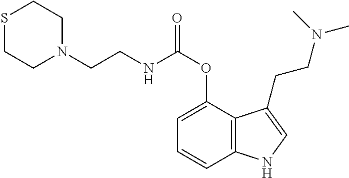 [SMILES: CN(C)CCc1c[nH]c2cccc(OC(=O)NCCN3CCSCC3)c12] |
| | O═C(NCCN1CCSCC1)OC2═CC═CC3═C2C(CCN(C)C)═CN3 |
| 76 | 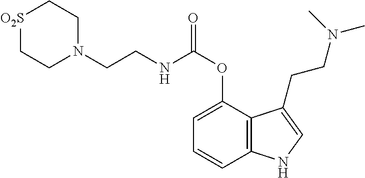 [SMILES: CN(C)CCc1c[nH]c2cccc(OC(=O)NCCN3CCS(=O)(=O)CC3)c12] |
| | O═C(NCCN1CCS(CC1)(═O)═O)OC2═CC═CC3═C2C(CCN(C)C)═CN3 |
| 77 | 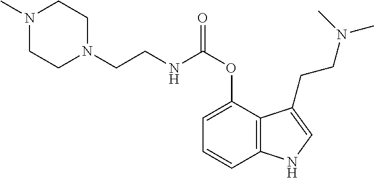 |
| | O═C(NCCN1CCN(C)CC1)OC2═CC═CC3═C2C(CCN(C)C)═CN3 |
| 78 | 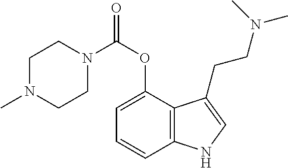 |
| | O═C(N1CCN(C)CC1)OC2═CC═CC3═CC2C(CCN(C)C)═CN3 |
| 79 | 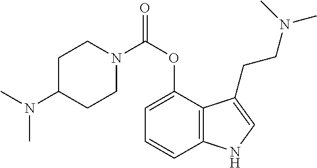 |
| | O═C(N1CCC(N(C)C)CC1)OC2═CC═CC3═C2C(CCN(C)C)═CN3 |
| 80 | 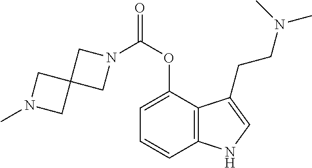 |
| | O═C(N1CC2(CN(C)C2)C1)OC3═CC═CC4═C3C(CCN(C)C)═CN4 |
| 81 | 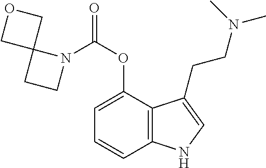 [SMILES: CN(C)CCc1c[nH]c2cccc(OC(=O)N3CCC34COC4)c12] |
| | O═C(N1C2(COC2)CC1)OC3═CC═CC4═C3C(CCN(C)C)═CN4 |
| 82 | 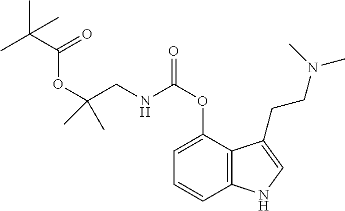 [SMILES: CN(C)CCc1c[nH]c2cccc(OC(=O)NCC(C)(C)OC(=O)C(C)(C)C)c12] |
| | O═C(NCC(C)(C)OC(C(C)(C)C)═O)OC1═CC═CC2═C1C(CCN(C)C)═CN2 |
| 83 | 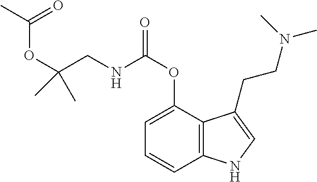 [SMILES: CC(=O)OC(C)(C)CNC(=O)Oc1cccc2[nH]cc(CCN(C)C)c12] |
| | O═C(NCC(C)(C)OC(C)═O)OC1═CC═CC2═C1C(CCN(C)C)═CN2 |
| 84 | 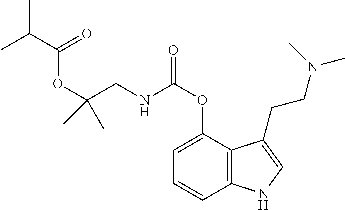 [SMILES: CC(C)C(=O)OC(C)(C)CNC(=O)Oc1cccc2[nH]cc(CCN(C)C)c12] |
| | O═C(NCC(C)(C)OC(C(C)C)═O) |
| | OC1═CC═CC2═C1C(CCN(C)C)═CN2 |
| 85 | 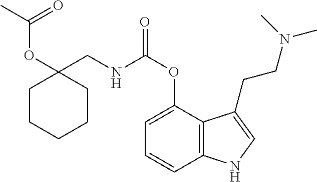 [SMILES: CC(=O)OC1(CNC(=O)Oc2cccc3[nH]cc(CCN(C)C)c23)CCCCC1] |
| | O═C(NCC1(CCCCC1)OC(C)═O) |
| | OC2═CC═CC3═C2C(CCN(C)C)═CN3 |
| 86 | 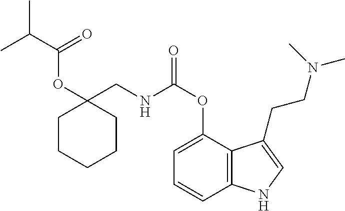 [SMILES: CC(C)C(=O)OC1(CNC(=O)Oc2cccc3[nH]cc(CCN(C)C)c23)CCCCC1] |
| | O═C(NCC1(CCCCC1)OC(C(C)C)═O) |
| | OCC2═CC═CC3═C2C(CCN(C)C)═CN3 |
| 87 | 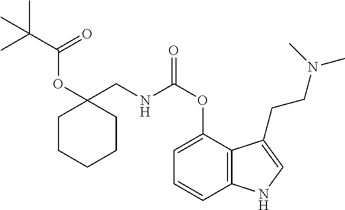 [SMILES: CN(C)CCc1c[nH]c2cccc(OC(=O)NCC3(OC(=O)C(C)(C)C)CCCCC3)c12] |
| | O═C(NCC1(CCCCC1)OC(C(C)(C)C)═O) |
| | OC2═CC═CC3═C2C(CCN(C)C)═CN3 |
| 88 | 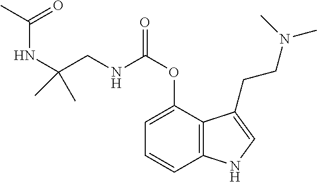 [SMILES: CC(=O)NC(C)(C)CNC(=O)Oc1cccc2[nH]cc(CCN(C)C)c12] |
| | O═C(NCC(C)(C)NC(C)═O)OC1═CC═CC2═C1C(CCN(C)C)═CN2 |
| 89 | 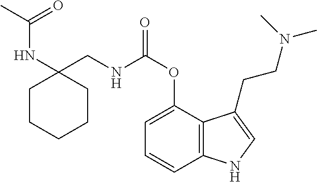 [SMILES: CC(=O)NC1(CNC(=O)Oc2cccc3[nH]cc(CCN(C)C)c23)CCCCC1] |
| | O═C(NCC1(CCCCC1)NC(C)═O)OC2═CC═CC3═C2C(CCN(C)C)═CN3 |
| 90 | 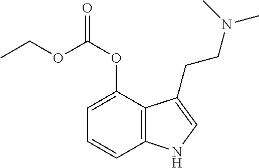 [SMILES: CCOC(=O)Oc1cccc2[nH]cc(CCN(C)C)c12] |
| | O═C(OCC)OC1═CC═CC2═C1C(CCN(C)C)═CN2 |
| 91 | 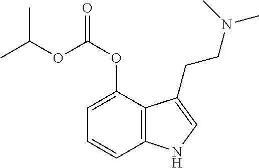 [SMILES: CC(C)OC(=O)Oc1cccc2[nH]cc(CCN(C)C)c12] |
| | O═C(OC(C)C)OC1═CC═CC2═C1C(CCN(C)C)═CN2 |
| 92 | 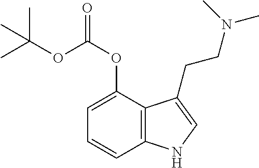 [SMILES: CN(C)CCc1c[nH]c2cccc(OC(=O)OC(C)(C)C)c12] |
| | O═C(OC(C)(C)C)OC1═CC═CC2═C1C(CCN(C)C)═CN2 |
| 93 | 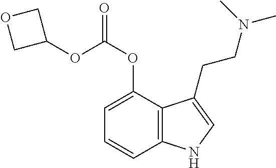 [SMILES: CN(C)CCc1c[nH]c2cccc(OC(=O)OC3COC3)c12] |
| | O═C(OC1COC1)OC2═CC═CC3═C2C(CCN(C)C)═CN3 |
| 94 | 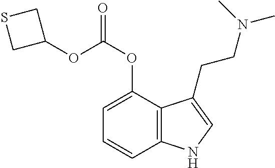 [SMILES: CN(C)CCc1c[nH]c2cccc(OC(=O)OC3CSC3)c12] |
| | O═C(OC1CSC1)OC2═CC═CC3═C2C(CCN(C)C)═CN3 |
| 95 | 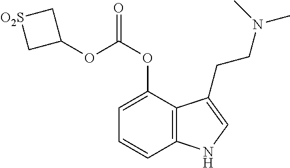 [SMILES: CN(C)CCc1c[nH]c2cccc(OC(=O)OC3CS(=O)(=O)C3)c12] |
| | O═C(OC1CS(C1)(═O)═O)OC2═CC═CC3═C2C(CCN(C)C)═CN3 |
| 96 | 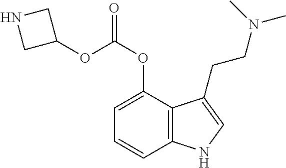 [SMILES: CN(C)CCc1c[nH]c2cccc(OC(=O)OC3CNC3)c12] |
| | O═C(OC1CNC1)OC2═CC═CC3═C2C(CCN(C)C)═CN3 |
| 97 | 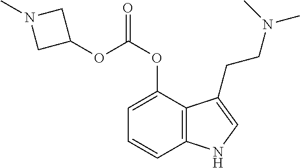 |
| | O═C(OC1CN(C)C1)OC2═CC═CC3═C2C(CCN(C)C)═CN3 |
| 98 | 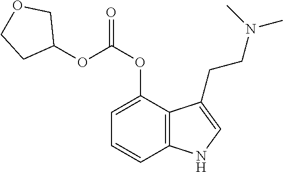 [SMILES: CN(C)CCc1c[nH]c2cccc(OC(=O)OC3CCOC3)c12] |
| | O═C(OC1COCC1)OC2═CC═CC3═C2C(CCN(C)C)═CN3 |
| 99 | 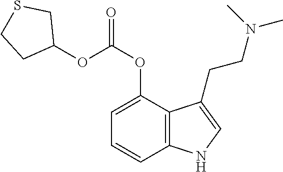 [SMILES: CN(C)CCc1c[nH]c2cccc(OC(=O)OC3CCSC3)c12] |
| | O═C(OC1CSCC1)OC2═CC═CC3═C2C(CCN(C)C)═CN3 |
| 100 | 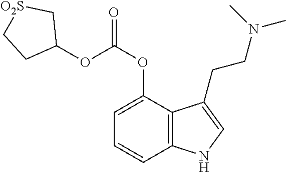 [SMILES: CN(C)CCc1c[nH]c2cccc(OC(=O)OC3CCS(=O)(=O)C3)c12] |
| | O═C(OC1CS(CC1)(═O)═O)OC2═CC═CC3═C2C(CCN(C)C)═CN3 |
| 101 | 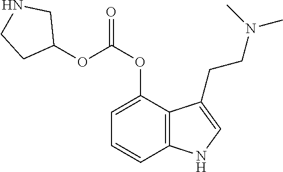 [SMILES: CN(C)CCc1c[nH]c2cccc(OC(=O)OC3CCNC3)c12] |
| | O═C(OC1CNCC1)OC2═CC═CC3═C2C(CCN(C)C)═CN3 |
| 102 | 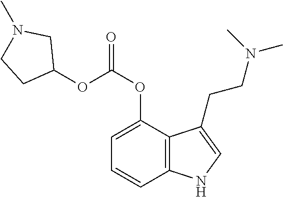 [SMILES: CN(C)CCc1c[nH]c2cccc(OC(=O)OC3CCN(C)C3)c12] |
| | O═C(OC1CN(C)CC1)OC2═CC═CC3═C2C(CCN(C)C)═CN3 |
| 103 | 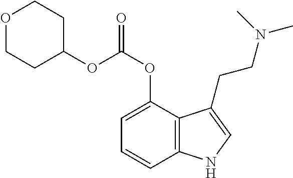 [SMILES: CN(C)CCc1c[nH]c2cccc(OC(=O)OC3CCOCC3)c12] |
| | O═C(OC1CCOCC1)OC2═CC═CC3═C2C(CCN(C)C)═CN3 |
| 104 | 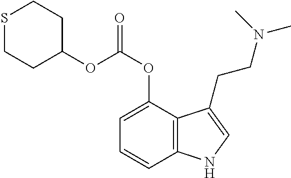 [SMILES: CN(C)CCc1c[nH]c2cccc(OC(=O)OC3CCSCC3)c12] |
| | O═C(OC1CCSCC1)OC2═CC═CC3═C2C(CCN(C)C)═CN3 |
| 105 | 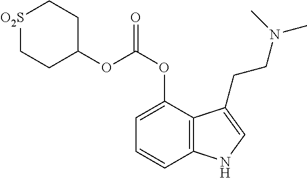 [SMILES: CN(C)CCc1c[nH]c2cccc(OC(=O)OC3CCS(=O)(=O)CC3)c12] |
| | O═C(OC1CCS(CC1)(═O)═O)OC2═CC═CC3═C2C(CCN(C)C)═CN3 |
| 106 | 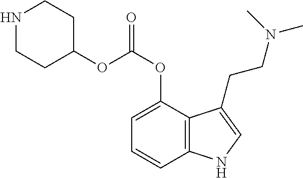 [SMILES: CN(C)CCc1c[nH]c2cccc(OC(=O)OC3CCNCC3)c12] |
| | O═C(OC1CCNCC1)OC2═CC═CC3═C2C(CCN(C)C)═CN3 |
| 107 | 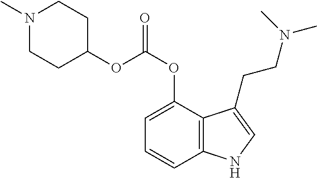 |
| | O═C(OC1CCN(C)CC1)OC2═CC═CC3═C2C(CCN(C)C)═CN3 |
| 108 | 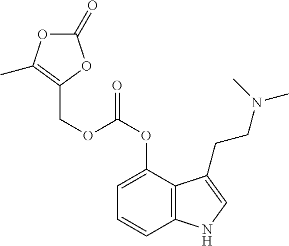 |
| | O═C(OCC(O1)═C(C)OC1═O)OC2═CC═CC3═C2C(CCN(C)C)═CN3 |
| 109 | 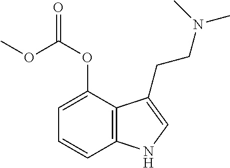 |
| | O═C(OC)OC1═CC═CC2═C1C(CCN(C)C)═CN2 |
| 110 | 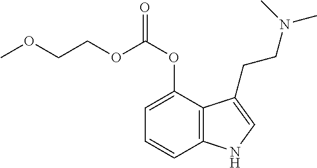 |
| | O═C(OCCOC)OC1═CC═CC2═C1C(CCN(C)C)═CN2 |
| 111 | 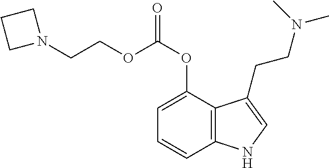 [SMILES: CN(C)CCc1c[nH]c2cccc(OC(=O)OCCN3CCC3)c12] |
| | O═C(OCCN1CCC1)OC2═CC═CC3═C2C(CCN(C)C)═CN3 |
| 112 | 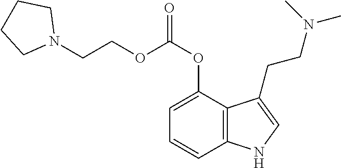 [SMILES: CN(C)CCc1c[nH]c2cccc(OC(=O)OCCN3CCCC3)c12] |
| | O═C(OCCN1CCCC1)OC2═CC═CC3═C2C(CCN(C)C)═CN3 |
| 113 | 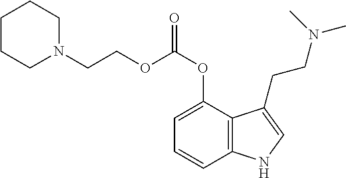 [SMILES: CN(C)CCc1c[nH]c2cccc(OC(=O)OCCN3CCCCC3)c12] |
| | O═C(OCCN1CCCC1)OC2═CC═CC3═C2C(CCN(C)C)═CN3 |
| 114 | 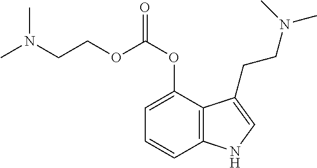 |
| | O═C(OCCN(C)C)OC1═CC═CC2═C1C(CCN(C)C)═CN2 |
| 115 | 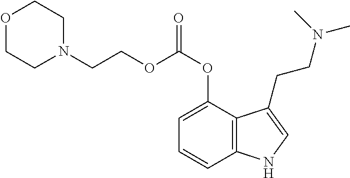 [SMILES: CN(C)CCc1c[nH]c2cccc(OC(=O)OCCN3CCOCC3)c12] |
| | O═C(OCCN1CCOCC1)OC2═CC═CC3═C2C(CCN(C)C)═CN3 |
| 116 | 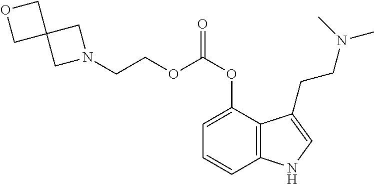 [SMILES: CN(C)CCc1c[nH]c2cccc(OC(=O)OCCN3CC4(COC4)C3)c12] |
| | O═C(OCCN1CC2(COC2)C1)OC3═CC═CC4═C3C(CCN(C)C)═CN4 |
| 117 | 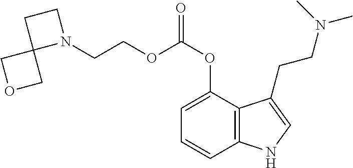 [SMILES: CN(C)CCc1c[nH]c2cccc(OC(=O)OCCN3CCC34COC4)c12] |
| | O═C(OCCN1CCC12COC2)OC3═CC═CC4═C3C(CCN(C)C)═CN4 |
| 118 | 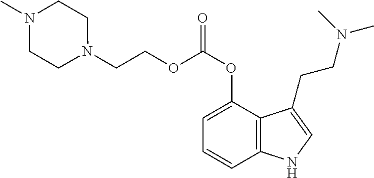 |
| | O═C(OCCN1CCN(C)CC1)OC2═CC═CC3═C2C(CCN(C)C)═CN3 |
| 119 | 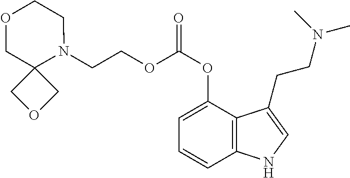 [SMILES: CN(C)CCc1c[nH]c2cccc(OC(=O)OCCN3CCOCC34COC4)c12] |
| | O═C(OCCN1CCOCC12COC2)OC3═CC═CC4═C3C(CCN(C)C)═CN4 |
| 120 | 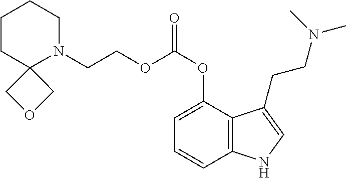 [SMILES: CN(C)CCc1c[nH]c2cccc(OC(=O)OCCN3CCCCC34COC4)c12] |
| | O═C(OCCN1CCCCC12COC2)OC3═CC═CC4═C3C(CCN(C)C)═CN4 |
| 121 | 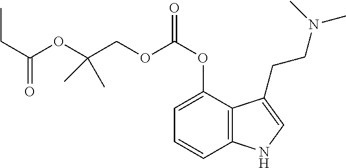 [SMILES: CCC(=O)OC(C)(C)COC(=O)Oc1cccc2[nH]cc(CCN(C)C)c12] |
| | O═C(OCC(C)(C)OC(CC)═O)OC1═CC═CC2═C1C(CCN(C)C)═CN2 |
| 122 | 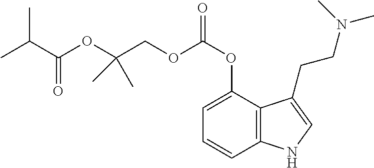 [SMILES: CC(C)C(=O)OC(C)(C)COC(=O)Oc1cccc2[nH]cc(CCN(C)C)c12] |
| | O═C(OCC(C)(C)OC(C(C)C)═O)OC1═CC═CC2═C1C(CCN(C)C)═CN2 |
| 123 | 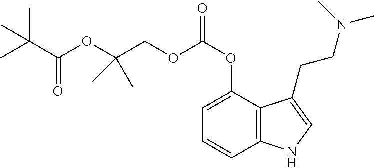 [SMILES: CN(C)CCc1c[nH]c2cccc(OC(=O)OCC(C)(C)OC(=O)C(C)(C)C)c12] |
| | O═C(OCC(C)(C)OC(C(C)(C)C)═O)OC1═CC═CC2═C1C(CCN(C)C)═CN2 |
| 124 | 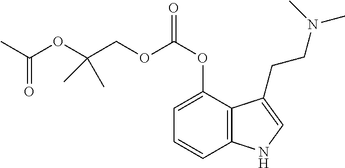 [SMILES: CC(=O)OC(C)(C)COC(=O)Oc1cccc2[nH]cc(CCN(C)C)c12] |
| | O═C(OCC(C)(C)OC(C)═O)OC1═CC═CC2═C1C(CCN(C)C)═CN2 |
| 125 | 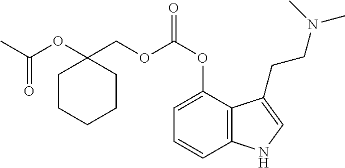 [SMILES: CC(=O)OC1(COC(=O)Oc2cccc3[nH]cc(CCN(C)C)c23)CCCCC1] |
| | O═C(OCC1(CCCCC1)OC(C)═O)OC2═CC═CC3═C2C(CCN(C)C)═CN3 |
| 126 | 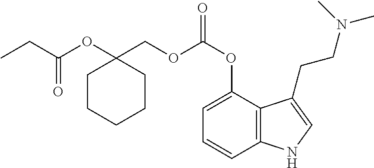 [SMILES: CCC(=O)OC1(COC(=O)Oc2cccc3[nH]cc(CCN(C)C)c23)CCCCC1] |
| | O═C(OCC1(CCCCC1)OC(CC)═O)OC2═CC═CC3═C2C(CCN(C)C)═CN3 |
| 127 | 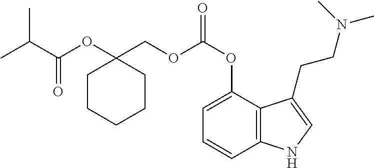 [SMILES: CC(C)C(=O)OC1(COC(=O)Oc2cccc3[nH]cc(CCN(C)C)c23)CCCCC1] |
| | O═C(OCC1(CCCCC1)OC(C(C)C)═O)OC2═CC═CC3═C2C(CCN(C)C)═CN3 |
| 128 | 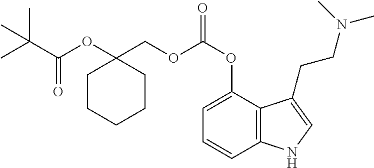 [SMILES: CN(C)CCc1c[nH]c2cccc(OC(=O)OCC3(OC(=O)C(C)(C)C)CCCCC3)c12] |
| | O═C(OCC1(CCCCC1)OC(C(C)(C)C)═O)OC2═CC═CC3═C2C(CCN(C)C)═CN3 |
| 129 | 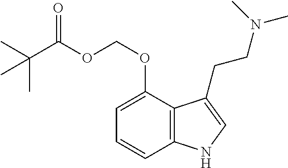 [SMILES: CN(C)CCc1c[nH]c2cccc(OCOC(=O)C(C)(C)C)c12] |
| | CN(C)CCC1═CNC2═C1C(OCOC(C(C)(C)C)═O)═CC═C2 |
| 130 | 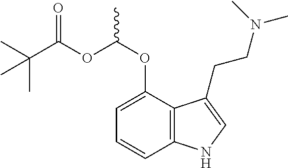 [SMILES: CC(OC(=O)C(C)(C)C)Oc1cccc2[nH]cc(CCN(C)C)c12] |
| | CN(C)CCC1═CNC2═C1C(OC(C)OC(C(C)(C)C)═O)═CC═C2 |
| 131 | 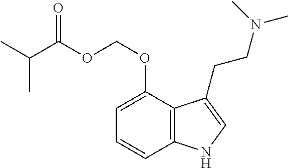 [SMILES: CC(C)C(=O)OCOc1cccc2[nH]cc(CCN(C)C)c12] |
| | CN(C)CCC1═CNC2═C1C(OCOC(C(C)C)═O)═CC═C2 |
| 132 | 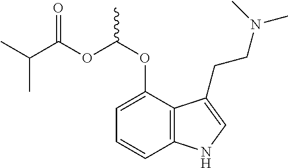 [SMILES: CC(OC(=O)C(C)C)Oc1cccc2[nH]cc(CCN(C)C)c12] |
| | CN(C)CCC1═CNC2═C1C(OC(C)OC(C(C)C)═O)═CC═C2 |
| 133 | 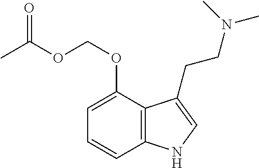 [SMILES: CC(=O)OCOc1cccc2[nH]cc(CCN(C)C)c12] |
| | CN(C)CCC1═CNC2═C1C(OCOC(C)═O)═CC═C2 |
| 134 | 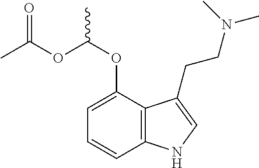 [SMILES: CC(=O)OC(C)Oc1cccc2[nH]cc(CCN(C)C)c12] |
| | CN(C)CCC1═CNC2═C1C(OC(C)OC(C)═O)═CC═C2 |
| 135 | 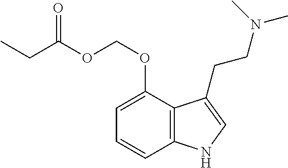 [SMILES: CCC(=O)OCOc1cccc2[nH]cc(CCN(C)C)c12] |
| | CN(C)CCC1═CNC2═C1C(OCOC(CC)═O)═CC═C2 |
| 136 | 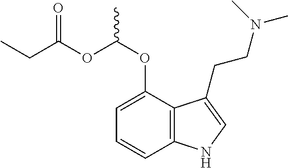 [SMILES: CCC(=O)OC(C)Oc1cccc2[nH]cc(CCN(C)C)c12] |
| | CN(C)CCC1═CNC2═C1C(OC(C)OC(CC)═O)═CC═C2 |
| 137 | 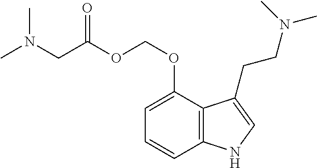 |
| | CN(C)CCC1═CNC2═C1C(OCOC(CN(C)C)═O)═CC═C2 |
| 138 | 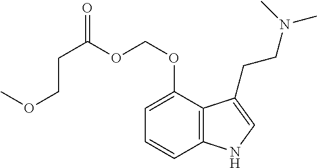 |
| | CN(C)CCC1═CNC2═C1C(OCOC(CCOC)═O)═CC═C2 |
| 139 | 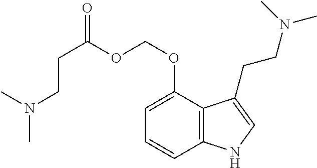 |
| | CN(C)CCC1═CNC2═C1C(OCOC(CCN(C)C)═O)═CC═C2 |
| 140 | 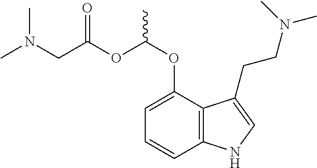 |
| | CN(C)CCC1═CNC2═C1C(OC(C)OC(CN(C)C)═O)═CC═C2 |
| 141 | 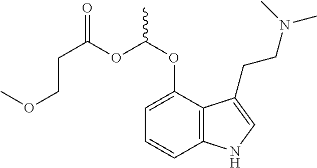 |
| | CN(C)CCC1═CNC2═C1C(OC(C)OC(CCOC)═O)═CC═C2 |
| 142 | 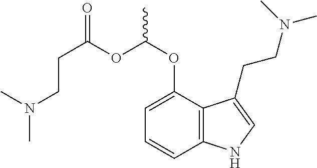 |
| | CN(C)CCC1═CNC2═C1C(OC(C)OC(CCN(C)C)═O)═CC═C2 |
| 143 | 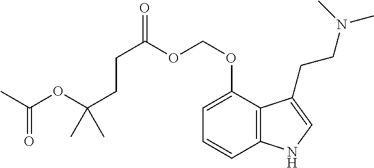 [SMILES: CC(=O)OC(C)(C)CCC(=O)OCOc1cccc2[nH]cc(CCN(C)C)c12] |
| | CN(C)CCC1═CNC2═C1C(OCOC(CCC(C)(C)OC(C)═O)═O)═CC═C2 |
| 144 | 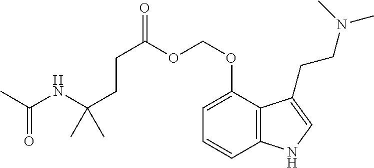 [SMILES: CC(=O)NC(C)(C)CCC(=O)OCOc1cccc2[nH]cc(CCN(C)C)c12] |
| | CN(C)CCC1═CNC2═C1C(OCOC(CCC(C)(C)NC(C)═O)═O)═CC═C2 |
| 145 | 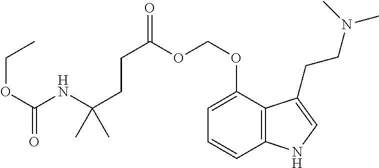 [SMILES: CCOC(=O)NC(C)(C)CCC(=O)OCOc1cccc2[nH]cc(CCN(C)C)c12] |
| | CN(C)CCC1═CNC2═C1C(OCOC(CCC(C)(C)NC(OCC)═O)═O)═CC═C |
| 146 | 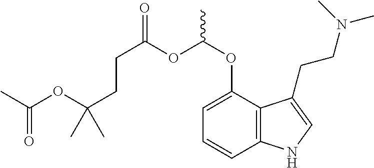 [SMILES: CC(=O)OC(C)(C)CCC(=O)OC(C)Oc1cccc2[nH]cc(CCN(C)C)c12] |
| | CN(C)CCC1═CNC2═C1C(OC(C)OC(CCC(C)(C)OC(C)═O)═O)═CC═C2 |
| 147 | 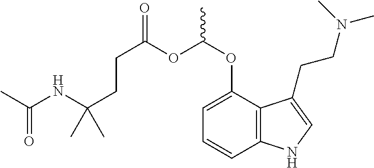 [SMILES: CC(=O)NC(C)(C)CCC(=O)OC(C)Oc1cccc2[nH]cc(CCN(C)C)c12] |
| | CN(C)CCC1═CNC2═C1C(OC(C)OC(CCC(C)(C)NC(C)═O)═O)═CC═C2 |
| 148 | 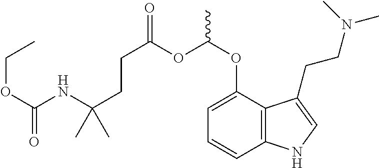 [SMILES: CCOC(=O)NC(C)(C)CCC(=O)OC(C)Oc1cccc2[nH]cc(CCN(C)C)c12] |
| | CN(C)CCC1═CNC2═C1C(OC(C)OC(CCC(C)(C)NC(OCC)═O)═O)═CC═C2 |
| 149 | 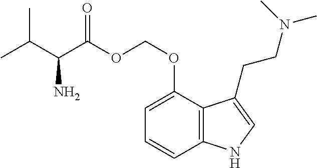 [SMILES: CC(C)[C@H](N)C(=O)OCOc1cccc2[nH]cc(CCN(C)C)c12] |
| | CN(C)CCC1═CNC2═C1C(OCOC([C@H](C(C)C)N)═O)═CC═C2 |
| 150 | 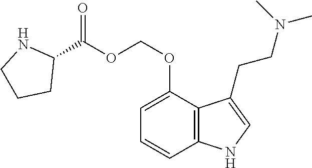 [SMILES: CN(C)CCc1c[nH]c2cccc(OCOC(=O)[C@@H]3CCCN3)c12] |
| | CN(C)CCC1═CNC2═C1C(OCOC([C@H]3NCCC3)═O)═CC═C2 |
| 151 | 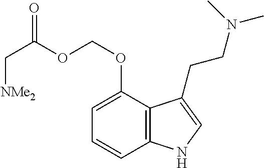 [SMILES: CN(C)CCc1c[nH]c2cccc(OCOC(=O)CN(C)C)c12] |
| | CN(C)CCC1═CNC2═C1C(OCOC(CN(C)C)═O)═CC═C2 |
| 152 | 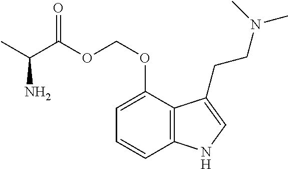 [SMILES: C[C@H](N)C(=O)OCOc1cccc2[nH]cc(CCN(C)C)c12] |
| | CN(C)CCC1═CNC2═C1C(OCOC([C@H](C)N)═O)═CC═C2 |
| 153 | 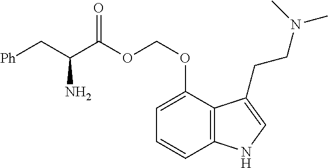 [SMILES: CN(C)CCc1c[nH]c2cccc(OCOC(=O)[C@@H](N)Cc3ccccc3)c12] |
| | CN(C)CCC1═CNC2═C1C(OCOC([C@H](CC3═CC═CC═C3)N)═O)═CC═C2 |
| 154 | 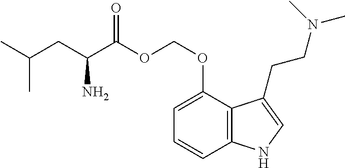 [SMILES: CC(C)C[C@H](N)C(=O)OCOc1cccc2[nH]cc(CCN(C)C)c12] |
| | CN(C)CCC1═CNC2═C1C(OCOC([C@H](CC(C)C)N)═O)═CC═C2 |
| 155 | 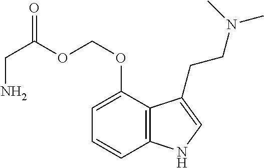 [SMILES: CN(C)CCc1c[nH]c2cccc(OCOC(=O)CN)c12] |
| | CN(C)CCC1═CNC2═C1C(OCOC(CN)═O)═CC═C2 |
| 156 | 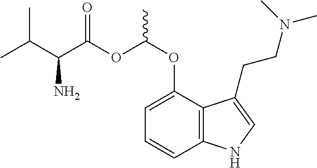 [SMILES: CC(OC(=O)[C@@H](N)C(C)C)Oc1cccc2[nH]cc(CCN(C)C)c12] |
| | CN(C)CCC1═CNC2═C1C(OC(C)OC([C@H](C(C)C)N)═O)═CC═C2 |
| 157 | 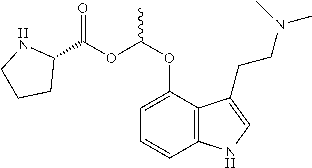 [SMILES: CC(OC(=O)[C@@H]1CCCN1)Oc1cccc2[nH]cc(CCN(C)C)c12] |
| | CN(C)CCC1═CNC2═C1C(OC(C)OC([C@H]3NCCC3)═O)═CC═C2 |
| 158 | 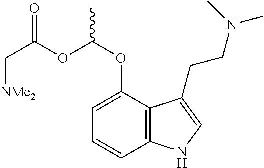 [SMILES: CC(OC(=O)CN(C)C)Oc1cccc2[nH]cc(CCN(C)C)c12] |
| | CN(C)CCC1═CNC2═C1C(OC(C)OC(CN(C)C)═O)═CC═C2 |
| 159 | 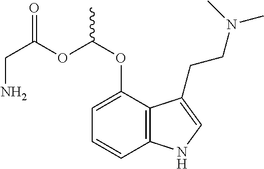 [SMILES: CC(OC(=O)CN)Oc1cccc2[nH]cc(CCN(C)C)c12] |
| | CN(C)CCC1═CNC2═C1C(OC(C)OC(CN)═O)═CC═C2 |
| 160 | 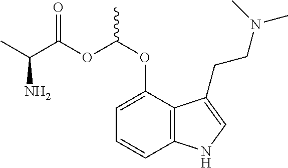 [SMILES: CC(OC(=O)[C@H](C)N)Oc1cccc2[nH]cc(CCN(C)C)c12] |
| | CN(C)CCC1═CNC2═C1C(OC(C)OC([C@H](C)N)═O)═CC═C2 |
| 161 | 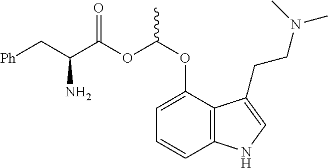 [SMILES: CC(OC(=O)[C@@H](N)Cc1ccccc1)Oc1cccc2[nH]cc(CCN(C)C)c12] |
| | CN(C)CCC1═CNC2═C1C(OC(C)OC([C@H](CC3═CC═CC═C3)N) |
| | ═O)═CC═C2 |
| 162 | 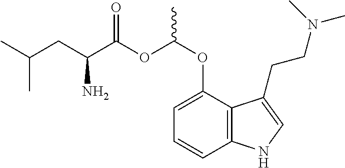 [SMILES: CC(C)C[C@H](N)C(=O)OC(C)Oc1cccc2[nH]cc(CCN(C)C)c12] |
| | CN(C)CCC1═CNC2═C1C(OC(C)OC([C@H](CC(C)C)N)═O)═CC═C2 |
| 163 | 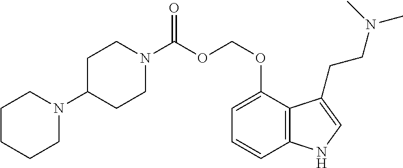 [SMILES: CN(C)CCc1c[nH]c2cccc(OCOC(=O)N3CCC(N4CCCCC4)CC3)c12] |
| | CN(C)CCC1═CNC2═C1C(OCOC(N3CCC(N4CCCCC4)CC3)═O)CC═C2 |
| 164 | 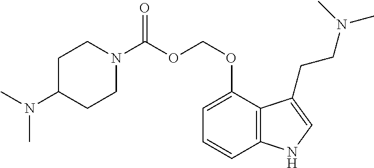 |
| | CN(C)CCC1═CNC2═C1C(OCOC(N3CCC(N(C)C)CC3)═O)═CC═C2 |
| 165 | 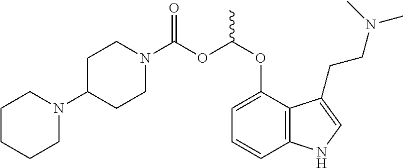 [SMILES: CC(OC(=O)N1CCC(N2CCCCC2)CC1)Oc1cccc2[nH]cc(CCN(C)C)c12] |
| | CN(C)CCC1═CNC2═C1C(OC(C)OC(N3CCC(N4CCCCC4)CC3)═O)═CC═C2 |
| 166 | 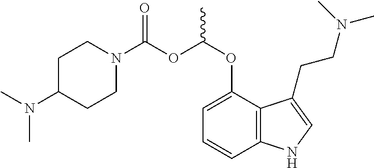 |
| | CN(C)CCC1═CNC2═C1C(OC(C)OC(N3CCC(N(C)C)CC3)═O)═CC═C2 |
| 167 | 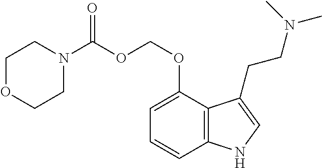 [SMILES: CN(C)CCc1c[nH]c2cccc(OCOC(=O)N3CCOCC3)c12] |
| | CN(C)CCC1═CNC2═C1C(OCOC(N3CCOCC3)═O)═CC═C2 |
| 168 | 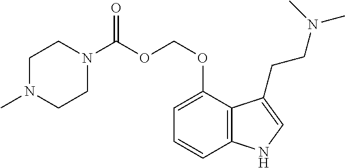 |
| | CN(C)CCC1═CNC2═C1C(OCOC(N3CCN(C)CC3)═O)═CC═C2 |
| 169 | 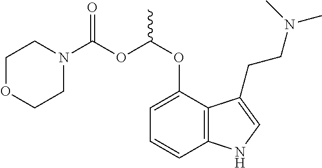 [SMILES: CC(OC(=O)N1CCOCC1)Oc1cccc2[nH]cc(CCN(C)C)c12] |
| | CN(C)CCC1═CNC2═C1C(OC(C)OC(N3CCOCC3)═O)═CC═C2 |
| 170 | 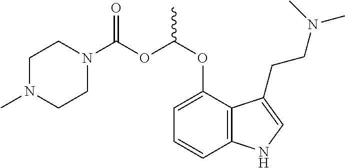 |
| | CN(C)CCC1═CNC2═C1C(OC(C)OC(N3CCN(C)CC3)═O)═CC═C2 |
| 171 | 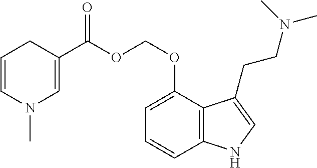 [SMILES: CN1C=CCC(C(=O)OCOc2cccc3[nH]cc(CCN(C)C)c23)=C1] |
| | CN(C)CCC1═CNC2═C1C(OCOC(C3═CN(C)C═CC3)═O)═CC═C2 |
| 172 | 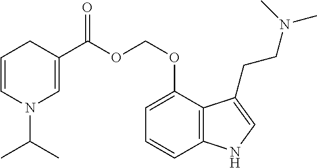 [SMILES: CC(C)N1C=CCC(C(=O)OCOc2cccc3[nH]cc(CCN(C)C)c23)=C1] |
| | CN(C)CCC1═CNC2═C1C(OCOC(C3═CN(C(C)C)C═CC3)═O)═CC═C2 |
| 173 | 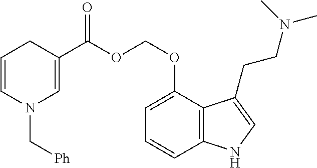 [SMILES: CN(C)CCc1c[nH]c2cccc(OCOC(=O)C3=CN(Cc4ccccc4)C=CC3)c12] |
| | CN(C)CCC1═CNC2═C1C(OCOC(C3═CN(CC4═CC═CC═C4) |
| | C═CC3)═O)═CC═C2 |
| 174 | 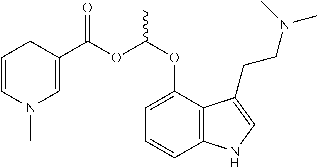 [SMILES: CC(OC(=O)C1=CN(C)C=CC1)Oc1cccc2[nH]cc(CCN(C)C)c12] |
| | CN(C)CCC1═CNC2═C1C(OC(C)OC(C3═CN(C)C═CC3)═O)═CC═C2 |
| 175 | 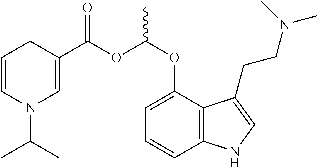 [SMILES: CC(OC(=O)C1=CN(C(C)C)C=CC1)Oc1cccc2[nH]cc(CCN(C)C)c12] |
| | CN(C)CCC1═CNC2═C1C(OC(C)OC(C3═CN(C(C)C)C═CC3)═O)═CC═C2 |
| 176 | 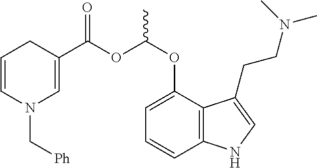 [SMILES: CC(OC(=O)C1=CN(Cc2ccccc2)C=CC1)Oc1cccc2[nH]cc(CCN(C)C)c12] |
| | CN(C)CCC1═CNC2═C1C(OC(C)OC(C3═CN(CC4═CC═CC═C4) |
| | C═CC3)═O)═CC═C2 |
| 177 | 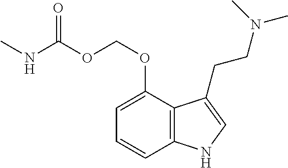 |
| | CN(C)CCC1═CNC2═C1C(OCOC(NC)═O)═CC═C2 |
| 178 | 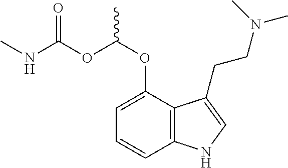 |
| | CN(C)CCC1═CNC2═C1C(OC(C)OC(NC)═O)═CC═C2 |
| 179 | 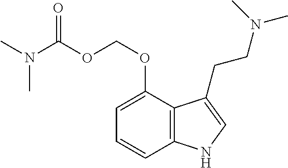 |
| | CN(C)CCC1═CNC2═C1C(OCOC(N(C)C)═O)═CC═C2 |
| 180 | 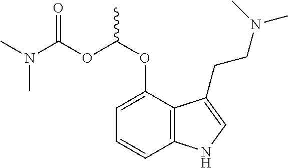 |
| | CN(C)CCC1═CNC2═C1C(OC(C)OC(N(C)C)═O)═CC═C2 |
| 181 | 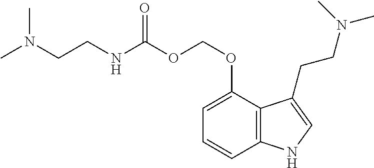 |
| | CN(C)CCC1═CNC2═C1C(OCOC(NCCN(C)C)═O)═CC═C2 |
| 182 | 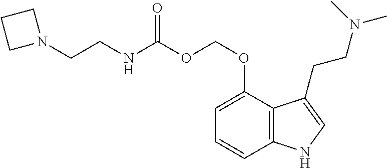 [SMILES: CN(C)CCc1c[nH]c2cccc(OCOC(=O)NCCN3CCC3)c12] |
| | CN(C)CCC1═CNC2═C1C(OCOC(NCCN3CCC3)═O)═CC═C2 |
| 183 | 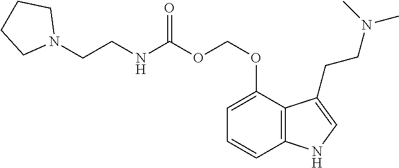 [SMILES: CN(C)CCc1c[nH]c2cccc(OCOC(=O)NCCN3CCCC3)c12] |
| | CN(C)CCC1═CNC2═C1C(OCOC(NCCN3CCCC3)═O)═CC═C2 |
| 184 | 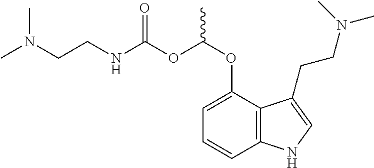 |
| | CN(C)CCC1═CNC2═C1C(OC(C)OC(NCCN(C)C)═O)═CC═C2 |
| 185 | 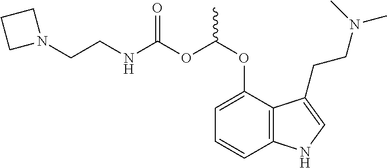 [SMILES: CC(OC(=O)NCCN1CCC1)Oc1cccc2[nH]cc(CCN(C)C)c12] |
| | CN(C)CCC1═CNC2═C1C(OC(C)OC(NCCN3CCC3)═O)═CC═C2 |
| 186 | 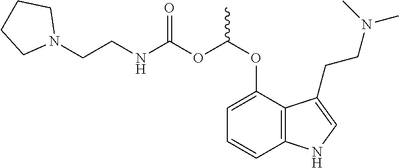 [SMILES: CC(OC(=O)NCCN1CCCC1)Oc1cccc2[nH]cc(CCN(C)C)c12] |
| | CN(C)CCC1═CNC2═C1C(OC(C)OC(NCCN3CCCC3)═O)═CC═C2 |
| 187 | 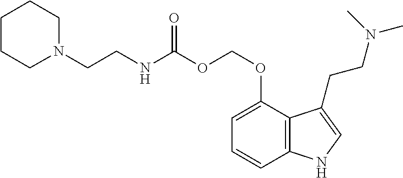 [SMILES: CN(C)CCc1c[nH]c2cccc(OCOC(=O)NCCN3CCCCC3)c12] |
| | CN(C)CCC1═CNC2═C1C(OCOC(NCCN3CC4(COC4)C3)═O)═CC═C2 |
| 188 | 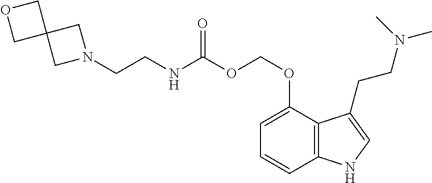 [SMILES: CN(C)CCc1c[nH]c2cccc(OCOC(=O)NCCN3CC4(COC4)C3)c12] |
| | CN(C)CCC1═CNC2═C1C(OCOC(NCCN3CC4(COC4)C3)═O)═CC═C2 |
| 189 | 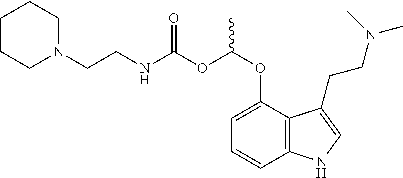 [SMILES: CC(OC(=O)NCCN1CCCCC1)Oc1cccc2[nH]cc(CCN(C)C)c12] |
| | CN(C)CCC1═CNC2═C1C(OC(C)OC(NCCN3CCCCC3)═O)═CC═C2 |
| 190 | 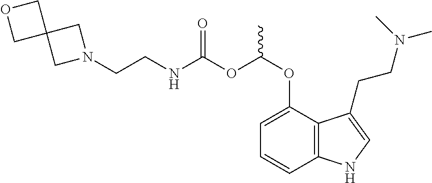 [SMILES: CC(OC(=O)NCCN1CC2(COC2)C1)Oc1cccc2[nH]cc(CCN(C)C)c12] |
| | CN(C)CCC1═CNC2═C1C(OC(C)OC(NCCN3CC4(COC4)C3)═O)═CC═C2 |
| 191 | 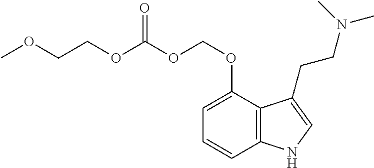 |
| | CN(C)CCC1═CNC2═C1C(OCOC(OCCOC)═O)═CC═C2 |
| 192 | 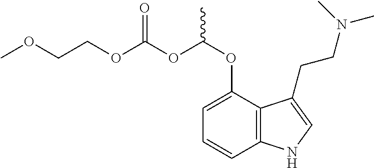 |
| | CN(C)CCC1═CNC2═C1C(OC(C)OC(OCCOC)═O)═CC═C2 |
| 193 | 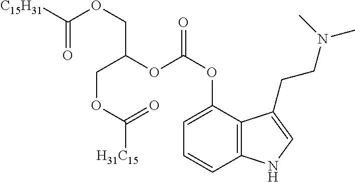 |
| | CN(C)CCC1═CNC2═C1C(OC(OC(COC(CCCCCCCCCCCCCCC) |
| | ═O)COC(CCCCCCCCCCCCCCC)═O)═O)═CC═C2 |
| 194 | 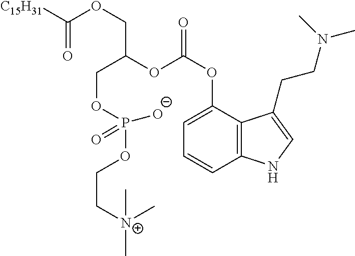 |
| | CN(C)CCC1═CNC2═C1C(OC(OC(COP(OCC[N+](C)(C)C)([O−])═O) |
| | COC(CCCCCCCCCCCCCCC)═O)═O)═CC═C2 |
| 195 | 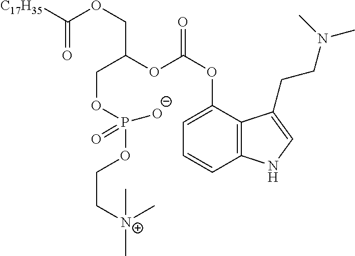 |
| | CN(C)CCC1═CNC2═C1C(OC(OC(COP(OCC[N+](C)(C)C)([O−])═O)COC |
| | (CCCCCCCCCCCCCCCCC)═O)═O)═CC═C2 |
| 196 | 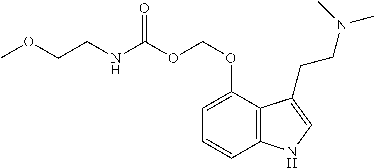 |
| | CN(C)CCC1═CNC2═C1C(OCOC(NCCOC)═O)═CC═C2 |
| 197 | 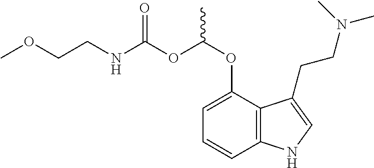 |
| | CN(C)CCC1═CNC2═C1C(OC(C)OC(NCCOC)═O)═CC═C2 |
| 198 | 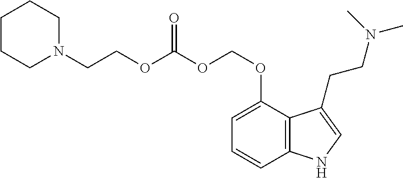 [SMILES: CN(C)CCc1c[nH]c2cccc(OCOC(=O)OCCN3CCCCC3)c12] |
| | CN(C)CCC1═CNC2═C1C(OCOC(OCCN3CCCCC3)═O)═CC═C2 |
| 199 | 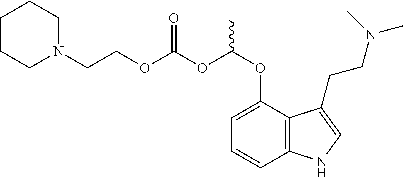 [SMILES: CC(OC(=O)OCCN1CCCCC1)Oc1cccc2[nH]cc(CCN(C)C)c12] |
| | CN(C)CCC1═CNC2═C1C(OC(C)(OCCN3CCCCC3)═O)═CC═C2 |
| 200 | 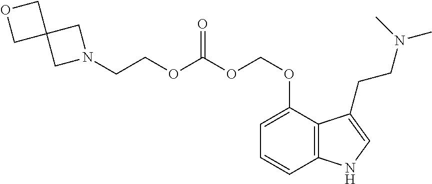 [SMILES: CN(C)CCc1c[nH]c2cccc(OCOC(=O)OCCN3CC4(COC4)C3)c12] |
| | CN(C)CCC1═CNC2═C1C(OCOC(OCCN3CC4(COC4)C3)═O)═CC═C2 |
| 201 | 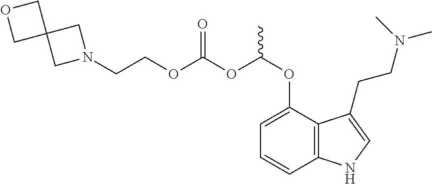 [SMILES: CC(OC(=O)OCCN1CC2(COC2)C1)Oc1cccc2[nH]cc(CCN(C)C)c12] |
| | CN(C)CCC1═CNC2═C1C(OC(C)OC(OCCNC3CC4(COC4)C3)═O)═CC═C2 |
| 202 | 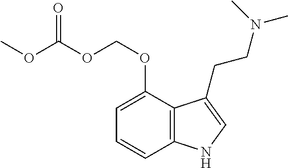 |
| | CN(C)CCC1═CNC2═C1C(OCOC(OC)═O)═CC═C2 |
| 203 | 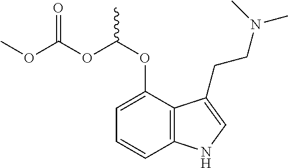 |
| | CN(C)CCC1═CNC2═C1C(OC(C)OC(OC)═O)═CC═C2 |
| 204 | 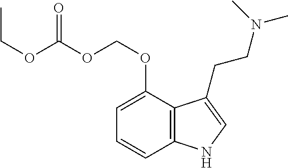 [SMILES: CCOC(=O)OCOc1cccc2[nH]cc(CCN(C)C)c12] |
| | CN(C)CCC1═CNC2═C1C(OCOC(OCC)═O)═CC═C2 |
| 205 | 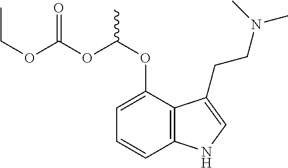 [SMILES: CCOC(=O)OC(C)Oc1cccc2[nH]cc(CCN(C)C)c12] |
| | CN(C)CCC1═CNC2═C1C(OC(C)OC(OCC)═O)═CC═C2 |
| 206 | 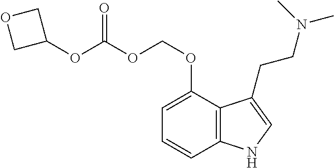 [SMILES: CN(C)CCc1c[nH]c2cccc(OCOC(=O)OC3COC3)c12] |
| | CN(C)CCC1═CNC2═C1C(OCOC(OC3COC3)═O)═CC═C2 |
| 207 | 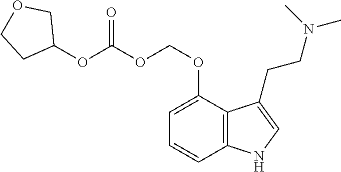 [SMILES: CN(C)CCc1c[nH]c2cccc(OCOC(=O)OC3CCOC3)c12] |
| | CN(C)CCC1═CNC2═C1C(OCOC(OC3COCC3)═O)═CC═C2 |
| 208 | 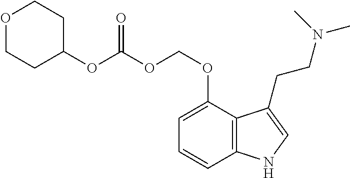 [SMILES: CN(C)CCc1c[nH]c2cccc(OCOC(=O)OC3CCOCC3)c12] |
| | CN(C)CCC1═CNC2═C1C(OCOC(OC3CCOCC3)═O)═CC═C2 |
| 209 | 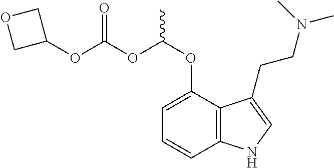 [SMILES: CC(OC(=O)OC1COC1)Oc1cccc2[nH]cc(CCN(C)C)c12] |
| | CN(C)CCC1═CNC2═C1C(OC(C)OC(OC3COC3)═O)═CC═C2 |
| 210 | 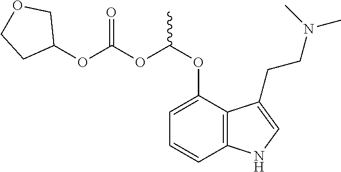 [SMILES: CC(OC(=O)OC1CCOC1)Oc1cccc2[nH]cc(CCN(C)C)c12] |
| | CN(C)CCC1═CNC2═C1C(OC(C)OC(OC3COCC3)═O)═CC═C2 |
| 211 | 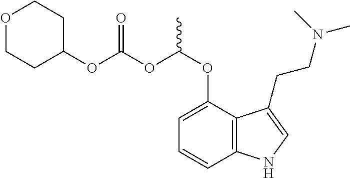 [SMILES: CC(OC(=O)OC1CCOCC1)Oc1cccc2[nH]cc(CCN(C)C)c12] |
| | CN(C)CCC1═CNC2═C1C(OC(C)OC(OC3CCOCC3)═O)═CC═C2 |
| 212 | 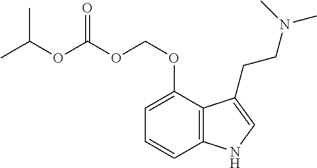 [SMILES: CC(C)OC(=O)OCOc1cccc2[nH]cc(CCN(C)C)c12] |
| | CN(C)CCC1═CNC2═C1C(OCOC(OC(C)C)═O)═CC═C2 |
| 213 | 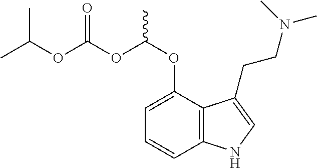 [SMILES: CC(C)OC(=O)OC(C)Oc1cccc2[nH]cc(CCN(C)C)c12] |
| | CN(C)CCC1═CNC2═C1C(OC(C)OC(OC(C)C)═O)═CC═C2 |
| 214 | 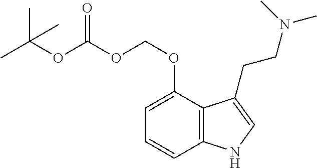 [SMILES: CN(C)CCc1c[nH]c2cccc(OCOC(=O)OC(C)(C)C)c12] |
| | CN(C)CCC1═CNC2═C1C(OCOC(OC(C)(C)C)═O)═CC═C2 |
| 215 | 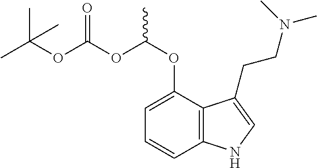 [SMILES: CC(OC(=O)OC(C)(C)C)Oc1cccc2[nH]cc(CCN(C)C)c12] |
| | CN(C)CCC1═CNC2═C1C(OC(C)OC(OC(C)(C)C)═CC═C2 |
| 216 | 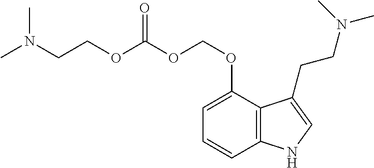 |
| | CN(C)CCC1═CNC2═C1C(OCOC(OCCN(C)C)═O═CC═C2 |
| 217 | 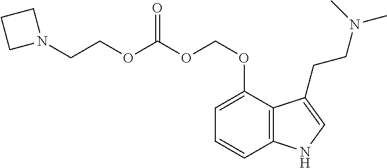 [SMILES: CN(C)CCc1c[nH]c2cccc(OCOC(=O)OCCN3CCC3)c12] |
| | CN(C)CCC1═CNC2═C1C(OCOC(OCCN3CCC3)═O)═CC═C2 |
| 218 | 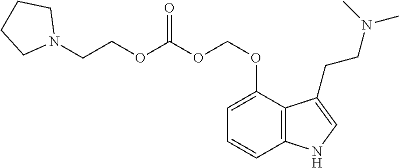 [SMILES: CN(C)CCc1c[nH]c2cccc(OCOC(=O)OCCN3CCCC3)c12] |
| | CN(C)CCC1═CNC2═C1C(OCOC(OCCN3CCC3)═O)═CC═C2 |
| 219 | 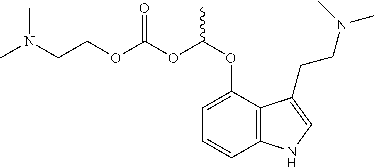 |
| | CN(C)CCC1═CNC2═C1C(OC(C)OC(OCCN(C)C)═O)═CC═C2 |
| 220 | 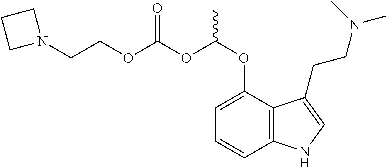 [SMILES: CC(OC(=O)OCCN1CCC1)Oc1cccc2[nH]cc(CCN(C)C)c12] |
| | CN(C)CCC1═CNC2═C1C(OC(C)OC(OCCN3CCC3)═O)═CC═C2 |
| 221 | 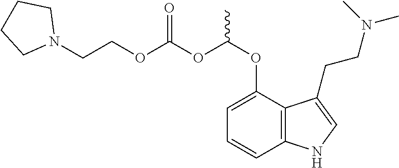 [SMILES: CC(OC(=O)OCCN1CCCC1)Oc1cccc2[nH]cc(CCN(C)C)c12] |
| | CN(C)CCC1═CNC2═C1C(OC(C)OC(OCCN3CCCC3)═O)═CC═C2 |
| 222 | 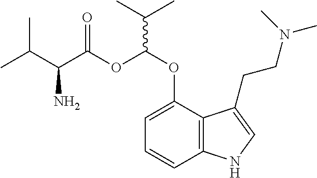 [SMILES: CC(C)C(OC(=O)[C@@H](N)C(C)C)Oc1cccc2[nH]cc(CCN(C)C)c12] |
| | CN(C)CCC1═CNC2═C1C(OC(C(C)C)OC([C@H](C(C)C)N)═O)═CC═C2 |
| 223 | 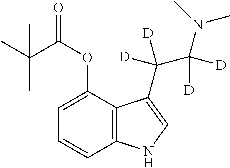 [SMILES: [2H]C([2H])(c1c[nH]c2cccc(OC(=O)C(C)(C)C)c12)C([2H])([2H])N(C)C] |
| | O═C(C(C)(C)C)OC1═CC═CC2═C1C(C(C[2H]) |
| | ([2H])C([2H])([2H])N(C)C)═CN2 |
| 224 | 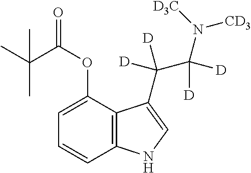 [SMILES: [2H]C([2H])([2H])N(C([2H])([2H])[2H])C([2H])([2H])C([2H])([2H])c1c[nH]c2cccc(OC(=O)C(C)(C)C)c12] |
| | O═C(C(C)(C)C)OC1═CC═CC2═C1C(C([2H])([2H])C([2H]) |
| | ([2H])N(C([2H])([2H])[2H])C([2H])([2H])[2H])═CN2 |
| 225 | 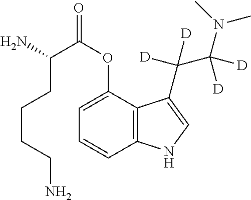 [SMILES: [2H]C([2H])(c1c[nH]c2cccc(OC(=O)[C@@H](N)CCCCN)c12)C([2H])([2H])N(C)C] |
| | O═C([C@@H](N)CCCCN)OC1═CC═CC2═C1C(C([2H]) |
| | ([2H])C([2H])([2H])N(C)C)═CN2 |
| 226 | 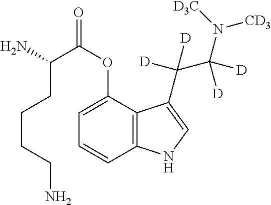 [SMILES: [2H]C([2H])([2H])N(C([2H])([2H])[2H])C([2H])([2H])C([2H])([2H])c1c[nH]c2cccc(OC(=O)[C@@H](N)CCCCN)c12] |
| | O═C([C@@H](N)CCCCN)OC1═CC═CC2═C1C(C([2H]) |
| | ([2H])C([2H])([2H])N(C([2H])([2H])[2H])C([2H])([2H])[2H])═CN2 |
| 227 | 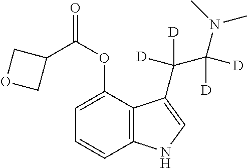 [SMILES: [2H]C([2H])(c1c[nH]c2cccc(OC(=O)C3COC3)c12)C([2H])([2H])N(C)C] |
| | O═C(C1COC1)OC2═CC═CC3═C2C(C([2H])([2H])C([2H])([2H])N(C)C)═CN3 |
| 228 | 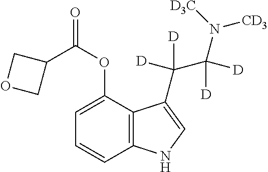 [SMILES: [2H]C([2H])([2H])N(C([2H])([2H])[2H])C([2H])([2H])C([2H])([2H])c1c[nH]c2cccc(OC(=O)C3COC3)c12] |
| | O═C(C1COC1)OC2═CC═CC3═C2C(C([2H])([2H]) |
| | C([2H])([2H])N(C([2H])([2H])[2H])C([2H])([2H])[2H]═CN3 |
| 229 | 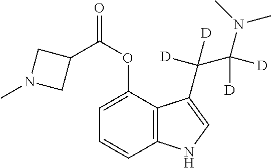 |
| | O═C(C1CN(C)C1)OC2═CC═CC3═C2C(C([2H])([2H]) |
| | C([2H])([2H])N(C)C)═CN3 |
| 230 | 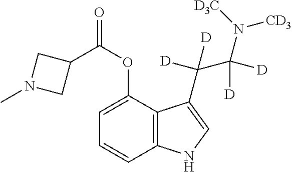 [SMILES: [2H]C([2H])([2H])N(C([2H])([2H])[2H])C([2H])([2H])C([2H])([2H])c1c[nH]c2cccc(OC(=O)C3CN(C)C3)c12] |
| | O═C(C1CN(C)C1)OC2═CC═CC3═C2C(C([2H])([2H]) |
| | C([2H])([2H])N(C([2H])([2H])[2H])C([2H])([2H][2H])═CN3 |
| 231 | 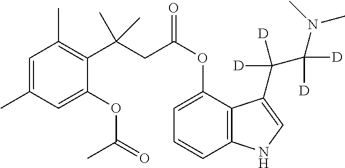 |
| | O═C(CC(C)(C)C1═C(C)C═C(C)C═C1OC(C)═O) |
| | OC2═CC═CC3═C2C(C([2H]([2H])C([2H])([2H])N(C)C)═CN3 |
| 232 | 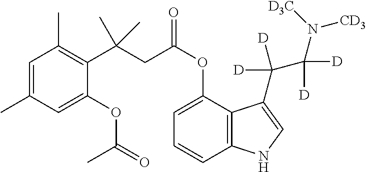 [SMILES: [2H]C([2H])([2H])N(C([2H])([2H])[2H])C([2H])([2H])C([2H])([2H])c1c[nH]c2cccc(OC(=O)CC(C)(C)c3c(C)cc(C)cc3OC(C)=O)c12] |
| | O═C(CC(C)(C)C1═C(C)C═C(C)C═C1OC(C)═O) |
| | OC2═CC═CC3═C2C(C([2H])([2H])C([2H])([2H]) |
| | N(C([2H])([2H])[2H])C([2H])([2H][2H]═CN3 |
| 233 | 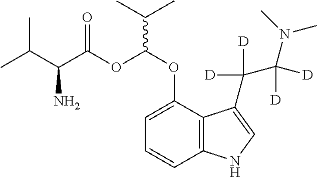 [SMILES: [2H]C([2H])(c1c[nH]c2cccc(OC(OC(=O)[C@@H](N)C(C)C)C(C)C)c12)C([2H])([2H])N(C)C] |
| | CN(C)C([2H])([2H])C([2H])([2H])C1═CNC2═C1C(OC(C(C)C) |
| | OC([C@H](C(C)C)N)═O)═CC═C2 |
| 234 | 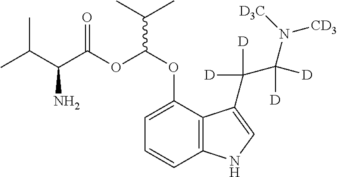 [SMILES: [2H]C([2H])([2H])N(C([2H])([2H])[2H])C([2H])([2H])C([2H])([2H])c1c[nH]c2cccc(OC(OC(=O)[C@@H](N)C(C)C)C(C)C)c12] |
| | O═([C@H](C(C)C)N)OC(C(C)C)OC1═CC═CC2═C1C(C([2H]) |
| | ([2H])C([2H])([2H])N(C([2H])([2H])[2H])C([2H])([2H])[2H])═CN2 |
| 235 | 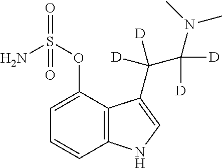 [SMILES: [2H]C([2H])(c1c[nH]c2cccc(OS(N)(=O)=O)c12)C([2H])([2H])N(C)C] |
| | NS(OC1═CC═CC2═C1C(C([2H])([2H])C([2H])([2H])N(C)C)═CN2)(═O)═O |
| 236 | 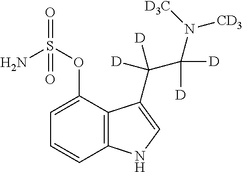 [SMILES: [2H]C([2H])([2H])N(C([2H])([2H])[2H])C([2H])([2H])C([2H])([2H])c1c[nH]c2cccc(OS(N)(=O)=O)c12] |
| | NS(OC1═CC═CC2═C1C(C([2H])([2H])C([2H]) |
| | ([2H])N(C([2H])([2H])[2H])C([2H])([2H])[2H])═CN2)(═O)═O |
| 237 | 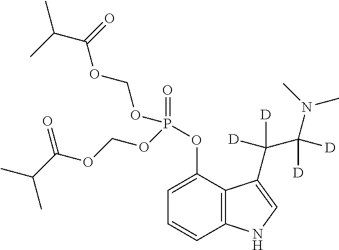 [SMILES: [2H]C([2H])(c1c[nH]c2cccc(OP(=O)(OCOC(=O)C(C)C)OCOC(=O)C(C)C)c12)C([2H])([2H])N(C)C] |
| | CN(C)C([2H])([2H])C([2H])([2H])C1═CNC2═C1C(OP(OCOC(C(C) |
| | C)═O)(OCOC(C(C)C)═O)═O)═CC═C2 |
| 238 | 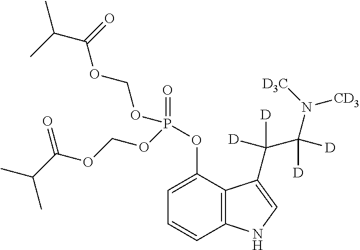 [SMILES: [2H]C([2H])([2H])N(C([2H])([2H])[2H])C([2H])([2H])C([2H])([2H])c1c[nH]c2cccc(OP(=O)(OCOC(=O)C(C)C)OCOC(=O)C(C)C)c12] |
| | O═P(OCOC(C(C)C)═O)(OCOC(C(C)C)═O)OC1═CC═CC2═C1C(C([2H]) |
| | ([2H])C([2H])([2H])N(C([2H])([2H])[2H])C([2H])([2H])[2H])═CN2 |
| 239 | 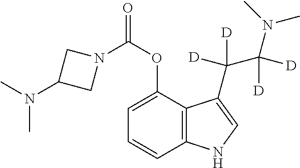 |
| | O═C(N1CC(N(C)C)C1)OC2═CC═CC3═C2C(C([2H])([2H])C([2H]) |
| | ([2H])N(C)C)═CN3 |
| 240 | 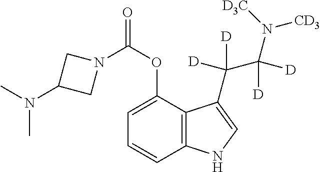 [SMILES: [2H]C([2H])([2H])N(C([2H])([2H])[2H])C([2H])([2H])C([2H])([2H])c1c[nH]c2cccc(OC(=O)N3CC(N(C)C)C3)c12] |
| | O═C(N1CC(N(C)C)C1)OC2═CC═CC3═C2C(C([2H])([2H])C([2H]) |
| | ([2H])N(C([2H])([2H])[2H]) |
| | C([2H])([2H])[2H])═CN3 |
| 241 | 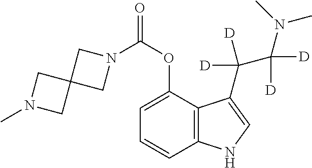 |
| | O═C(N1CC2(CN(C)C2)C1)OC3═CC═CC4═C3C(C([2H])([2H]) |
| | C([2H])([2H])N(C)C)═CN4 |
| 242 | 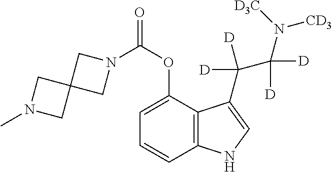 [SMILES: [2H]C([2H])([2H])N(C([2H])([2H])[2H])C([2H])([2H])C([2H])([2H])c1c[nH]c2cccc(OC(=O)N3CC4(CN(C)C4)C3)c12] |
| | O═C(N1CC2(CN(C)C2)C1)OC3═CC═CC4═C3C(C([2H]) |
| | ([2H])C([2H])([2H])N(C([2H])([2H])[2H])C([2H])([2H])[2H])═CN4 |
| 243 | 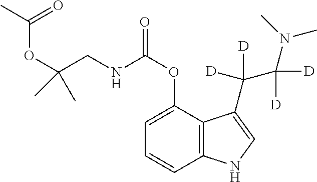 [SMILES: [2H]C([2H])(c1c[nH]c2cccc(OC(=O)NCC(C)(C)OC(C)=O)c12)C([2H])([2H])N(C)C] |
| | O═C(NCC(C)(C)OC(C)=50 O)OC1═CC═CC2═C1C(C([2H]) |
| | ([2H])C([2H])([2H])N(C)C)═CN2 |
| 244 | 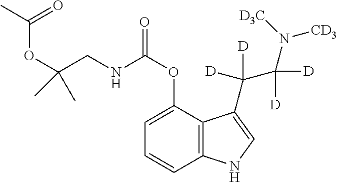 [SMILES: [2H]C([2H])([2H])N(C([2H])([2H])[2H])C([2H])([2H])C([2H])([2H])c1c[nH]c2cccc(OC(=O)NCC(C)(C)OC(C)=O)c12] |
| | O═C(NCC(C)(C)OC(C)═O)OC1═CC═CC2═C1C(C([2H])([2H])C([2H]) |
| | ([2H])N(C([2H])([2H])[2H])C([2H])([2H])[2H])═CN2 |
| 245 | 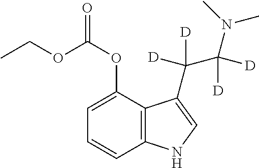 [SMILES: [2H]C([2H])(c1c[nH]c2cccc(OC(=O)OCC)c12)C([2H])([2H])N(C)C] |
| | O═C(OCC)OC1═CC═CC2═C1C(C([2H])([2H])C([2H])([2H])N(C)C)═CN2 |
| 246 | 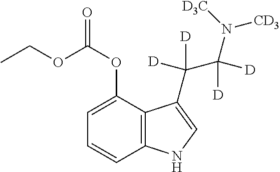 [SMILES: [2H]C([2H])([2H])N(C([2H])([2H])[2H])C([2H])([2H])C([2H])([2H])c1c[nH]c2cccc(OC(=O)OCC)c12] |
| | O═C(OCC)OC1═CC═CC2═C1C(C([2H])([2H])C([2H]) |
| | ([2H])N(C([2H])([2H])[2H])C([2H])([2H])[2H])═CN2 |
| 247 | 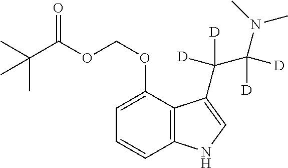 [SMILES: [2H]C([2H])(c1c[nH]c2cccc(OCOC(=O)C(C)(C)C)c12)C([2H])([2H])N(C)C] |
| | CN(C)C([2H])([2H])C([2H])([2H])C1═CNC2═C1C(OCOC(C(C)(C)C) |
| | ═O)═CC═C2 |
| 248 | 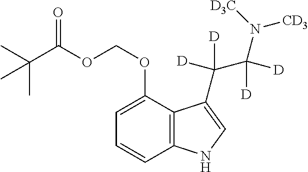 [SMILES: [2H]C([2H])([2H])N(C([2H])([2H])[2H])C([2H])([2H])C([2H])([2H])c1c[nH]c2cccc(OCOC(=O)C(C)(C)C)c12] |
| | O═C(C(C)(C)C)OCOC1═CC═CC2═C1C(C[2H])([2H])C([2H])([2H])N(C([2H]) |
| | ([2H])[2H])C([2H])([2H])[2H])═CN2 |
| 249 | 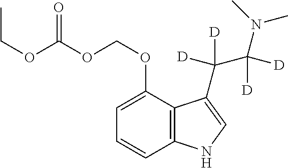 [SMILES: [2H]C([2H])(c1c[nH]c2cccc(OCOC(=O)OCC)c12)C([2H])([2H])N(C)C] |
| | CN(C)C([2H])([2H])C([2H])([2H]) |
| | C1═CNC2═C1C(OCOC(OCC)═O)═CC═C2 |
| 250 | 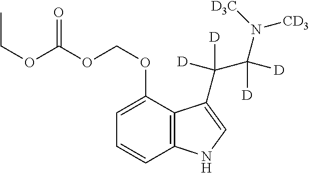 [SMILES: [2H]C([2H])([2H])N(C([2H])([2H])[2H])C([2H])([2H])C([2H])([2H])c1c[nH]c2cccc(OCOC(=O)OCC)c12] |
| | O═C(OCC)OCOC1═CC═CC2═C1C(C([2H])([2H]) |
| | C([2H])([2H])N(C([2H])([2H])[2H])C([2H])([2H])[2H])═CN2 |
| 251 | 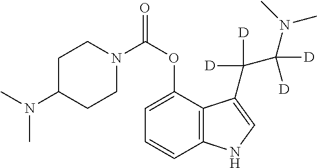 |
| | O═C(N1CCC(N(C)C)CC1)OC2═CC═CC3═C2C(C([2H]) |
| | ([2H])C([2H])([2H])N(C)C)═CN3 |
| 252 | 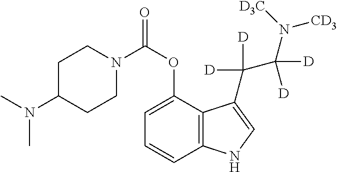 [SMILES: [2H]C([2H])([2H])N(C([2H])([2H])[2H])C([2H])([2H])C([2H])([2H])c1c[nH]c2cccc(OC(=O)N3CCC(N(C)C)CC3)c12] |
| | O═C(N1CCC(N(C)C)CC1)OC2═CC═CC3═C2C(C([2H]) |
| | ([2H])C([2H])([2H])N(C([2H])([2H])[2H])C([2H])([2H])[2H])═CN3 |
| 253 | 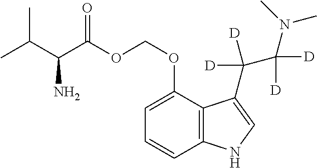 [SMILES: [2H]C([2H])(c1c[nH]c2cccc(OCOC(=O)[C@@H](N)C(C)C)c12)C([2H])([2H])N(C)C] |
| | CN(C)C([2H])([2H])C([2H])([2H]) |
| | C1═CNC2═C1C(OCOC([C@H](C(C)C)N)═O)═CC═C2 |
| 254 | 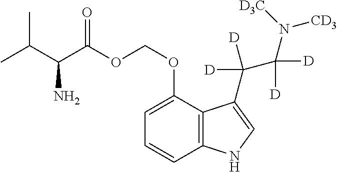 [SMILES: [2H]C([2H])([2H])N(C([2H])([2H])[2H])C([2H])([2H])C([2H])([2H])c1c[nH]c2cccc(OCOC(=O)[C@@H](N)C(C)C)c12] |
| | O═C([C@H](C(C)C)N)OCOC1═CC═CC2═C1C(C([2H]) |
| | ([2H])C([2H])([2H])N(C([2H])([2H])[2H])C([2H])([2H])[2H])═CN2 |

In certain embodiments, the compound of Formula (I) or pharmaceutically acceptable salt thereof is selected from the group consisting of:

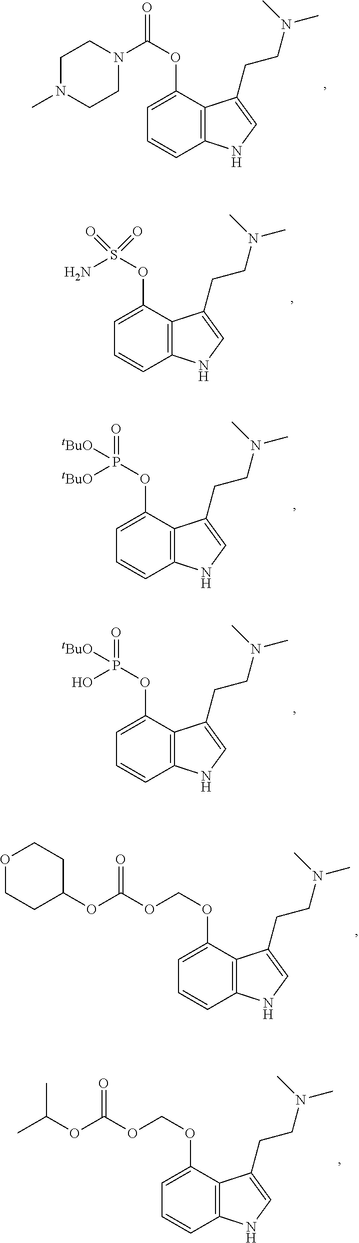

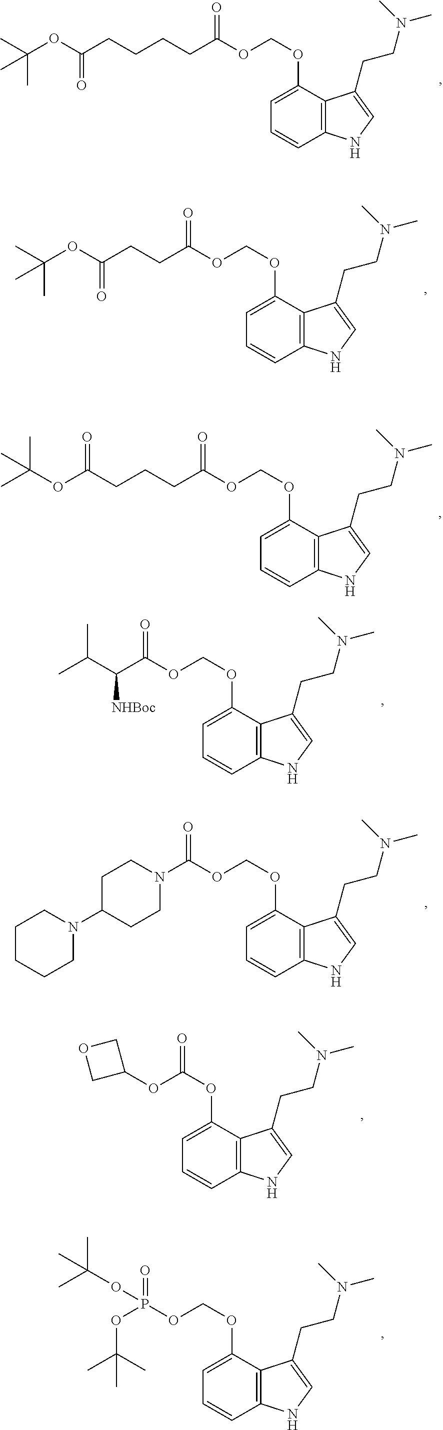

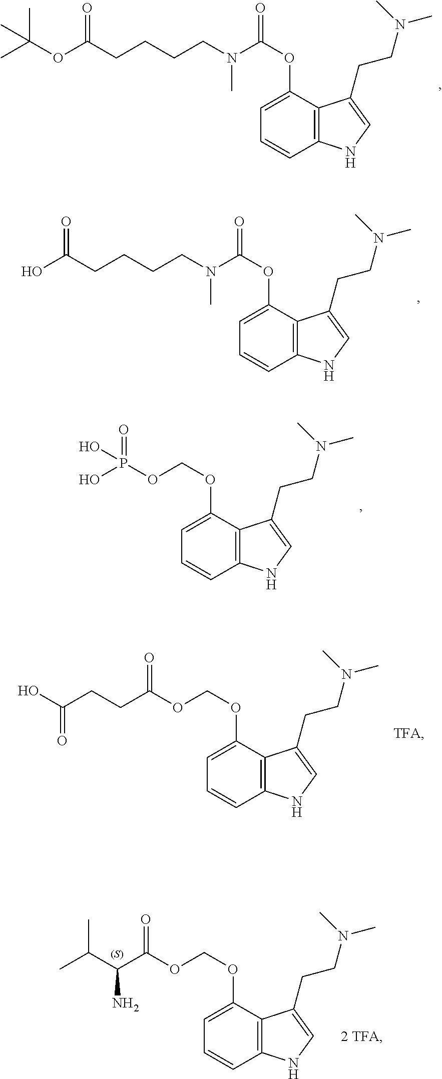

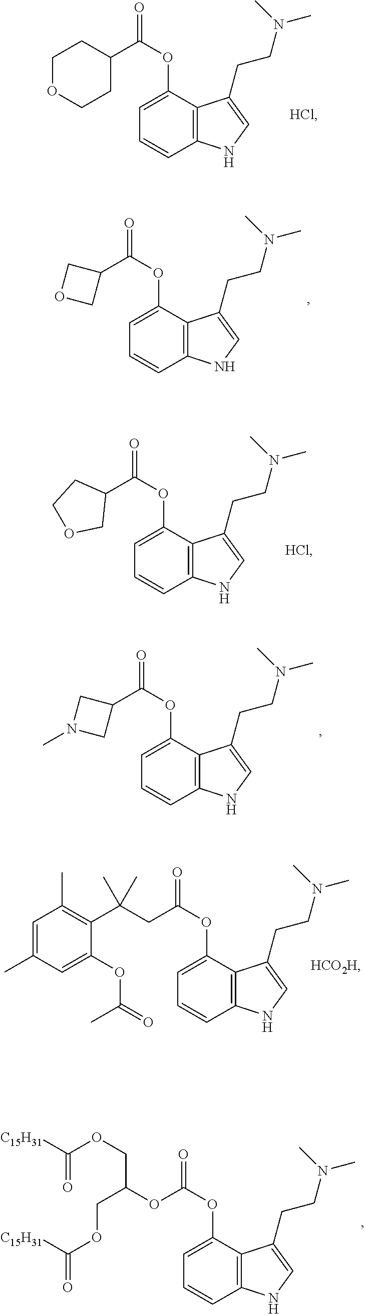

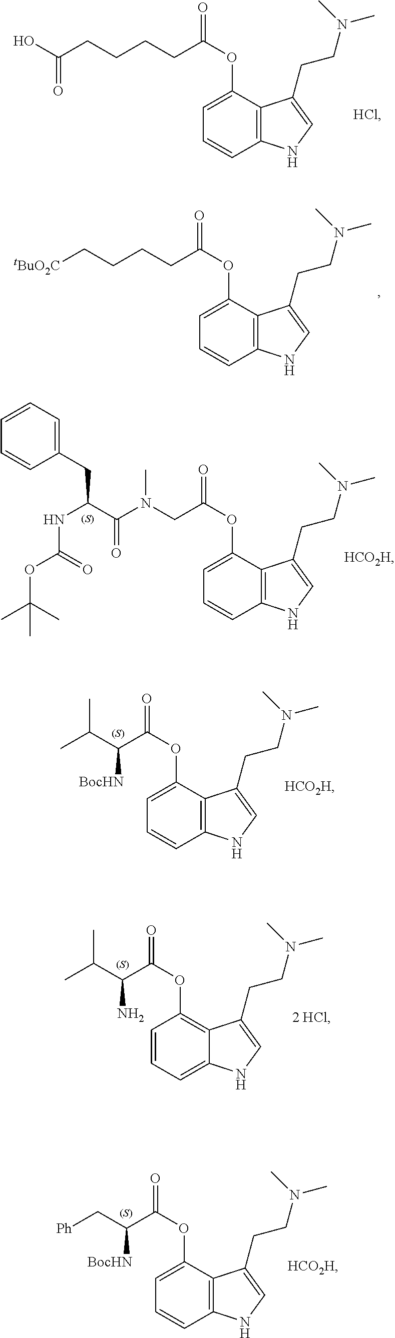

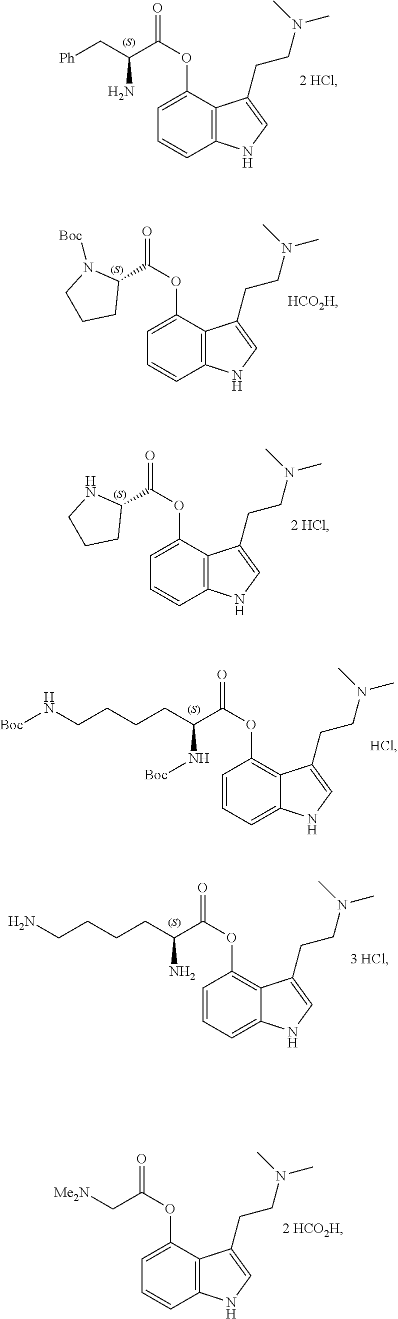

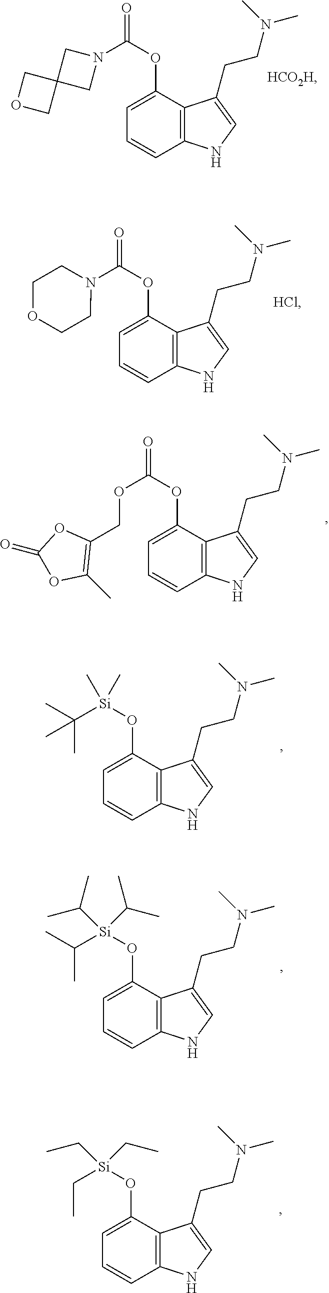

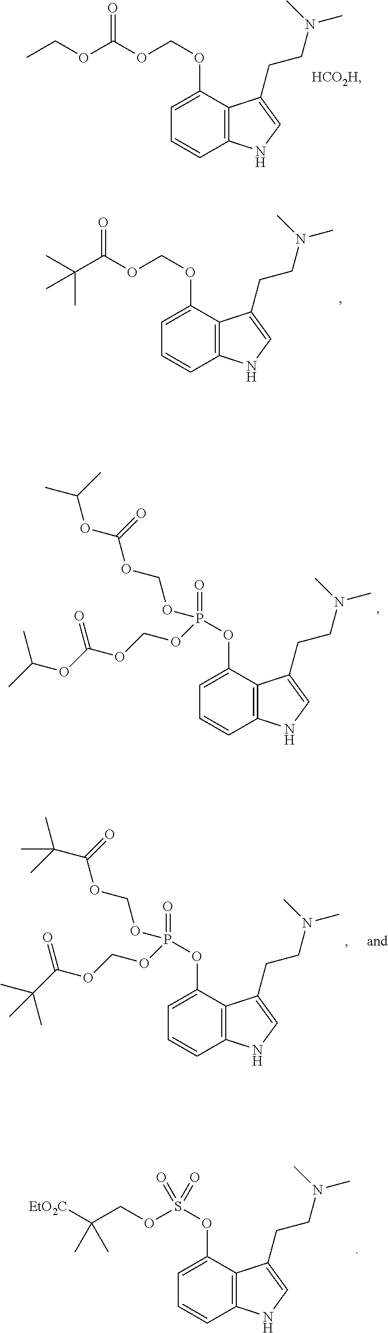

### Deuterated Analogues of O-Acetylpsilocin

In another aspect, provided herein are compounds of Formula (II), or a pharmaceutically acceptable salt, solvate, or isotopolog thereof:

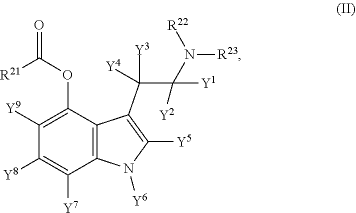

wherein:
R²¹ is —CH₃, —CH₂D, —CHD₂, or —CD₃;
each of R²² and R²³ is independently hydrogen or alkyl, wherein one or more of the hydrogens in the alkyl is optionally substituted with deuterium;
each of Y¹, Y², Y³, Y⁴, Y⁵, Y⁶, Y⁷, Y⁸, and Y⁹ is independently hydrogen or deuterium; and
wherein when R²¹ is CH₃, and R²² and R²³ do not comprise deuterium, at least one of Y¹, Y², Y³, Y⁴, Y⁵, Y⁶, Y⁷, Y⁸, and Y⁹ is deuterium.

In certain embodiments, R²¹ is —CH₃, —CH₂D, —CHD₂, or —CD₃. In certain embodiments, R²¹ is —CH₂D, —CHD₂, or —CD₃. In certain embodiments, R²¹ is —CH₃, —CHD₂, or —CD₃. In certain embodiments, R²¹ is —CH₃, —CH₂D, or —CD₃. In certain embodiments, R²¹ is —CH₃, —CH₂D, or —CHD₂.

In certain embodiments, R²¹ is —CH₃. In certain embodiments, R²¹ is —CD₃. In certain embodiments, R²¹ is —CH₂D. In certain embodiments, R²¹ is —CHD₂.

In certain embodiments, R²² and R²³ are each independently —CH₃, —CH₂D, —CHD₂, or —CD₃. In certain embodiments, at least one of R²² and R²³ comprises deuterium. In certain embodiments, one of R²² and R²³ is —CD₃. In certain embodiments, both R²² and R²³ are —CD₃.

In certain embodiments, Y¹ is D. In certain embodiments, Y³ is D. In certain embodiments, Y¹ and Y² are each D. In certain embodiments, Y³ and Y⁴ are each D. In certain embodiments, Y¹, Y², Y³, and Y⁴ are each D.

In certain embodiments, Y⁶ is H.

In some embodiments, disclosed herein are deuterated analogs of O-acetyl psilocin.

In some embodiments, the compound of Formula (II) is a compound of Formula (IIa) or Formula (IIb):

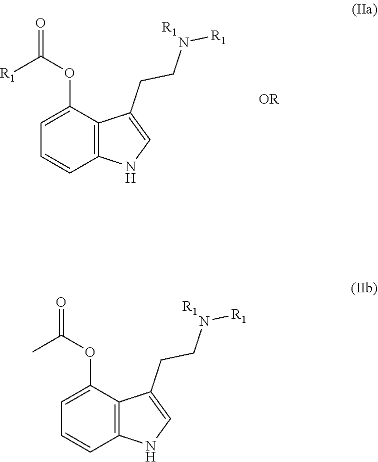

wherein
R²¹ is CH₃, CH₂D, CHD₂, or CD₃;
each of R²² and R²³ is independently hydrogen or alkyl, wherein one or more of the hydrogens in the alkyl is optionally substituted with deuterium; and
at least one of R²¹, R²², and R²³ comprises one or more deuterium.

In some embodiments, the compound of Formula (II) is a compound selected from the group consisting of:

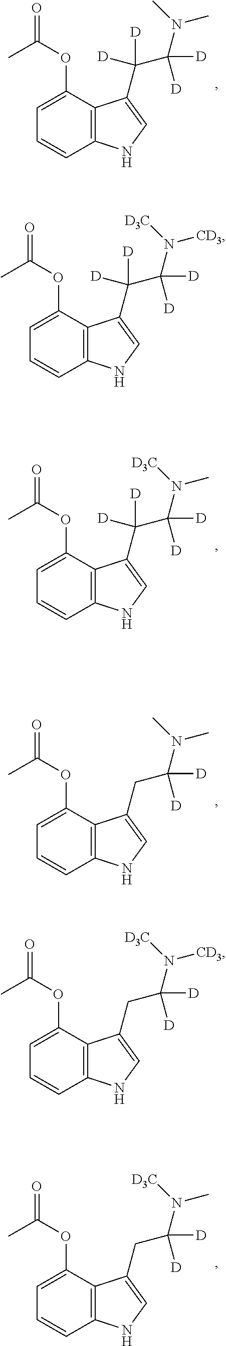

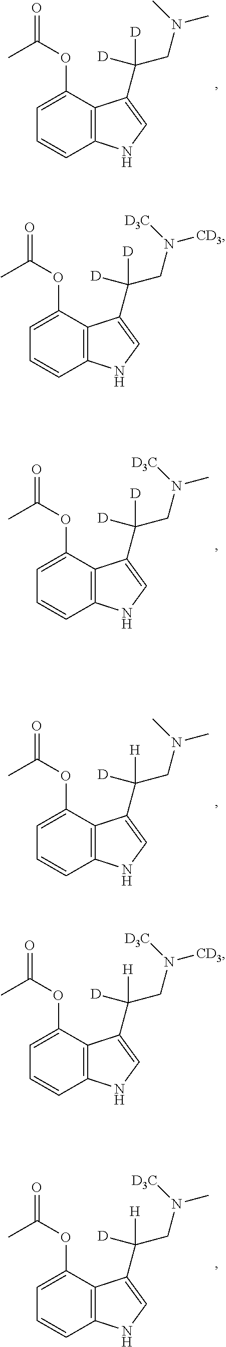

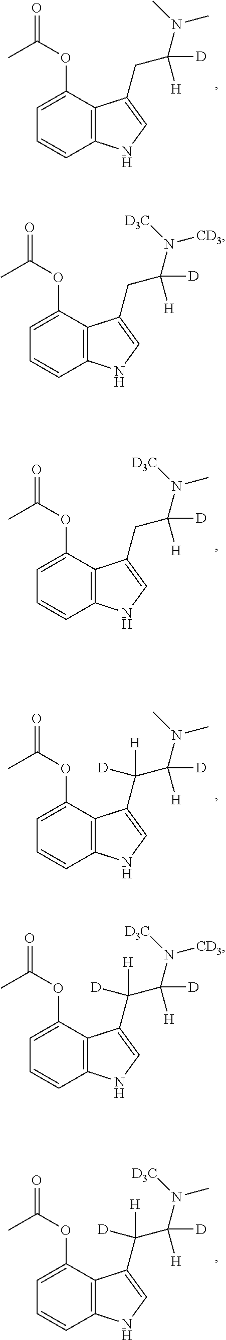

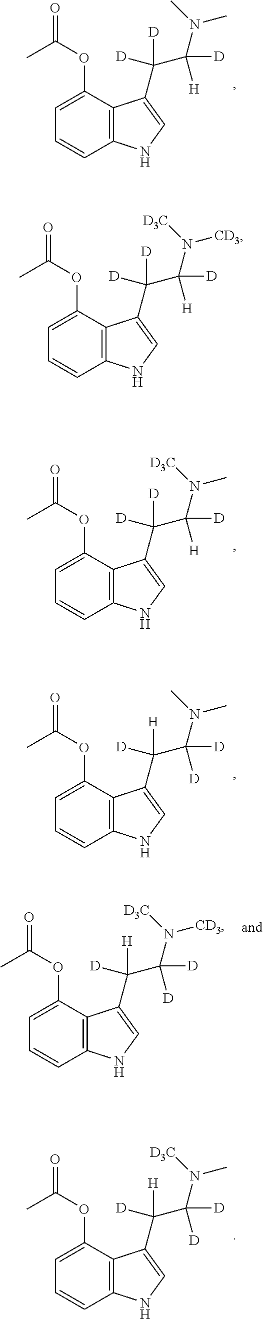

In another aspect, the present disclosure provides a pharmaceutically acceptable composition comprising a compound according to any of Formula (I), (Ia), (Ia′), (Ib), (Ib′), (Ib″), (Ic), (Id), (Ie), (If), (Ig), (Ih), (Ih′), (Ia-1), (lb-1), (Ic-1), or (II), or a pharmaceutically acceptable salt thereof, and a pharmaceutically acceptable excipient or carrier.

In another aspect, the present disclosure provides a pharmaceutically acceptable composition comprising a compound according to any of Formula (Ii) or (Ii), or a pharmaceutically acceptable salt thereof, and a pharmaceutically acceptable excipient or carrier.

Pharmaceutical compositions of the present disclosure can comprise racemic, scalemic, or diastereomerically enriched mixtures of any compound described herein comprising a stereogenic center.

### Methods of Treatment.

In yet another aspect, the present disclosure provides a method of treating or preventing a disease, disorder, or condition in which an increased level of a phenethylamine psychedelic such as MDMA is beneficial, comprising administering to a subject in need thereof an effective amount of a compound of Formula (I), (Ia), (Ia′), (Ib), (Ib′), (Ib″), (Ic), (Id), (Ie), (If), (Ig), (Ih), (Ih′), (Ia-1), (lb-1), (Ic-1), or (II), or a pharmaceutically acceptable salt thereof. In some embodiments, the condition comprises post-traumatic stress disorder, major depression, schizophrenia, Alzheimer's disease, frontotemporal dementia, Parkinson's disease, Parkinson's dementia, dementia, Lewy body dementia, multiple system atrophy, or substance abuse. In some embodiments, the condition comprises musculoskeletal pain disorder including fibromyalgia, muscle pain, joint stiffness, osteoarthritis, rheumatoid arthritis, muscle cramps. In some embodiments, the present disclosure provides a method of treating a disease of women's reproductive health including premenstrual dysphoric disorder (PMDD), premenstrual syndrome (PMS), post-partum depression, and menopause. The compounds of the present invention can also be used to treat any brain disease.

In one embodiment, the present disclosure provides a method of treating or preventing a disease, disorder, or condition in which an increased level of a phenethylamine psychedelic such as MDMA is beneficial, comprising administering to a subject in need thereof an effective amount of a compound of Formula (Ii) or (Ij), or a pharmaceutically acceptable salt thereof. In some embodiments, the condition comprises post-traumatic stress disorder, major depression, schizophrenia, Alzheimer's disease, frontotemporal dementia, Parkinson's disease, Parkinson's dementia, dementia, Lewy body dementia, multiple system atrophy, or substance abuse. In some embodiments, the condition comprises musculoskeletal pain disorder including fibromyalgia, muscle pain, joint stiffness, osteoarthritis, rheumatoid arthritis, muscle cramps. In some embodiments, the present disclosure provides a method of treating a disease of women's reproductive health including premenstrual dysphoric disorder (PMDD), premenstrual syndrome (PMS), post-partum depression, and menopause. The compounds of the present invention can also be used to treat any brain disease.

In some embodiments, a compound disclosed herein has activity as a 5-HT_{2A} modulator. In some embodiments a compound disclosed herein elicits a biological response by activating the 5-HT_{2A} receptor (e.g., allosteric modulation or modulation of a biological target that activates the 5-HT_{2A} receptor). 5-HT_{2A} agonism has been correlated with the promotion of neural plasticity. 5-HT_{2A} antagonists abrogate the neuritogenesis and spinogenesis effects of hallucinogenic compounds with 5-HT_{2A} agonist activity, for example, DMT, LSD, and DOI. In some embodiments, a compound disclosed herein is a 5-HT_{2A} modulator and promotes neural plasticity (e.g., cortical structural plasticity). In some embodiments, a compound disclosed herein is a selective 5-HT_{2A} modulator and promotes neural plasticity (e.g., cortical structural plasticity). Promotion of neural plasticity can include, for example, increased dendritic spine growth, increased synthesis of synaptic proteins, strengthened synaptic responses, increased dendritic arbor complexity, increased dendritic branch content, increased spinogenesis, increased neuritogenesis, or any combination thereof. In some embodiments, increased neural plasticity includes increased cortical structural plasticity in the anterior parts of the brain.

In some embodiments, the 5-HT_{2A} modulators (e.g., 5-HT_{2A} agonists) are non-hallucinogenic. In some embodiments, non-hallucinogenic 5-HT_{2A} modulators (e.g., 5-HT_{2A} agonists) are used to treat neurological diseases, which modulators do not elicit dissociative side-effects. In some embodiments, the hallucinogenic potential of the compounds described herein is assessed in vitro. In some embodiments, the hallucinogenic potential assessed in vitro of the compounds described herein is compared to the hallucinogenic potential assessed in vitro of hallucinogenic homologs. In some embodiments, the compounds described herein elicit less hallucinogenic potential in vitro than the hallucinogenic homologs.

In some embodiments, serotonin receptor modulators, such as modulators of serotonin receptor 2A (5-HT_{2A} modulators, e.g., 5-HT_{2A} agonists), are used to treat a brain disorder. In some embodiments, a compound of the present disclosure functions as a 5-HT_{2A} agonist alone, or in combination with a second therapeutic agent that also is a 5-HT_{2A} modulator. In such cases the second therapeutic agent can be an agonist or an antagonist. In some instances, it may be helpful administer a 5-HT_{2A} antagonist in combination with a compound of the present disclosure to mitigate undesirable effects of 5-HT_{2A} agonism, such as potential hallucinogenic effects. Serotonin receptor modulators useful as second therapeutic agents for combination therapy as described herein are known to those of skill in the art and include, without limitation, ketanserin, volinanserin (MDL-100907), eplivanserin (SR-46349), pimavanserin (ACP-103), glemanserin (MDL-11939), ritanserin, flibanserin, nelotanserin, blonanserin, mianserin, mirtazapine, roluperiodone (CYR-101, MIN-101), quetiapine, olanzapine, altanserin, acepromazine, nefazodone, risperidone, pruvanserin, AC-90179, AC-279, adatanserin, fananserin, HY10275, benanserin, butanserin, manserin, iferanserin, lidanserin, pelanserin, seganserin, tropanserin, lorcaserin, ICI-169369, methiothepin, methysergide, trazodone, cinitapride, cyproheptadine, brexpiprazole, cariprazine, agomelatine, setoperone, 1-(1-Naphthyl)piperazine, LY-367265, pirenperone, metergoline, deramciclane, amperozide, AMDA, cinanserin, LY-86057, GSK-215083, cyamemazine, mesulergine, BF-1, LY-215840, sergolexole, spiramide, LY-53857, amesergide, LY-108742, pipamperone, LY-314228, 5-I-R91150, 5-MeO-NBpBrT, 9-Aminomethyl-9,10-dihydroanthracene, niaprazine, SB-215505, SB-204741, SB-206553, SB-242084, LY-272015, SB-243213, SB-200646, RS-102221, zotepine, clozapine, chlorpromazine, sertindole, iloperidone, risperidone, paliperidone, asenapine, amisulpride, aripiprazole, brexpiprazole, lurasidone, ziprasidone, or lumateperone, or a pharmaceutically acceptable salt, solvate, metabolite, deuterated analogue, derivative, prodrug, or combinations thereof. In some embodiments, the serotonin receptor modulator used as a second therapeutic is pimavanserin or a pharmaceutically acceptable salt, solvate, metabolite, derivative, or prodrug thereof. In some embodiments, the serotonin receptor modulator is administered prior administration of a compound disclosed herein, such as about three or about hours prior administration of the compound. In some embodiments, the serotonin receptor modulator is administered at most about one hour prior to the compound. In some embodiments, the second therapeutic agent is a serotonin receptor modulator. In some embodiments, the serotonin receptor modulator is provided at a dose of from about 10 mg to about 350 mg. In some embodiments, the serotonin receptor modulator is provided at a dose of from about 20 mg to about 200 mg. In some embodiments, the serotonin receptor modulator is provided at a dose of from about 10 mg to about 100 mg. In certain such embodiments, a compound of the present disclosure is provided at a dose of from about 10 mg to about 100 mg, or from about 20 to about 200 mg, or from about 15 to about 300 mg, and the serotonin receptor modulator is provided at a dose of about 10 mg to about 100 mg.

In some embodiments, non-hallucinogenic 5-HT2_{A} modulators (e.g., 5-HT2_{A} agonists) are used to treat neurological diseases. In some embodiments, the neurological diseases comprise decreased neural plasticity, decreased cortical structural plasticity, decreased 5-HT_{2A} receptor content, decreased dendritic arbor complexity, loss of dendritic spines, decreased dendritic branch content, decreased spinogenesis, decreased neuritogenesis, retraction of neurites, or any combination thereof.

In some embodiments, non-hallucinogenic 5-HT_{2A} modulators (e.g., 5-HT_{2A} agonists) are used for increasing neuronal plasticity. In some embodiments, non-hallucinogenic 5-HT_{2A} modulators (e.g., 5-HT_{2A} agonists) are used for treating a brain disorder. In some embodiments, non-hallucinogenic 5-HT_{2A} modulators (e.g., 5-HT_{2A} agonists) are used for increasing at least one of translation, transcription, or secretion of neurotrophic factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21^{st} Ed. Mack Pub. Co., Easton, PA (2005)).

In some embodiments, a compound described herein is given to patients in a low dose that is lower than would produce noticeable psychedelic effects but high enough to provide a therapeutic benefit. This dose range is predicted to be between 200 μg (micrograms) and 2 mg.

In some embodiments, oral doses typically range from about 1.0 mg to about 350 mg, one to four times, or more, per day. In certain embodiments, the compounds are administered to a subject at a daily dosage of between 0.01 mg/kg to about 50 mg/kg of body weight. In other embodiments, the dose is from 1 to 350 mg/day. In certain embodiments, the daily dose is from 1 to 750 mg/day; or from 10 to 350 mg/day. In certain embodiments, the compounds disclosed herein, including those described in Table 1, are provided at a daily dose of from about 2 mg to about 5 mg, or from about 5 mg to about 10 mg, or from about 10 mg to about 100 mg, or from about 20 to about 200 mg, or from about 15 to about 300 mg, or 10 mg, or 15 mg, or 20 mg, or 25 mg, or 30 mg, or 35 mg, or 40 mg, or 45 mg, or 50 mg, or 55 mg, or 60 mg, or 65 mg, or 70 mg, or 75 mg, or 80 mg, or 85 mg, or 90 mg, or 95 mg, or 100 mg.

In some embodiments, a compound described herein is used to treat a neurological disease. For example, a compound provided herein can exhibit, anti-addictive properties, antidepressant properties, anxiolytic properties, or a combination thereof. In some embodiments, the neurological disease is a neuropsychiatric disease. In some embodiments, the neuropsychiatric disease is a mood or anxiety disorder. In some embodiments, the neurological disease is a migraine, headaches (e.g., cluster headache), post-traumatic stress disorder (PTSD), anxiety, depression, neurodegenerative disorder, Alzheimer's disease, Parkinson's disease, psychological disorder, treatment resistant depression, suicidal ideation, major depressive disorder, bipolar disorder, schizophrenia, stroke, traumatic brain injury, and addiction (e.g., substance use disorder). In some embodiments, the neurological disease is a migraine or cluster headache. In some embodiments, the neurological disease is a neurodegenerative disorder, Alzheimer's disease, or Parkinson's disease. In some embodiments, the neurological disease is a psychological disorder, treatment resistant depression, suicidal ideation, major depressive disorder, bipolar disorder, schizophrenia, post-traumatic stress disorder (PTSD), addiction (e.g., substance use disorder), depression, or anxiety. In some embodiments, the neuropsychiatric disease is a psychological disorder, treatment resistant depression, suicidal ideation, major depressive disorder, bipolar disorder, schizophrenia, post-traumatic stress disorder (PTSD), addiction (e.g., substance use disorder), depression, or anxiety. In some embodiments, the neuropsychiatric disease or neurological disease is post-traumatic stress disorder (PTSD), addiction (e.g., substance use disorder), schizophrenia, depression, or anxiety. In some embodiments, the neuropsychiatric disease or neurological disease is addiction (e.g., substance use disorder). In some embodiments, the neuropsychiatric disease or neurological disease is depression. In some embodiments, the neuropsychiatric disease or neurological disease is anxiety. In some embodiments, the neuropsychiatric disease or neurological disease is post-traumatic stress disorder (PTSD). In some embodiments, the neurological disease is stroke or traumatic brain injury. In some embodiments, the neuropsychiatric disease or neurological disease is schizophrenia.

In some embodiments, a compound of the present disclosure is used for increasing neuronal plasticity. In some embodiments, a compound described herein is used for treating a brain disorder. In some embodiments, a compound described herein is used for increasing translation, transcription, or secretion of neurotrophic factors.

A compound disclosed herein can also be useful for increasing neuronal plasticity in a subject. As used herein, “neuronal plasticity” can refer to the ability of the brain to change structure and/or function throughout a subject's life. New neurons can be produced and integrated into the central nervous system throughout the subject's life. Increasing neuronal plasticity can include, but is not limited to, promoting neuronal growth, promoting neuritogenesis, promoting synaptogenesis, promoting dendritogenesis, increasing dendritic arbor complexity, increasing dendritic spine density, and increasing excitatory synapsis in the brain. In some embodiments, increasing neuronal plasticity comprises promoting neuronal growth, promoting neuritogenesis, promoting synaptogenesis, promoting dendritogenesis, increasing dendritic arbor complexity, and increasing dendritic spine density.

In some embodiments, increasing neuronal plasticity by treating a subject with a compound the present disclosure can treat neurodegenerative disorder, Alzheimer's, Parkinson's disease, psychological disorder, depression, addiction, anxiety, post-traumatic stress disorder, treatment resistant depression, suicidal ideation, major depressive disorder, bipolar disorder, schizophrenia, stroke, traumatic brain injury, or substance use disorder.

In some embodiments, the present disclosure provides a method for increasing neuronal plasticity, comprising contacting a neuronal cell with a compound of the present disclosure. In some embodiments, increasing neuronal plasticity improves a brain disorder described herein.

In some embodiments, a compound disclosed herein is used to increase neuronal plasticity and has, for example, anti-addictive properties, antidepressant properties, anxiolytic properties, or a combination thereof. In some embodiments, decreased neuronal plasticity is associated with a neuropsychiatric disease. In some embodiments, the neuropsychiatric disease is a mood or anxiety disorder. In some embodiments, the neuropsychiatric disease includes, for example, migraine, cluster headache, post-traumatic stress disorder (PTSD), schizophrenia, anxiety, depression, and addiction (e.g., substance abuse disorder). Brain disorders can include, for example, migraines, addiction (e.g., substance use disorder), depression, and anxiety.

In some embodiments, the experiment or assay to determine increased neuronal plasticity derived from the administration of any compound of the present disclosure is a phenotypic assay, a dendritogenesis assay, a spinogenesis assay, a synaptogenesis assay, a Sholl analysis, a concentration-response experiment, a 5-HT_{2A} agonist assay, a 5-HT_{2A} antagonist assay, a 5-HT_{2A} binding assay, or a 5-HT_{2A} blocking experiment (e.g., ketanserin blocking experiments). In some embodiments, the experiment or assay to determine the hallucinogenic potential of any compound of the present disclosure is a mouse head-twitch response (HTR) assay.

In some embodiments, the condition is a musculoskeletal pain disorder including fibromyalgia, muscle pain, joint stiffness, osteoarthritis, rheumatoid arthritis, muscle cramps. In some embodiments, the present disclosure provides a method of treating a disease of women's reproductive health including premenstrual dysphoric disorder (PMDD), premenstrual syndrome (PMS), post-partum depression, and menopause. In some embodiments, the present disclosure provides a method of treating a brain disorder, including administering to a subject in need thereof, a therapeutically effective amount of a compound of the present disclosure. In some embodiments, the present disclosure provides a method of treating a brain disorder with combination therapy, including administering to a subject in need thereof, a therapeutically effective amount of a compound of the present disclosure and at least one additional therapeutic agent.

In some embodiments, a compound of the present disclosure is used to treat brain disorders. In some embodiments, the compound has, for example, anti-addictive properties, antidepressant properties, anxiolytic properties, or a combination thereof. In some embodiments, the brain disorder is a neuropsychiatric disease. In some embodiments, the neuropsychiatric disease is a mood or anxiety disorder. In some embodiments, brain disorders include, for example, migraine, cluster headache, post-traumatic stress disorder (PTSD), anxiety, depression, panic disorder, suicidality, schizophrenia, and addiction (e.g., substance abuse disorder). In some embodiments, brain disorders include, for example, migraines, addiction (e.g., substance use disorder), depression, and anxiety.

In some embodiments, the present disclosure provides a method of treating a brain disorder, comprising administering to a subject in need thereof a therapeutically effective amount of a compound disclosed herein. In some embodiments, the brain disorder is a neurodegenerative disorder, Alzheimer's disease, Parkinson's disease, a psychological disorder, depression, addiction, anxiety, post-traumatic stress disorder, treatment resistant depression, suicidal ideation, major depressive disorder, bipolar disorder, schizophrenia, stroke, traumatic brain injury, or a substance use disorder.

In some embodiments, the brain disorder is a neurodegenerative disorder, Alzheimer's disease or Parkinson's disease. In some embodiments, the brain disorder is a psychological disorder, depression, addiction, anxiety, or a post-traumatic stress disorder. In some embodiments, the brain disorder is depression. In some embodiments, the brain disorder is addiction. In some embodiments, the brain disorder is treatment resistant depression, suicidal ideation, major depressive disorder, bipolar disorder, schizophrenia, stroke, traumatic brain injury or substance use disorder. In some embodiments, the brain disorder is treatment resistant depression, suicidal ideation, major depressive disorder, bipolar disorder, schizophrenia, or substance use disorder. In some embodiments, the brain disorder is stroke or traumatic brain injury. In some embodiments, the brain disorder is treatment resistant depression, suicidal ideation, major depressive disorder, bipolar disorder, or substance use disorder. In some embodiments, the brain disorder is schizophrenia. In some embodiments, the brain disorder is alcohol use disorder.

In some embodiments, the method further comprises administering one or more additional therapeutic agent. Non-limiting examples of additional therapeutics suitable for administration with a compound of the present disclosure can include lithium, olanzapine (Zyprexa), quetiapine (Seroquel), risperidone (Risperdal), aripiprazole (Abilify), ziprasidone (Geodon), clozapine (Clozaril), divalproex sodium (Depakote), lamotrigine (Lamictal), valproic acid (Depakene), carbamazepine (Equetro), topiramate (Topamax), levomilnacipran (Fetzima), duloxetine (Cymbalta, Yentreve), venlafaxine (Effexor), citalopram (Celexa), fluvoxamine (Luvox), escitalopram (Lexapro), fluoxetine (Prozac), paroxetine (Paxil), sertraline (Zoloft), clomipramine (Anafranil), amitriptyline (Elavil), desipramine (Norpramin), imipramine (Tofranil), nortriptyline (Pamelor), phenelzine (Nardil), tranylcypromine (Parnate), diazepam (Valium), alprazolam (Xanax), or clonazepam (Klonopin).

In some embodiments, the additional therapeutic agent is a monoamine oxidase inhibitor (MAOI), which can be, for example, moclobemide, caroxazone (Surodil, Timostenil), brofaromine (Consonar), methylene blue, pirlindole (Pirazidol), minaprine (Cantor), metralindole (Inkazan), eprobemide, tetrindole, harmine, harmaline, amiflamine, befloxatone (MD-370,503), cimoxatone (MD-780,515), sercloremine (CGP-4718-A), esuprone, or CX157. In some embodiments, the additional therapeutic agent is a phenethylamine, such as 3,4-methylene-dioxymethamphetamine (MDMA) and analogs thereof. Other suitable empathogenic agents for use in combination a compound of the present disclosure include, without limitation, N-Allyl-3,4-methylenedioxy-amphetamine (MDAL), N-Butyl-3,4-methylenedioxyamphetamine (MDBU), N-Benzyl-3,4-methylenedioxyamphetamine (MDBZ), N-Cyclopropylmethyl-3,4-methylenedioxyamphetamine (MDCPM), N,N-Dimethyl-3,4-methylenedioxyamphetamine (MDDM), N-Ethyl-3,4-methylenedioxyamphetamine (MDE; MDEA), N-(2-Hydroxyethyl)-3,4-methylenedioxy amphetamine (MDHOET), N-Isopropyl-3,4-methylenedioxyamphetamine (MDIP), N-Methyl-3,4-ethylenedioxyamphetamine (MDMC), N-Methoxy-3,4-methylenedioxyamphetamine (MDMEO), N-(2-Methoxyethyl)-3,4-methylenedioxyamphetamine (MDMEOET), alpha,alpha,N-Trimethyl-3,4-methylenedioxyphenethylamine (MDMP), 3,4-Methylenedioxy-N-methylphentermine, N-Hydroxy-3,4-methylenedioxyamphetamine (MDOH), 3,4-Methylenedioxyphenethylamine (MDPEA), alpha,alpha-Dimethyl-3,4-methylenedioxyphenethylamine (MDPH; 3,4-methylenedioxyphentermine), N-Propargyl-3,4-methylenedioxyamphetamine (MDPL), Methylenedioxy-2-aminoindane (MDAI), 1,3-Benzodioxolyl-N-methylbutanamine (MBDB), N-methyl-1,3-benzodioxolylbutanamine, 3,4-methylenedioxy-N-methyl-α-ethylphenylethylamine, 3,4-Methylenedioxyamphetamine (MDA), Methylone (3,4-methylenedioxy-N-methylcathinone), Ethylone (3,4-methylenedioxy-N-ethylcathinone), GHB or Gamma Hydroxybutyrate or sodium oxybate, N-Propyl-3,4-methylenedioxyamphetamine (MDPR), and the like.

In some embodiments, a compound of the present disclosure is used in combination with the standard of care therapy for a neurological disease described herein. Non-limiting examples of the standard of care therapies, may include, for example, lithium, olanzapine, quetiapine, risperidone, ariprazole, ziprasidone, clozapine, divalproex sodium, lamotrigine, valproic acid, carbamazepine, topiramate, levomilnacipran, duloxetine, venlafaxine, citalopram, fluvoxamine, escitalopram, fluoxetine, paroxetine, sertraline, clomipramine, amitriptyline, desipramine, imipramine, nortriptyline, phenelzine, tranylcypromine, diazepam, alprazolam, clonazepam, or any combination thereof. Nonlimiting examples of standard of care therapy for depression are sertraline, fluoxetine, escitalopram, venlafaxine, or aripiprazole. Non-limiting examples of standard of care therapy for depression are citralopram, escitalopram, fluoxetine, paroxetine, diazepam, or sertraline. Additional examples of standard of care therapeutics are known to those of ordinary skill in the art.

### Methods of Increasing at Least One of Translation, Transcription, or Secretion of Neurotrophic Factors.

As used herein, the term “neurotrophic factor” can refer to a family of soluble peptides or proteins which support the survival, growth, and differentiation of developing and mature neurons. Increasing at least one of translation, transcription, or secretion of neurotrophic factors can be useful for, for example, increasing neuronal plasticity, promoting neuronal growth, promoting neuritogenesis, promoting synaptogenesis, promoting dendritogenesis, increasing dendritic arbor complexity, increasing dendritic spine density, and increasing excitatory synapsis in the brain. In some embodiments, increasing at least one of translation, transcription, or secretion of neurotrophic factors increases neuronal plasticity. In some embodiments, increasing at least one of translation, transcription, or secretion of neurotrophic factors promotes neuronal growth, promotes neuritogenesis, promotes synaptogenesis, promotes dendritogenesis, increases dendritic arbor complexity, and/or increases dendritic spine density.

In some embodiments, a 5-HT_{2A} modulators (e.g., 5-HT_{2A} agonists) is used to increase at least one of translation, transcription, or secretion of neurotrophic factors. In some embodiments, a compound of the present disclosure is used to increase translation, transcription, or secretion of neurotrophic factors. In some embodiments, increasing translation, transcription or secretion of neurotrophic factors is sufficient for the treatment of migraine, headaches (e.g., cluster headache), post-traumatic stress disorder (PTSD), anxiety, depression, neurodegenerative disorder, Alzheimer's disease, Parkinson's disease, psychological disorder, treatment resistant depression, suicidal ideation, major depressive disorder, bipolar disorder, schizophrenia, stroke, traumatic brain injury, or addiction (e.g., substance use disorder).

An experiment or assay can be used to detect increased translation of neurotrophic factors, which can include, for example, ELISA, western blot, an immunofluorescence assay, a proteomic experiment, and mass spectrometry. In some embodiments, the experiment or assay used to detect increased transcription of neurotrophic factors is a gene expression assay, PCR, or microarray. In some embodiments, the experiment or assay used to detect increased secretion of neurotrophic factors is ELISA, western blot, an immunofluorescence assay, a proteomic experiment, or a mass spectrometry assay.

In some embodiments, the present disclosure provides a method for increasing translation, transcription, or secretion of neurotrophic factors, wherein the method comprises contacting a neuronal cell with a compound disclosed herein.

### Examples

The following examples are intended to illustrate the invention and are not to be construed as being limitations thereon. Temperatures are given in degrees centigrade. If not mentioned otherwise, all evaporations are performed in vacuo, preferably between about 15 mm Hg and 100 mm Hg (=20-133 mbar). The structure of final products, intermediates and starting materials is confirmed by standard analytical methods, e.g., MS and NMR. Abbreviations used are those conventional in the art. If not defined, the terms have their generally accepted meanings.

### Preparation of Selected Compounds and Intermediates.

The following preparations of compounds and intermediates are given to enable those of skill in the art to more clearly understand and to practice the present disclosure. They should not be considered as limiting the scope of the disclosure, but merely as illustrative and representative thereof.

### Abbreviations

app apparent
Boc tert-butyl carbamate
Boc-Lys(Boc)-OSu Nα,Nε-Di-Boc-L-lysine hydroxysuccinimide ester
Boc-Phe-OSu Boc-L-phenylalanine N-hydroxysuccinimide ester
Boc-Pro-OSu Boc-L-proline N-hydroxysuccinimide ester
Boc-Val-OSu Boc-L-valine hydroxysuccinimide ester
br broad
CCl₄ carbon tetrachloride
CDCl₃ d-chloroform
CD₃OD methanol-d4
D₂O deuterium oxide
d doublet
dd doublet of doublets
DCM dichloromethane
DIPEA diisopropylethylamine
DMA dimethylacetamide
DMAP 4-dimethylaminopyridine
DMF N,N-dimethylformamide
DMSO dimethyl sulfoxide
EDCI·HCl N-(3-Dimethylaminopropyl)-N′-ethylcarbodiimide hydrochloride
Et₂O diethyl ether
EtOAc ethyl acetate
HCl hydrochloric acid or hydrogen chloride
h hextet; sextet
HBTU O-(Benzotriazol-1-yl)-N,N,N′,N′-tetramethyluronium
hexafluorophosphate
HPLC high pressure liquid chromatography
LC-MS liquid chromatography and mass spectrometry
MeOH methanol
MeCN acetonitrile
MgSO₄ magnesium sulfate
MS mass spectrometry
m multiplet
min(s) minute(s)
mL milliliter(s)
μL microliter(s)
m z mass to charge ratio
mol mole
NHS N-Hydroxysuccinimide
p pentet
q quartet
N₂ nitrogen
NaHCO₃ sodium hydrogen carbonate
NaOH sodium hydroxide
Na₂SO₄ sodium sulfate
NH₄Cl ammonium chloride
NMP N-methyl-2-pyrrolidone
NMR nuclear magnetic resonance
Rt retention time
s singlet
t triplet
tert tertiary
TBDMSCl tert-Butyldimethylsilyl chloride
TESCl Chlorotriethylsilane
TFA Trifluoroacetic acid
THF tetrahydrofuran
TIPSCl Triisopropylsilyl chloride

The various starting materials, intermediates, and compounds of the preferred embodiments may be isolated and purified, where appropriate, using conventional techniques such as precipitation, filtration, crystallization, evaporation, distillation, and chromatography. Salts may be prepared from compounds by known salt-forming procedures. Unless otherwise stated, all starting materials are obtained from commercial suppliers and used without further purification.

### General Conditions for Characterization:

Mass spectra were run on LC-MS systems using electrospray ionization. These were run using a Waters Acquity Classic UPLC with PDA and SQ mass detection or a Waters Acquity H-Class UPLC with PDA and QDA mass detection. [M+H]⁺ refers to mono-isotopic molecular weights.

### NMR Analysis

NMR spectra were run on either a Bruker Ultrashield 400 MHz or 500 MHz NMR spectrometer. Spectra were recorded at 298 K, unless otherwise stated, and were referenced using the solvent peak. The shift (d) of each signal was measured in parts per million (ppm) relative the residual solvent peak, and the multiplicity reported together with the associated coupling constant (J), where applicable.

If not indicated otherwise, the analytical HPLC conditions are as follows:

### Instrument: LC-MS-1:

### Method 2A

Column: Acquity UPLC BEH C18 2.1×50 mm 1.7 μm
Column Temp: 50° C.
Flow rate: 0.8 mL/min.
Eluents: A: H₂O, 0.1% formic acid, B: MeCN
Gradient: 0.0-1.8 min 2-98% B, 1.8-2.1 min 98% B, 2.1-2.5 98% A.

### Method 2B

Column: Acquity UPLC BEH C18 2.1×50 mm 1.7 μm
Column Temp: 50° C.
Flow rate: 0.8 mL/min.
Eluents: A: H₂O, 0.1% ammonia B: MeCN
Gradient: 0.0-1.8 min 2-98% B, 1.8-2.1 min 98% B, 2.1-2.5 98% A.

### Instrument: LC-MS-2:

### Method 2A

Column: Acquity UPLC BEH C18 2.1×50 mm 1.7 μm
Column Temp: 50° C.
Flow rate: 0.8 mL/min.
Eluents: A: H₂O, B: MeCN, C: 50% H₂O/50% MeCN+2.0% formic acid
Gradient: 0.0-1.7 mins 0-95% B, 5% C; 1.7-2.1 mins 95% B, 5% C 2.1-2.5 mins 95% A, 5% C.

### Method 2B

Column: Acquity UPLC BEH C18 2.1×50 mm 1.7 μm
Column Temp: 50° C.
Flow rate: 0.8 mL/min.
Eluents: A: H₂O, B: MeCN, C: 50% H2O/50% MeCN+2.0% ammonia (aq.)
Gradient: 0.0-1.7 mins 0-95% B, 5% D; 1.7-2.1 mins 95% B, 5% D 2.1-2.5 mins 95% A, 5% D.

### Example 1: [3-[2-(Dimethylamino)ethyl]-1H-indol-4-yl] 4-methylpiperazine-1-carboxylate

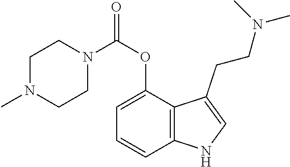

A suspension of 3-[2-(dimethylamino)ethyl]-1H-indol-4-ol (201 mg, 0.98 mmol) and 4-methylpiperazine-1-carbonyl chloride hydrochloride (196 mg, 0.98 mmol) in pyridine (4 mL) at rt under N₂ was stirred overnight. The mixture was concentrated in vacuo and the residue was purified by column chromatography on silica gel, eluting with a gradient of 5-15% MeOH in CHCl₃ and then 15% MeOH in CHCl₃ with 1% TEA, to leave a solid. The solid was dissolved in H₂O (2 mL) and freeze dried to leave [3-[2-(dimethylamino)ethyl]-1H-indol-4-yl] 4-methylpiperazine-1-carboxylate (50 mg, 15% yield) as a solid. LC-MS (LCMS2: Method 2B): Rt 1.20 mins; MS m/z 331.1=[M+H]⁺; ¹H NMR (400 MHz, DMSO-d6) δ 10.96 (br. s, 1H), 7.19 (d, J=7.8 Hz, 1H), 7.10 (d, J=2.3 Hz, 1H), 7.00 (t, J=7.8 Hz, 1H), 6.61 (d, J=7.8 Hz, 1H), 3.66 (br. s, 2H), 3.44 (br. s, 2H), 2.80-2.75 (m, 2H), 2.46-2.35 (m, 6H), 2.23 (s, 3H), 2.19 (s, 6H).

Alternatively, the compound could be purified by column chromatography on KP-Amino D silica, eluting with a gradient of petrol in EtOAc to MeOH to give the product (14.7 mg) as a diformate salt. LC-MS (+ve mode): m/z=331.20 [M+H]⁺; ¹H NMR (300 MHz, CDCl₃) δ 8.20 (s, 2H, HCO), 7.13 (m, 2H, 2×ArH), 6.92 (s, 1H, ArH), 6.78 (d, J=7.5 Hz, 1H, ArH), 3.80 (br. s, 2H, CH₂), 3.64 (br. s, 2H, CH₂), 2.96 (m, 2H, CH₂), 2.64 (m, 2H, CH₂), 2.49 (br. s, 4H, 2×CH₂), 2.36 (s, 3H, NMe) 2.32 (s, 6H, 2×NMe); ¹³C NMR (75.5 MHz, CDCl₃) δ 154.2, 144.8, 138.6, 128.1, 122.1, 120.4, 112.5, 108.9, 108.8, 68.0, 60.5, 46.2, 45.3, 45.3, 24.5.

### Example 2: [3-[2-(Dimethylamino)ethyl]-1H-indol-4-yl] sulfamate

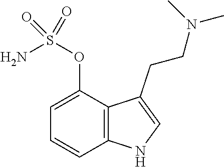 [SMILES: CN(C)CCc1c[nH]c2cccc(OS(N)(=O)=O)c12]

Sulfamoyl chloride (135 mg, 1.17 mmol) was added in one portion to a stirred suspension of 3-[2-(dimethylamino)ethyl]-1H-indol-4-ol (199 mg, 0.97 mmol) and K₂CO₃ (404 mg, 2.92 mmol) in THF (2 mL) at rt under N₂. The mixture was stirred at rt overnight, then the liquid was decanted away from the solid and the solid was purified directly by column chromatography on silica gel, eluting with 10% MeOH in DCM and then 15% MeOH in DCM with 2% TEA, to leave a solid. The solid was dissolved in H₂O (2 mL) and freeze dried to leave [3-[2-(dimethylamino)ethyl]-1H-indol-4-yl] sulfamate (19 mg, 7% yield) as a solid. LC-MS (LCMS2: Method 2B): Rt 0.85 mins; MS m/z 283.9=[M+H]⁺; ¹H NMR (400 MHz, D₂O) δ 7.48 (d, J=7.9 Hz, 1H), 7.36 (s, 1H), 7.26 (t, J=7.9 Hz, 1H), 7.19 (d, J=7.9 Hz, 1H), 3.50 (t, J=7.5 Hz, 2H), 3.36 (t, J=7.5 Hz, 2H), 2.94 (s, 6H). NH and NH₂ not observed; ¹H NMR (400 MHz, DMSO-d6) δ 11.27 (br. s, 1H), 7.29 (d, J=7.8 Hz, 1H), 7.24 (d, J=2.3 Hz, 1H), 7.08 (t, J=7.8 Hz, 1H), 7.02 (d, J=7.8 Hz, 1H), 3.16 (s, 6H), 3.00-2.95 (m, 2H), 2.63-2.60 (m, 2H). NH₂ not observed.

### Example 3: Di-tert-butyl [3-[2-(dimethylamino)ethyl]-1H-indol-4-yl] phosphate hydrochloride

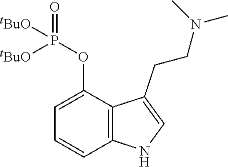 [SMILES: CN(C)CCc1c[nH]c2cccc(OP(=O)(OC(C)(C)C)OC(C)(C)C)c12]

NaOH (40 mg, 1.0 mmol) was added in one portion to a stirred solution of di-tert-butyl phosphite (194 mg, 1.00 mmol, 195 μL) and DMAP (123 mg, 1.00 mmol) in THF (1.5 mL) and CCI₄ (0.50 mL) at 0° C. under an atmosphere of N₂. The mixture was warmed to rt and stirred for 10 min and then a solution of 3-[2-(dimethylamino)ethyl]-1H-indol-4-ol (205 mg, 1.00 mmol) in THF (1.5 mL) was added dropwise over 5 min. The mixture was heated to 50° C. and stirred overnight, then cooled, filtered and the filtrate was concentrated in vacuo. The residue was purified by column chromatography on silica gel, eluting with 10% MeOH in CHCl₃ with 2% TEA, to leave a solid. The solid was triturated with Et₂O (3×5 mL) and then dried under vacuum to leave di-tert-butyl [3-[2-(dimethylamino)ethyl]-1H-indol-4-yl] phosphate hydrochloride (40 mg, 9% yield) as a solid. LC-MS (LCMS2: Method 2B): Rt 1.62 mins; MS m z 397.3=[M+H]⁺; ¹H NMR (400 MHz, D₂O) δ 7.30-7.24 (m, 2H), 7.18 (t, J=7.9 Hz, 1H), 7.07 (d, J=7.9 Hz, 1H), 3.52 (t, J=7.4 Hz, 2H), 3.36 (t, J=7.4 Hz, 2H), 2.93 (s, 6H), 1.45 (s, 9H), 1.25 (s, 9H). NH and HCl not observed; ¹H NMR (400 MHz, DMSO-d₆) δ 11.17 (br. s, 1H), 9.60 (br. s, 1H), 7.25 (d, J=2.4 Hz, 1H), 7.18 (d, J=7.9 Hz, 1H), 7.05 (t, J=7.9 Hz, 1H), 6.93 (d, J=7.9 Hz, 1H), 3.30-3.24 (m, 2H), 3.21-3.14 (m, 2H), 2.82 (s, 6H), 1.42 (s, 18H).

### Example 4: tert-Butyl [3-[2-(dimethylamino)ethyl]-1H-indol-4-yl] hydrogen phosphate

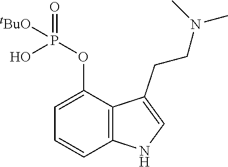 [SMILES: CN(C)CCc1c[nH]c2cccc(OP(=O)(O)OC(C)(C)C)c12]

NaOH (81 mg, 2.04 mmol) was added in one portion to a stirred solution of di-tert-butyl phosphite (216 mg, 1.12 mmol, 217 μL) and DMAP (12 mg, 0.10 mmol) in THF (1 mL) and CCI₄ (0.50 mL) at 0° C. under an atmosphere of N₂. The mixture was stirred at rt for 10 min and then a solution of 3-[2-(dimethylamino)ethyl]-1H-indol-4-ol (208 mg, 1.02 mmol) in THE (2 mL) was added dropwise over 5 min. The mixture was heated to 50° C. and stirred overnight, then cooled to rt, filtered and the filtrate was concentrated in vacuo. The residue was dissolved in DMSO (2 mL) and then purified by reverse phase chromatography, eluting with a gradient of 5-95% MeCN in H₂O with 0.1% ammonia, to leave a solid. The solid was purified by chromatography on silica, eluting with a gradient of 5-10% MeOH in DCM, and then 15% MeOH in DCM with 2% TEA, to give the product (41 mg, 10%) as a solid. LC-MS (LCMS2: Method 2B): Rt 0.95 mins; MS m/z 341.1=[M+H]⁺; ¹H NMR (400 MHz, DMSO-d₆) δ 10.78 (br. s, 1H), 7.03 (d, J=2.2 Hz, 1H), 7.00 (d, J=7.8 Hz, 1H), 6.93 (t, J=7.8 Hz, 1H), 6.66 (d, J=7.8 Hz, 1H), 3.24-3.20 (m, 2H), 3.18-3.12 (m, 2H), 2.71 (s, 6H), 1.39 (s, 9H). P(═O)OH not observed.

### Example 5: Chloromethyl Isopropyl Carbonate

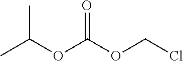

Chloromethyl chloroformate (2.29 g, 17.8 mmol, 1.58 mL) was added dropwise over 15 min to a stirred solution of iso-propanol (785 mg, 13.1 mmol, 1.00 mL) and pyridine (1.54 g, 19.4 mmol, 1.57 mL) in DCM (15 mL) at rt under an atmosphere of N₂. The mixture was warmed to rt and stirred overnight, then diluted with DCM (15 mL) and washed with 1M aqueous HCl (30 mL) and H₂O (2×30 mL). The organic layer was dried over Na₂SO₄ and concentrated in vacuo to leave the crude chloromethyl isopropyl carbonate (1.93 g, 97% yield) as an oil. The title compound was used without further purification. ¹H NMR (400 MHz, CDCl₃) δ 5.72 (s, 2H), 4.96 (hept, J=6.3 Hz, 1H), 1.33 (d, J=6.3 Hz, 6H).

The compound of the following tabulated example (Table Ex5) was prepared analogously to Example 5 from chloromethyl chloroformate and the appropriate alcohol.

TABLE
Ex 5

| Ex | Structure and Name | Characterization |
|---|---|---|
| 5.1 | 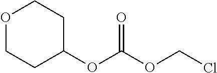 [SMILES: O=C(OCCl)OC1CCOCC1] | ¹H NMR (400 MHz, CDCl₃) δ 5.74 (s, 2H), 4.90 (tt, J = 8.6, 4.1 Hz, 1H), 3.97-3.90 (m, 2H), 3.55 (ddd, J = 11.9, 8.6, 3.1 Hz, 2H), 2.05-1.97 (m, 2H), 1.82-1.72 (m, 2H). |
| | Chloromethyl tetrahydropyran-4-yl | |
| | carbonate | |

### Example 6: [3-[2-(Dimethylamino)ethyl]-1H-indol-4-yl]oxymethyl tetrahydropyran-4-yl carbonate

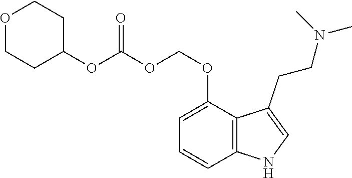 [SMILES: CN(C)CCc1c[nH]c2cccc(OCOC(=O)OC3CCOCC3)c12]

A solution of 3-[2-(dimethylamino)ethyl]-1H-indol-4-ol (209 mg, 1.02 mmol), tetrabutylammonium bromide (330 mg, 1.02 mmol) and TEA (310 mg, 3.07 mmol, 428 μL) in MeCN (5 mL) at rt under an atmosphere of N₂ was stirred for 15 min and then a solution of chloromethyl tetrahydropyran-4-yl carbonate (398 mg, 2.05 mmol) in MeCN (5 mL) was added dropwise over 10 min. The mixture was stirred at rt for 30 min and then NaI (15 mg, 0.10 mmol) was added in one portion. The mixture was stirred at rt for 2 days, then concentrated in vacuo. EtOAc (50 mL), H₂O (50 mL) and brine (50 mL) were added to the residue. The separated aqueous phase was extracted with EtOAc (50 mL) and the combined organic layers were then dried over MgSO₄, filtered and the filtrate was concentrated in vacuo to leave the crude [3-[2-(dimethylamino)ethyl]-1H-indol-4-yl]oxymethyl tetrahydropyran-4-yl carbonate as an oil. LC-MS (LCMS2: Method 2B): Rt 1.31 mins; MS m z 363.2 [M+H]⁺.

### Alternative Procedure:

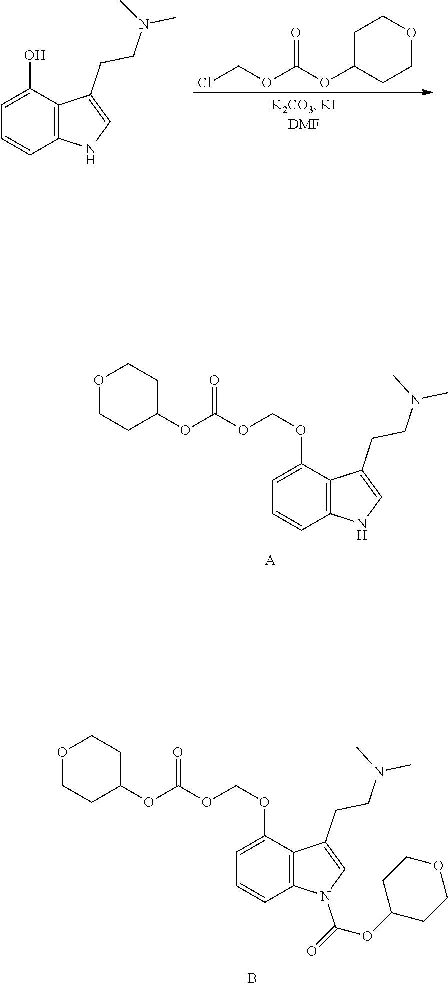

Potassium carbonate (149 mg, 1.08 mmol) and potassium iodide (18 mg, 0.11 mmol) were added to a stirred solution of psilocin (220 mg, 1.08 mmol) in anhydrous DMF (3 mL) at rt under an atmosphere of N₂. The resulting suspension was stirred at rt for 15 min, then a solution of chloromethyl (tetrahydro-2H-pyran-4-yl) carbonate (210 mg, 1.08 mmol) in anhydrous DMF (3 mL) was added dropwise to this suspension and the mixture was stirred at rt for 16 h. The solvent was removed under reduced pressure and the residual material was purified by column chromatography on silica gel, eluting with a gradient of MeOH in EtOAc to afford a fraction product A (54 mg) as a semi-solid and a fraction containing tetrahydro-2H-pyran-4-yl 3-(2-(dimethylamino)ethyl)-4-(((((tetrahydro-2H-pyran-4-yl)oxy)carbonyl)oxy)methoxy)-1H-indole-1-carboxylate (B) (192 mg) as a semi-solid. LC-MS (+ve mode): m/z=363.10 (A) and 491.10 (B) [M+H]⁺. The product A fraction (54 mg) was purified by reversed-phase chromatography on Cis silica, eluting with a gradient of MeCN in H₂O to give the desired product (9 mg) as a solid. LC-MS (+ve mode): m/z=363.15 [M+H]⁺.

### Example 7: [3-[2-(Dimethylamino)ethyl]-1H-indol-4-yl]oxymethyl isopropyl carbonate

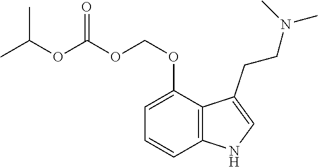 [SMILES: CC(C)OC(=O)OCOc1cccc2[nH]cc(CCN(C)C)c12]

A solution of 3-[2-(dimethylamino)ethyl]-1H-indol-4-ol (201 mg, 0.98 mmol), Bu₄NBr (317 mg, 0.98 mmol) and TEA (298 mg, 2.95 mmol, 411 μL) in MeCN (5 mL) was stirred at rt under an atmosphere of N₂ for 15 min and then a solution of chloromethyl isopropyl carbonate (300 mg, 1.97 mmol) in MeCN (5 mL) was added dropwise over 10 min. The mixture was stirred at rt for 30 min and then NaI (15 mg, 0.10 mmol) was added in one portion. The mixture was stirred at rt for 3 days, then concentrated in vacuo. EtOAc (50 mL), H₂O (50 mL) and brine (50 mL) were added to the residue. The separated aqueous phase was extracted with EtOAc (50 mL) and the combined organic layers were then dried over MgSO₄, filtered and the filtrate was concentrated in vacuo. The residue was purified by column chromatography on silica gel, eluting with a gradient of 10-20% MeOH in DCM, and then 20% MeOH in DCM with 2% TEA, to give the product (5 mg, 2%) as a solid. LC-MS (LCMS2: Method 2B): Rt 1.35 mins; MS m z 321.1=[M+H]⁺; ¹H NMR (400 MHz, CD₃OD) δ 7.42 (d, J=8.0 Hz, 1H), 7.23-7.14 (m, 2H), 6.90 (d, J=8.0 Hz, 1H), 5.55 (s, 2H), 4.98 (hept, J=6.2 Hz, 1H), 3.06-2.95 (m, 4H), 2.59 (s, 6H), 1.38 (d, J=6.2 Hz, 6H). NH not observed.

### Example 8: tert-Butyl Chloromethyl Succinate

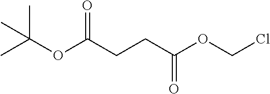

Chloromethyl chlorosulfate (3.41 g, 20.7 mmol, 2.09 mL) was added dropwise over 10 min to a vigorously stirred bi-phasic mixture of 4-tert-butoxy-4-oxo-butanoic acid (3.00 g, 17.2 mmol), sodium bicarbonate (5.79 g, 68.9 mmol) and tetrabutylammonium hydrogen sulfate (585 mg, 1.72 mmol) in DCM (45 mL) and H₂O (30 mL) at rt under an atmosphere of N₂. The mixture was stirred vigorously at rt overnight, then diluted with DCM (40 mL) and H₂O (40 mL). The separated organic phase was washed with saturated aqueous NaHCO₃ (40 mL), dried over Na₂SO₄, filtered and the filtrate was concentrated in vacuo. The residue was purified by column chromatography on silica gel, eluting with a gradient of 0-15% EtOAc in petroleum ether, to leave tert-butyl chloromethyl succinate (3.15 g, 820 yield) as an oil. ¹H NMR (400 MHz, CDCl₃) δ 5.71 (s, 2H), 2.69-2.61 (m, 2H), 2.62-2.54 (m, 2H), 1.45 (s, 9H).

The compounds of the following tabulated Examples (Table Ex8) were prepared analogously to Example 8 from the appropriate carboxylic acid.

TABLE
Ex 8

| Ex. | Structure and Name | Characterization |
|---|---|---|
| 8.1 | 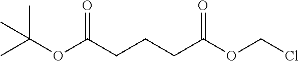 | ¹H NMR (400 MHz, CDCl₃) δ 5.70 (s, 2H), 2.45 (t, J = 7.4 Hz, 2H), 2.29 (t, J = 7.4 Hz, 2H), 1.94 (p, J = 7.4 Hz, 2H), 1.44 (s, 9H). |
| | tert-Butyl chloromethyl glutarate | |
| 8.2 | 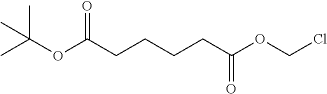 [SMILES: CC(C)(C)OC(=O)CCCCC(=O)OCCl] | ¹H NMR (400 MHz, CDCl₃) δ 5.70 (s, 2H), 2.41 (t, J = 7.2 Hz, 2H), 2.24 (t, J = 7.2 Hz, 2H), 1.73-1.58 (m, 4H), 1.44 (s, 9H). |
| | tert-Butyl chloromethyl adipate | |
| 8.3 | 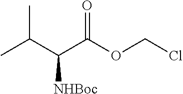 [SMILES: CC(C)[C@H](NC(=O)OC(C)(C)C)C(=O)OCCl] | LC-MS (LCMS2: Method 2A): Rt 1.64 mins; MS m/z 210.1 and 212.1 = [M = ^{t}Bu + H]⁺ ¹H NMR (400 MHz, CDCl₃) δ 5.87 (d, J = 6.1 Hz, 1H), 5.62 (d, J = 6.1 Hz, 1H), 4.97 (br. d, J = 9.1 Hz, 1H), 4.27 (dd, J = 9.1, 4.8 Hz, 1H), 2.24-2.14 (m, 1H), 1.45 (s, 9H), 1.00 (d, J = 6.9 Hz, 3H), 0.92 (d, J = 6.9 Hz, 3H). |
| | Chloromethyl (S)-2-(tert- | |
| | butoxycarbonylamino)-3-methyl-butyrate | |

### Example 9: Chloromethyl [1,4′-bipiperidyl]-1′-carboxylate hydrochloride

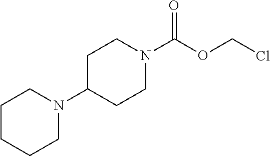 [SMILES: O=C(OCCl)N1CCC(N2CCCCC2)CC1]

A solution of chloromethyl chloroformate (500 mg, 3.88 mmol, 345 μL) in DCM (3 mL) was added dropwise over 15 min to a stirred solution of 1,4′-bipiperidine (544 mg, 3.23 mmol) in DCM (3 mL) at 5° C. under an atmosphere of N₂. The mixture was stirred at 5° C. for 30 min and then warmed to rt and stirred overnight. H₂O (0.2 mL) was added to the mixture and the mixture was then stirred vigorously at rt for 30 min. The mixture was concentrated in vacuo and the residue was azeotroped with THF (2×4 mL) and then dried under vacuum to leave chloromethyl [1,4′-bipiperidyl]-1′-carboxylate hydrochloride (960 mg, assume 10000 yield) as a solid. The title compound was used without further purification. ¹H NMR (400 MHz, CD₃OD) δ 5.93-5.73 (m, 2H), 4.41-4.22 (m, 2H), 3.55-3.46 (m, 2H), 3.42 (tt, J=12.2, 3.8 Hz, 1H), 3.09-2.83 (m, 4H), 2.22-2.07 (m, 2H), 2.03-1.95 (m, 2H), 1.89-1.62 (m, 5H), 1.58-1.46 (m, 1H). HCl not observed; ¹H NMR (400 MHz, DMSO-d6) δ 10.20 (br. s, 1H), 5.95-5.81 (m, 2H), 4.17-4.02 (m, 2H), 3.42-3.35 (m, 3H), 2.98-2.82 (m, 4H), 2.16-2.05 (m, 2H), 1.87-1.53 (m, 7H), 1.44-1.32 (m, 1H).

### Example 10: tert-Butyl {3-[2-(dimethylamino)ethyl]-4-indolyloxy}methyl adipate

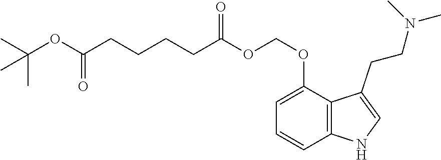 [SMILES: CN(C)CCc1c[nH]c2cccc(OCOC(=O)CCCCC(=O)OC(C)(C)C)c12]

An aqueous solution of NaOH (3.75 M, 4.73 mmol, 1.26 mL) was added dropwise over 5 min to a stirred suspension of 3-[2-(dimethylamino)ethyl]-1H-indol-4-ol (322 mg, 1.58 mmol) and tetrabutylammonium hydrogen sulfate (107 mg, 0.32 mmol) in DCM (3 mL) at rt under an atmosphere of N₂. The mixture was stirred vigorously at rt for 5 min and then a solution of tert-butyl chloromethyl adipate (395 mg, 1.58 mmol) in DCM (2 mL) was added dropwise over 2 min. The mixture was stirred vigorously at rt for 15 min, then diluted with DCM (20 mL) and H₂O (20 mL). The separated aqueous phase was extracted with DCM (2×15 mL) and then the combined organic layers were washed with brine (20 mL), dried over Na₂SO₄, filtered and the filtrate was concentrated in vacuo to leave crude tert-butyl {3-[2-(dimethylamino)ethyl]-4-indolyloxy}methyl adipate as a gum. LC-MS (LCMS2: Method 2B): Rt 1.72 mins; MS m/z 419.3=[M+H]⁺. A sample of material was purified by column chromatography on silica gel, eluting with a gradient of MeOH in DCM (with 0.1% Et₃N) to afford the product semi-solid (41 mg). LC-MS (+ve mode): m/z=419.20 [M+H]⁺; ¹H NMR (300 MHz, CDCl₃) δ 7.12 (t, J=7.9 Hz, 1H, ArH), 6.94 (dd, J=8.2, 0.9 Hz, 1H, ArH), 6.90 (s, 1H, ArH), 6.61 (dd, J=7.7, 0.9 Hz, 1H, ArH), 5.98 (s, 2H, CH₂), 2.93 (m, 2H, CH₂), 2.74 (m, 2H, CH₂), 2.40 (s, 6H, 2×NCH₃), 2.31 (t, J=7.1 Hz, 2H, CH₂), 2.18 (t, J=7.0 Hz, 2H, CH₂), 1.59 (m, 4H, 2×CH₂), 1.42 (s, 9H, 3×CH₃).

The compounds of the following tabulated Examples (Table Ex10) were prepared analogously to Example 10 from 3-[2-(dimethylamino)ethyl]-1H-indol-4-ol and the appropriate chloride.

TABLE
Ex 10

| Ex. | Structure and Name | Characterization |
|---|---|---|
| 10.1 | 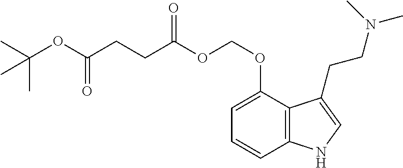 [SMILES: CN(C)CCc1c[nH]c2cccc(OCOC(=O)CCC(=O)OC(C)(C)C)c12] | LC-MS (LCMS2: Method 2B): Rt 1.58 mins; MS m/z 391.3 = [M + H]⁺ Product purified by column chromatography on KP-Amino D silica, eluting with a gradient of petrol in EtOAc to MeOH to give a colourless semi-solid (84.7 mg). LC- MS (+ve mode): m/z = 391.20 [M + H]⁺; ¹H NMR (300 MHz, CDCl₃) δ 7.09 (m, 1H, ArH), 6.93 (m, 2H, 2 × ArH) 6.61 (d, J = 8.5 Hz, 2H, ArH), 6.00 (s, 2H, CH₂), 2.88 (m, 2H, CH₂), 2.70 (m, 2H, CH₂), 2.52 (m, |
| | tert-Butyl {3-[2-(dimethylamino)ethyl]-4-indolyloxy}methyl succinate | 4H, 2 × CH₂), 2.38 (s, 6H, 2 × NMe), 1.40 |
| | | (s, 9H, 3 × CH₃); ¹³C NMR (75.5 MHz, |
| | | CDCl₃) δ 172.4, 171.2, 152.3, 139.2, 125.2, |
| | | 124.2, 118.4, 115.4, 107.9, 100.7, 80.9, 68.7, |
| | | 61.3, 45.3, 30.1, 29.2, 28.0, 24.9. |
| 10.2 | 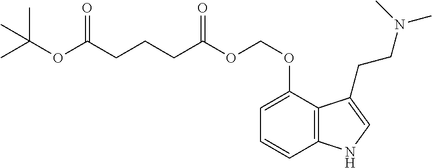 [SMILES: CN(C)CCc1c[nH]c2cccc(OCOC(=O)CCCC(=O)OC(C)(C)C)c12] | LC-MS (LCMS2: Method 2B): Rt 1.66 mins; MS m/z 405.3 = [M + H]⁺ Purified by column chromatography on silica gel, eluting with a gradient of MeOH in DCM (0.1% Et₃N) to afford a semi-solid (37 mg). LC-MS (+ve mode): m/z = 405.25 [M + H]⁺; ¹H NMR (300 MHz, CDCl₃) δ 7.12 (t, J = 7.9 Hz, 1H, ArH), 6.93 (dd, J = 8.2, 0.9 Hz, 1H, ArH), 6.90 (s, 1H, ArH), 6.62 (dd, J = 7.7, 0.9 Hz, 1H, ArH), 5.98 (s, 2H, CH₂), 2.92 (m, 2H, CH₂), 2.73 (m, 2H, |
| | tert-Butyl {3-[2-(dimethylamino)ethyl]-4- | CH₂), 2.39 (s, 6H, 2 × NCH₃), 2.34 (m, 2H, |
| | indolyloxy}methyl glutarate | CH₂), 2.22 (m, 2H, CH₂), 1.97 (m, 2H, |
| | | CH₂), 1.42 (s, 9 H, 3 × CH₃); ¹³C NMR |
| | | (75.5 MHz, CDCl₃) δ 173.1, 172.3, 152.4, |
| | | 139.3, 125.4, 124.3, 118.5, 115.4, 10.0, |
| | | 100.8, 80.6, 68.7, 61.4, 45.3, 34.5, 33.3, |
| | | 28.2, 24.9, 20.1. |
| 10.3 | 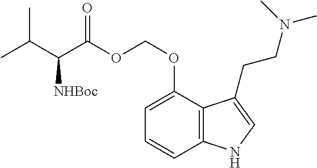 [SMILES: CC(C)[C@H](NC(=O)OC(C)(C)C)C(=O)OCOc1cccc2[nH]cc(CCN(C)C)c12] | LC-MS (LCMS2: Method 2A): Rt 1.27 mins; MS m/z 434.3 = [M + H]⁺ |
| | {3-[2-(Dimethylamino)ethyl]-4-indolyloxy}methyl (S)-2-(tert- | |
| | butoxycarbonylamino)-3-methyl-butyrate | |
| 10.4 | 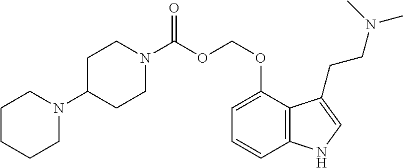 [SMILES: CN(C)CCc1c[nH]c2cccc(OCOC(=O)N3CCC(N4CCCCC4)CC3)c12] | LC-MS (LCMS2: Method 2A): Rt 0.65 mins; MS m/z 429.4 = [M + H]⁺ |
| | [3-[2-(Dimethylamino)ethyl]-1H-indol-4-yl]oxymethyl 4-(1- | |
| | piperidyl)piperidine-1-carboxylate | |

### Example 11: [3-[2-(Dimethylamino)ethyl]-1H-indol-4-yl] oxetan-3-yl carbonate

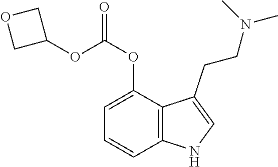 [SMILES: CN(C)CCc1c[nH]c2cccc(OC(=O)OC3COC3)c12]

Oxetan-3-ol (145 mg, 1.96 mmol, 124 μL) was added dropwise over 2 min to a stirred solution of bis(4-nitrophenyl) carbonate (328 mg, 1.08 mmol) and DMAP (12 mg, 0.10 mmol) in DCM (3 mL) at rt under an atmosphere of N₂. The mixture was stirred at rt for 1 h, then 3-[2-(dimethylamino)ethyl]-1H-indol-4-ol (200 mg, 0.98 mmol) was added in one portion to the mixture at rt, followed by DIPEA (127 mg, 0.98 mmol, 171 μL) which was added dropwise over 2 min. The mixture was stirred at rt for 2 days, then H₂O (4 mL) and EtOAc (4 mL) were added to the mixture. The separated organic layer was washed with brine (10 mL), dried over Na₂SO₄ and concentrated in vacuo to leave crude [3-[2-(dimethylamino)ethyl]-1H-indol-4-yl] oxetan-3-yl carbonate as a gum. LC-MS (LCMS2: Method 2B): Rt 1.29 mins; MS m z 305.2=[M+H]⁺.

### Example 12: Di-tert-butyl [3-[2-(dimethylamino)ethyl]-1H-indol-4-yl]oxymethyl phosphate

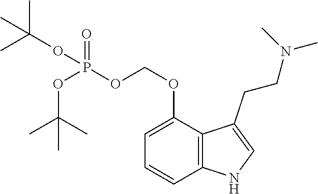 [SMILES: CN(C)CCc1c[nH]c2cccc(OCOP(=O)(OC(C)(C)C)OC(C)(C)C)c12]

Sodium hydride, 60% dispersion in mineral oil (60 mg, 1.51 mmol) was added in one portion to a stirred solution of 3-[2-(dimethylamino)ethyl]-1H-indol-4-ol (205 mg, 1.00 mmol) in anhydrous DMF (2.5 mL) at rt under an atmosphere of N₂. The mixture was stirred at rt for 10 min, then di-tert-butyl chloromethyl phosphate (317 mg, 1.10 mmol, 305 μL) was added dropwise over 2 min, followed by potassium iodide (500 mg, 3.01 mmol) which was added in one portion. The mixture was stirred at rt overnight, then H₂O (1 mL) and EtOAc (20 mL) were added and then the mixture was washed with 90% aqueous brine (20 mL), 50% aqueous brine (3×20 mL) and saturated aqueous sodium thiosulfate (20 mL). The organic layer was dried over Na₂SO₄, filtered and the filtrate was concentrated in vacuo. The residue was purified by chromatography on silica gel, eluting with a gradient of 0-5% MeOH in DCM, and then a gradient of 5-10% MeOH in DCM with 0.5% TEA, to leave the product (76 mg, 16%) as a gum. LC-MS (LCMS2: Method 2B): Rt 1.82 mins; MS m z 427.3=[M+H]⁺; ¹H NMR (400 MHz, CDCl₃) δ 8.24 (br. s, 1H), 7.09-7.01 (m, 2H), 6.95-6.88 (m, 1H), 6.80 (dd, J=6.6, 1.9 Hz, 1H), 5.76 (d, J=11.4 Hz, 2H), 3.13-3.04 (m, 2H), 2.79-2.69 (m, 2H), 2.42 (s, 6H), 1.44 (s, 18H); ¹H-³¹P coupled: ³¹P NMR (162 MHz, CDCl₃) δ −11.83 (t, J=11.4 Hz); ¹H-³¹P decoupled: ³¹P NMR (162 MHz, CDCl₃) δ −11.83 (s).

### Example 13: tert-Butyl 5-({3-[2-(dimethylamino)ethyl]-4-indolyloxycarbonyl}-N-methylamino)valerate formate

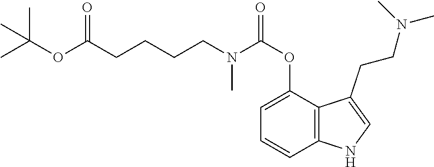 [SMILES: CN(C)CCc1c[nH]c2cccc(OC(=O)N(C)CCCCC(=O)OC(C)(C)C)c12]

Triphosgene (109 mg, 0.37 mmol) was added in one portion to a stirred suspension of 3-[2-(dimethylamino)ethyl]-1H-indol-4-ol (203 mg, 0.99 mmol) and DMAP (389 mg, 3.18 mmol) in DCM (10 mL) at rt under an atmosphere of N₂. The mixture was stirred at rt for 1 h, then tert-butyl 5-(methylamino)pentanoate hydrochloride (222 mg, 0.99 mmol) was added in one portion to the mixture at rt followed by TEA (211 mg, 2.09 mmol, 291 μL) which was added dropwise over 2 min. The mixture was stirred at rt for 2 h, then concentrated in vacuo. The residue was purified by column chromatography on silica gel, eluting with a gradient of 0-10% MeOH in DCM, to leave an oil. The crude material was dissolved in DMSO (1.5 mL) and re-purified by reverse phase chromatography, eluting with a gradient of 10-40% MeCN in H₂O with 0.1% formic acid, to leave tert-butyl 5-({3-[2-(dimethylamino)ethyl]-4-indolyloxycarbonyl}-N-methylamino)valerate formate (63 mg, 13% yield) as a gum. Spectroscopic data of the title compound was obtained as a mixture of rotational isomers. LC-MS (LCMS2: Method 2A): Rt 1.17 mins; MS m/z 418.2=[M+H]⁺; ¹H NMR (400 MHz, DMSO-d₆) δ 10.99 (br. s, 1H), 8.19 (s, 1H), 7.19 (d, J=8.0 Hz, 1H), 7.13 (d, J=2.3 Hz, 1H), 7.00 (t, J=8.0 Hz, 1H), 6.62-6.55 (m, 1H), 3.47 (t, J=7.0 Hz, 0.8H), 3.30 (t, J=6.6 Hz, 1.2H), 3.10 (s, 1.8H), 2.91 (s, 1.2H), 2.84-2.78 (m, 2H), 2.62-2.54 (m, 2H), 2.30-2.21 (m, 8H), 1.69-1.49 (m, 4H), 1.39 (s, 5.4H), 1.38 (s, 3.6H). CO₂H not observed.

### Example 14: 5-({3-[2-(Dimethylamino)ethyl]-4-indolyloxycarbonyl}-N-methylamino)valeric acid formate trifluoroacetate

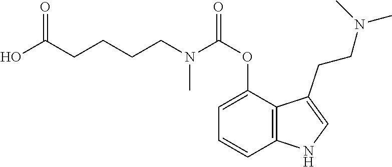 [SMILES: CN(C)CCc1c[nH]c2cccc(OC(=O)N(C)CCCCC(=O)O)c12]

TFA (1.48 g, 13.0 mmol, 1.00 mL) was added dropwise over 5 min to a stirred solution of tert-butyl 5-({3-[2-(dimethylamino)ethyl]-4-indolyloxycarbonyl}-N-methylamino)valerate formate (38 mg, 0.08 mmol) in DCM (1 mL) at rt under an atmosphere of N₂. The mixture was at rt for 1 h, then concentrated in vacuo to give the product (43 mg, 94%) as a gum. Spectroscopic data of the title compound was obtained as a mixture of rotational isomers. LC-MS (LCMS1: Method 2A): Rt 0.76 mins; MS m z 362.4=[M+H]⁺; ¹H NMR (400 MHz, DMSO-d₆) δ 12.07 (br. s, 1H), 11.20 (s, 1H), 9.37 (br. s, 1H), 7.29-7.21 (m, 2H), 7.05 (t, J=7.9 Hz, 1H), 6.68-6.61 (m, 1H), 3.52-3.47 (m, 0.8H), 3.38-3.29 (m, 3.2H), 3.12 (s, 1.8H), 3.07-2.99 (m, 2H), 2.93 (s, 1.2H), 2.85-2.77 (m, 6H), 2.32-2.24 (m, 2H), 1.67-1.48 (m, 4H). Two CO₂H not observed; ¹⁹F NMR (376 MHz, DMSO-d6) δ −73.9 (s).

The compound of the following tabulated Example (Table Ex14) was prepared analogously to Example 14 from the appropriate tert-butyl protected compound.

TABLE
Ex 14

| Ex. | Structure and Name | Characterization |
|---|---|---|
| 14.1 | 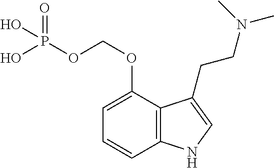 [SMILES: CN(C)CCc1c[nH]c2cccc(OCOP(=O)(O)O)c12] | LC-MS (LCMS2: Method 2B): Rt 0.27 mins; MS m/z 315.2 = [M + H]⁺ |
| | [3-[2-(Dimethylamino)ethyl]-1H-indol-4-yl] | |
| | oxymethyl dihydrogen phosphate | |

### Example 15: 4-(((3-(2-(dimethylamino)ethyl)-1H-indol-4-yl)oxy)methoxy)-4-oxobutanoic acid trifluoroacetate

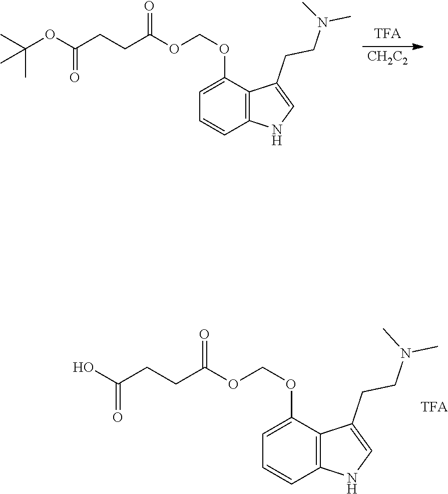

To tert-butyl (((3-(2-(dimethylamino)ethyl)-1H-indol-4-yl)oxy)methyl) succinate (68 mg, 0.17 mmol) in DCM (5 mL) was added TFA (0.69 mL, 0.96 g, 8.5 mmol) and the mixture was stirred at rt for 3 h. The mixture was concentrated under reduced pressure and the residue was azeotroped with MeOH (3×10 mL) to afford the product (122 mg) as a semi-solid. LC-MS (+ve mode): m/z=335.15 [M+H]⁺.

### Example 16: (S)-((3-(2-(dimethylamino)ethyl)-1H-indol-4-yl)oxy)methyl 2-amino-3-methylbutanoate

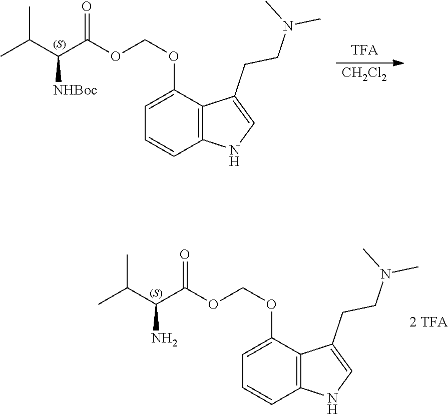

{3-[2-(Dimethylamino)ethyl]-4-indolyloxy}methyl (S)-2-(tert-butoxycarbonylamino)-3-methyl-butyrate (16 mg, 40 μmol) was dissolved in anhydrous dichloromethane (0.5 mL) and TFA (0.5 mL) was added. The reaction mixture was stirred at room temperature under nitrogen. After 5 min the mixture turned dark blue. LC-MS (+ve mode): m/z=334.18 [M+H]⁺.

The following UPLC-MS methods and conditions were used in Examples 17-45.

UPLC-MS analysis was carried out on a Waters Acquity UPLC system consisting of an Acquity I-Class Sample Manager-FL, Acquity I-Class Binary Solvent Manager and an Acquity UPLC Column Manager. UV detection was afforded using an Acquity UPLC PDA detector (scanning from 210 to 400 nm), whilst mass detection was achieved using an Acquity Quad detector (mass scanning from 100-1250 Da; positive and negative modes simultaneously), and ELS detection was achieved using an Acquity UPLC ELS Detector. A Waters Acquity UPLC BEH C18 column (2.1×50 mm, 1.7 mm) was used to separate the analytes.

Samples were prepared by dissolution (with or without sonication) into 1 mL of 50% (v/v) MeCN in water. The resulting solutions were then filtered through a 0.2 mm syringe filter before submitting for analysis. All of the solvents, including formic acid and 36% ammonia solution, were purchased as the HPLC grade.

Conditions (Acidic 2 min): 0.1% v/v Formic acid in water [Eluent A]; 0.1% v/v Formic acid in MeCN [Eluent B]; flow rate 0.8 mL/min; column oven 50° C.; sample manager 20° C.; injection volume 2 mL and 1.5 minutes equilibration time between samples. Gradient parameters are provided in Table 2:

TABLE 2

| Time (min) | Eluent A (%) | Eluent B (%) |
|---|---|---|
| 0.00 | 95 | 5 |
| 0.25 | 95 | 5 |
| 1.25 | 5 | 95 |
| 1.55 | 5 | 95 |
| 1.65 | 95 | 5 |
| 2.00 | 95 | 5 |

Conditions (Acidic 4 min): 0.100 v/v formic acid in water [Fluent A]; 0.1% v/v formic acid in MeCN [Eluent B]; flow rate 0.8 mL/min; column oven 50° C.; sample manager 20° C.; injection volume 2 mL and 1.5 minutes equilibration time between samples. Gradient parameters are provided in Table 3.

TABLE 3

| Time (min) | Eluent A (%) | Eluent B (%) |
|---|---|---|
| 0.00 | 95 | 5 |
| 0.25 | 95 | 5 |
| 2.75 | 5 | 95 |
| 3.25 | 5 | 95 |
| 3.35 | 95 | 5 |
| 4.00 | 95 | 5 |

Conditions (Acidic 6 min): 0.1% v/v formic acid in water [Fluent A]; 0.1% v/v formic acid in MeCN [Eluent B]; flow rate 0.8 mL/min; column oven 50° C.; sample manager 20° C.; injection volume 2 mL and 1.5 minutes equilibration time between samples. Gradient parameters are provided in Table 4.

TABLE 4

| Time (min) | Eluent A (%) | Eluent B (%) |
|---|---|---|
| 0.00 | 95 | 5 |
| 0.30 | 95 | 5 |
| 6.00 | 5 | 95 |
| 6.10 | 95 | 5 |
| 7.00 | 95 | 5 |

Conditions (Basic 2 min): 0.1% ammonia in water [Eluent A]; 0.1% ammonia in MeCN [Eluent B], flow rate 0.8 mL/min; column oven 50° C.; sample manager 20° C.; injection volume 2 mL and 1.5 minutes equilibration time between samples. Gradient parameters are provided in Table 5.

TABLE 5

| Time (min) | Eluent A (%) | Eluent B (%) |
|---|---|---|
| 0.00 | 95 | 5 |
| 0.25 | 95 | 5 |
| 1.25 | 5 | 95 |
| 1.55 | 5 | 95 |
| 1.65 | 95 | 5 |
| 2.00 | 95 | 5 |

Conditions (Basic 4 min): 0.1% ammonia in water [Eluent A]; (%) ammonia in MeCN [Eluent B], flow rate 0.8 mL/min; column oven 50° C.; sample manager 20° C.; injection volume 2 mL and 1.5 minutes equilibration time between samples. Gradient parameters are provided in PUP-177, 11 Table 6.

TABLE 6

| Time (min) | Eluent A (%) | Eluent B (%) |
|---|---|---|
| 0.00 | 95 | 5 |
| 0.25 | 95 | 5 |
| 2.75 | 5 | 95 |
| 3.25 | 5 | 95 |
| 3.35 | 95 | 5 |
| 4.00 | 95 | 5 |

Conditions (Basic 6 min): 0.1% ammonia in water [Eluent A]; 0.1% ammonia in MeCN [Eluent B], flow rate 0.8 mL/min; column oven 50° C.; sample manager 20° C.; injection volume 2 mL and 1.5 minutes equilibration time between samples. Gradient parameters are provided in Table 7.

TABLE 7

| Time (min) | Eluent A (%) | Eluent B (%) |
|---|---|---|
| 0.00 | 95 | 5 |
| 0.30 | 95 | 5 |
| 6.00 | 5 | 95 |
| 6.10 | 95 | 5 |
| 7.00 | 95 | 5 |

### Example 17: 3-(2-(Dimethylamino)ethyl)-1H-indol-4-yl tetrahydro-2H-pyran-4-carboxylate hydrochloride

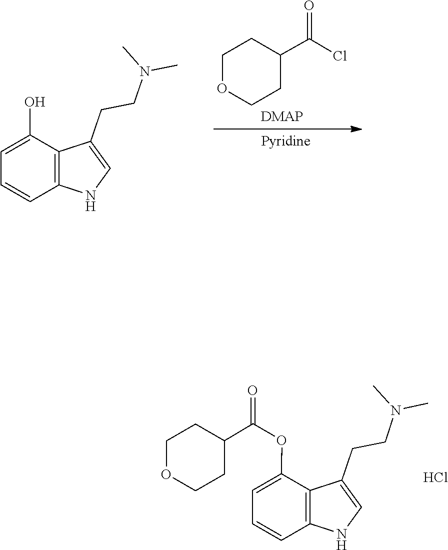

To a mixture of psilocin (170 mg, 0.83 mmol) in anhydrous pyridine (10 mL) was added DMAP (10 mg, 0.08 mmol) and the mixture was cooled to 0° C. Oxane-3-carbonyl chloride (117 mg, 97 μL, 0.79 mmol) was added cautiously and the mixture was warmed to rt and stirred for 16 h. The mixture was concentrated under reduced pressure and the residue was purified by reversed-phase chromatography on C₁₈ silica, eluting with a gradient of acetonitrile in 0.02% hydrochloric acid to afford the product (231 mg, 84%) as a glassy solid. LC-MS (+ve mode): m/z=317.15 [M+H]⁺; ¹H NMR (300 MHz, CD₃OD) δ 7.23 (m, 2H, 2×ArH), 7.07 (t, J=9.0 Hz, 1H, ArH), 6.67 (dd, J=7.7, 0.8 Hz, 1H, ArH), 3.97 (m, 2H, CH₂), 3.54 (td, J=11.5, 2.5 Hz, 2H, CH₂), 3.43 (t, J=7.3 Hz, 2H, CH₂), 3.15 (t, J=7.2 Hz, 2H, CH₂), 2.86 (s, 6H, 2×NMe), 2.03 (m, 2H, CH₂), 1.87 (m, 2H, CH₂); ¹³C NMR (75.5 MHz, CDCl₃) δ 200.5, 174.0, 143.9, 139.3, 123.9, 121.7, 111.7, 109.4, 107.0, 66.6, 58.2, 42.4, 39.9, 28.6, 21.6.

### Example 18: 3-(2-(Dimethylamino)ethyl)-1H-indol-4-yl oxetane-3-carboxylate

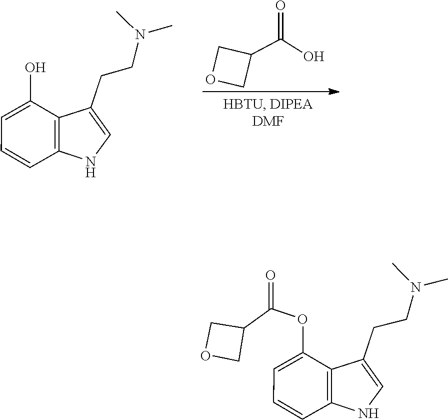 [SMILES: CN(C)CCc1c[nH]c2cccc(O)c12 ; O=C(O)C1COC1 ; CN(C)CCc1c[nH]c2cccc(OC(=O)C3COC3)c12]

Oxetane-3-carboxylic acid (79 mg, 0.77 mmol) was dissolved in anhydrous DMF (5 mL) under an atmosphere of N₂ and N,N-diisopropylethylamine (108 mg, 146 μL, 0.84 mmol) was added followed by psilocin (132 mg, 0.65 mmol) and HBTU (269 mg, 0.71 mmol). The mixture was stirred at rt for 20 h, then the volatiles were removed under reduced pressure and saturated aqueous NaHCO₃ (20 mL) was added. The resulting mixture was extracted with EtOAc (50 mL) and the organic layer was washed with H₂O (20 mL), saturated brine (20 mL), dried (MgSO₄), filtered and the filtrate was concentrated to give the product (200 mg). LC-MS (+ve mode): m/z=289.15 [M+H]⁺.

### Example 19: 3-(2-(Dimethylamino)ethyl)-1H-indol-4-yl tetrahydrofuran-3-carboxylate hydrochloride

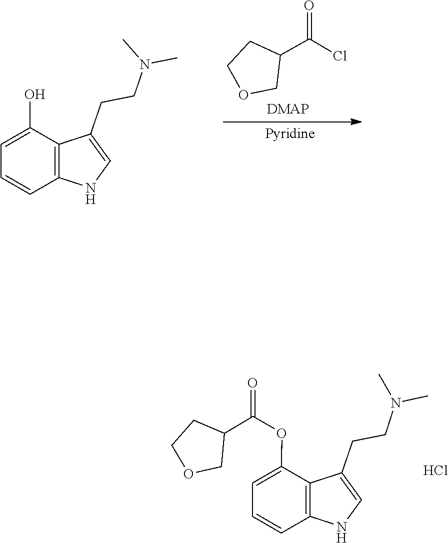 [SMILES: CN(C)CCc1c[nH]c2cccc(O)c12 ; O=C(Cl)C1CCOC1 ; CN(C)CCc1c[nH]c2cccc(OC(=O)C3CCOC3)c12 ; Cl]

Tetrahydrofuran-3-carbonyl chloride (184 mg, 145 μL, 1.37 mmol) was added to a stirred solution of psilocin (200 mg, 0.98 mmol) in anhydrous pyridine (2.4 mL) at rt. The mixture was heated to 40° C. and stirred for 16 h, then the volatiles were removed under reduced pressure. The residue was purified by reversed-phase chromatography on C₁₈ silica, eluting with a gradient of acetonitrile in 0.02% hydrochloric acid to afford the product (286 mg, 86%) as a solid. LC-MS (+ve mode): m/z=303.10 [M+H]⁺; ¹H NMR (300 MHz, D₂O) δ 7.46 (dd, J=8.3, 0.6 Hz, 1H, ArH), 7.30 (s, 1H, ArH), 7.25 (t, J=8.0 Hz, 1H, ArH), 6.87 (dd, J=7.7, 0.7 Hz, 1H, ArH), 4.23 (dd, J=9.1, 4.7 Hz, 1H, CH), 4.05 (m, 2H, CH₂), 3.90 (m, 1H, CH), 3.63 (m, 1H, CH), 3.45 (t, J=7.0 Hz, 2H, CH₂), 3.10 (d, J=7.0 Hz, 2H, CH₂), 2.86 (s, 6H, 2×NCH₃), 2.40 (m, 2H, CH₂).

### Example 20: 3-(2-(Dimethylamino)ethyl)-1H-indol-4-yl 1-methylazetidine-3-carboxylate

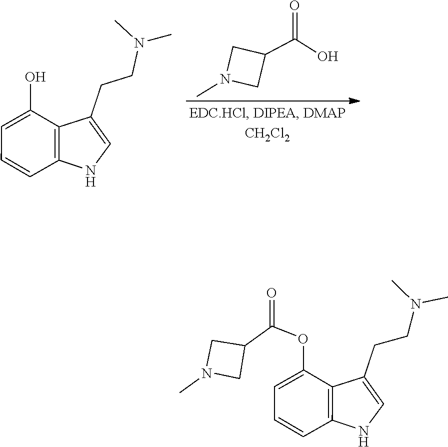

N,N-diisopropylethylamine (278 mg, 384 μL, 2.15 mmol), dimethylaminopyridine (27 mg, 0.22 mmol) and 1-methylazetidine-3-carboxylic acid (248 mg, 2.15 mmol) were added to a stirred mixture of psilocin (220 mg, 1.08 mmol) in DCM (5 mL) under an atmosphere of N₂. The resulting suspension was stirred at rt for 15 min, then N-(3-dimethylaminopropyl)-N′-ethylcarbodiimide hydrochloride (454 mg, 2.37 mmol) was added to this suspension and the reaction mixture was heated to 35° C. and stirred for 16 h. The solvent was removed under reduced pressure, to give a crude residue containing the product. LC-MS (+ve mode): m/z=302.10 [M+H]⁺.

### Example 21: 3-(2-(Dimethylamino)ethyl)-1H-indol-4-yl 3-(2-acetoxy-4,6-dimethylphenyl)-3-methylbutanoate formate

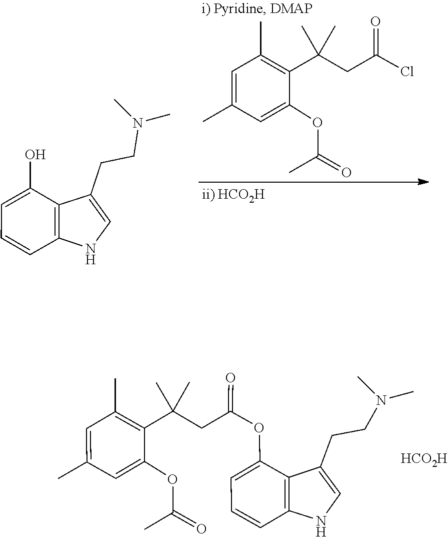

### Step 1: Preparation of 2-(4-chloro-2-methyl-4-oxobutan-2-yl)-3,5-dimethylphenyl acetate

To a mixture of 3-(2-acetoxy-4,6-dimethylphenyl)-3-methylbutyric acid (311 mg, 1.78 mmol) in anhydrous DCM (10 mL) at 0° C. under an atmosphere of N₂ was added oxalyl chloride (255 mg, 152 μL, 1.78 mmol). The mixture was warmed to rt and stirred 2 h 45 min, then the volatiles were removed under reduced pressure to afford the product as a semi-solid, which was used directly in the next step.

### Step 2: Preparation of 3-(2-(dimethylamino)ethyl)-1H-indol-4-yl 3-(2-acetoxy-4,6-dimethylphenyl)-3-methylbutanoate formate

To a mixture of psilocin (200 mg, 0.98 mmol) and DMAP (12 mg, 0.098 mmol) in anhydrous pyridine (5 mL) was added a solution of 3-(2-acetoxy-4,6-dimethylphenyl)-3-methylbutyric acid chloride (1.78 mmol) in anhydrous pyridine (5 mL). The mixture was stirred at rt for 16 h, then concentrated to give a semi-solid (0.75 g). The crude product was purified by reversed-phase chromatography on Cis silica, eluting with a gradient of acetonitrile in 0.1% formic acid in water to afford the product (274 mg, 62%) as a solid. LC-MS (+ve mode): m/z=451.20 [M+H]⁺; ¹H NMR (300 MHz, CD₃OD) δ 8.41 (s, 1H, HCO), 7.23 (m, 2H, 2×ArH), 7.04 (t, J=8.1 Hz, 1H, ArH), 6.89 (s, 1H, ArH), 6.69 (s, 1H, ArH), 6.42 (dd, J=7.3, 0.7 Hz, 1H, ArH) 3.34 (t, J 7.1 Hz, 2H, CH₂), 3.20 (s, 2H, CH₂), 3.04 (t, J 6.9 Hz, 2H, CH₂), 2.81 (s, 6H, 2×NMe), 2.61 (s, 3H, CH₃), 2.32 (s, 3H, CH₃), 2.24 (s, 3H, CH₃) 1.73 (s, 6H, 2×CH₃); ¹³C NMR (75.5 MHz, CD₃OD) δ 171.2, 170.3, 167.2, 149.8, 143.6, 139.3, 138.3, 136.3, 132.9, 131.9, 124.0, 123.0, 121.6, 119.0, 111.7, 109.3, 106.8, 58.3, 42.3, 39.0, 30.9, 24.3, 21.5, 20.5, 18.8.

### Example 22: 2-((((3-(2-(Dimethylamino)ethyl)-1H-indol-4-yl)oxy)carbonyl)oxy)propane-1,3-diyl dipalmitate

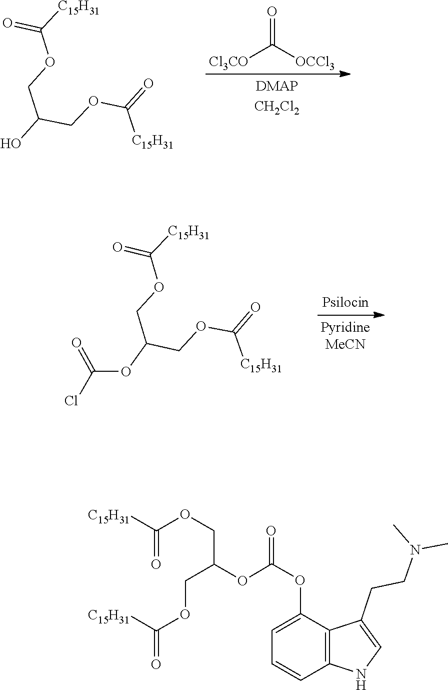

To 2-hydroxypropane-1,3-diyl dipalmitate (200 mg, 0.35 mmol) in DCM (10 mL) was added DMAP (136 mg, 1.13 mmol) and triphosgene (40 mg, 0.13 mmol), and the mixture was stirred at rt for 1 h. To the resulting mixture of 2-((chlorocarbonyl)oxy)propane-1,3-diyl dipalmitate (0.35 mmol) was added a solution of psilocin (71 mg, 0.35 mmol) and TEA (40 mg, 55 μL, 0.42 mmol) in MeCN (5 mL), and the mixture was stirred for 16 h. The mixture was filtered through Celite, the filter cake was washed with DCM (3×20 mL) and the combined filtrates were concentrated to give a solid. The crude product was purified by column chromatography on silica gel, eluting with a gradient of EtOAc in petrol, then purified further by column chromatography on silica gel, eluting with a gradient of EtOAc in MeOH to afford the product (47.3 mg, 16%) as a semi-solid. LC-MS (+ve mode): m/z=799.55 [M+H]⁺; ¹H NMR (300 MHz, CD₃OD) δ 7.24 (dd, J=8.7, 0.7 Hz, 1H, ArH), 7.07 (m, 2H, 2×ArH), 6.84 (m, 1H, ArH), 5.24 (m, 1H, CH), 4.48 (dd, J=12.2, 3.7 Hz, 2H, CH₂), 4.26 (dd, J 12.0, 6.3 Hz, 2H, CH₂), 2.93 (m, 2H, CH₂), 2.68 (m, 2H, CH₂), 2.34 (s, 6H, 2×NMe), 1.60 (m, 4H, 2×CH₂) 1.26 (m, 42H, 21×CH₂), 0.89 (t, J=6.5 Hz, 6H, 2×CH₃).

### Example 23: 6-((3-(2-(Dimethylamino)ethyl)-1H-indol-4-yl)oxy)-6-oxohexanoic acid

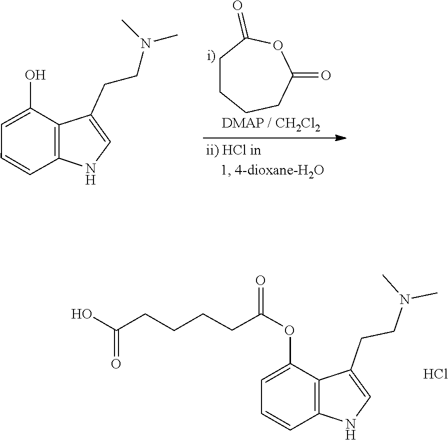 [SMILES: CN(C)CCc1c[nH]c2cccc(O)c12 ; O=C1CCCCC(=O)O1 ; CN(C)CCc1c[nH]c2cccc(OC(=O)CCCCC(=O)O)c12 ; Cl]

Adipic anhydride (tech. grade, 90%, 143 mg, 1.00 mmol) was added to a suspension of psilocin (158 mg, 0.77 mmol) in anhydrous DCM (7.5 mL) under an atmosphere of N₂ containing DMAP (19 mg, 0.16 mmol), and the mixture was stirred at rt for 20 h. The volatiles were removed under reduced pressure and the residue was purified using reversed-phase chromatography on C₁₈ column, eluting with a gradient of acetonitrile in H₂O to afford, after freeze-drying, a solid (158 mg), which was purified further in the same manner to give the free base of the target compound as an extremely hygroscopic solid. The free base was dissolved in a mixture of 1,4-dioxane (5 mL) and H₂O (0.5 mL) and treated with 4 M HCl in 1,4-dioxane (193 μL) at rt. The volatiles were removed under reduced pressure to afford the desired product (153 mg, 54%) as a solid. LC-MS (+ve mode): m/z=333.10 [M+H]⁺; ¹H NMR (300 MHz, D₂O) δ 7.46 (dd, J=8.3, 0.8 Hz, 1H, ArH), 7.32 (s, 1H, ArH), 7.25 (t, J=8.0 Hz, 1H, ArH) 6.45 (dd, J=7.7, 0.8 Hz, 1H, ArH), 3.46 (t, J=7.0 Hz, 2H, CH₂), 3.12 (t, J=7.0 Hz, 2H, CH₂), 2.87 (s, 6H, 2×NCH₃), 2.79 (m, 2H, CH₂), 2.47 (m, 2H, CH₂), 1.78 (m, 4H, 2×CH₂); ¹³C NMR (75.5 MHz, D₂O) δ 178.6, 175.8, 142.9, 138.8, 125.2, 122.4, 118.5, 112.1, 110.5, 106.5, 58.2, 42.9, 33.4, 23.7, 23.5, 21.3.

### Example 24: tert-Butyl (3-(2-(dimethylamino)ethyl)-1H-indol-4-yl) adipate

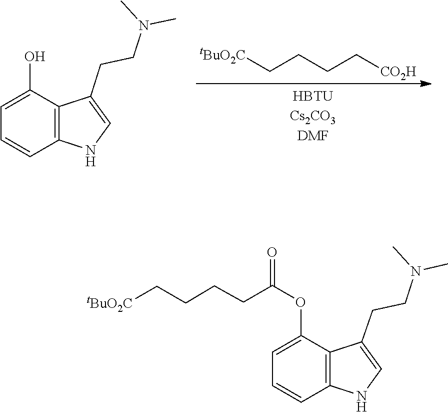 [SMILES: CN(C)CCc1c[nH]c2cccc(O)c12 ; CC(C)(C)OC(=O)CCCCC(=O)O ; CN(C)CCc1c[nH]c2cccc(OC(=O)CCCCC(=O)OC(C)(C)C)c12]

A mixture of psilocin (155 mg, 0.76 mmol), HBTU (345 mg, 0.91 mmol), 6-(tert-butoxy)-6-oxohexanoic acid (184 mg, 0.91 mmol) and Cs₂CO₃ (297 mg, 0.91 mmol) in anhydrous DMF (10 mL) was stirred at rt under an atmosphere of nitrogen overnight. The mixture was concentrated under reduced pressure and the residue was dissolved in a mixture of H₂O (30 mL) and EtOAc (20 mL). The layers were separated, and the aqueous layer was extracted with EtOAc (2×20 mL). The combined organic layers were washed with saturated brine (20 mL), dried (MgSO₄), filtered and the filtrate was concentrated to give an oil (1.13 g), which was purified by column chromatography on silica gel, eluting with a gradient of MeOH in DCM (containing 0.1% Et₃N) to give the product (180 mg, 61%) as an oil. LCMS (+ve mode): m/z=389.25 [M+H]⁺; ¹H NMR (300 MHz, CDCl₃) δ 8.79 (br. s, 1H, NH), 7.24 (dd, J=8.2, 0.8 Hz, 1H, ArH), 7.11 (app t, 1H, ArH), 6.92 (d, J=1.7 Hz, 1H, ArH), 6.78 (dd, J=7.6, 0.8 Hz, 1H, ArH), 3.04 (m, 2H, CH₂), 2.84 (m, 2H, CH₂) 2.72 (m, 2H, CH₂), 2.49 (s, 6H, 2×NCH₃), 2.30 (t, J=7.2 Hz, 2H, CH₂), 1.78 (m, 4H, 2×CH₂), 1.45 (s, 9H, C(CH₃)₃); ¹³C NMR (75.5 MHz, CDCl₃) δ 172.9, 172.6, 143.9, 138.6, 123.2, 122.2, 119.5, 112.4, 110.5, 109.5, 80.3, 59.6, 44.2, 35.2, 34.1, 28.1, 24.6, 24.3, 23.2.

### Example 25: 3-(2-(Dimethylamino)ethyl)-1H-indol-4-yl N-((tert-butoxycarbonyl)-L-phenylalanyl)-N-methylglycinate formate

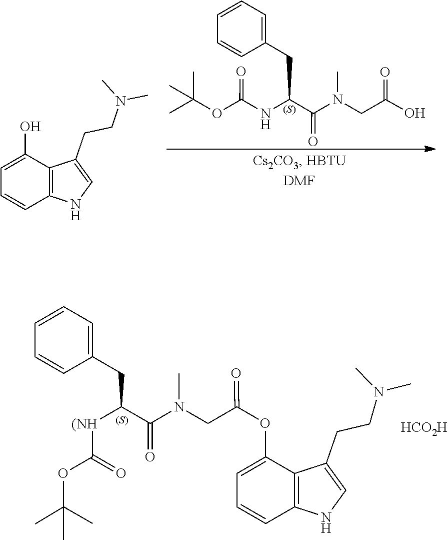

N-Boc-L-phenylalanine-sarcosine (120 mg, 0.36 mmol), HBTU (164 mg, 0.43 mmol) and Cs₂CO₃ (190 mg, 0.58 mmol) were dissolved in anhydrous DMF (4 mL) under an atmosphere of N₂. The mixture was stirred at rt for 30 min, then a mixture of psilocin (35 mg, 0.16 mmol) in anhydrous DMF (0.5 mL) was added. The mixture was stirred at rt for 20 h, then filtered through Celite and the filtrate was concentrated under reduced pressure. The residue was purified by reversed-phase chromatography on C₁₈ silica, eluting with a gradient of acetonitrile in 0.1% formic acid in H₂O to afford the product (32.5 mg, 39%) as a semi-solid. LC-MS (+ve mode): m/z=523.30 [M+H]⁺; ¹H NMR (300 MHz, CD₃CN) S 9.55 (s, 1H, NH⁺), 8.36 (s, 1H, HCO), 7.35 (m, 1H, ArH), 7.26 (m, 5H, 5×ArH), 7.12 (m, 2H, 2×ArH), 6.76 (dd, J=7.7, 0.8 Hz, 1H, ArH), 5.88 (d, J=8.8 Hz, 1H, NH), 4.82 (m, 1H, CH), 4.43 (s, 2H, CH₂), 3.11 (7H, NCH₃, 2×CH₂), 2.75 (s, 6H, 2×NCH₃), 1.31 (s, 9H, 3×Boc CH₃).

### Example 26: 3-(2-(Dimethylamino)ethyl)-1H-indol-4-yl (tert-butoxycarbonyl)-L-valinate formate

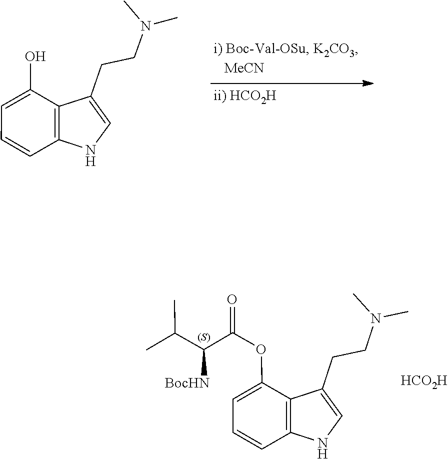

To a mixture of psilocin (200 mg, 0.98 mmol) in MeCN (10 mL) was added K₂CO₃ (149 mg, 1.08 mmol) and Boc-Val-OSu (292 mg, 0.93 mmol). The mixture was heated to 80° C. and stirred for 1 h, allowed to cool to rt and stirred for 16 h. The mixture was filtered through Celite and the filter pad was washed with MeCN (2×20 mL), and the combined filtrates were concentrated. The crude product was purified using reversed-phase chromatography on Cis silica, eluting with a gradient of MeCN in 0.1% formic acid in H₂O to afford the product (184 mg, 46%) as a foam. LC-MS (+ve mode): m/z=404.25 [M+H]⁺; ¹H NMR (300 MHz, CD₃OD) δ 8.42 (s, 1H, HCO), 7.32 (dd, J=8.2, 0.8 Hz, 1H, ArH), 7.26 (s, 1H, ArH), 7.14 (t, J=7.9 Hz, 1H, ArH), 6.76 (dd, J=7.7, 0.8 Hz, 1H, ArH), 4.36 (d, J=5.9 Hz, 1H, CH), 3.50 (m, 2H, CH₂), 3.26 (t, J=7.5 Hz, 2H), 2.99 (s, 6H, 2×NMe), 2.42 (m, 1H, CH), 1.50 (s, 9H, 3×Boc CH₃), 1.19 (d, J=6.9 Hz, 3H, CH₃), 1.15 (d, J=6.9 Hz, 3H, CH₃); ¹³C NMR (75.5 MHz, CD₃OD) δ 210.6, 202.1, 174.1, 167.4, 159.0, 145.6, 141.2, 126.3, 123.4, 121.0, 113.2, 111.5, 109.4, 81.3, 61.5, 60.4, 44.2, 32.3, 29.4, 23.3, 20.4, 19.2.

### Example 27: 3-(2-(Dimethylamino)ethyl)-1H-indol-4-yl L-valinate dihydrochloride

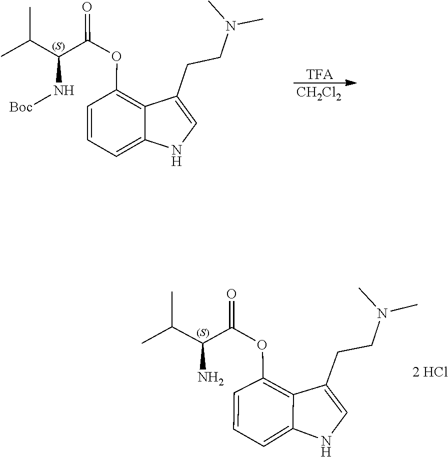

A mixture of 3-(2-(dimethylamino)ethyl)-1H-indol-4-yl (tert-butoxycarbonyl)-L-valinate formate (140 mg, 0.35 mmol) in DCM (10 mL) under an atmosphere of N₂ was treated with TFA (1.33 mL, 17.4 mmol). The mixture was stirred at rt for 3 h, then the volatiles were removed under reduced pressure and the crude residue was purified by reversed-phase chromatography on C₁₈ silica, eluting with a gradient of MeCN in 0.02% hydrochloric acid to afford the product (19 mg, 14%) as a semi-solid. LC-MS (+ve mode): m/z=304.15 [M+H]⁺; ¹H NMR (300 MHz, CDCl₃) δ 7.34 (dd, J=8.2, 0.8 Hz, 1H, ArH), 7.26 (s, 1H, ArH), 7.13 (t, J=8.0 Hz, 1H, ArH), 6.80 (dd, J=7.8, 0.8 Hz, 1H, ArH), 4.75 (d, J=4.0 Hz, 1H, CH), 3.47 (m, 2H, CH₂), 3.29 (m, 2H, CH₂), 2.93 (s, 3H, NCH₃), 2.92 (s, 3H, NCH₃), 2.62 (m, 1H, CH), 1.27 (dd, J=7.8, 1.7 Hz, 6H, 2×CH₃).

### Example 28: 3-(2-(Dimethylamino)ethyl)-1H-indol-4-yl (tert-butoxycarbonyl)-L-phenylalaninate formate

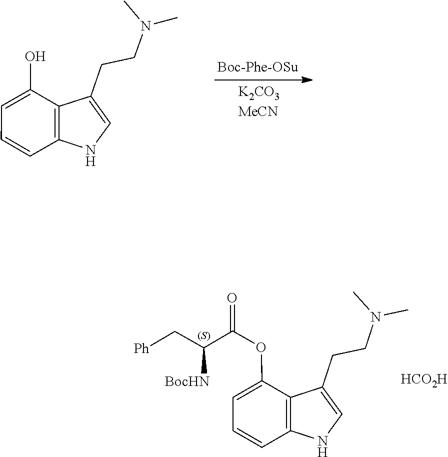

A suspension of Boc-Phe-OSu (0.67 mg, 1.84 mmol), psilocin (342 mg, 1.68 mmol) and K₂CO₃ (254 mg, 1.84 mmol) in anhydrous MeCN (10 mL) under an atmosphere of N₂ was stirred at rt for 1 h, followed by heating to reflux and stirring for 30 min, then storing at rt for 20 h. The mixture was poured into H₂O (40 mL) and extracted with EtOAc (3×40 mL). The combined organic layers were washed with saturated brine (40 mL), dried (MgSO₄), filtered and the filtrate was concentrated to an oil (804 mg). The residue was purified by column chromatography on silica gel, eluting with a gradient of MeOH in DCM to afford a mixture of the desired product (as its free base) and psilocin (130 mg). This material was purified further using reversed-phase chromatography on C₁₈ silica, eluting with a gradient of MeCN in 0.1% formic acid in H₂O to afford the product (174 mg, 21%) as a solid. LC-MS (+ve mode): m/z=452.25 [M+H]⁺; ¹H NMR (300 MHz, DMSO-d₆) δ 11.05 (s, 1H, NH), 8.21 (s, 1H, HCO), 7.71 (d, J=8.5 Hz, 1H, ArH), 7.32 (m, 4H, ArH), 7.24 (m, 2H, ArH+NH⁺), 7.16 (d, J=2.1 Hz, 1H, ArH), 7.03 (m, 1H, ArH), 6.60 (d, J=7.4 Hz, 1H, ArH), 4.58 (m, 1H, CH), 3.30 (dd, J=13.7, 4.7 Hz, 1H, CHₐH_{b} (Phe)), 3.05 (dd, J=13.7, 10.4 Hz, 1H, CHₐH_{b} (Phe)), 2.85 (m, 2H, CH₂), 2.59 (m, 2H, CH₂), 2.27 (s, 6H, 2×NCH₃), 1.34 (s, 9H, C(CH₃)₃); ¹³C NMR (75.5 MHz, DMSO-d₆) δ 171.7, 164.2, 156.0, 144.2, 139.1, 138.1, 129.7, 128.7, 127.0, 124.1, 121.4, 119.8, 111.5, 111.4, 109.9, 78.9, 60.6, 55.8, 45.3, 40.8, 28.6.

### Example 29: 3-(2-(Dimethylamino)ethyl)-1H-indol-4-yl L-phenylalaninate 2HCl salt

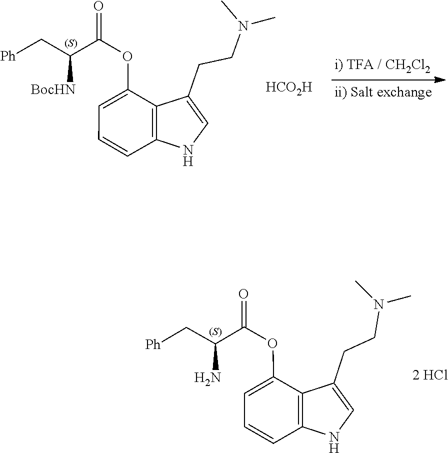

3-(2-(Dimethylamino)ethyl)-1H-indol-4-yl (tert-butoxycarbonyl)-L-phenylalaninate formate (110 mg, 0.22 mmol) was dissolved in anhydrous DCM (2 mL) and TFA (0.5 mL) was added dropwise. The mixture was stirred for 30 min, then the volatiles were removed under reduced pressure and the residue was purified by reversed-phase chromatography on C₁₈ silica, eluting with a gradient of acetonitrile in 0.02% hydrochloric acid to afford the product (70 mg, 75%) as a solid. LC-MS (+ve mode): m/z=352.20 [M+H]⁺; ¹H NMR (300 MHz, D₂O) δ 7.45 (m, 6H, ArH), 7.35 (s, 1H, ArH), 7.28 (m, 1H, ArH), 6.93 (d, J=0.6 Hz, 1H, ArH), 4.82 (m, 1H, CH), 3.62 (dd, J=14.3, 7.2 Hz, 1H, CHₐH_{b}(Phe)), 3.50 (dd, J=14.3, 6.9 Hz, 1H, CHₐH_{b} (Phe)), 3.35 (m, 2H, CH₂), 3.09 (m, 2H, CH₂), 2.86 (s, 3H, NCH₃), 2.79 (s, 3H, NCH₃); ¹³C NMR (75.5 MHz, D₂O) δ 169.5, 142.2, 139.0, 133.8, 129.5, 129.3, 128.2, 125.5, 122.3, 117.7, 111.6, 111.1, 106.3, 58.0, 54.2, 43.2, 42.6, 35.9, 21.2.

### Example 30: 1-(tert-Butyl) 2-(3-(2-(dimethylamino)ethyl)-1H-indol-4-yl) L-pyrrolidine-1,2-dicarboxylate formate

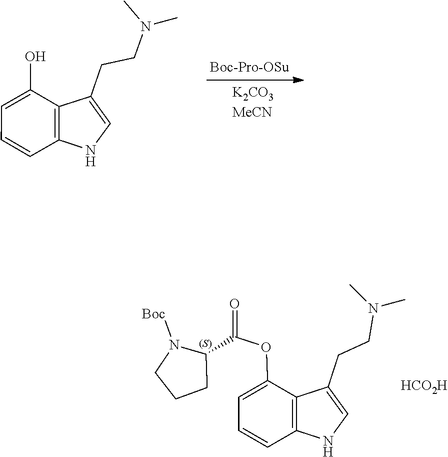

A suspension of Boc-Pro-OSu (261 mg, 0.84 mmol), psilocin (155 mg, 0.76 mmol) and K₂CO₃ (115 mg, 0.84 mmol) in anhydrous MeCN (5 mL) was heated to 90° C. and stirred for 18 h. The mixture was poured into H₂O (20 mL) and extracted with EtOAc (3×20 mL). The combined organic layers were washed with saturated brine (20 mL), dried (MgSO₄), filtered and the filtrate was concentrated to give a crude oil which was purified using reversed-phase chromatography on C₁₈ silica. Elution with a gradient of MeCN in 0.1% formic acid in H₂O afforded the product (54 mg, 16%) as a semi-solid. LC-MS (+ve mode): m/z=402.25 [M+H]⁺; ¹H NMR (300 MHz, CD₃CN) (mixture of two rotamers) δ 9.45 (br. s, 1H, NH⁺), 8.36 (s, 1H, HCO), 7.34 (m, 1H, ArH), 7.15 (m, 2H, ArH), 6.89-6.77 (m, 1H, ArH), 4.63 (m, 1H, CH), 3.47 (m, 2H, CH₂), 3.10 (m, 4H, 2×CH₂), 2.69 (s, 3H, NCH₃), 2.48 (s, 3H, NCH₃), 2.28 (m, 2H, CH₂), 2.04 (obs in, 2H, CH₂), 1.47 (s, 9H, C(CH₃)₃).

### Example 31: 3-(2-(Dimethylamino)ethyl)-1H-indol-4-yl L-prolinate dihydrochloride

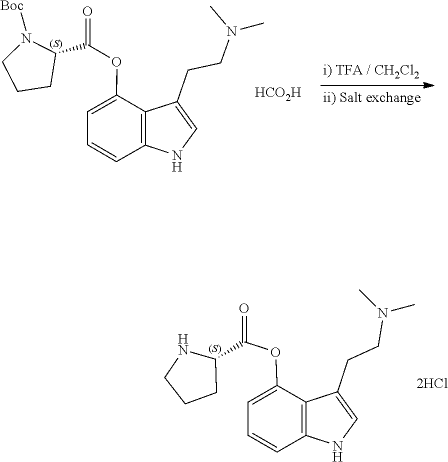

Boc-Pro-psilocin formate (242 mg, 0.54 mmol) was dissolved in anhydrous DCM (4 mL) and TFA (1 mL) was added dropwise. The reaction mixture was stirred at rt for 2 h, then the volatiles were removed under reduced pressure and the residue was purified by reversed-phase chromatography on C₁₈ silica, eluting with a gradient of MeCN in 0.02% hydrochloric acid to afford the product (179 mg, 70%) as a semi-solid. LC-MS (+ve mode): m/z=302.15 [M+H]⁺; ¹H NMR (300 MHz, D₂O) δ 7.50 (d, J=8.2 Hz, 1H, ArH), 7.36 (s, 1H, ArH), 7.27 (t, J=7.8 Hz, 1H, ArH), 6.98 (d, J=7.8 Hz, 1H, ArH), 4.92 (t, J=8.2 Hz, 1H, CH), 3.52 (m, 4H, 2×CH₂), 3.19 (t, J=6.7 Hz, 2H, CH₂), 2.89 (s, 6H, 2×NCH₃), 2.72 (m, 1H, 0.5×CH₂), 2.45 (m, 1H, 0.5×CH₂), 2.23 (m, 2H, CH₂); ¹³C NMR (75.5 MHz, D₂O) δ 169.6, 142.5, 139.0, 125.4, 122.3, 117.9, 111.7, 111.0, 106.4, 59.6, 58.0, 46.3, 42.9, 42.8, 28.3, 23.5, 21.4.

### Example 32: 3-(2-(Dimethylamino)ethyl)-1H-indol-4-yl N2,N6-bis(tert-butoxycarbonyl)-L-lysinate hydrochloride

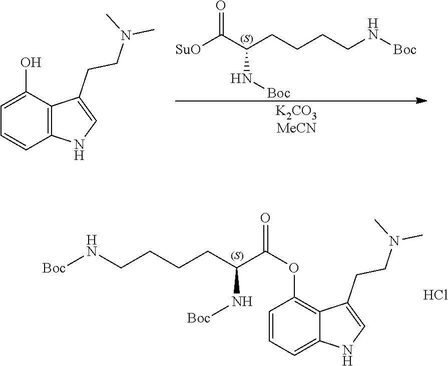

To a mixture of psilocin (200 mg, 0.98 mmol) in anhydrous MeCN (10 mL) was added K₂CO₃ (149 mg, 1.08 mmol) followed by Boc-Lys(Boc)-OSu (413 mg, 0.93 mmol) portion wise. The mixture was stirred at room temperature for 16 h, the solvent was removed under reduced pressure and the crude residue was purified by reversed-phase chromatography on Cis silica, eluting with a gradient of MeCN in 0.02% hydrochloric acid to afford the product (243 mg, 47%) as a solid. LC-MS (+ve mode): m/z=533.35 [M+H]⁺.

### Example 33: 3-(2-(Dimethylamino)ethyl)-1H-indol-4-yl L-lysinate trihydrochloride

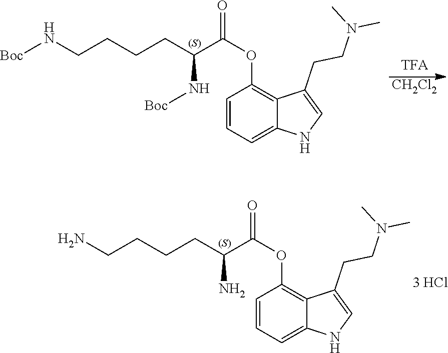

A mixture of 3-(2-(dimethylamino)ethyl)-1H-indol-4-yl N2,N6-bis(tert-butoxycarbonyl)-L-lysinate hydrochloride (243 mg, 0.46 mmol) in DCM (10 mL) under an atmosphere of N₂ was treated with TFA (1.76 mL, 23.0 mmol). The mixture was stirred at rt for 16 h, then the volatiles were removed under reduced pressure and the crude residue was purified by reversed-phase chromatography on C₁₈ silica, eluting with a gradient of MeCN in 0.02% hydrochloric acid to afford the product (141 mg, 70%) as a solid. LC-MS (+ve mode): m/z=333.20 [M+H]⁺; ¹H NMR (300 MHz, D₂O) δ 7.43 (d, J=8.2 Hz, 1H, ArH), 7.30 (s, 1H, ArH), 7.20 (t, J=8.0 Hz, 1H, ArH), 6.85 (d, J=7.8 Hz, 1H, ArH), 4.57 (m, 1H, CH), 3.44 (t, J=7.8 Hz, 2H, CH₂), 3.13 (t, J=6.0 Hz, 2H, CH₂), 3.00 (t, J=7.5 Hz, 2H, CH₂), 2.82 (d, J=6.0 Hz, 6H, 2×NMe), 2.22 (m, 2H, CH₂), 1.69 (m, 4H, 2×CH₂); ¹³C NMR (75.5 MHz, CDCl₃) δ 170.1, 142.4, 139.0, 125.5, 122.4, 118.0, 111.7, 111.2, 106.8, 58.1, 52.7, 43.1, 42.7, 39.0, 29.4, 26.4, 21.7, 21.3.

### Example 34: 3-(2-(Dimethylamino)ethyl)-1H-indol-4-yl dimethylglycinate diformate

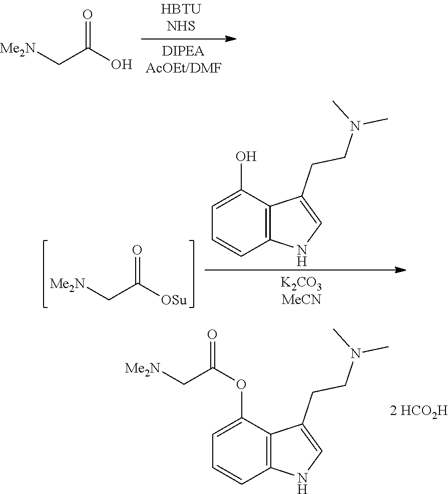

A suspension of N,N-dimethylglycine (113 mg, 1.10 mmol), N-hydroxysuccinimide (139 mg, 1.21 mmol), N,N-diisopropylethylamine (156 mg, 211 μL, 1.21 mmol) and HBTU (417 mg, 1.10 mmol) in a mixture of EtOAc (10 mL) and DMF (5 mL) was stirred at rt for 18 h. The solvents were removed under reduced pressure and the residual material was dissolved in anhydrous MeCN (10 mL) and placed under an atmosphere of N₂. K₂CO₃ (167 mg, 1.21 mmol) and psilocin (202 mg, 0.99 mmol) were added, and the mixture was heated to reflux and stirred for 30 min, followed by stirring at rt for 18 h. The mixture was filtered through Celite, and the filter cake was washed with MeCN. The combined filtrates were concentrated under reduced pressure and the residue was purified using reversed-phase chromatography on C₁₈ silica, eluting with a gradient of MeCN in 0.1% formic acid in H₂O to afford the product (79 mg, 21%) as an oil. LC-MS (+ve mode): m/z=290.15 [M+H]⁺; ¹H NMR (300 MHz, D₂O) S 8.35 (br. s, 2H, 2×HCO), 7.42 (d, J=8.2 Hz, 1H, ArH), 7.29 (s, 1H, ArH), 7.20 (m, 1H, ArH), 6.93 (d, J=7.8 Hz, 1H, ArH), 4.50 (s, 2H, CH₂), 3.43 (m, 2H, CH₂), 3.11 (m, 2H, CH₂), 3.00 (s, 6H, 2×NCH₃), 3.81 (s, 6H, 2×NCH₃).

### Example 35: 3-(2-(Dimethylamino)ethyl)-1H-indol-4-yl 2-oxa-6-azaspiro[3.3]heptane-6-carboxylate formate

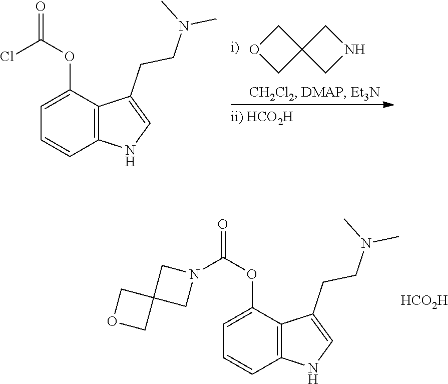 [SMILES: CN(C)CCc1c[nH]c2cccc(OC(=O)Cl)c12 ; C1NCC12COC2 ; CN(C)CCc1c[nH]c2cccc(OC(=O)N3CC4(COC4)C3)c12 ; "HCO2H"]

### Step 1: Preparation of 3-(2-(Dimethylamino)ethyl)-1H-indol-4-yl carbonochloridate (Psilocin Carbonochloridate)

To a solution of psilocin (200 mg, 0.98 mmol) in DCM (10 mL) was added DMAP (338 mg, 3.2 mmol) and triphosgene (85 mg, 0.36 mmol). The reaction mixture was stirred at rt for 1 h and used directly in the next step.

### Step 2: Preparation of 3-(2-(Dimethylamino)ethyl)-1H-indol-4-yl 2-oxa-6-azaspiro[3.3]heptane-6-carboxylate formate

To the psilocin carbonochloridate solution was added 2-oxa-azaspiro [3,3] heptane (194 mg, 196 mmol) and TEA (118 mg, 156 μL, 1.17 mmol) and the mixture was stirred at rt for 16 h. H₂O (1 mL) was added and the mixture was filtered through Celite, washing the filter cake with DCM (10 mL) and MeCN (10 mL). The combined filtrates were concentrated to give a solid (0.80 g) that was purified by reversed-phase chromatography on Cis silica, eluting with a gradient of MeCN in 0.1% formic acid in H₂O to afford the product (167 mg, 51%) as a semi-solid. LC-MS (+ve mode): m/z=330.10 [M+H]⁺; ¹H NMR (300 MHz, DMSO-d₆) δ 8.24 (s, 1H, HCO), 7.20 (dd, J=8.1, 0.9 Hz, 1H, ArH), 7.13 (d, J=2.4 Hz, 1H, ArH), 7.00 (t, J=7.8 Hz, 1H, ArH), 6.67 (dd, J=7.6, 0.8 Hz, 1H, ArH), 4.72 (s, 4H, 2×CH₂), 4.43 (br. s, 2H, CH₂), 4.18 (br. s, 2H, CH₂), 2.86 (m, 2H, CH₂), 2.61 (m, 2H, CH₂), 2.34 (s, 6H, 2×NMe); ¹³C NMR (75.5 MHz, DMSO-d₆) δ 164.5, 154.5, 154.3, 144.4, 138.9, 123.9, 121.4, 112.1, 110.9, 109.5, 80.3, 80.0, 60.5, 59.4, 58.7, 44.8, 38.1, 24.1.

### Example 36: 3-(2-(Dimethylamino)ethyl)-1H-indol-4-yl morpholine-4-carboxylate hydrochloride

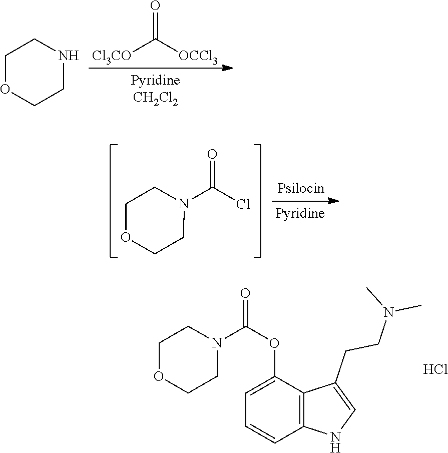

To triphosgene (297 mg, 1.00 mmol) in anhydrous DCM (5 mL) at 0° C. under an atmosphere of N₂ was added pyridine (0.79 g, 809 μL, 10.0 mmol) dropwise. After stirring until a precipitate dissolved (about 20 min) morpholine (93 mg, 92 μL, 1.07 mmol) was introduced to the flask dropwise. The mixture was stirred at 0° C. for 15 min and at rt for 1 h, then the DCM was removed under reduced pressure and additional pyridine (3 mL) was added followed by psilocin (204 mg, 1.00 mmol). The mixture was heated to 80° C. and stirred for 16 h, then the volatiles were removed under reduced pressure and the residue was dissolved in MeOH (5 mL). Addition of EtOAc (20 mL) gave a solid, which was removed by filtration. The filtrate was concentrated under reduced pressure and the residue was purified by reversed-phase chromatography on C₁₈ silica, eluting with a gradient of MeCN in 0.02% hydrochloric acid to give the product (75 mg, 22%) as a solid. LC-MS (+ve mode): m/z=318.15 [M+H]⁺; ¹H NMR (300 MHz, D₂O) δ 7.44 (d, J=8.2 Hz, 1H, ArH), 7.32 (s, 1H, ArH), 7.24 (t, J=7.7 Hz, 1H, ArH), 6.85 (d, J=7.7 Hz, 1H, ArH), 3.83 (br. s, 6H, 3×CH₂), 3.59 (m, 2H, CH₂), 3.44 (m, 2H, CH₂), 3.13 (m, 2H, CH₂), 2.87 (s, 6H, 2×NCH₃); ¹³C NMR (75.5 MHz, D₂O) δ 155.8, 143.5, 138.78, 125.1, 122.5, 119.1, 112.3, 110.3, 106.6, 66.2, 58.2, 44.6, 43.9, 42.8, 21.3.

### Example 37: 3-(2-(Dimethylamino)ethyl)-1H-indol-4-yl ((5-methyl-2-oxo-1,3-dioxol-4-yl)methyl) carbonate

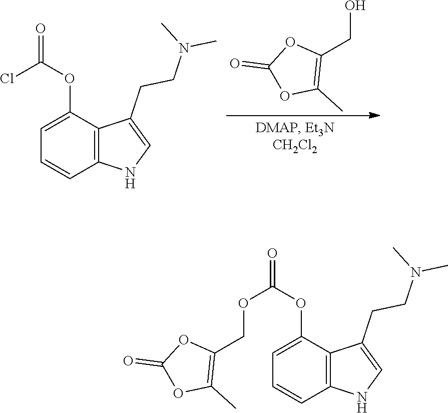

To a psilocin carbonochloridate solution (containing ca. 1 mmol chloride) was added a solution of 4-(hydroxymethyl)-5-methyl-1,3-dioxol-2-one (127 mg, 0.98 mmol) and TEA (118 mg, 156 μL, 1.17 mmol) in DCM (5 mL). The mixture was stirred at rt for 16 h, then filtered through Celite, and the filter cake was washed with DCM (10 mL) and MeCN (10 mL). The combined filtrates were concentrated to give a (0.63 g). LC-MS (+ve mode): m/z=361.10 [M+H]⁺.

### Example 38: 2-(4-((tert-Butyldimethylsilyl)oxy)-1H-indol-3-yl)-N,N-dimethylethan-1-amine

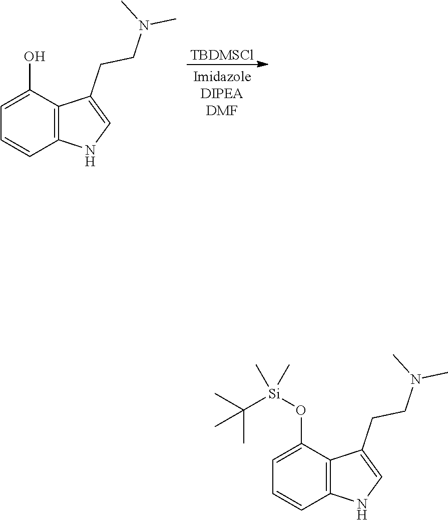

A solution of psilocin (98 mg, 0.48 mmol) in anhydrous DMF (3.5 mL) under an atmosphere of N₂ was treated with imidazole (65 mg, 0.96 mmol) and TBDMSCl (174 mg, 1.15 mmol) followed by dropwise addition of N,N-diisopropylethylamine (149 mg, 200 μL, 1.15 mmol). The mixture was stirred at rt for 24 h, then the volatiles were removed under reduced pressure and EtOAc (50 mL) and saturated aqueous NaHCO₃ (20 mL) were added. The organic layer was separated, washed with H₂O (20 mL), saturated brine (20 mL), dried (MgSO₄) and concentrated to give a solid which was purified by column chromatography on silica gel, eluting with a gradient of MeOH in DCM to afford the product (112 mg, 73%) as a solid. LC-MS (+ve mode): m/z=319.20 [M+H]⁺; ¹H NMR (300 MHz, CDCl₃) δ 7.98 (br. s, 1H, NH), 6.94 (m, 3H, 3×ArH), 6.47 (dd, J=7.2, 1.3 Hz, 1H, ArH), 3.12 (m, 2H, CH₂), 2.68 (m, 2H, CH₂), 2.32 (s, 6H, 2×NCH₃), 1.04 (s, 9H, C(CH₃)₃), 0.34 (s, 6H, 2×CH₃); ¹³C NMR (75.5 MHz, CDCl₃) δ 150.6, 138.8, 122.5, 120.2, 119.6, 115.0, 107.8, 104.5, 60.8, 45.7, 26.3, 25.2, 18.8, −3.6.

### Example 39: N,N-Dimethyl-2-(4-((triisopropylsilyl)oxy)-1H-indol-3-yl)ethan-1-amine

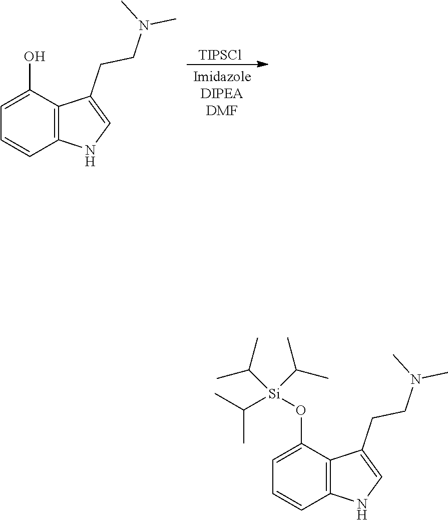

A mixture of psilocin (155 mg, 0.76 mmol) in anhydrous DMF (3.5 mL) under an atmosphere of N₂ was treated with imidazole (103 mg, 1.52 mmol) and TIPSCl (351 mg, 390 μL, 1.82 mmol) followed by dropwise addition of N,N-diisopropylethylamine (235 mg, 317 μL, 1.82 mmol). The mixture was stirred at rt for 24 h, then the volatiles were removed under reduced pressure and EtOAc (50 mL) and saturated aqueous NaHCO₃ (20 mL) were added. The organic layer was separated, washed with H₂O (20 mL), saturated brine (20 mL), dried (MgSO₄), filtered and the filtrate was concentrated to give a solid, which was purified by column chromatography on silica gel, eluting with a gradient of MeOH in DCM to afford the product (253 mg, 92%) as a solid. LC-MS (+ve mode): m/z=361.25 [M+H]⁺; ¹H NMR (300 MHz, CDCl₃) δ 7.96 (br. s, 1H, NH), 6.94 (m, 3H, 3×ArH), 6.46 (dd, J=7.2, 1.3 Hz, 1H, ArH), 3.19 (m, 2H, CH₂), 2.76 (m, 2H, CH₂), 2.35 (s, 6H, 2×NCH₃), 1.41 (septet, J=7.5 Hz, 3H, 3×CH(CH₃)₂), 1.04 (d, J=7.5 Hz, 18H, 3×CH(CH₃)₂); ¹³C NMR (75.5 MHz, CDCl₃) δ 151.0, 138.8, 122.6, 120.2, 119.7, 114.7, 107.5, 104.4, 60.5, 45.5, 24.9, 18.3, 13.7.

### Example 40: N,N-Dimethyl-2-(4-((triethylsilyl)oxy)-1H-indol-3-yl)ethan-1-amine

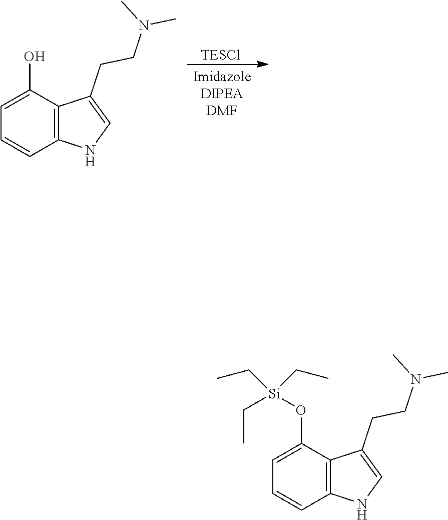

To a mixture of psilocin (190 mg, 0.93 mmol) in anhydrous DMF (4.2 mL) under an atmosphere of N₂ was added imidazole (127 mg, 1.86 mmol) and TESCl (336 mg, 375 μL, 2.23 mmol), followed by dropwise addition of N,N-diisopropylethylamine (288 mg, 388 μL, 2.23 mmol). The mixture was stirred at rt for 18 h, then the volatiles were removed under reduced pressure and EtOAc (75 mL) and saturated aqueous NaHCO₃ (20 mL) were added. The organic layer was separated, washed with H₂O (20 mL), saturated brine (20 mL), dried (MgSO₄), filtered and the filtrate was concentrated to give a crude oil (324 mg). This material was purified by column chromatography on silica gel, eluting with a gradient of MeOH in DCM to afford the product (236 mg, 80%) as a semi-solid. LC-MS (+ve mode): m/z=319.15 [M+H]⁺; ¹H NMR (300 MHz, CDCl₃) δ 8.00 (br. s, 1H, NH), 6.93 (m, 3H, 3×ArH), 6.45 (br. d, J=7.2 Hz, 1H, ArH), 3.10 (m, 2H, CH₂), 2.68 (m, 2H, CH₂), 2.33 (s, 6H, 2×NCH₃), 1.02 (m, 9H, 3×CH₂CH₃), 0.87 (m, 6H, 3×CH₂CH₃); ¹³C NMR (75.5 MHz, CDCl₃) δ 150.5, 138.8, 122.6, 120.4, 119.5, 115.0, 107.2, 104.6, 61.2, 45.7, 25.2, 6.9, 5.4.

### Example 41: ((3-(2-(Dimethylamino)ethyl)-1H-indol-4-yl)oxy)methyl ethyl carbonate formate

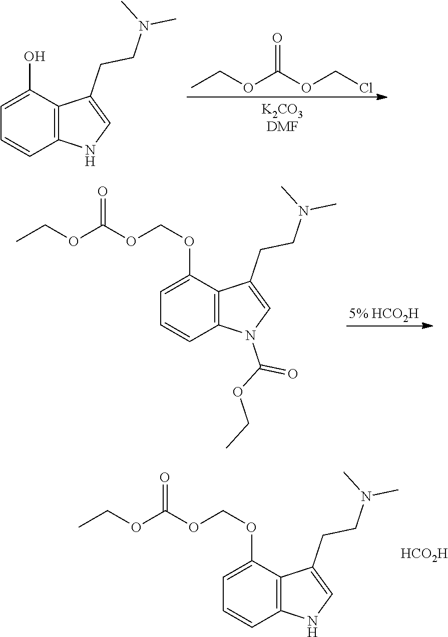

Chloromethyl ethyl carbonate (234 mg, 196 μL, 1.69 mmol) was added to a suspension of psilocin (157 mg, 0.77 mmol) and K₂CO₃ (265 mg, 1.92 mmol) in anhydrous DMF (6 mL) at rt under an atmosphere of N₂. The mixture was stirred at rt for 24 h, the solids were removed by filtration through Celite, and the filter cake was washed with MeCN. The combined filtrates were concentrated to give an oil (265 mg), that was dissolved in 5% aqueous formic acid and the resulting mixture was stirred at rt for 16 h. The mixture was concentrated under reduced pressure and the residue was purified using reversed-phase chromatography on C₁₈ silica, eluting with a gradient of MeCN in 0.1% formic acid in H₂O to give the product (127 mg, 47%) as a semi-solid. LC-MS (+ve mode): m/z=307.10 [M+H]⁺; ¹H NMR (300 MHz, D₂O) S 8.42 (br. s, 1H, HCO), 7.53 (d, J=8.3 Hz, 1H, ArH), 7.34 (m, 2H, ArH), 7.05 (d, J=7.8 Hz, 1H, ArH), 5.63 (s, 2H, OCH₂O), 4.40 (q, J=7.1 Hz, 2H, CH₂), 3.44 (m, 2H, CH₂), 3.14 (m, 2H, CH₂), 2.92 (s, 6H, 2×NCH₃), 1.38 (t, J=7.1 Hz, 3H, CH₃); ¹³C NMR (75.5 MHz, D₂O) δ 170.3, 154.9, 143.4, 138.3, 128.3, 123.0, 119.6, 112.7, 108.9, 107.7, 68.3, 66.4, 58.2, 42.7, 21.1, 13.4.

### Example 42: ((3-(2-(Dimethylamino)ethyl)-1H-indol-4-yl)oxy)methyl pivalate

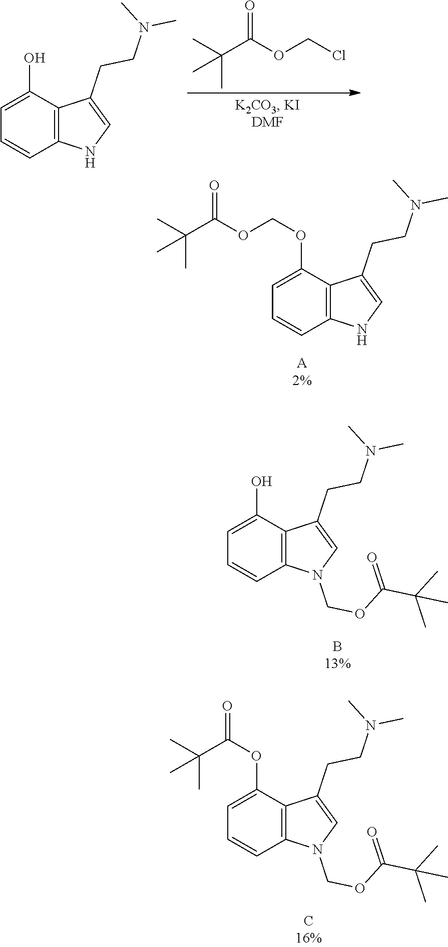

Potassium carbonate (406 mg, 2.94 mmol), potassium iodide (49 mg, 0.29 mmol) and chloromethyl pivalate (443 mg, 424 μL, 2.94 mmol) were added to a stirred mixture of psilocin (0.60 g, 2.94 mmol) in anhydrous DMF (15 mL) at rt under an atmosphere of N₂. The mixture was stirred at rt for 16 h, then the solvent was removed under reduced pressure. The residual material was purified by column chromatography on silica gel, eluting with a gradient of MeOH in EtOAc to afford 3-(2-(dimethylamino)ethyl)-1-((pivaloyloxy)methyl)-1H-indol-4-yl pivalate C (192 mg, 16%) as a semi-solid, (3-(2-(dimethylamino)ethyl)-4-hydroxy-1H-indol-1-yl)methyl pivalate B (117 mg, 13%) as a solid (117 mg) and a fraction containing impure ((3-(2-(dimethylamino)ethyl)-1H-indol-4-yl)oxy)methyl pivalate A (170 mg) as an oil. The fraction containing compound A (170 mg) was purified by column chromatography on silica gel, eluting with a gradient of MeOH in EtOAc to give a fraction containing compound A (82 mg) as an oil. This material (82 mg) was purified by reversed-phase chromatography on C₁₈ silica, eluting with a gradient of MeCN in water to afford the title compound (22 mg, 2%) as a semi-solid. LC-MS (+ve mode): m/z=319.15 [M+H]⁺; ¹H NMR (300 MHz, CDCl₃) δ 7.12 (t, J=7.9 Hz, 1H, ArH), 6.94 (dd, J=8.2, 0.8 Hz, 1H, ArH), 6.89 (s, 1H, ArH), 6.61 (dd, J=7.7, 0.9 Hz, 1H, ArH), 5.96 (s, 2H, CH₂), 2.91 (m, 2H, CH₂), 2.70 (m, 2H, CH₂), 2.38 (s, 6H, 2×NCH₃), 1.15 (s, 9H, 3×Boc CH₃).

### Data for (3-(2-(dimethylamino)ethyl)-4-hydroxy-1H-indol-1-yl)methyl pivalate (B)

LC-MS (+ve mode): m/z=319.15 [M+H]⁺; ¹H NMR (300 MHz, CDCl₃) δ 7.37 (dd, J=8.3, 0.5 Hz, 1H, ArH), 7.15 (t, J=8.0 Hz, 1H, ArH), 6.98 (s, 1H, ArH), 6.68 (dd, J=7.7, 0.6 Hz, 1H, ArH), 5.52 (s, 2H, CH₂), 2.86 (t, J=7.3 Hz, 2H, CH₂), 2.63 (t, J=7.3 Hz, 2H, CH₂), 2.25 (s, 6H, 2×NCH₃), 1.41 (s, 9H, 3×Boc CH₃); ¹³C NMR (75.5 MHz, CDCl₃) δ 177.9, 144.9, 138.5, 126.3, 122.5, 121.1, 112.7, 110.5, 108.3, 70.0, 58.9, 44.7, 39.4, 27.5, 23.4.

### Data for 3-(2-(dimethylamino)ethyl)-1-((pivaloyloxy)methyl)-1H-indol-4-yl pivalate (C)

LC-MS (+ve mode): m/z=403.25 [M+H]⁺; ¹H NMR (300 MHz, CDCl₃) δ 7.30 (dd, J=8.2, 0.8 Hz, 1H, ArH), 7.19 (t, J=8.0 Hz, 1H, ArH), 7.03 (s, 1H, ArH), 6.75 (dd, J=7.7, 0.8 Hz, 1H, ArH), 6.01 (s, 1H, CH₂), 2.92 (m, 2H, CH₂), 2.64 (m, 2H, CH₂), 2.30 (s, 6H, 2×NCH₃), 1.43 (s, 9H, 3×Boc CH₃), 1.14 (s, 9H, 3×Boc CH₃); ¹³C NMR (75.5 MHz, CDCl₃) δ 178.4, 177.7, 145.1, 139.0, 126.15, 122.9, 121.5, 114.2, 113.3, 107.4, 68.9, 59.9, 45.7, 39.4, 39.1, 27.5, 27.1, 24.7.

### Example 43: 3-(2-(Dimethylamino)ethyl)-1H-indol-4-yl di-(hydroxymethyl isopropyl carbonate) phosphate

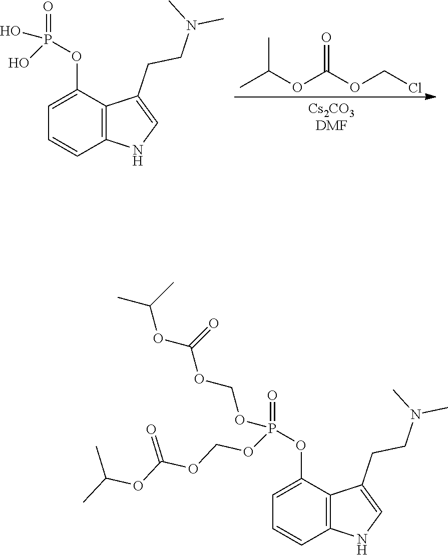

Cesium carbonate (88 mg, 0.27 mmol) was added to a stirred suspension of psilocybin (70 mg, 0.25 mmol) in DMF (3 mL) at rt under an atmosphere of N₂. After 15 min, chloromethyl isopropyl carbonate (56 mg, 49 μL, 0.37 mmol) was added to the suspension and the mixture was stirred at rt for 16 h. The solvent was removed under reduced pressure and the residual material was purified by reversed-phase chromatography on C₁₈ silica, eluting with a gradient of MeCN in 0.02% hydrochloric acid to afford the product (17 mg) as a semi solid. LC-MS (+ve mode): m/z=517.20 [M+H]⁺.

### Example 44: 3-(2-(Dimethylamino)ethyl)-1H-indol-4-yl bis(di-methyl pivalate) phosphate

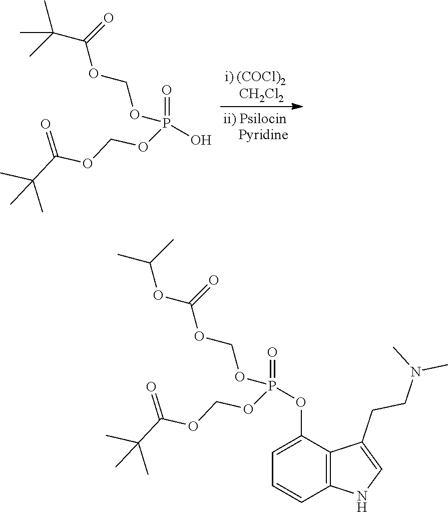 [SMILES: CC(C)(C)C(=O)OCOP(=O)(O)OCOC(=O)C(C)(C)C ; CC(C)OC(=O)OCOP(=O)(OCOC(=O)C(C)(C)C)Oc1cccc2[nH]cc(CCN(C)C)c12]

((Hydroxyphosphoryl)bis(oxy))bis(methylene) bis(2,2-dimethylpropanoate) (194 mg, 0.60 mmol) was dissolved in anhydrous DCM (5 mL) containing DMF (5 μL) at rt under an atmosphere of N₂ and a solution of (COCl)₂ (453 mg, 306 μL, 3.57 mmol) in anhydrous DCM (5 mL) was added dropwise. The mixture was stirred at rt under for 1 h, then the volatiles were removed under reduced pressure. The residue was dissolved in DCM (5 mL) and added to a mixture of psilocin (101 mg, 496 μmol) in anhydrous pyridine at 0° C. The mixture was warmed to rt and stirred for 18 h, then concentrated under reduced pressure to give an oil containing the product (359 mg). LC-MS (+ve mode): m/z=513.25 [M+H]⁺.

### Alternative Procedure:

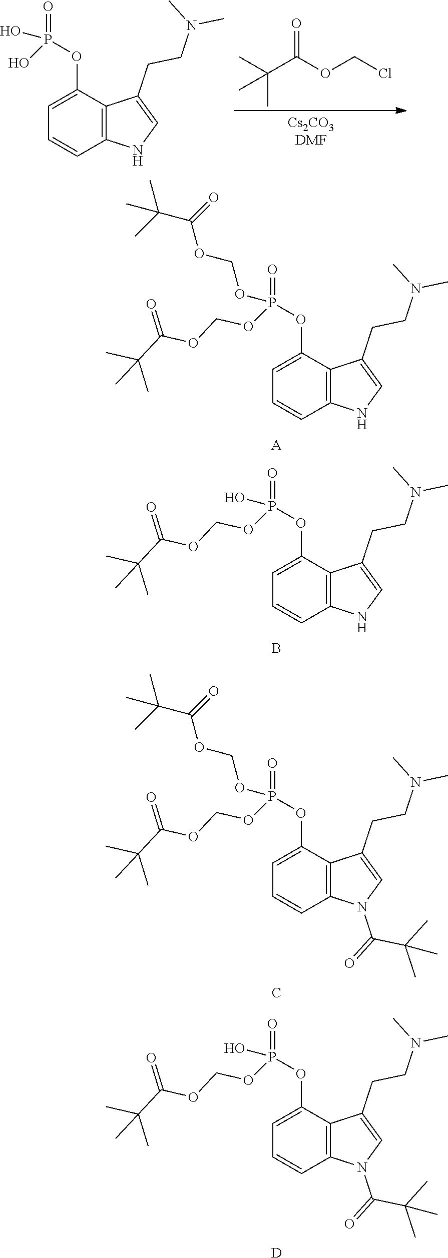

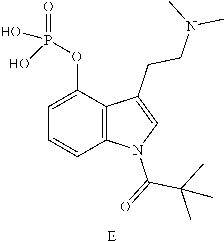

Cesium carbonate (161 mg, 0.49 mmol) was added to a stirred suspension of psilocybin (70 mg, 0.25 mmol) in anhydrous DMF (3 mL) under an atmosphere of N₂. After 15 min, chloromethyl pivalate (74 mg, 71 μL, 0.49 mmol) was added to the suspension and the mixture was heated to 50° C. and stirred for 16 h. The solvent was removed under reduced pressure, to afford a semi-solid, containing the above compounds. LC-MS (+ve mode): m/z=513.25 (A), 399.15 (B), 597.30 (C), 483.20 (D) and 389.30 (E) [M+H]⁺.

### Example 45: Ethyl 3-((((3-(2-(dimethylamino)ethyl)-1H-indol-4-yl)oxy)sulfonyl)oxy)-2,2-dimethylpropanoate

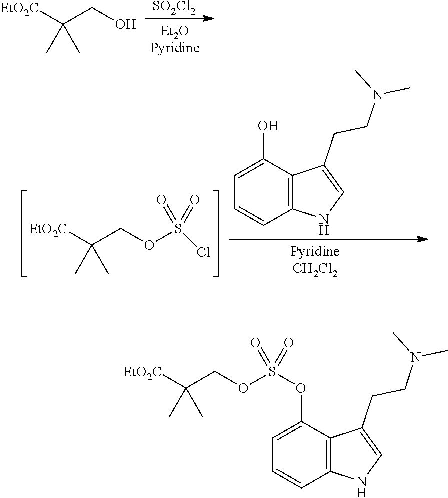

Ethyl 3-hydroxy-2,2-dimethyl propanoate (351 mg, 2.39 mmol) and pyridine (115 mg, 118 μL, 1.46 mmol) were dissolved in anhydrous Et₂O (3 mL) under an atmosphere of N₂ and the mixture was cooled to −78° C. A solution of SO₂Cl₂ (200 mg, 118 μL, 1.46 mmol) in anhydrous Et₂O (10 mL) was added dropwise and the mixture was stirred at −78° C. for 30 min. The prepared suspension containing the chlorosulfonyloxy intermediate was added dropwise to a solution of psilocin (136 mg, 0.664 mmol) in anhydrous pyridine (10 mL) at 0° C. under an atmosphere of N₂, the mixture was warmed up to rt and stirred for 16 h. The precipitate was removed by filtration, the filter cake was washed with DCM, and the filtrate was concentrated to give a semi-solid. LC-MS (+ve mode): m/z=413.15 [M+H]⁺.

### Example 46: Pharmacokinetics of Selected Psilocin Prodrugs Following a Single Intravenous or Oral Administration in Rats

The example protocol used in the PK study is summarized in Table 8 below.

TABLE 8
PO or IV PK Study Protocol Summary

| Test Compound(s) | | Psilocin Prodrugs | |
|---|---|---|---|
| Dosing Route | | PO or IV | |
| Overnight food withdrawal | | Yes, if PO | |
| Animals | Type | rat | |
| | Strain | Sprague Dawley rats | |
| | Sex | male | |
| | Weight (g) | 160-300 | g |
| | N per | 3 | |
| | compound | | |

| | |
|---|---|
| Preparation | None |
| Cage | PK cages |

| Dose | 10 | mg/kg |
|---|---|---|
| Dosing Soln. Conc. | 2 | mg/mL |
| Dosing Volume | 5 | mL/kg |
| Formulation checks | No | |
| required? | | |
| Vehicle | DMSO/PEG-400/water [10:40:50] | |
| Sampling time points (h) | PO: 0.5, 1, 2, 4, 7 & 24 h | |
| | IV only: 0.25 & 0.45 min samples also collected | |
| Blood sampling method | Serial via JVC or tail vein as warranted | |
| Alternative method if | Tail vein if catheter fails | |
| required | | |
| Sample format required | >230 μL blood + 5 μL EDTA (93 mg/mL) to give 2 × 50 | |
| | μL plasma | |
| Sample processing | Centrifugation for plasma ASAP at 4° C. Place 110 μL | |
| | plasma into Eppendorf tube on ice containing 11 μL 10% | |
| | phosphoric acid. Gently mix before taking 2 × 50 μL | |
| | aliquots into duplicate 96 well plates on dry ice. | |
| Anticoagulant | EDTA (93 mg/mL): 5 μL per tube | |
| Centrifugation | 10,000 rpm × 3 min at 4° C. | |
| Additional samples | n/a | |
| Perfusion/rinsing required | n/a | |
| Euthanasia method | n/a | |
| Plasma sample tubes | 96 well plates | |
| Pre-freezer storage | Blood: ice (<30 min), | |
| | Acidified Plasma: dry ice | |
| Freezer storage | −80° | C. |

| Dose formulation samples | 100 μL from vortex dose solution in Eppendorf |
|---|---|
| Number of samples per cpd | PO: 18 × acidified plasma (50 μL in duplicate), 1 dose |
| at 1 dose level | solution |
| | IV only: 24 × acidified plasma (50 μL in duplicate), 1 |
| | dose solution |

### Analysis

Samples were sent for method optimisation and measurement of both prodrug and parent compound (psilocin) via unique calibration lines and following acceptance QC's. Dose formulation concentrations were also measured, and PK parameters were determined (Cmax (ng/mL), Tmax (hr), Cl (ml/min/kg), Vdss (L/kg), t1/2(hr), AUC0-t (ng/mL*hr), AUC0-inf (ng/mL*hr), MRT (hr), Bioavailability (% F) where warranted) using WinNon Lin software. Data (to include bioanalytical results and assay performance) were reported in a tabulated format.

### Additional Formulation Details for PK Study

Phosphoric acid. Dilute 85% phosphoric acid 8.5-fold to give a 10% solution.

Formulation for PO Administration: For PO dosing, the prodrug was formulated in 10% DMSO/40% PEG-400/water to a concentration of 2 mg/mL of psilocin. This provides a dose of 10 mg/kg of psilocin when the prodrug was administered PO in 5 mL/kg dosing volumes.

Formulation for IV Administration: For IV dosing, the prodrug was formulated in 10% DMSO/90% hydroxypropyl-β-cyclodextrin (HIPCD, 20% w/v in water) to a concentration of 0.5 mg/mL of psilocin. This provides a dose of 1 mg/kg of psilocin when the prodrug was administered IV in 2 mL/kg dosing volumes.

NB: The prodrugs (psilocin free) were first dissolved in DMSO, and then was added PEG then water/0.5% methylcellulose as warranted.

Measurement of Concentration of Psilocin after IV or Oral Administration of Psilocin Prodrugs or Derivatives In Vivo

The pharmacokinetic properties of the synthesized psilocin prodrugs or derivatives after IV or oral administration in a rat model were assessed. The concentration of psilocin was measured in each rat at various sampling timepoints after IV or oral administration of the synthesized psilocin prodrugs or derivatives to rats.

Dose formulations were made at equivalent concentrations of psilocin adjusted for molecular weight of the tested compounds. Nominal doses (1 mg/kg for IV and 2 mg/kg for PO) were used in PK parameter determinations.

### Example 2-1. Psilocin Parent Compound (IV)

| | |
|---|---|
| Species | Rat |
| Dosed Test Article: | Arm-4 |
| Dose Route: | IV |
| Nominal Dose Concentration: | 1 mg/kg |

Chemical name: Psilocin
Structural class: parent
Mechanistic class: n/a—parent compound

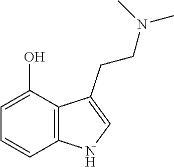

TABLE 2-1
Psilocin (IV) PK Parameters
Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| Psilocin | IV | R10 | 0.413 | 0.250 | 1110 | 24.00 | 1340 | 982 |
| | | R11 | 0.353 | 0.250 | 2450 | 24.00 | 3780 | 3390 |
| | | R12 | 0.668 | 0.250 | 340 | 24.00 | 646 | 368 |
| | | Mean | 0.478 | 0.250 | 1300 | 24.00 | 1920 | 1580 |

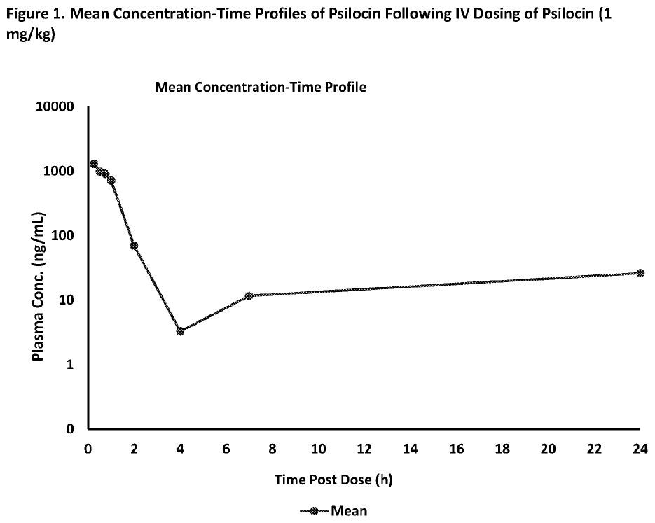

FIG. 1 shows mean concentration-time profiles of psilocin following IV dosing of Psilocin (1 mg/kg).

### Example 2-2. Psilocin Parent Compound (PO)

| | |
|---|---|
| Species | Rat |
| Dosed Test Article: | Arm-5 |
| Dose Route: | PO |
| Nominal Dose Concentration: | 2 mg/kg |

Chemical name: Psilocin
Structural class: parent
Mechanistic class: n/a—parent compound

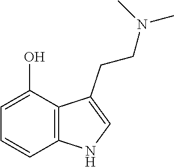

TABLE 2-2
Psilocin (PO) PK Parameters
Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| Psilocin | PO | R13 | NR | 0.50 | 13.7 | 24.0 | 97.2 | NR |
| | | R14 | 2.18 | 1.00 | 17.7 | 7.00 | 52.1 | 58.4 |
| | | R15 | 2.05 | 1.00 | 22.4 | 7.00 | 60.2 | 67.0 |
| | | Mean | 2.12 | 0.833 | 17.9 | 12.7 | 69.8 | 62.7 |

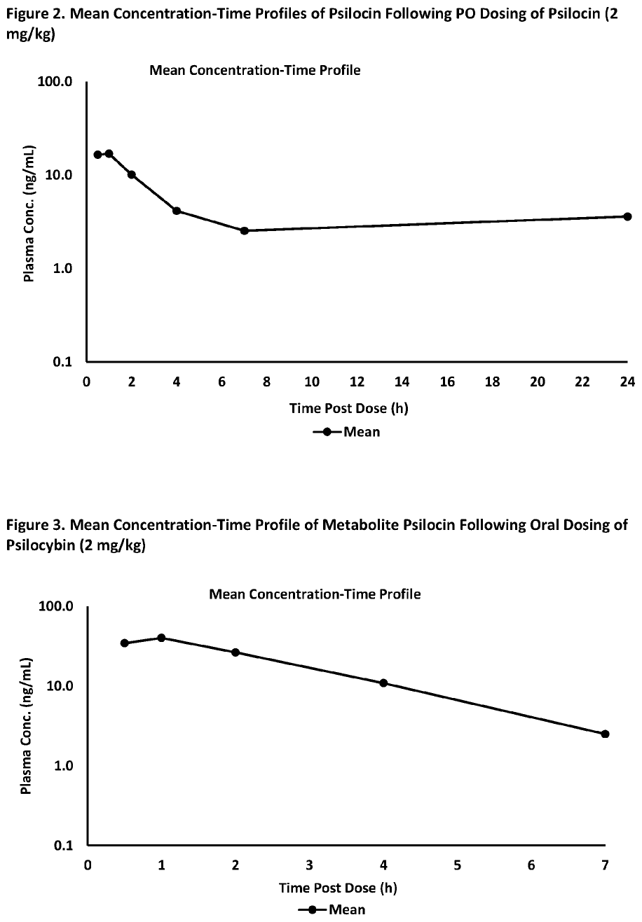

FIG. 2 shows mean concentration-time profiles of psilocin following oral dosing of Psilocin (2 mg/kg).

### Example 2-3. Psilocybin

| | |
|---|---|
| Species | Rat |
| Dosed Test Article: | Arm-6 |
| Dose Route: | PO |
| Nominal Dose Concentration: | 2 mg/kg |

Chemical name: Psilocybin
Structural class: Phosphate prodrug
Mechanistic class: Phosphatase

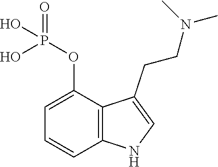 [SMILES: CN(C)CCc1c[nH]c2cccc(OP(=O)(O)O)c12]

TABLE 2-3
Psilocin PK Parameters
Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| Psilocybin | Oral | R16 | NR | 1.00 | 42.4 | 4.00 | 87.4 | NR |
| | | R17 | 1.37 | 1.00 | 45.1 | 7.00 | 139 | 144 |
| | | R18 | 2.24 | 0.50 | 34.6 | 7.00 | 112 | 128 |
| | | Mean | 1.81 | 0.833 | 40.7 | 6.00 | 113 | 136 |

FIG. 3 shows mean concentration-time profile of metabolite psilocin following oral dosing of Psilocybin (2 mg/kg).

### Example 2-4. Psilocin-O-TBDMS Ether Prodrug

| | |
|---|---|
| Species | Rat |
| Dosed Test Article: | Arm-1 |
| Dose Route: | PO |
| Nominal Dose Concentration: | 2 mg/kg |

Chemical name: 2-(4-((tert-Butyldimethylsilyl)oxy)-1H-indol-3-yl)-N,N-dimethylethan-1-amine
Structural class: Silyl ether
Mechanistic class: Presumed non-enzymatic breakdown

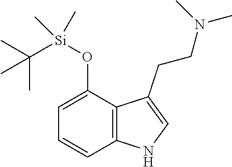 [SMILES: CN(C)CCc1c[nH]c2cccc(O[Si](C)(C)C(C)(C)C)c12]

TABLE 2-4
Psilocin PK Parameters
Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| NCT- | Oral | R1 | NR | 2.00 | 11.0 | 7.00 | 48.1 | NR |
| 1670-01 | | R2 | 2.90 | 1.00 | 14.2 | 7.00 | 54.6 | 70.6 |
| | | R3 | 2.99 | 1.00 | 19.3 | 7.00 | 80.0 | 106 |
| | | Mean | 2.95 | 1.33 | 14.8 | 7.00 | 60.9 | 88.1 |

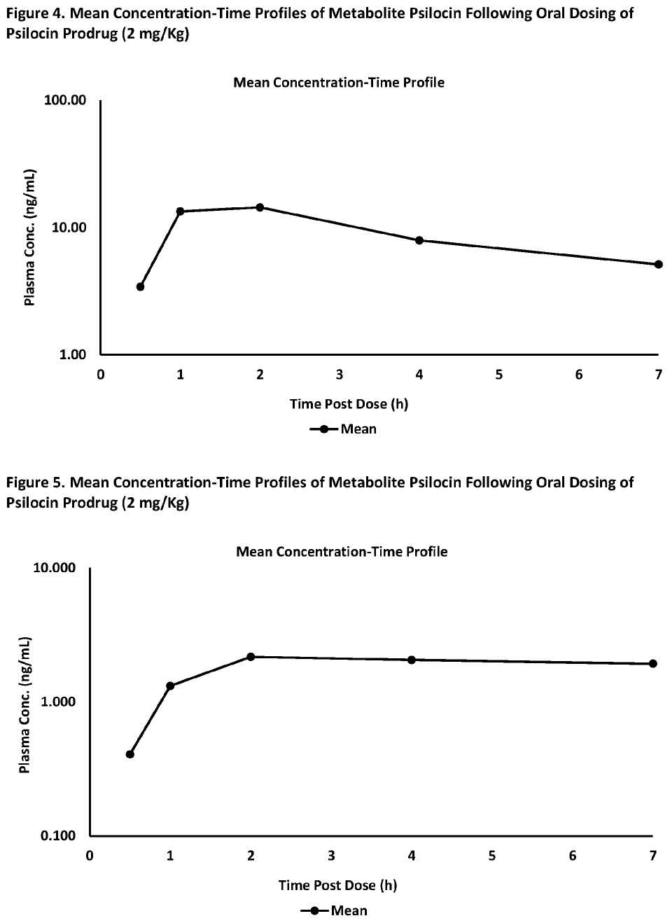

FIG. 4 shows mean concentration-time profiles of metabolite psilocin following oral dosing of psilocin prodrug (2 mg/Kg).

### Example 2-5. Psilocin-O-TIPS Ether Prodrug

| | |
|---|---|
| Species | Rat |
| Dosed Test Article: | Arm-2 |
| Dose Route: | PO |
| Nominal Dose Concentration: | 2 mg/kg |

Chemical name: N,N-Dimethyl-2-(4-((triisopropylsilyl)oxy)-1H-indol-3-yl)ethan-1-amine
Structural class: Silyl ether
Mechanistic class: Presumed non-enzymatic breakdown

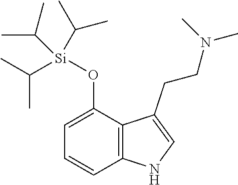 [SMILES: CC(C)[Si](Oc1cccc2[nH]cc(CCN(C)C)c12)(C(C)C)C(C)C]

TABLE 2-5
Psilocin PK Parameters
Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| NCT- | Oral | R4 | NR | 4.00 | 2.31 | 7.00 | 12.3 | NR |
| 1672-01 | | R5 | NR | 7.00 | 1.48 | 7.00 | 6.02 | NR |
| | | R6 | NR | 4.00 | 3.27 | 7.00 | 19.2 | NR |
| | | Mean | NR | 5.00 | 2.35 | 7.00 | 12.5 | NR |

FIG. 5 shows mean concentration-time profiles of metabolite psilocin following oral dosing of psilocin prodrug (2 mg/Kg).

### Example 2-6. Psilocin O-Adipate Ester Hydrochloride Prodrug

| | |
|---|---|
| Species | Rat |
| Dosed Test Article: | Arm-3 |
| Dose Route: | PO |
| Nominal Dose Concentration: | 2 mg/kg |

Chemical name: 6-((3-(2-(Dimethylamino)ethyl)-1H-indol-4-yl)oxy)-6-oxohexanoic acid HCl salt
Structural class: hemi-ester
Mechanistic class: Presumed esterase, and/or, presumed pH-dependent intramolecularcyclization

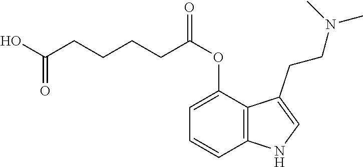 [SMILES: CN(C)CCc1c[nH]c2cccc(OC(=O)CCCCC(=O)O)c12]

TABLE 2-6
Psilocin PK Parameters
Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| NCT- | Oral | R7 | NR | 2.00 | 25.6 | 7.00 | 117 | NR |
| 1696-01 | | R8 | NR | 2.00 | 32.5 | 7.00 | 158 | NR |
| | | R9 | NR | 4.00 | 48.3 | 7.00 | 214 | NR |
| | | Mean | NR | 2.67 | 35.5 | 7.00 | 163 | NR |

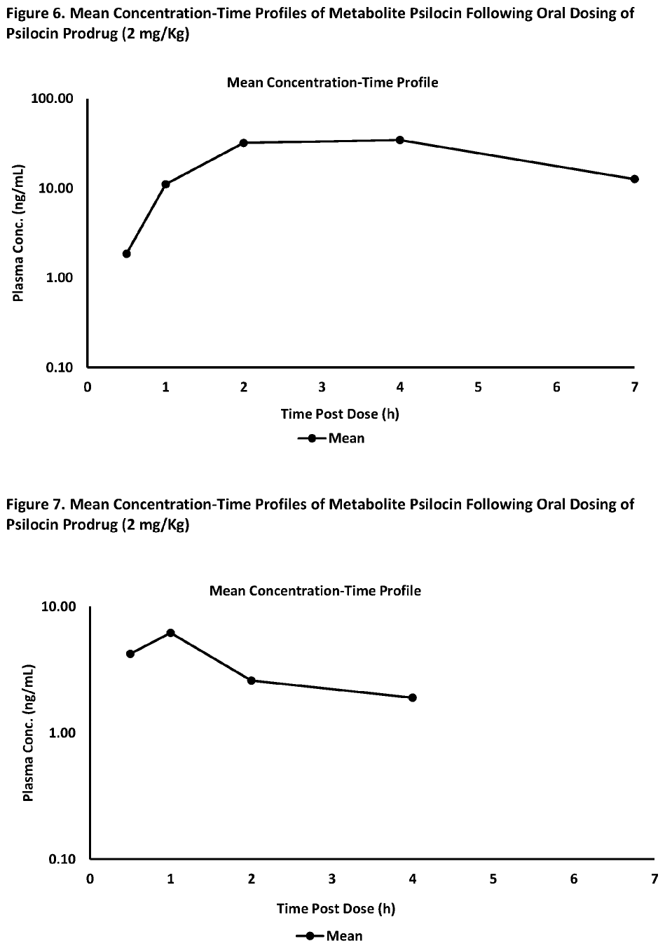

FIG. 6 shows mean concentration-time profiles of metabolite psilocin following oral dosing of psilocin prodrug (2 mg/Kg).

### Example 2-7. Psilocin tetrahydrofuran-3-ester Hydrochloride Prodrug

| | |
|---|---|
| Species | Rat |
| Dosed Test Article: | Arm-7 |
| Dose Route: | PO |
| Nominal Dose Concentration: | 2 mg/kg |

Chemical name: 3-(2-(Dimethylamino)ethyl)-1H-indol-4-yl tetrahydrofuran-3-carboxylate HCl salt
Structural class: Ester
Mechanistic class: Presumed esterase

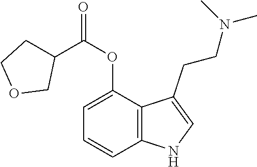 [SMILES: CN(C)CCc1c[nH]c2cccc(OC(=O)C3CCOC3)c12]

TABLE 2-7
Psilocin PK Parameters
Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| Batch | Oral | R19 | NR | 1.00 | 8.30 | 2.00 | 10.6 | NR |
| NCT- | | R20 | NR | 1.00 | 2.80 | 2.00 | 2.85 | NR |
| 1726-01 | | R21 | NR | 1.00 | 7.50 | 4.00 | 17.2 | NR |
| | | Mean | NR | 1.00 | 6.20 | 2.67 | 10.2 | NR |

FIG. 7 shows mean concentration-time profiles of metabolite psilocin following oral dosing of psilocin prodrug (2 mg/Kg).

### Example 2-8. Psilocin Trimethyl Lock Formate Prodrug

| | |
|---|---|
| Species | Rat |
| Dosed Test Article: | Arm-8 |
| Dose Route: | PO |
| Nominal Dose Concentration: | 2 mg/kg |

Chemical name: 3-(2-(Dimethylamino)ethyl)-1H-indol-4-yl 3-(2-acetoxy-4,6-dimethylphenyl)-3-methylbutanoate formate salt
Structural class: Ester
Mechanistic class: Presumed esterase+intramolecular cyclization

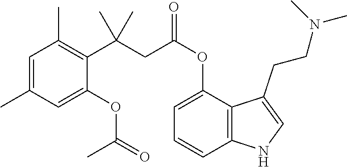

TABLE 2-8
Psilocin PK Parameters
Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| Batch | Oral | R22 | 14.4 | 1.00 | 27.4 | 7.00 | 86.3 | 313 |
| NCT- | | R23 | 68.3 | 1.00 | 19.6 | 7.00 | 84.4 | 1350 |
| 1727-01 | | R24 | 8.57 | 1.00 | 18.3 | 7.00 | 74.6 | 169 |
| | | Mean | 30.5 | 1.00 | 21.8 | 7.00 | 81.8 | 609 |

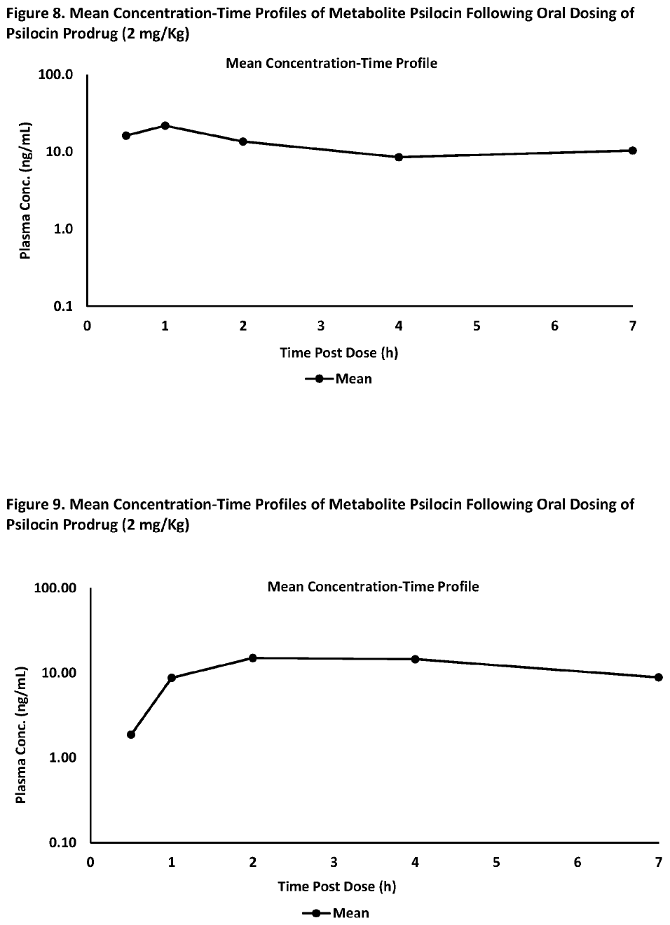

FIG. 8 shows mean concentration-time profiles of metabolite psilocin following oral dosing of psilocin prodrug (2 mg/Kg).

### Example 2-9. Psilocin 2-oxa-6-azaspiro[3.3]heptane Carboxalate Formate Prodrug

| | |
|---|---|
| Species | Rat |
| Dosed Test Article: | Arm-9 |
| Dose Route: | PO |
| Nominal Dose Concentration: | 2 mg/kg |

Chemical name: 3-(2-(Dimethylamino)ethyl)-1H-indol-4-yl 2-oxa-6-azaspiro[3.3]heptane-6-carboxylate formate salt
Structural class: carbamate
Mechanistic class: Presumed enzymatic hydrolysis

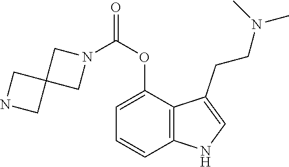 [SMILES: CN(C)CCc1c[nH]c2cccc(OC(=O)N3CC4(CNC4)C3)c12]

TABLE 2-9
Psilocin PK Parameters
Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| NCT- | Oral | R25 | NR | 2.00 | 15.0 | 7.00 | 75.8 | NR |
| 1728-01 | | R26 | NR | 2.00 | 19.7 | 7.00 | 93.1 | NR |
| | | R27 | NR | 4.00 | 15.3 | 7.00 | 70.3 | NR |
| | | Mean | NR | 2.67 | 16.7 | 7.00 | 79.7 | NR |

FIG. 9 shows mean concentration-time profiles of metabolite psilocin following oral dosing of psilocin prodrug (2 mg/Kg).

### Example 2-10. Psilocin O-TES Ether Prodrug

| | |
|---|---|
| Species | Rat |
| Dosed Test Article: | Arm 10 |
| Dose Route: | PO |
| Nominal Dose Concentration: | 2 mg/kg |

Chemical name: N,N-Dimethyl-2-(4-((triethylsilyl)oxy)-1H-indol-3-yl)ethan-1-amine
Structural class: Silyl ether
Mechanistic class: Presumed non-enzymatic breakdown

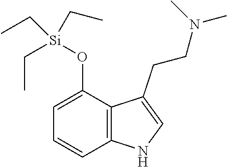 [SMILES: CC[Si](CC)(CC)Oc1cccc2[nH]cc(CCN(C)C)c12]

TABLE 2-10
Psilocin PK Parameters
Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| NCT- | Oral | R28 | 4.97 | 1.00 | 25.9 | 7.00 | 101 | 181 |
| 1729-01 | | R29 | 2.09 | 1.00 | 43.5 | 7.00 | 139 | 157 |
| | | R30 | 15.1 | 1.00 | 24.2 | 7.00 | 139 | 535 |
| | | Mean | 7.38 | 1.00 | 31.2 | 7.00 | 126 | 291 |

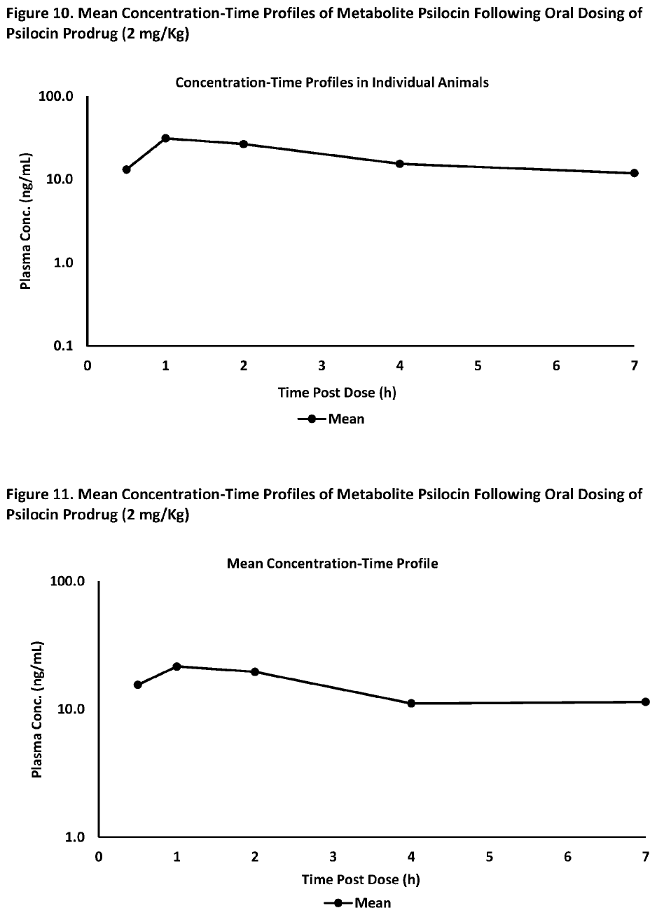

FIG. 10 shows mean concentration-time profiles of metabolite psilocin following oral dosing of psilocin prodrug (2 mg/Kg).

### Example 2-11. Psilocin Lysine Trihydrochloride Prodrug

| | |
|---|---|
| Species | Rat |
| Dosed Test Article: | Arm-11 |
| Dose Route: | PO |
| Nominal Dose Concentration: | 2 mg/kg |

Chemical name: 3-(2-(Dimethylamino)ethyl)-1H-indol-4-yl L-lysinate trihydrochloride
Structural class: Ester of amino acid
Mechanistic class: Presumed esterase

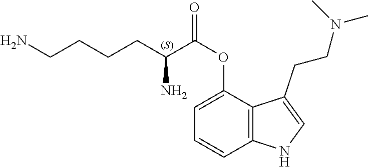

TABLE 2-11
Psilocin PK Parameters
Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| NCT- | Oral | R1 | NR | 1.00 | 21.7 | 7.00 | 82.5 | NR |
| 1736-01 | | R2 | NR | 2.00 | 26.3 | 7.00 | 121 | NR |
| | | R3 | 4.30 | 1.00 | 21.0 | 7.00 | 90.8 | 143 |
| | | Mean | 4.30 | 1.33 | 23.0 | 7.00 | 98.1 | 143 |

FIG. 11 shows mean concentration-time profiles of metabolite psilocin following oral dosing of psilocin prodrug (2 mg/Kg).

### Example 2-12. Psilocin Oxane Hydrochloride Prodrug

| | |
|---|---|
| Species | Rat |
| Dosed Test Article: | Arm-12 |
| Dose Route: | PO |
| Nominal Dose Concentration: | 2 mg/kg |

Chemical name: 3-(2-(Dimethylamino)ethyl)-1H-indol-4-yl tetrahydro-2H-pyran-4-carboxylate hydrochloride
Structural class: Ester
Mechanistic class: Presumed esterase

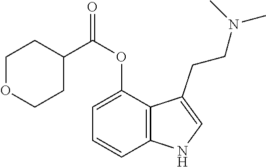 [SMILES: CN(C)CCc1c[nH]c2cccc(OC(=O)C3CCOCC3)c12]

TABLE 2-12
Psilocin PK Parameters
Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| NCT- | Oral | R4 | 2.57 | 0.500 | 13.9 | 7.00 | 45.7 | 54.7 |
| 1737-01 | | R5 | NR | 0.500 | 6.94 | 7.00 | 24.6 | NR |
| | | R6 | NR | 2.00 | 7.64 | 7.00 | 41.7 | NR |
| | | Mean | 2.6 | 1.00 | 9.49 | 7.00 | 37.3 | 55 |

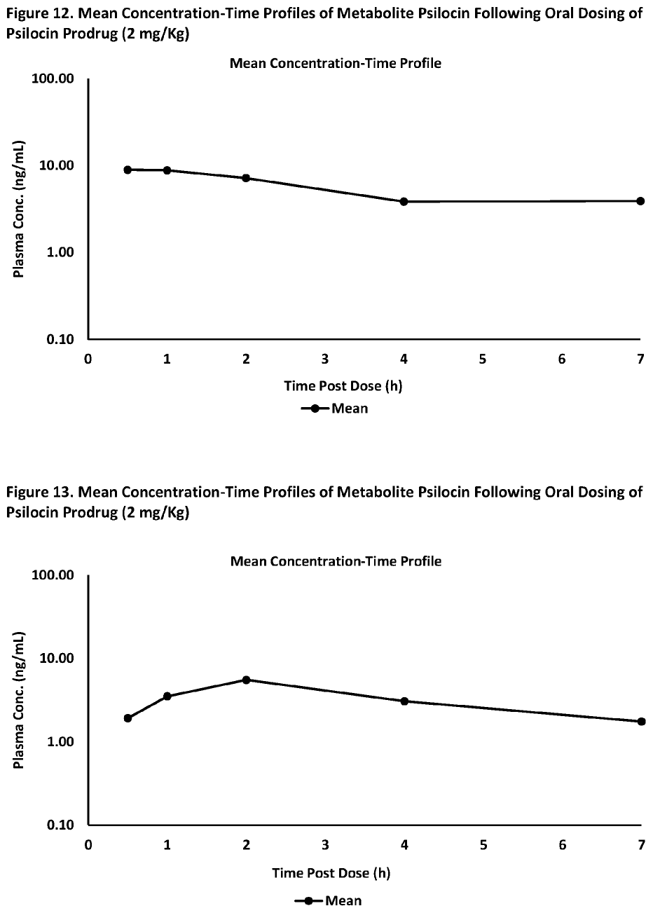

FIG. 12 shows mean concentration-time profiles of metabolite psilocin following oral dosing of psilocin prodrug (2 mg/Kg).

### Example 2-13. Psilocin Morpholine Carbamate Hydrochloride Prodrug

| | |
|---|---|
| Species | Rat |
| Dosed Test Article: | Arm-13 |
| Dose Route: | PO |
| Nominal Dose Concentration: | 2 mg/kg |

Chemical name: 3-(2-(Dimethylamino)ethyl)-1H-indol-4-yl morpholine-4-carboxylate HCl salt
Structural class: carbamate
Mechanistic class: Presumed enzymatic hydrolysis

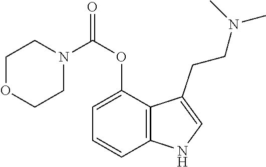 [SMILES: CN(C)CCc1c[nH]c2cccc(OC(=O)N3CCOCC3)c12]

TABLE 2-13
Psilocin PK Parameters
Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| NCT- | Oral | R7 | 2.92 | 1.00 | 2.27 | 7.00 | 8.96 | 11.8 |
| 1740-01 | | R8 | NR | 2.00 | 2.87 | 7.00 | 12.8 | NR |
| | | R9 | NR | 2.00 | 11.5 | 7.00 | 43.8 | NR |
| | | Mean | 2.92 | 1.67 | 5.55 | 7.00 | 21.9 | 11.8 |

FIG. 13 shows Mean Concentration-Time Profiles of Metabolite Psilocin Following Oral Dosing of Psilocin Prodrug (2 mg/Kg).

### Example 2-14. Psilocin O-methyl Ethyl Carbonate Formate Prodrug

| | |
|---|---|
| Species | Rat |
| Dosed Test Article: | Arm-14 |
| Dose Route: | PO |
| Nominal Dose Concentration: | 2 mg/kg |

Chemical name: ((3-(2-(Dimethylamino)ethyl)-1H-indol-4-yl)oxy)methyl ethyl carbonate formate
Structural class: Methyleneoxy carbonate
Mechanistic class: Presumed esterase+chemical breakdown

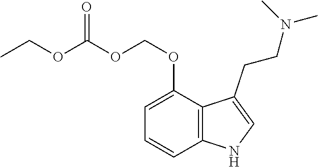 [SMILES: CCOC(=O)OCOc1cccc2[nH]cc(CCN(C)C)c12]

TABLE 2-14
Psilocin PK Parameters
Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| NCT- | Oral | R10 | 1.16 | 0.500 | 5.57 | 4.00 | 12.8 | 14.4 |
| 1741-01 | | R11 | NR | 2.00 | 3.45 | 4.00 | 9.69 | NR |
| | | R12 | NR | 1.00 | 4.93 | 4.00 | 12.1 | NR |
| | | Mean | 1.16 | 1.17 | 4.65 | 4.00 | 11.5 | 14.4 |

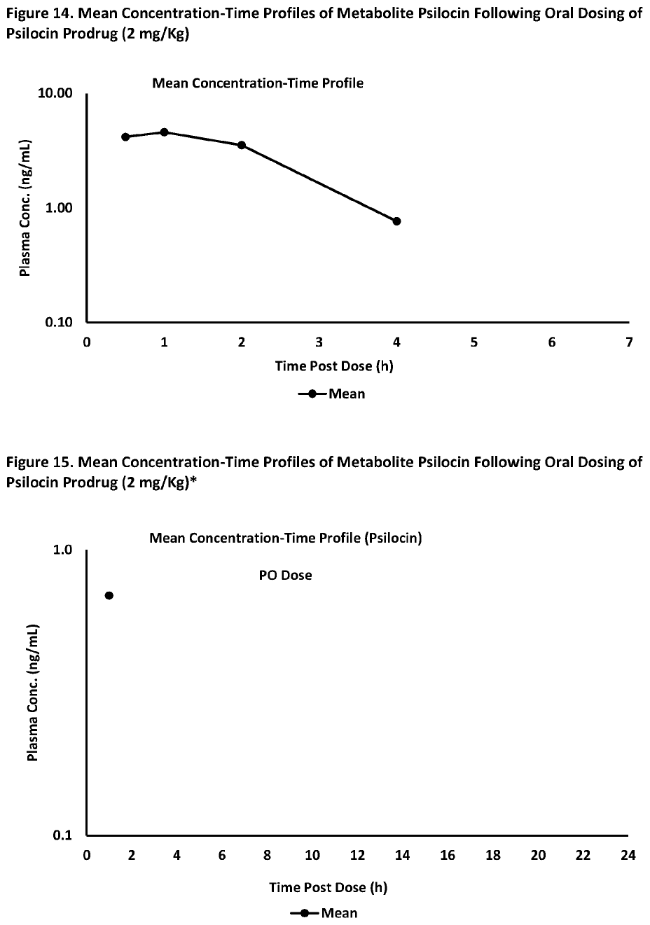

FIG. 14 shows Mean Concentration-Time Profiles of Metabolite Psilocin Following Oral Dosing of Psilocin Prodrug (2 mg/Kg).

### Example 2-15. Psilocin di-tert-butyl Phosphonate Hydrochloride Prodrug

| | |
|---|---|
| Species | Rat |
| Dosed Test Article: | Arm-15 |
| Dose Route: | PO |
| Nominal Dose Concentration: | 2 mg/kg |

Chemical name: Di-tert-butyl [3-[2-(dimethylamino)ethyl]-1H-indol-4-yl] phosphate HCl salt
Structural class: Phosphonate
Mechanistic class: Presumed phosphatase

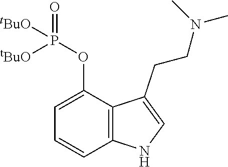 [SMILES: CN(C)CCc1c[nH]c2cccc(OP(=O)(OC(C)(C)C)OC(C)(C)C)c12]

TABLE 2-15
Psilocin PK Parameters
Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| APEX- | Oral | R13 | NR | 1.00 | 0.692 | 1.00 | NR | NR |
| 000059- | | R14 | NR | 0.500 | 0.00 | NR | NR | NR |
| 114-1 | | R15 | NR | 0.500 | 0.00 | NR | NR | NR |
| | | Mean | NR | 0.667 | 0.231 | 1.00 | NR | NR |

FIG. 15 shows mean concentration-time profiles of metabolite psilocin following oral dosing of psilocin prodrug (2 mg/Kg). *Plasma concentrations were below limit of quantification (BLQ) at other time points.

### Example 2-16. Psilocin Boc-Valine Formate Prodrug

| | |
|---|---|
| Species | Rat |
| Dosed Test Article: | Arm-16 |
| Dose Route: | PO |
| Nominal Dose Concentration: | 2 mg/kg |

Chemical name: 3-(2-(Dimethylamino)ethyl)-1H-indol-4-yl (tert-butoxycarbonyl)-L-valinate formate
Structural class: Protected amino acid ester prodrug
Mechanistic class: Presumed esterase

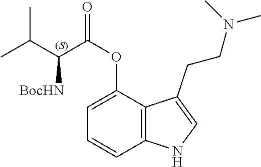

TABLE 2-16
Psilocin PK Parameters
Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| NCT- | Oral | R16 | 1.73 | 1.00 | 7.34 | 7.00 | 23.4 | 25.4 |
| 1742-01 | | R17 | NR | 2.00 | 26.1 | 7.00 | 92.5 | NR |
| | | R18 | 9.30 | 1.00 | 19.1 | 24.0 | 93.1 | 107 |
| | | Mean | 5.52 | 1.33 | 17.5 | 12.7 | 69.7 | 66.2 |

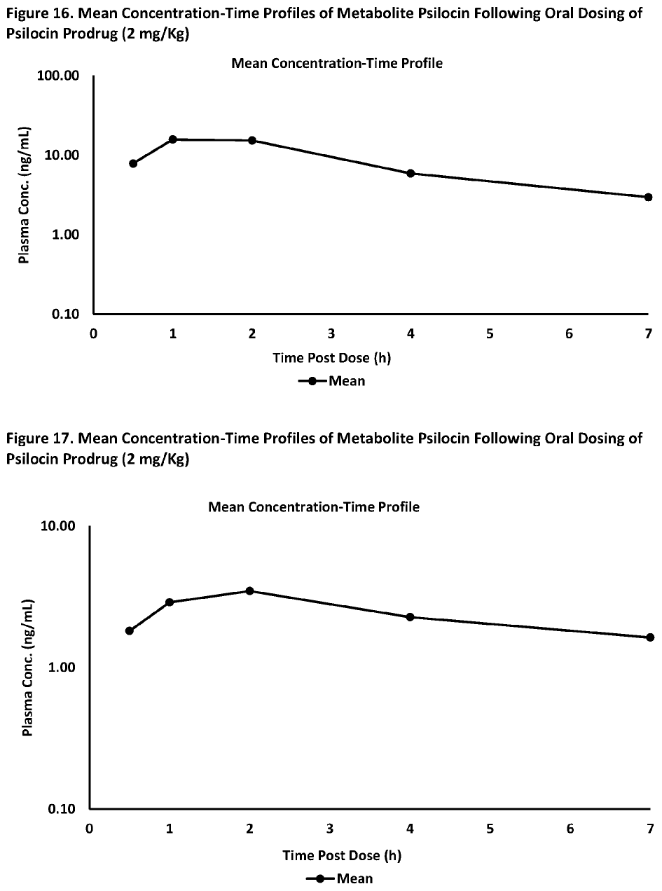

FIG. 16 shows mean concentration-time profiles of metabolite psilocin following oral dosing of psilocin prodrug (2 mg/Kg).

### Example 2-17. Psilocin Boc-Proline Formate Prodrug

| | |
|---|---|
| Species | Rat |
| Dosed Test Article: | Arm-17 |
| Dose Route: | PO |
| Nominal Dose Concentration: | 2 mg/kg |

Chemical name: 1-(tert-Butyl) 2-(3-(2-(dimethylamino)ethyl)-1H-indol-4-yl) L-pyrrolidine-1,2-dicarboxylate formate
Structural class: Protected amino acid ester
Mechanistic class: Presumed esterase

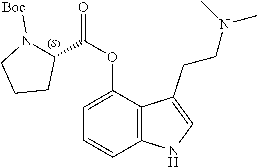

TABLE 2-17
Psilocin PK Parameters
Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| NCT- | Oral | R19 | NR | 2.00 | 3.47 | 7.00 | 15.6 | NR |
| 1743-01 | | R20 | 14.2 | 1.00 | 3.62 | 7.00 | 18.4 | 65.6 |
| | | R21 | NR | 2.00 | 3.99 | 7.00 | 15.2 | NR |
| | | Mean | 14.2 | 1.67 | 3.69 | 7.00 | 16.4 | 65.6 |

FIG. 17 shows mean concentration-time profiles of metabolite psilocin following oral dosing of psilocin prodrug (2 mg/Kg).

| | |
|---|---|
| Species | Rat |
| Dosed Test Article: | Arm-18 |
| Dose Route: | PO |
| Nominal Dose Concentration: | 2 mg/kg |

### Example 2-18. Psilocin Phenylalanine Dihydrochloride Prodrug

Chemical name: 3-(2-(Dimethylamino)ethyl)-1H-indol-4-yl L-phenylalaninate 2HCl salt
Structural class: Amino acid ester
Mechanistic class: Presumed esterase

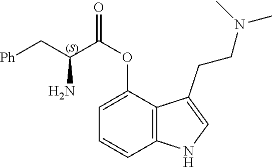

TABLE 2-18
Psilocin PK Parameters
Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| NCT- | Oral | R22 | 8.81 | 0.500 | 20.3 | 7.00 | 79.9 | 193 |
| 1744-01 | | R23 | NR | 1.00 | 33.1 | 7.00 | 110 | NR |
| | | R24 | 12.6 | 1.00 | 36.9 | 7.00 | 144 | 500 |
| | | Mean | 10.7 | 0.833 | 30.1 | 7.00 | 111 | 347 |

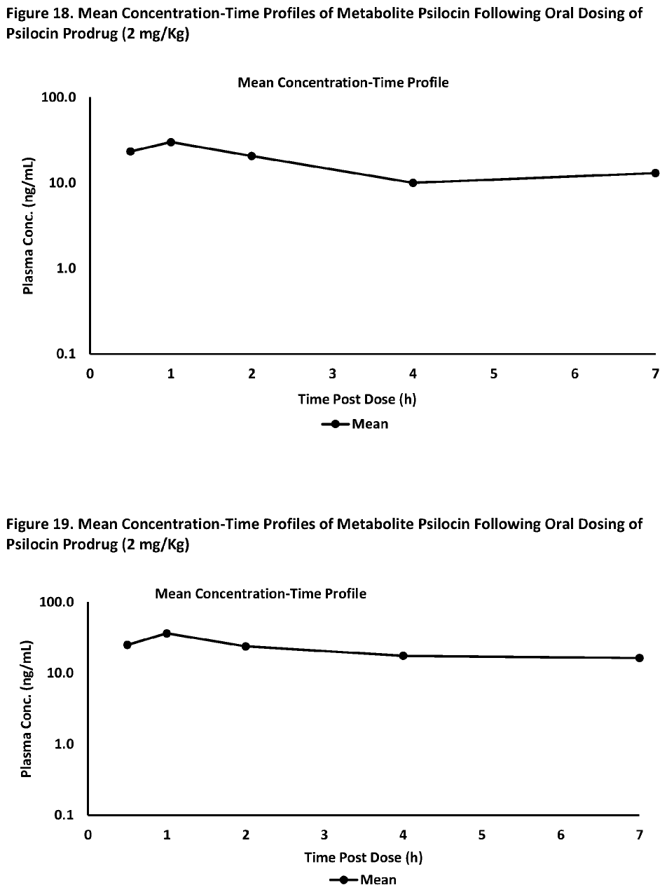

FIG. 18 shows mean concentration-time profiles of metabolite psilocin following oral dosing of psilocin prodrug (2 mg/Kg).

### Example 2-19. Psilocin Boc-Phenylalanine Formate Prodrug

| | |
|---|---|
| Species | Rat |
| Dosed Test Article: | Arm-19 |
| Dose Route: | PO |
| Nominal Dose Concentration: | 2 mg/kg |

Chemical name: 3-(2-(Dimethylamino)ethyl)-1H-indol-4-yl (tert-butoxycarbonyl)-L-phenylalaninate formate
Structural class: Protected amino acid ester
Mechanistic class: Presumed esterase

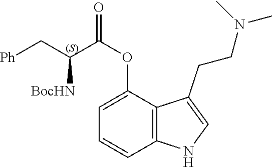

TABLE 2-19
Psilocin PK Parameters
Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| NCT- | Oral | R25 | 2.93 | 1.00 | 46.4 | 7.00 | 163 | 206 |
| 1697-01 | | R26 | 5.48 | 1.00 | 35.5 | 7.00 | 123 | 215 |
| | | R27 | NR | 7.00 | 27.3 | 7.00 | 145 | NR |
| | | Mean | 4.21 | 3.00 | 36.4 | 7.00 | 144 | 211 |

FIG. 19 shows mean concentration-time profiles of metabolite psilocin following oral dosing of psilocin prodrug (2 mg/Kg).

### Example 2-20. Psilocin Pivaloyloxymethyl (POM) Prodrug

| | |
|---|---|
| Species | Rat |
| Dosed Test Article: | Arm 20 |
| Dose Route: | PO |
| Nominal Dose Concentration: | 2 mg/kg |

Chemical name: ((3-(2-(Dimethylamino)ethyl)-1H-indol-4-yl)oxy)methyl pivalate
Structural class: Pivaloyloxymethyl (POM) prodrug
Mechanistic class: Presumed esterase+chemical breakdown

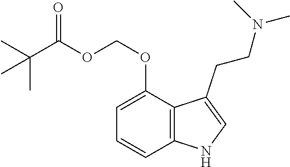 [SMILES: CN(C)CCc1c[nH]c2cccc(OCOC(=O)C(C)(C)C)c12]

TABLE 2-20
Psilocin PK Parameters
Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| NCT- | Oral | R28 | NR | 1.00 | 1.47 | 2.00 | 2.12 | NR |
| 1745-01 | | R29 | NR | 1.00 | 3.26 | 4.00 | 7.95 | NR |
| | | R30 | 2.11 | 0.500 | 3.18 | 4.00 | 7.06 | 9.93 |
| | | Mean | 2.11 | 0.833 | 2.64 | 3.33 | 5.71 | 9.93 |

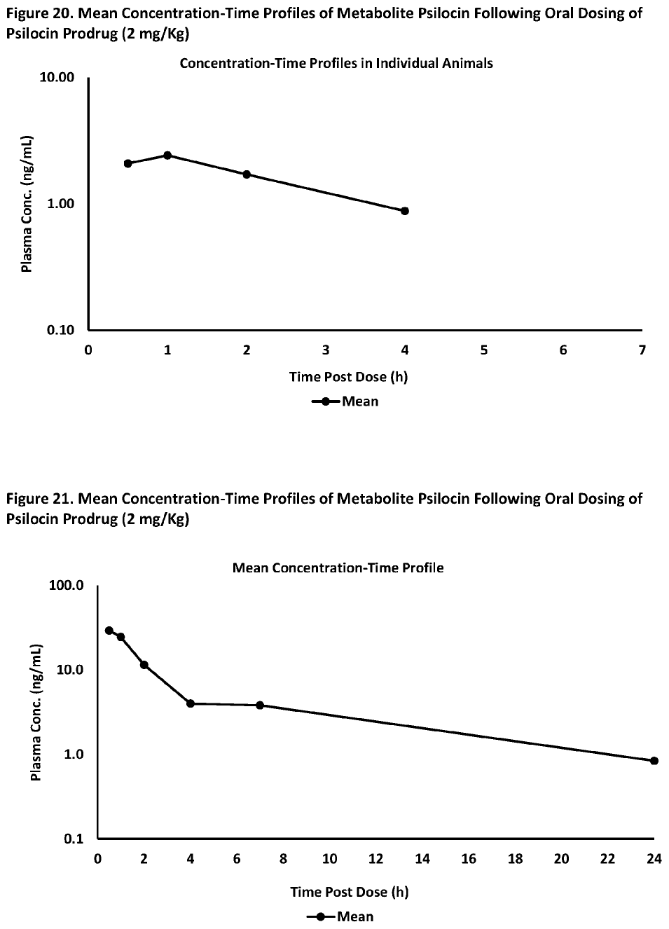

FIG. 20 shows mean concentration-time profiles of metabolite psilocin following oral dosing of psilocin prodrug (2 mg/Kg).

### Example 2-21. Psilocin O-Proline Ester Dihydrochloride Prodrug

| Species: | Rat |
|---|---|
| Dosed Test Article: | Arm-21 |
| Dose Route: | PO |
| Nominal Dose Concentration (mg/KG): | 2 |

Chemical name: 3-(2-(Dimethylamino)ethyl)-1H-indol-4-yl L-prolinate 2HCl salt
Structural class: Amino acid ester
Mechanistic class: Presumed esterase

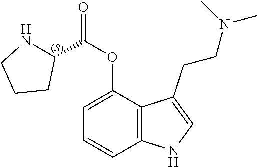

TABLE 2-21
Psilocin PK Parameters
Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| NCT- | Oral | R1 | 9.71 | 1.00 | 19.7 | 24.0 | 80.9 | 92.6 |
| 1746-01 | | R2 | 2.48 | 0.500 | 22.0 | 7.00 | 49.9 | 57.8 |
| | | R3 | 4.13 | 0.500 | 46.7 | 7.00 | 98.1 | 136 |
| | | Mean | 5.44 | 0.667 | 29.5 | 12.7 | 76.3 | 95.5 |

FIG. 21 shows mean concentration-time profiles of metabolite psilocin following oral dosing of psilocin prodrug (2 mg/Kg).

### Example 2-22. Psilocin N-POM Ether Prodrug

| Species: | Rat |
|---|---|
| Dosed Test Article: | Arm-22 |
| Dose Route: | PO |
| Nominal Dose Concentration (mg/KG): | 2 |

Chemical name: (3-(2-(dimethylamino)ethyl)-4-hydroxy-1H-indol-1-yl)methyl pivalate
Structural class: N-Pivaloyloxymethyl (POM)
Mechanistic class: Presumed esterase+chemical breakdown

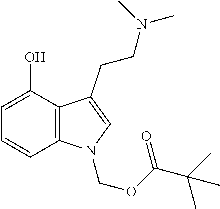 [SMILES: CN(C)CCc1cn(COC(=O)C(C)(C)C)c2cccc(O)c12]

TABLE 2-22
Psilocin PK Parameters
Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| NTC- | Oral | R4 | NR | 0.500 | 14.6 | 2.00 | 15.3 | NR |
| 1748-01 | | R5 | 0.940 | 0.500 | 20.4 | 4.00 | 30.4 | 32.4 |
| | | R6 | 1.01 | 0.500 | 19.9 | 4.00 | 26.8 | 28.7 |
| | | Mean | 0.975 | 0.500 | 18.3 | 3.33 | 24.2 | 30.6 |

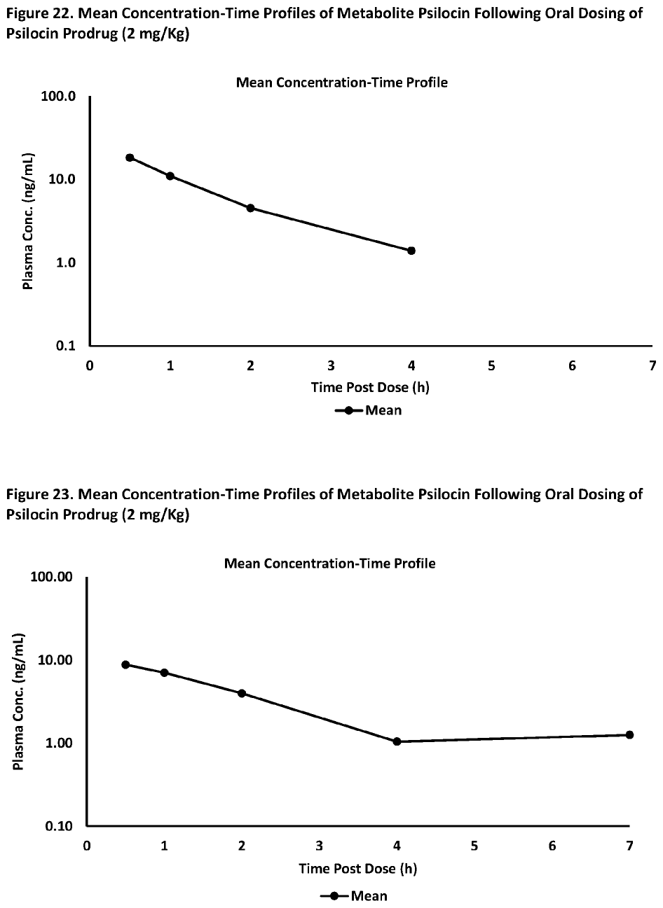

FIG. 22 shows mean concentration-time profiles of metabolite psilocin following oral dosing of psilocin prodrug (2 mg/Kg).

### Example 2-23. Psilocin N-POM Ether O-Pivaloyl Prodrug

| Species: | Rat |
|---|---|
| Dosed Test Article: | Arm-23 |
| Dose Route: | PO |
| Nominal Dose Concentration (mg/KG): | 2 |

Chemical name: 3-(2-(dimethylamino)ethyl)-1-((pivaloyloxy)methyl)-1H-indol-4-yl pivalate
Structural class: N-Pivaloyloxymethyl (POM) and ester
Mechanistic class: Presumed esterase+chemical breakdown

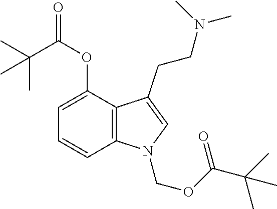 [SMILES: CN(C)CCc1cn(COC(=O)C(C)(C)C)c2cccc(OC(=O)C(C)(C)C)c12]

TABLE 2-23
Psilocin PK Parameters
Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| NCT- | Oral | R7 | 1.03 | 0.500 | 9.94 | 4.00 | 18.8 | 20.4 |
| 1749-01 | | R8 | 4.77 | 0.500 | 7.43 | 7.00 | 16.3 | 24.9 |
| | | R9 | NR | 0.500 | 9.04 | 2.00 | 12.4 | NR |
| | | Mean | 2.90 | 0.500 | 8.80 | 4.33 | 15.8 | 22.7 |

FIG. 23 shows mean concentration-time profiles of metabolite psilocin following oral dosing of psilocin prodrug (2 mg/Kg).

### Example 2-24. Psilocin O-Methyl Glutarate Ether t-Butyl Ester Prodrug

| Species: | Rat |
|---|---|
| Dosed Test Article: | Arm-24 |
| Dose Route: | PO |
| Nominal Dose Concentration (mg/KG): | 2 |

Chemical name: tert-Butyl {3-[2-(dimethylamino)ethyl]-4-indolyloxy}methyl glutarate
Structural class: Acyloxymethyl (AOM)
Mechanistic class: Presumed esterase, and/or, pH-dependent intramolecular cyclization, + chemical breakdown

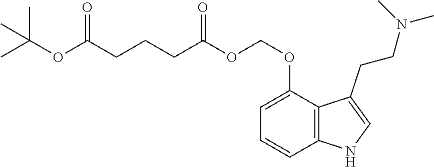 [SMILES: CN(C)CCc1c[nH]c2cccc(OCOC(=O)CCCC(=O)OC(C)(C)C)c12]

TABLE 2-24
Psilocin PK Parameters
Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| NCT- | Oral | R10 | NR | 0.500 | 14.9 | 2.00 | 12.5 | NR |
| 1750-01 | | R11 | 1.25 | 0.500 | 12.7 | 4.00 | 16.0 | 18.0 |
| | | R12 | 1.12 | 0.500 | 5.99 | 4.00 | 11.6 | 13.0 |
| | | Mean | 1.19 | 0.500 | 11.2 | 3.33 | 13.4 | 15.5 |

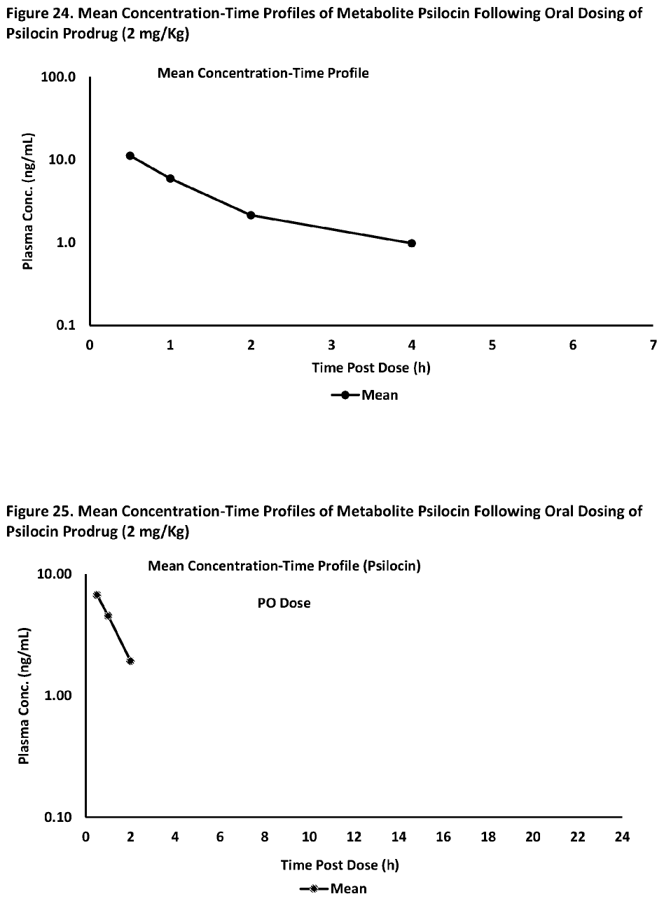

FIG. 24 shows mean concentration-time profiles of metabolite psilocin following oral dosing of psilocin prodrug (2 mg/Kg).

### Example 2-25. Psilocin O-Methyl Succinate Ether t-Butyl Ester Prodrug

| Species: | Rat |
|---|---|
| Dosed Test Article: | Arm-25 |
| Dose Route: | PO |
| Nominal Dose Concentration (mg/KG): | 2 |

Chemical name: tert-Butyl {3-[2-(dimethylamino)ethyl]-4-indolyloxy}methyl succinate
Structural class: Acyloxymethyl (AOM)
Mechanistic class: Presumed esterase, and/or, pH-dependent intramolecular cyclization, + chemical breakdown

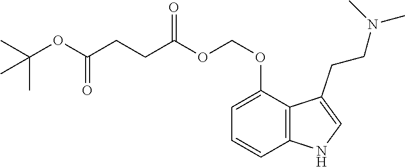 [SMILES: CN(C)CCc1c[nH]c2cccc(OCOC(=O)CCC(=O)OC(C)(C)C)c12]

TABLE 2-25
Psilocin PK Parameters
Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| NCT- | Oral | R13 | NR | 0.500 | 6.18 | 2.00 | 6.79 | NR |
| 1751-01 | | R14 | NR | 0.500 | 9.22 | 2.00 | 10.6 | NR |
| | | R15 | NR | 0.500 | 4.85 | 2.00 | 5.86 | NR |
| | | Mean | NR | 0.500 | 6.75 | 2.00 | 7.75 | NR |

FIG. 25 shows mean concentration-time profiles of metabolite psilocin following oral dosing of psilocin prodrug (2 mg/Kg).

### Example 2-26. Psilocin O-methylpiperazine Carbamate Diformate Prodrug

| Species: | Rat |
|---|---|
| Dosed Test Article: | Arm-26 |
| Dose Route: | PO |
| Nominal Dose Concentration (mg/KG): | 2 |

Chemical name: [3-[2-(Dimethylamino)ethyl]-1H-indol-4-yl] 4-methylpiperazine-1-carboxylate diformate
Structural class: Carbamate
Mechanistic class: Presumed enzymatic hydrolysis

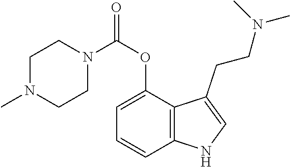

TABLE 2-26
Psilocin PK Parameters
Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| NCT- | Oral | R16 | NR | NR | NR | NR | NR | NR |
| 1752-01 | | R17 | NR | NR | NR | NR | NR | NR |
| | | R18 | NR | NR | NR | NR | NR | NR |
| | | Mean | NR | NR | NR | NR | NR | NR |

*Plasma concentrations BLQ—Below limit of quantification

### Example 2-27. Psilocin O-Methyl Adipate Ether t-Butyl Ester Prodrug

| Species: | Rat |
|---|---|
| Dosed Test Article: | Arm-27 |
| Dose Route: | PO |
| Nominal Dose Concentration (mg/KG): | 2 |

Chemical name: tert-Butyl {3-[2-(dimethylamino)ethyl]-4-indolyloxy}methyl adipate
Structural class: Acyloxymethyl (AOM)
Mechanistic class: Presumed esterase, and/or, pH-dependent intramolecular cyclization, + chemical breakdown

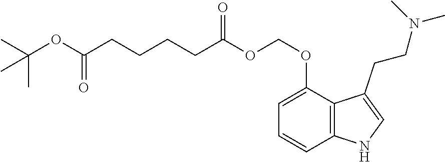 [SMILES: CN(C)CCc1c[nH]c2cccc(OCOC(=O)CCCCC(=O)OC(C)(C)C)c12]

TABLE 2-27
Psilocin PK Parameters
Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| NCT- | Oral | R19 | NR | 0.500 | 12.3 | 2.00 | 14.6 | NR |
| 1753-01 | | R20 | 1.19 | 0.500 | 4.18 | 4.00 | 7.53 | 8.45 |
| | | R21 | 0.722 | 0.500 | 14.4 | 4.00 | 20.0 | 20.6 |
| | | Mean | 0.956 | 0.500 | 10.3 | 3.33 | 14.0 | 14.5 |

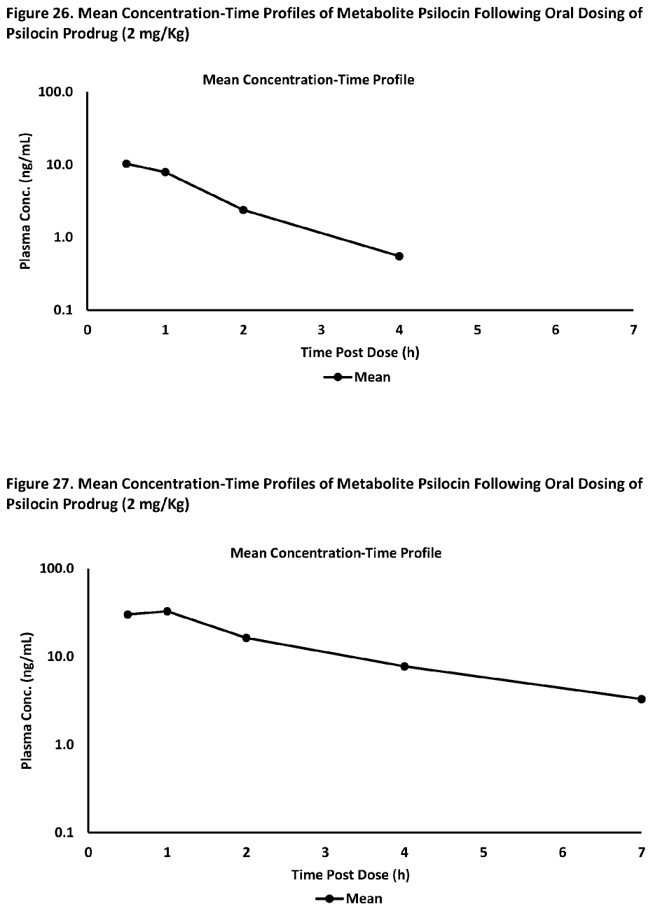

FIG. 26 shows mean concentration-time profiles of metabolite psilocin following oral dosing of psilocin prodrug (2 mg/Kg).

### Example 2-28. Psilocin Valine Dihydrochloride Prodrug

| Species: | Rat |
|---|---|
| Dosed Test Article: | Arm-28 |
| Dose Route: | PO |
| Nominal Dose Concentration (mg/KG): | 2 |

Chemical name: 3-(2-(Dimethylamino)ethyl)-1H-indol-4-yl L-valinate 2HCl
Structural class: Amino acid ester
Mechanistic class: Presumed esterase

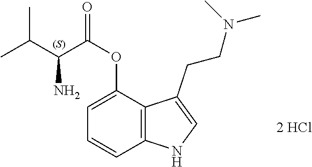

TABLE 2-28
Psilocin PK Parameters
Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| NCT- | Oral | R22 | 3.04 | 0.500 | 30.6 | 7.00 | 72.2 | 89.0 |
| 1756-01 | | R23 | 1.96 | 1.00 | 32.2 | 7.00 | 73.4 | 79.7 |
| | | R24 | 1.92 | 1.00 | 40.3 | 7.00 | 119 | 130 |
| | | Mean | 2.31 | 0.833 | 34.4 | 7.00 | 88.2 | 99.6 |

FIG. 27 shows mean concentration-time profiles of metabolite psilocin following oral dosing of psilocin prodrug (2 mg/Kg).

### Example 2-29. Psilocin N-Boc-L-Phenylalanine-Sarcosine Ester Formate Prodrug

| Species: | Rat |
|---|---|
| Dosed Test Article: | Arm-29 |
| Dose Route: | PO |
| Nominal Dose Concentration (mg/KG): | 2 |

Chemical name: 3-(2-(Dimethylamino)ethyl)-1H-indol-4-yl N-((tert-butoxycarbonyl)-L-phenylalanyl)-N-methylglycinate formate
Structural class: Dipeptide
Mechanistic class: Presumed esterase, and/or, pH-dependent intramolecular cyclization

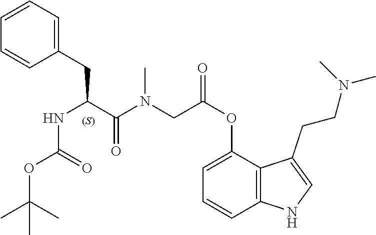

TABLE 2-29
Psilocin PK Parameters
Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| NCT- | Oral | R25 | 1.89 | 1.00 | 61.9 | 7.00 | 163 | 177 |
| 1757-01 | | R26 | NR | 2.00 | 16.5 | 7.00 | 56.5 | NR |
| | | R27 | NR | 2.00 | 10.9 | 7.00 | 55.8 | NR |
| | | Mean | 1.89 | 1.67 | 29.8 | 7.00 | 91.8 | 177 |

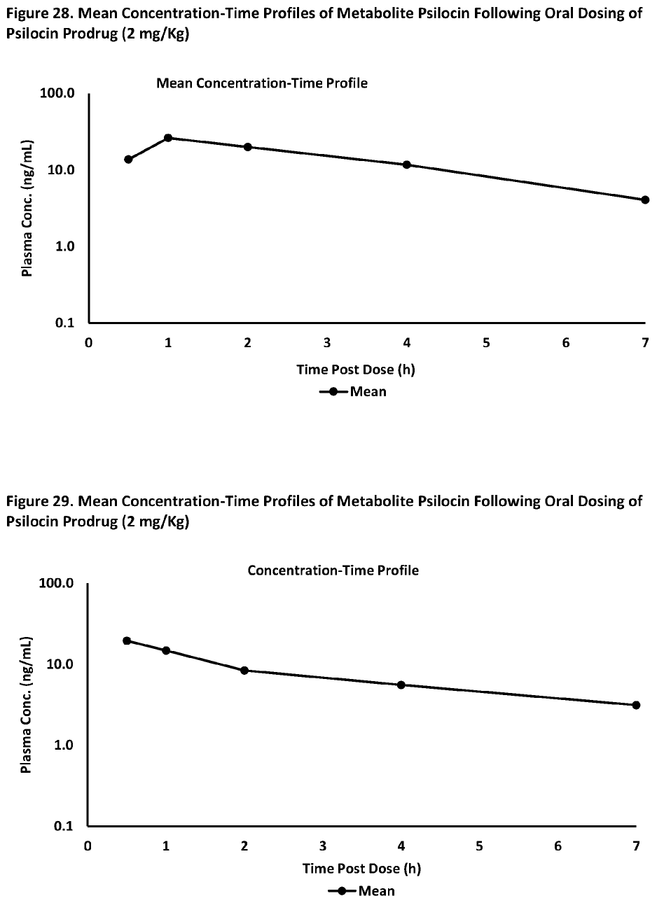

FIG. 28 shows mean concentration-time profiles of metabolite psilocin following oral dosing of psilocin prodrug (2 mg/Kg).

### Example 2-30. Psilocin Dimethylglycine Ester Diformate Prodrug

| Species: | Rat |
|---|---|
| Dosed Test Article: | Arm 30 |
| Dose Route: | PO |
| Nominal Dose Concentration (mg/KG): | 2 |

Chemical name: 3-(2-(Dimethylamino)ethyl)-1H-indol-4-yl dimethylglycinate diformate
Structural class: Ester
Mechanistic class: Presumed esterase

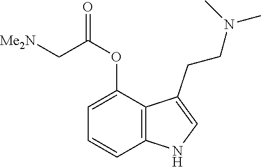 [SMILES: CN(C)CCc1c[nH]c2cccc(OC(=O)CN(C)C)c12]

TABLE 2-30
Psilocin PK Parameters
Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| NCT- | Oral | R28 | 2.25 | 0.500 | 23.2 | 7.00 | 38.9 | 42.9 |
| 1758-01 | | R29 | 11.1 | 0.500 | 14.4 | 7.00 | 58.0 | 156 |
| | | R30 | 2.05 | 0.500 | 21.0 | 7.00 | 59.2 | 65.3 |
| | | Mean | 5.13 | 0.500 | 19.5 | 7.00 | 52.0 | 88.1 |

FIG. 29 shows mean concentration-time profiles of metabolite psilocin following oral dosing of psilocin prodrug (2 mg/Kg).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A compound of Formula (I), or a pharmaceutically acceptable salt, solvate, or isotopolog thereof: 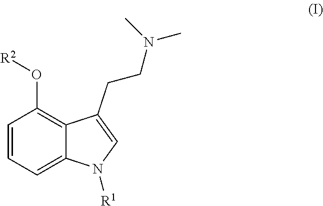
wherein:
R¹ is hydrogen, —OH, unsubstituted or substituted alkyl, OR, or C(O)OR; wherein R is unsubstituted alkyl;
R² is —C(O)R³, —C(O)OR³, —CH(R⁴)OC(O)R⁵, —CH(R⁴)OC(O)OR⁵, —C(O)NR⁶R⁷, —CH(R⁴)OC(O)NR⁶R⁷, —S(O)₂NR⁶R⁷, —S(O)₂OR⁵, —P(O)OR⁸(NR⁹R¹⁰), —P(O)(OR¹¹)(OR¹²), —CH(R⁴)OP(O)(OR¹¹)(OR¹²), or —Si(R³)(R⁴)(R⁵);
each of R³, R⁴, R⁵, and R⁸ is independently hydrogen, alkyl, heteroalkyl, cycloalkyl, heterocyclylalkyl, aryl, or heteroaryl, wherein alkyl, heteroalkyl, cycloalkyl, heterocyclylalkyl, aryl, or heteroaryl is unsubstituted or substituted with one or more R^{A};
each of R⁶ and R⁷ is independently hydrogen, alkyl, heteroalkyl, cycloalkyl, heterocyclylalkyl, aryl, or heteroaryl, wherein alkyl, heteroalkyl, cycloalkyl, heterocyclylalkyl, aryl, or heteroaryl is unsubstituted or substituted with one or more R^{A}; or R⁶ and R⁷ together with the atom to which they are attached form a heterocyclylalkyl ring or a heteroaryl ring that is unsubstituted or substituted with one or more R^{A};
each of R⁹ and R¹⁰ is independently hydrogen, alkyl, heteroalkyl, cycloalkyl, heterocyclylalkyl, aryl, heteroaryl, or hydrogen, wherein alkyl, heteroalkyl, cycloalkyl, heterocyclylalkyl, aryl, or heteroaryl is unsubstituted or substituted with one or more R^{A}, or R⁹ and R¹⁰ together with the atom to which they are attached form a heterocyclylalkyl ring or a heteroaryl ring that is unsubstituted or substituted with one or more R^{A};
each of R¹¹ and R¹² is independently hydrogen, alkyl, heteroalkyl, cycloalkyl, heterocyclylalkyl, aryl, heteroaryl, or hydrogen, wherein alkyl, heteroalkyl, cycloalkyl, heterocyclylalkyl, aryl, or heteroaryl is unsubstituted or substituted with one or more R^{A};
each R^{A} is independently alkyl, heteroalkyl, cycloalkyl, heterocyclylalkyl, aryl, heteroaryl, an amino acid side chain, —OR¹³, —N(R¹⁸)R¹⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R⁸)R¹⁹], —C(O)N(R¹⁸)R¹⁹, —OC(O)N(R¹⁸)R¹⁹, or —OP(O)OR²⁰(OR²¹), wherein alkyl, heteroalkyl, cycloalkyl, heterocyclylalkyl, aryl, or heteroaryl is unsubstituted or substituted with one or more alkyl, aryl, halogen, —S—R¹³, —OR¹³, —NR(R¹⁸)R¹⁹, —C(O)R¹⁴, —OC(O)R¹⁴, —OC(O)OR¹⁶, or —OC(O)N(R¹⁸)R¹⁹;
each of R¹³, R¹⁴, R¹⁵, R¹⁶, or R¹⁷ is independently hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, and heteroaryl is unsubstituted or substituted with one or more R^{B};
each of R¹⁸ and R¹⁹ is independently hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, or heteroaryl is unsubstituted or substituted with one or more R^{B};
or R¹⁸ and R¹⁹ together with the atom to which they are attached form a heterocyclylalkyl ring or heteroaryl ring, each of which is unsubstituted or substituted with one or more R^{B};
each R^{B} is independently halogen, amino, cyano, hydroxyl, alkyl, alkenyl, heteroalkyl, cycloalkyl, heterocyclylalkyl, aryl, heteroaryl, arylalkyl, —OC(O)R¹⁸, —C(O)R¹⁸, —C(O)OR¹⁸, NHC(O)OR¹⁸, or heteroarylalkyl, wherein cycloalkyl, heterocyclylalkyl, aryl, or heteroaryl is unsubstituted or substituted with one or more halogen, amino, cyano, hydroxyl, alkyl, acetyl, or benzoyl.

2. The compound or pharmaceutically acceptable salt, solvate, or isotopolog of claim 1, wherein the compound has the structure of Formula (Ia):

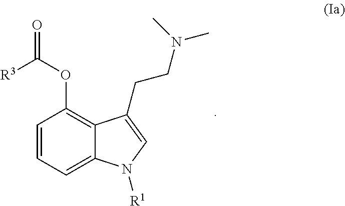

3. The compound or pharmaceutically acceptable salt, solvate, or isotopolog of claim 1 or claim 2, wherein R³ is unsubstituted or substituted alkyl, heteroalkyl, cycloalkyl, or heterocyclylalkyl.

4. The compound or pharmaceutically acceptable salt, solvate, or isotopolog of any one of claims 1 to 3, wherein R³ is unsubstituted or substituted alkyl.

5. The compound or pharmaceutically acceptable salt, solvate, or isotopolog of claim 4, wherein R³ is alkyl substituted with one or more substituent R^{A}, and wherein each R^{A} is independently selected from alkyl, heteroalkyl, cycloalkyl, heterocyclylalkyl, aryl, heteroaryl, an amino acid side chain, —OR¹³, —N(R¹⁸)R¹⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹], —C(O)N(R¹⁸)R¹⁹, —OC(O)N(R¹⁸)R¹⁹, or —OP(O)OR²⁰(OR²¹).

6. The compound or pharmaceutically acceptable salt, solvate, or isotopolog of claim 4 or claim 5, wherein R³ is unsubstituted alkyl.

7. The compound or pharmaceutically acceptable salt, solvate, or isotopolog of claim 6, wherein R³ is methyl, ethyl, n-propyl, isopropyl, n-butyl, tert-butyl, n-pentyl, 3-methyl-1-butyl, or —C₁₀H₂₁.

8. The compound or pharmaceutically acceptable salt, solvate, or isotopolog of claim 4 or claim 5, wherein R³ is alkyl substituted with —C(O)OR¹³.

9. The compound or pharmaceutically acceptable salt, solvate, or isotopolog of claim 8, wherein R¹³ is hydrogen or alkyl.

10. The compound or pharmaceutically acceptable salt, solvate, or isotopolog of claim 8, wherein R¹³ is hydrogen, methyl, ethyl, or tert-butyl.

11. The compound or pharmaceutically acceptable salt, solvate, or isotopolog of claim 4 or claim 5, wherein R³ is alkyl substituted with —N(R¹⁸)R¹⁹, wherein each of R¹⁸ and R¹⁹ is independently hydrogen or methyl.

12. The compound or pharmaceutically acceptable salt, solvate, or isotopolog of claim 4 or claim 5, wherein R³ is alkyl substituted with —N(R¹³)C(O)R¹⁴, wherein each of R¹³ and R¹⁴ is independently hydrogen or methyl.

13. The compound or pharmaceutically acceptable salt, solvate, or isotopolog of claim 4 or claim 5, wherein R³ is alkyl substituted with —N(R¹³)C(O)R¹⁴, wherein each of R¹³ is hydrogen or methyl, and R¹⁴ is hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, and heteroaryl is unsubstituted or further substituted with one or more halogen, amino, cyano, hydroxyl, alkyl, acetyl, benzoyl, phenyl, or NH-Boc.

14. The compound or pharmaceutically acceptable salt, solvate, or isotopolog of claim 4 or claim 5, wherein R³ is alkyl substituted with —N(R¹³)C(O)OR¹⁴, wherein each of R¹³ and R¹⁴ is independently hydrogen, methyl, ethyl, isopropyl, or tert-butyl.

15. The compound or pharmaceutically acceptable salt, solvate, or isotopolog of claim 4 or claim 5, wherein R³ is

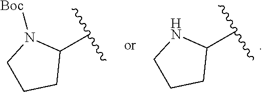 [SMILES: *C1CCCN1C(=O)OC(C)(C)C ; *C1CCCN1]

16. The compound or pharmaceutically acceptable salt, solvate, or isotopolog of claim 4 or claim 5, wherein R³ is 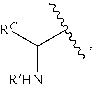
wherein R^{C} is a natural amino acid side chain, and R′ is hydrogen or -Boc.

17. The compound or pharmaceutically acceptable salt, solvate, or isotopolog of claim 16, wherein R³ is

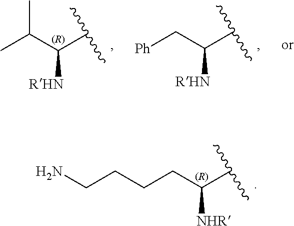

18. The compound or pharmaceutically acceptable salt, solvate, or isotopolog of any one of claims 1 to 3, wherein R³ is heterocyclylalkyl.

19. The compound or pharmaceutically acceptable salt, solvate, or isotopolog of claim 18, wherein R³ is selected from aziridinyl, oxetanyl, azetidinyl, tetrahydrofuranyl, pyrrolidinyl, piperidinyl, piperazinyl, morpholinyl, thiomorpholinyl, thiomorpholinyl dioxide, diazinanyl, 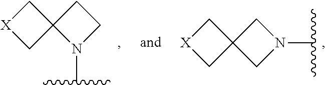
wherein X is —CH₂—, —O—, —S—, —SO₂, —NH—, or —NMe.

20. The compound or pharmaceutically acceptable salt, solvate, or isotopolog of claim 4 or claim 5, wherein R³ is

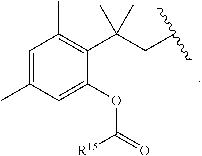

21. The compound or pharmaceutically acceptable salt, solvate, or isotopolog of claim 4 or claim 5, wherein R³ is alkyl substituted with —OC(O)R¹⁵, wherein R¹⁵ is hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, and heteroaryl is unsubstituted or further substituted with one or more halogen, amino, cyano, hydroxyl, alkyl, acetyl, or benzoyl.

22. The compound or pharmaceutically acceptable salt, solvate, or isotopolog of any one of claims 1 to 3, wherein R³ is heteroalkyl.

23. The compound or pharmaceutically acceptable salt, solvate, or isotopolog of any one of claims 1 to 3, wherein R³ is unsubstituted or substituted aryl (e.g., phenyl).

24. The compound or pharmaceutically acceptable salt, solvate, or isotopolog of claim 23, wherein R³ is substituted phenyl.

25. The compound or pharmaceutically acceptable salt, solvate, or isotopolog of claim 24, wherein R³ is phenyl substituted with —OC(O)R¹⁸, wherein R¹⁸ is hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl.

26. The compound or pharmaceutically acceptable salt, solvate, or isotopolog of claim 1, wherein the compound has the structure of Formula (Ib):

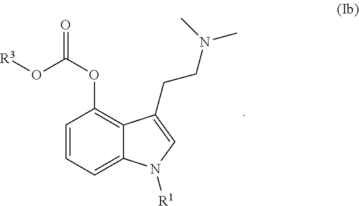

27. The compound or pharmaceutically acceptable salt, solvate, or isotopolog of claim 26, wherein R³ is unsubstituted or substituted alkyl, heteroalkyl, cycloalkyl, or heterocyclylalkyl.

28. The compound or pharmaceutically acceptable salt, solvate, or isotopolog of claim 26 or claim 20, wherein R³ is unsubstituted or substituted alkyl.

29. The compound or pharmaceutically acceptable salt, solvate, or isotopolog of any one of claims 19 to 21, wherein R³ is alkyl substituted with one or more substituent R^{A}, and wherein each R^{A} is independently selected from alkyl, heteroalkyl, cycloalkyl, heterocyclylalkyl, aryl, heteroaryl, an amino acid side chain, —OR¹³, —N(R¹⁸)R¹⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹], —C(O)N(R¹⁸)R¹⁹, —OC(O)N(R¹⁸)R¹⁹, or —OP(O)OR²⁰(OR²¹).

30. The compound or pharmaceutically acceptable salt, solvate, or isotopolog of claim 29, wherein R³ is alkyl substituted with heterocyclylalkyl.

31. The compound or pharmaceutically acceptable salt, solvate, or isotopolog of claim 30, wherein R³ is alkyl substituted with aziridinyl, oxetanyl, azetidinyl, tetrahydrofuranyl, pyrrolidinyl, piperidinyl, piperazinyl, morpholinyl, thiomorpholinyl, thiomorpholinyl dioxide, diazinanyl, 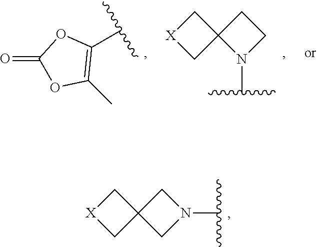
wherein X is —CH₂—, —O—, —S—, —SO₂, —NH—, or —NMe.

32. The compound or pharmaceutically acceptable salt, solvate, or isotopolog of any one of claims 26 to 28, wherein R³ is alkyl substituted with one or more —OC(O)R¹⁵.

33. The compound or pharmaceutically acceptable salt, solvate, or isotopolog of any one of claims 26 to 28, wherein R³ is isopropyl substituted with two —OC(O)R¹⁵ wherein each R¹⁵ is alkyl.

34. The compound or pharmaceutically acceptable salt, solvate, or isotopolog of any one of claims 26 to 28, wherein R³ is unsubstituted alkyl.

35. The compound or pharmaceutically acceptable salt, solvate, or isotopolog of claim 34, wherein R³ is methyl, ethyl, n-propyl, isopropyl, n-butyl, tert-butyl, n-pentyl, 3-methyl-1-butyl, or —C₁₀H₂₁.

36. The compound or pharmaceutically acceptable salt, solvate, or isotopolog of claim 26 or 27, wherein R³ is heteroalkyl.

37. The compound or pharmaceutically acceptable salt, solvate, or isotopolog of claim 26 or 27, wherein R³ is heterocyclylalkyl.

38. The compound or pharmaceutically acceptable salt, solvate, or isotopolog of claim 37, wherein R³ is selected from aziridinyl, oxetanyl, azetidinyl, tetrahydrofuranyl, pyrrolidinyl, piperidinyl, piperazinyl, morpholinyl, thiomorpholinyl, thiomorpholinyl dioxide, diazinanyl, 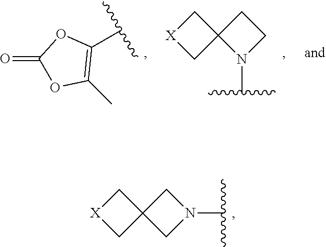
wherein X is —CH₂—, —O—, —S—, —SO₂, —NH—, or —NMe.

39. The compound or pharmaceutically acceptable salt, solvate, or isotopolog of claim 37, wherein R³ is oxetanyl.

40. The compound or pharmaceutically acceptable salt, solvate, or isotopolog of claim 26 or 27, wherein R³ is alkyl substituted with —N(R¹³)C(O)OR¹⁴, wherein each of R¹³ and R¹⁴ is independently hydrogen, methyl, or ethyl.

41. The compound or pharmaceutically acceptable salt, solvate, or isotopolog of claim 26 or 27, wherein R³ is alkyl substituted with —OC(O)R¹⁵, wherein R¹⁵ is hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, and heteroaryl is unsubstituted or further substituted with one or more halogen, amino, cyano, hydroxyl, alkyl, acetyl, or benzoyl.

42. The compound or pharmaceutically acceptable salt, solvate, or isotopolog of claim 1, wherein the compound has the structure of Formula (Ic):

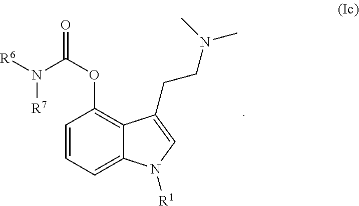

43. The compound or pharmaceutically acceptable salt, solvate, or isotopolog of claim 42, wherein each of R⁶ and R⁷ is independently hydrogen, alkyl, heteroalkyl, cycloalkyl, heterocyclylalkyl, aryl, or heteroaryl, wherein alkyl, heteroalkyl, cycloalkyl, heterocyclylalkyl, aryl, or heteroaryl is unsubstituted or substituted with one or more R^{A}.

44. The compound or pharmaceutically acceptable salt, solvate, or isotopolog of claim 42, wherein R⁶ and R⁷ together with the atom to which they are attached form a heterocyclylalkyl ring or a heteroaryl ring that is unsubstituted or substituted with one or more R^{A}.

45. The compound or pharmaceutically acceptable salt, solvate, or isotopolog of claim 44, wherein R⁶ and R⁷ together with the atom to which they are attached form aziridinyl, azetidinyl, pyrrolidinyl, piperidinyl, piperazinyl, morpholinyl, thiomorpholinyl, thiomorpholinyl dioxide, diazinanyl, 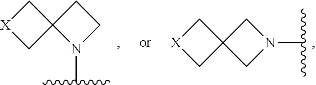
wherein X is —CH₂—, —O—, —S—, —SO₂, —NH—, or —NMe.

46. The compound or pharmaceutically acceptable salt, solvate, or isotopolog of claim 45, wherein R⁶ and R⁷ together with the atom to which they are attached form

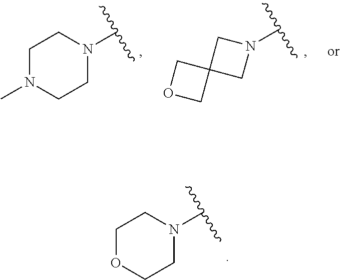

47. The compound or pharmaceutically acceptable salt, solvate, or isotopolog of claim 42 or 43, wherein R⁶ is methyl.

48. The compound or pharmaceutically acceptable salt, solvate, or isotopolog of claim 47 wherein R⁷ is alkyl substituted with —C(O)OR³, wherein R¹³ is hydrogen or alkyl.

49. The compound or pharmaceutically acceptable salt, solvate, or isotopolog of claim 47 wherein R⁷ is alkyl substituted with —C(O)OR¹³, wherein R¹³ is hydrogen, methyl, ethyl, or tert-butyl.

50. The compound or pharmaceutically acceptable salt, solvate, or isotopolog of claim 1, wherein the compound has the structure of Formula (Id):

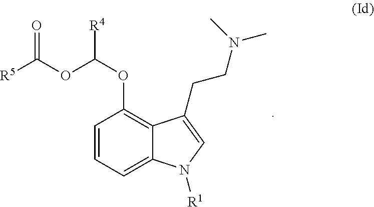

51. The compound or pharmaceutically acceptable salt, solvate, or isotopolog of claim 50, wherein R⁴ is hydrogen, unsubstituted or substituted alkyl, heteroalkyl, cycloalkyl, or heterocyclylalkyl.

52. The compound or pharmaceutically acceptable salt, solvate, or isotopolog of claim 50 or claim 51, wherein R⁴ is hydrogen or unsubstituted or substituted alkyl.

53. The compound or pharmaceutically acceptable salt, solvate, or isotopolog of claim 52, wherein R⁴ is hydrogen, methyl, ethyl, n-propyl, isopropyl, n-butyl, tert-butyl, n-pentyl, 3-methyl-1-butyl, or —C₁₀H₂₁.

54. The compound or pharmaceutically acceptable salt, solvate, or isotopolog of claim 52, wherein R⁴ is hydrogen.

55. The compound or pharmaceutically acceptable salt, solvate, or isotopolog of any one of claims 50 to 54, wherein R⁵ is unsubstituted or substituted alkyl, heteroalkyl, cycloalkyl, or heterocyclylalkyl.

56. The compound or pharmaceutically acceptable salt, solvate, or isotopolog of any one of claims 50 to 55, wherein R⁵ is unsubstituted or substituted alkyl.

57. The compound or pharmaceutically acceptable salt, solvate, or isotopolog of claim 56, wherein R⁵ is alkyl substituted with one or more substituent R^{A}, and wherein each R^{A} is independently selected from alkyl, heteroalkyl, cycloalkyl, heterocyclylalkyl, aryl, heteroaryl, an amino acid side chain, —OR¹³, —N(R¹⁸)R¹⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹], —C(O)N(R¹⁸)R¹⁹, —OC(O)N(R¹⁸)R¹⁹, or —OP(O)OR²⁰(OR²¹).

58. The compound or pharmaceutically acceptable salt, solvate, or isotopolog of any one of claims 50 to 56, wherein R⁵ is unsubstituted alkyl.

59. The compound or pharmaceutically acceptable salt, solvate, or isotopolog of claim 58, wherein R⁵ is methyl, ethyl, n-propyl, isopropyl, n-butyl, tert-butyl, n-pentyl, 3-methyl-1-butyl, or —C₁₀H₂₁.

60. The compound or pharmaceutically acceptable salt, solvate, or isotopolog of any one of claims 50 to 55, wherein R⁵ is alkyl substituted with C(O)OR¹³.

61. The compound or pharmaceutically acceptable salt, solvate, or isotopolog of claim 60, wherein R¹³ is hydrogen or alkyl.

62. The compound or pharmaceutically acceptable salt, solvate, or isotopolog of claim 60, wherein R¹³ is hydrogen, methyl, ethyl, or tert-butyl.

63. The compound or pharmaceutically acceptable salt, solvate, or isotopolog of any one of claims 50 to 55, wherein R⁵ is alkyl substituted with —N(R¹⁸)R¹⁹, wherein each of R¹⁸ and R¹⁹ is independently hydrogen or methyl.

64. The compound or pharmaceutically acceptable salt, solvate, or isotopolog of any one of claims 50 to 55, wherein R⁵ is alkyl substituted with —N(R¹³)C(O)R¹⁴, wherein each of R¹³ and R¹⁴ is independently hydrogen or methyl.

65. The compound or pharmaceutically acceptable salt, solvate, or isotopolog of any one of claims 50 to 55, wherein R⁵ is alkyl substituted with —N(R¹³)C(O)R¹⁴, wherein each of R¹³ is hydrogen or methyl, and R¹⁴ is hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, and heteroaryl is unsubstituted or further substituted with one or more halogen, amino, cyano, hydroxyl, alkyl, acetyl, or benzoyl.

66. The compound or pharmaceutically acceptable salt, solvate, or isotopolog of any one of claims 50 to 55, wherein R⁵ is alkyl substituted with —N(R¹³)C(O)OR¹⁴, wherein each of R¹³ and R¹⁴ is independently hydrogen, methyl, or ethyl.

67. The compound or pharmaceutically acceptable salt, solvate, or isotopolog of any one of claims 50 to 55, wherein R⁵ is alkyl substituted with —N(R¹³)C(O)OR¹⁴, wherein R³ is hydrogen or methyl, and R¹⁴ is hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, and heteroaryl is unsubstituted or further substituted with one or more halogen, amino, cyano, hydroxyl, alkyl, acetyl, or benzoyl.

68. The compound or pharmaceutically acceptable salt, solvate, or isotopolog of any one of claims 50 to 54, wherein R⁵ is heterocyclylalkyl.

69. The compound or pharmaceutically acceptable salt, solvate, or isotopolog of claim 68, wherein R⁵ is selected from aziridinyl, oxetanyl, azetidinyl, tetrahydrofuranyl, pyrrolidinyl, piperidinyl, piperazinyl, morpholinyl, thiomorpholinyl, thiomorpholinyl dioxide, diazinanyl, 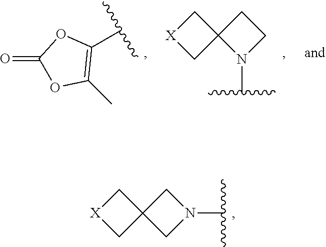
wherein X is —CH₂—, —O—, —S—, —SO₂, —NH—, or —NMe.

70. The compound or pharmaceutically acceptable salt, solvate, or isotopolog of claim 68, wherein R⁵ is optionally substituted piperidinyl.

71. The compound or pharmaceutically acceptable salt, solvate, or isotopolog of claim 68, wherein R⁵ is

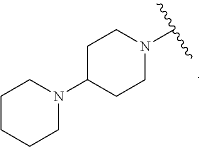 [SMILES: *N1CCC(N2CCCCC2)CC1]

72. The compound or pharmaceutically acceptable salt, solvate, or isotopolog of any one of claims 50 to 55, wherein R³ is alkyl substituted with —OC(O)R¹⁵, wherein R¹⁵ is hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, and heteroaryl is unsubstituted or further substituted with one or more halogen, amino, cyano, hydroxyl, alkyl, acetyl, or benzoyl.

73. The compound or pharmaceutically acceptable salt, solvate, or isotopolog of any one of claims 50 to 54, wherein R⁵ is heteroalkyl.

74. The compound or pharmaceutically acceptable salt, solvate, or isotopolog of any one of claims 50 to 54, wherein R⁵ is unsubstituted or substituted aryl (e.g., phenyl).

75. The compound or pharmaceutically acceptable salt, solvate, or isotopolog of any one of claims 50 to 54, wherein R⁵ is t-butyl, —CH(NH₂)CH(CH₃)₂, —CH₂N(CH₃)₂, —CH₂CH₂OCH₃, —CH₂CH₂NH(CH₃)₂, —CH₂CH₂C(CH₃)₂₀C(O)CH₃, —CH₂CH₂C(CH₃)₂NHC(O)CH₃, or —CH₂CH₂C(CH₃)₂NHC(O)OCH₂CH₃.

76. The compound or pharmaceutically acceptable salt, solvate, or isotopolog of claim 1, wherein the compound has the structure of Formula (Ie):

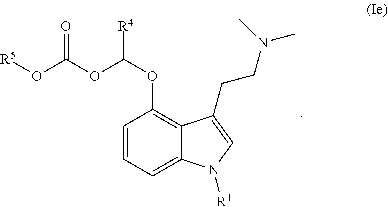

77. The compound or pharmaceutically acceptable salt, solvate, or isotopolog of claim 76, wherein R⁴ is hydrogen, unsubstituted or substituted alkyl, heteroalkyl, cycloalkyl, or heterocyclylalkyl.

78. The compound or pharmaceutically acceptable salt, solvate, or isotopolog of claim 76 or claim 77, wherein R⁴ is unsubstituted or substituted alkyl.

79. The compound or pharmaceutically acceptable salt, solvate, or isotopolog of claim 78, wherein R⁴ is methyl, ethyl, n-propyl, isopropyl, n-butyl, tert-butyl, n-pentyl, 3-methyl-1-butyl, or —C₁₀H₂₁.

80. The compound or pharmaceutically acceptable salt, solvate, or isotopolog of claim 76 or claim 77, wherein R⁴ is hydrogen.

81. The compound or pharmaceutically acceptable salt, solvate, or isotopolog of any one of claims 76 to 79, wherein R⁵ is unsubstituted or substituted alkyl, heteroalkyl, cycloalkyl, or heterocyclylalkyl.

82. The compound or pharmaceutically acceptable salt, solvate, or isotopolog of any one of claims 76 to 81, wherein R⁵ is unsubstituted or substituted alkyl.

83. The compound or pharmaceutically acceptable salt, solvate, or isotopolog of claim 82, wherein R⁵ is alkyl substituted with one or more substituent R^{A}, and wherein each R^{A} is independently selected from alkyl, heteroalkyl, cycloalkyl, heterocyclylalkyl, aryl, heteroaryl, an amino acid side chain, —OR¹³, —N(R¹⁸)R⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹], —C(O)N(R¹⁸)R¹⁹, —OC(O)N(R¹⁸)R¹⁹, or —OP(O)OR²⁰(OR²¹).

84. The compound or pharmaceutically acceptable salt, solvate, or isotopolog of any one of claims 76 to 82, wherein R⁵ is unsubstituted alkyl.

85. The compound or pharmaceutically acceptable salt, solvate, or isotopolog of claim 84, wherein R⁵ is methyl, ethyl, n-propyl, isopropyl, n-butyl, tert-butyl, n-pentyl, 3-methyl-1-butyl, or —C₁₀H₂₁.

86. The compound or pharmaceutically acceptable salt, solvate, or isotopolog of any one of claims 76 to 83, wherein R⁵ is alkyl substituted with —N(R¹⁸)R¹⁹, wherein each of R¹⁸ and R¹⁹ is independently hydrogen or methyl.

87. The compound or pharmaceutically acceptable salt, solvate, or isotopolog of any one of claims 76 to 83, wherein R⁵ is alkyl substituted with —N(R¹³)C(O)R¹⁴, wherein each of R¹³ and R¹⁴ is independently hydrogen or methyl.

88. The compound or pharmaceutically acceptable salt, solvate, or isotopolog of any one of claims 76 to 83, wherein R⁵ is alkyl substituted with —N(R¹³)C(O)R¹⁴, wherein each of R¹³ is hydrogen or methyl, and R¹⁴ is hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, and heteroaryl is unsubstituted or further substituted with one or more halogen, amino, cyano, hydroxyl, alkyl, acetyl, or benzoyl.

89. The compound or pharmaceutically acceptable salt, solvate, or isotopolog of any one of claims 76 to 83, wherein R⁵ is alkyl substituted with —N(R¹³)C(O)OR¹⁴, wherein each of R¹³ and R¹⁴ is independently hydrogen, methyl, or ethyl.

90. The compound or pharmaceutically acceptable salt, solvate, or isotopolog of any one of claims 76 to 79, wherein R⁵ is heterocyclylalkyl.

91. The compound or pharmaceutically acceptable salt, solvate, or isotopolog of claim 90, wherein R⁵ is selected from aziridinyl, oxetanyl, azetidinyl, tetrahydrofuranyl, pyrrolidinyl, piperidinyl, piperazinyl, morpholinyl, thiomorpholinyl, thiomorpholinyl dioxide, diazinanyl, 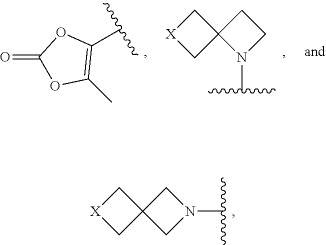
wherein X is —CH₂—, —O—, —S—, —SO₂, —NH—, or —NMe.

92. The compound or pharmaceutically acceptable salt, solvate, or isotopolog of claims 76 to 83, wherein R³ is alkyl substituted with —OC(O)R¹⁵, wherein R¹⁵ is hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, and heteroaryl is unsubstituted or further substituted with one or more halogen, amino, cyano, hydroxyl, alkyl, acetyl, or benzoyl.

93. The compound or pharmaceutically acceptable salt, solvate, or isotopolog of any one of claims 76 to 79, wherein R⁵ is heteroalkyl.

94. The compound or pharmaceutically acceptable salt, solvate, or isotopolog of any one of claims 76 to 79, wherein R⁵ is unsubstituted or substituted aryl (e.g., phenyl).

95. The compound or pharmaceutically acceptable salt, solvate, or isotopolog of any one of claims 76 to 79, wherein R⁵ is morpholinyl, isopropyl, or ethyl.

96. The compound or pharmaceutically acceptable salt, solvate, or isotopolog of claim 1, wherein the compound has the structure of Formula (If):

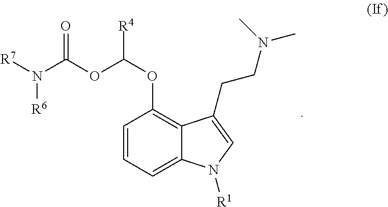

97. The compound or pharmaceutically acceptable salt, solvate, or isotopolog of claim 96, wherein R⁴ is hydrogen, unsubstituted or substituted alkyl, heteroalkyl, cycloalkyl, or heterocyclylalkyl.

98. The compound or pharmaceutically acceptable salt, solvate, or isotopolog of claim 96 or claim 97, wherein R⁴ is unsubstituted or substituted alkyl.

99. The compound or pharmaceutically acceptable salt, solvate, or isotopolog of claim 98, wherein R⁴ is methyl, ethyl, n-propyl, isopropyl, n-butyl, tert-butyl, n-pentyl, 3-methyl-1-butyl, or —C₁₀H₂₁.

100. The compound or pharmaceutically acceptable salt, solvate, or isotopolog of claim 96 or 97, wherein R⁴ is hydrogen.

101. The compound or pharmaceutically acceptable salt, solvate, or isotopolog of any one of claim 96 to 100, wherein each of R⁶ and R⁷ is independently alkyl, heteroalkyl, cycloalkyl, heterocyclylalkyl, aryl, or heteroaryl, wherein alkyl, heteroalkyl, cycloalkyl, heterocyclylalkyl, aryl, or heteroaryl is unsubstituted or substituted with one or more R^{A}.

102. The compound or pharmaceutically acceptable salt, solvate, or isotopolog of claim 101, wherein R⁶ is hydrogen or methyl, and R⁷ is hydrogen alkyl, heteroalkyl, cycloalkyl, heterocyclylalkyl, aryl, or heteroaryl, wherein alkyl, heteroalkyl, cycloalkyl, heterocyclylalkyl, aryl, or heteroaryl is unsubstituted or substituted with one or more R^{A}.

103. The compound or pharmaceutically acceptable salt, solvate, or isotopolog of any one of claim 96 to 100, wherein R⁶ and R⁷ together with the atom to which they are attached form a heterocyclylalkyl ring or a heteroaryl ring that is unsubstituted or substituted with one or more R^{A}.

104. The compound or pharmaceutically acceptable salt, solvate, or isotopolog of claim 103, wherein R⁶ and R⁷ together with the atom to which they are attached form aziridinyl, azetidinyl, pyrrolidinyl, piperidinyl, piperazinyl, morpholinyl, thiomorpholinyl, thiomorpholinyl dioxide, diazinanyl, 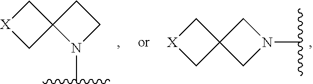
wherein X is —CH—, —O—, —S—, —SO₂, —NH—, or —NMe.

105. The compound or pharmaceutically acceptable salt, solvate, or isotopolog of claim 103, wherein R⁶ and R⁷ together with the atom to which they are attached form optionally substituted piperidinyl.

106. The compound or pharmaceutically acceptable salt, solvate, or isotopolog of claim 103, wherein R⁶ and R⁷ together with the atom to which they are attached form

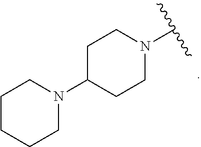 [SMILES: *N1CCC(N2CCCCC2)CC1]

107. The compound or pharmaceutically acceptable salt, solvate, or isotopolog of claim 1, wherein the compound has the structure of Formula (Ig):

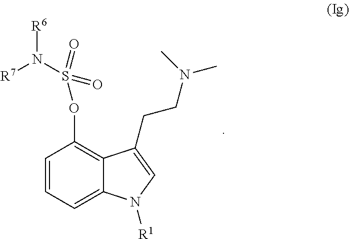

108. The compound or pharmaceutically acceptable salt, solvate, or isotopolog of claim 107, wherein each of R⁶ and R⁷ is independently hydrogen, alkyl, heteroalkyl, cycloalkyl, heterocyclylalkyl, aryl, or heteroaryl, wherein alkyl, heteroalkyl, cycloalkyl, heterocyclylalkyl, aryl, or heteroaryl is unsubstituted or substituted with one or more R^{A}.

109. The compound or pharmaceutically acceptable salt, solvate, or isotopolog of claim 107, wherein R⁶ and R⁷ are each independently hydrogen or alkyl.

110. The compound or pharmaceutically acceptable salt, solvate, or isotopolog of claim 107, wherein R⁶ and R⁷ are each independently hydrogen or methyl.

111. The compound or pharmaceutically acceptable salt, solvate, or isotopolog of claim 107, wherein R⁶ and R⁷ are each hydrogen.

112. The compound or pharmaceutically acceptable salt, solvate, or isotopolog of claim 107, wherein R⁶ and R⁷ together with the atom to which they are attached form a heterocyclylalkyl ring or a heteroaryl ring that is unsubstituted or substituted with one or more R^{A}.

113. The compound or pharmaceutically acceptable salt, solvate, or isotopolog of claim 112, wherein R⁶ and R⁷ together with the atom to which they are attached form aziridinyl, azetidinyl, pyrrolidinyl, piperidinyl, piperazinyl, morpholinyl, thiomorpholinyl, thiomorpholinyl dioxide, diazinanyl, 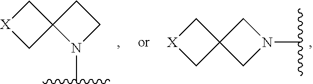
wherein X is —CH₂—, —O—, —S—, —SO₂, —NH—, or —NMe.

114. The compound or pharmaceutically acceptable salt, solvate, or isotopolog of claim 1, wherein the compound has the structure of Formula (Ih):

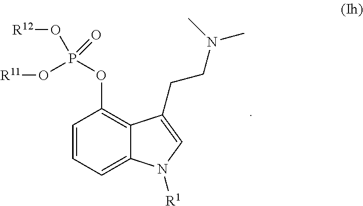

115. The compound or pharmaceutically acceptable salt, solvate, or isotopolog of claim 114, wherein each of R¹¹ and R¹² is independently hydrogen, unsubstituted or substituted alkyl, heteroalkyl, cycloalkyl, or heterocyclylalkyl.

116. The compound or pharmaceutically acceptable salt, solvate, or isotopolog of claim 114 or 115, wherein each of R¹¹ and R¹² is independently hydrogen or unsubstituted or substituted alkyl.

117. The compound or pharmaceutically acceptable salt, solvate, or isotopolog of claim 115, wherein each of R¹¹ and R¹² is independently alkyl substituted with one or more substituent R^{A}, and wherein each R^{A} is independently selected from alkyl, heteroalkyl, cycloalkyl, heterocyclylalkyl, aryl, heteroaryl, an amino acid side chain, —OR¹³, —N(R¹⁸)R¹⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹], —C(O)N(R¹⁸)R¹⁹, —OC(O)N(R¹⁸)R¹⁹, or —OP(O)OR²⁰(OR²¹).

118. The compound or pharmaceutically acceptable salt, solvate, or isotopolog of claim 114 or 115, wherein each of R¹¹ and R¹² is independently unsubstituted alkyl.

119. The compound or pharmaceutically acceptable salt, solvate, or isotopolog of claim 93, wherein each of R¹¹ and R¹² is independently methyl, ethyl, n-propyl, isopropyl, n-butyl, tert-butyl, n-pentyl, 3-methyl-1-butyl, or —C₁₀H₂₁.

120. The compound or pharmaceutically acceptable salt, solvate, or isotopolog of claim 114 or 115, wherein each of R¹¹ and R¹² is independently alkyl substituted with —OC(O)R¹⁵, wherein R¹⁵ is hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, and heteroaryl is unsubstituted or further substituted with one or more halogen, amino, cyano, hydroxyl, alkyl, acetyl, or benzoyl.

121. The compound or pharmaceutically acceptable salt, solvate, or isotopolog of claim 114 or 115, wherein each of R¹¹ and R¹² is independently alkyl substituted with —OC(O)OR¹⁶, wherein R¹⁶ is hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, and heteroaryl is unsubstituted or further substituted with one or more halogen, amino, cyano, hydroxyl, alkyl, acetyl, or benzoyl.

122. The compound or pharmaceutically acceptable salt, solvate, or isotopolog of claim 121, wherein R¹⁶ is hydrogen or alkyl.

123. The compound or pharmaceutically acceptable salt, solvate, or isotopolog of claim 121, wherein R¹⁶ is hydrogen, methyl, ethyl, isopropyl or tert-butyl.

124. The compound or pharmaceutically acceptable salt, solvate, or isotopolog of claim 89 or claim 114, wherein each of R¹¹ and R¹² is independently heteroalkyl.

125. The compound or pharmaceutically acceptable salt, solvate, or isotopolog of claim 89 or claim 114, wherein each of R¹¹ and R¹² is independently unsubstituted or substituted aryl (e.g., phenyl).

126. The compound or pharmaceutically acceptable salt, solvate, or isotopolog of claim 114, wherein the compound has the structure of Formula (Ih′): 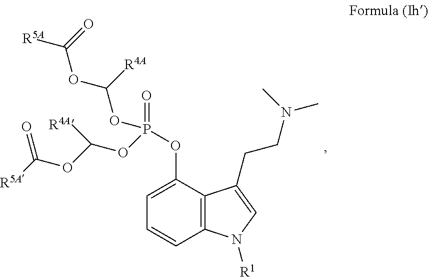
wherein R^{4A} and R^{4A}′ are each independently hydrogen or alkyl, and R^{5A} and R^{5A}′ are each independently hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, and heteroaryl is unsubstituted or further substituted with one or more halogen, amino, cyano, hydroxyl, alkyl, acetyl, or benzoyl.

127. The compound or pharmaceutically acceptable salt, solvate, or isotopolog of claim 126, wherein R^{4A} and R^{4A}′ are each hydrogen.

128. The compound or pharmaceutically acceptable salt, solvate, or isotopolog of claim 126 or 127, wherein R^{5A} and R^{5A}′ are each methyl, ethyl, n-propyl, isopropyl, n-butyl, tert-butyl, n-pentyl, 3-methyl-1-butyl, or —C₁₀H₂₁.

129. The compound or pharmaceutically acceptable salt, solvate, or isotopolog of claim 126 or 127, wherein R^{5A} and R^{5A}′ are each isopropyl or tert-butyl.

130. The compound or pharmaceutically acceptable salt, solvate, or isotopolog of claim 26, wherein the compound has the structure of Formula (Ib′): 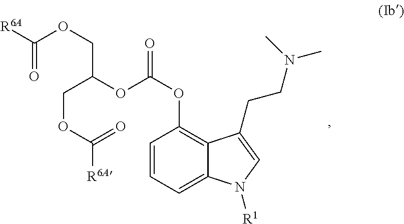
wherein R^{6A} and R^{6A}′ are each independently hydrogen or alkyl.

131. The compound or pharmaceutically acceptable salt, solvate, or isotopolog of claim 130, wherein R^{6A} and R^{6A}′ are each independently —CH₃, —C₂H₅, C₃H₇, C₄H₉, C₅H₁₁, C₆H₁₃, C₇H₁₅, C₈H₁₇, C₉H₁₉, C₁₀H₂₁, C₁₁H₂₃, C₁₂H₂₅, C₁₃H₂₇, C₁₄H₂₉, C₁₅H₃₁, C₁₆H₃₃, or C₁₇H₃₅.

132. The compound or pharmaceutically acceptable salt, solvate, or isotopolog of claim 130 or 131, wherein R^{6A} and R^{6A}′ are the same.

133. The compound or pharmaceutically acceptable salt, solvate, or isotopolog of claim 26, wherein the compound has the structure of Formula (Ib″): 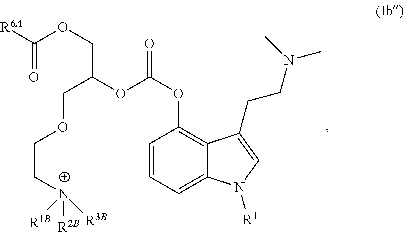
wherein each of R^{6A}, R^{1B}, R^{2B}, and R^{3B} are independently hydrogen or alkyl.

134. The compound or pharmaceutically acceptable salt, solvate, or isotopolog of claim 133, wherein R^{6A} is —CH₃, —C₂H₅, C₃H₇, C₄H₉, C₅H₁₁, C₆H₁₃, C₇H₁₅, C₈H₁₇, C₉H₁₉, C₁₀H₂₁, CnH₂₃, C₁₂H₂₅, C₁₃H₂₇, C₁₄H₂₉, C₁₅H₃₁, C₁₆H₃₃, or C₁₇H₃₅.

135. The compound or pharmaceutically acceptable salt, solvate, or isotopolog of claim 133 or 134, wherein R^{1B}, R^{2B}, and R^{3B} are each independently alkyl.

136. The compound or pharmaceutically acceptable salt, solvate, or isotopolog of claim 135, wherein each of R^{1B}, R^{2B}, and R^{3B} are independently methyl, ethyl, n-propyl, isopropyl, n-butyl, tert-butyl, n-pentyl, 3-methyl-1-butyl, or —C₁₀H₂₁.

137. The compound or pharmaceutically acceptable salt, solvate, or isotopolog of claim 135, wherein R^{1B}, R^{2B}, and R^{3B} are each methyl.

138. The compound or pharmaceutically acceptable salt, solvate, or isotopolog of claim 1, wherein the compound has the structure of Formula (Ii):

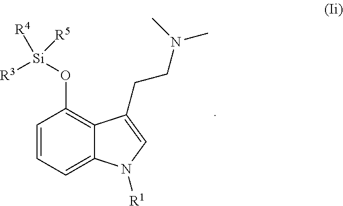

139. The compound or pharmaceutically acceptable salt, solvate, or isotopolog of claim 138, wherein each of R³, R⁴ and R⁵ is independently hydrogen, unsubstituted or substituted alkyl, heteroalkyl, cycloalkyl, or heterocyclylalkyl.

140. The compound or pharmaceutically acceptable salt, solvate, or isotopolog of claim 138 or 139, wherein each of R³, R⁴ and R⁵ is unsubstituted or substituted alkyl.

141. The compound or pharmaceutically acceptable salt, solvate, or isotopolog of any one of claims 138 to 140, wherein each of R³, R⁴ and R⁵ is independently alkyl substituted with one or more substituent R^{A}, and wherein each R^{A} is independently selected from alkyl, heteroalkyl, cycloalkyl, heterocyclylalkyl, aryl, heteroaryl, an amino acid side chain, —OR¹³, —N(R¹⁸)R¹⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹], —C(O)N(R¹⁸)R¹⁹, —OC(O)N(R¹⁸)R¹⁹, or —OP(O)OR²⁰(OR²¹).

142. The compound or pharmaceutically acceptable salt, solvate, or isotopolog of any one of claims 138 to 140, wherein each of R³, R⁴ and R⁵ is independently unsubstituted alkyl.

143. The compound or pharmaceutically acceptable salt, solvate, or isotopolog of claim 142, wherein each of R³, R⁴ and R⁵ is independently methyl, ethyl, n-propyl, isopropyl, n-butyl, tert-butyl, n-pentyl, 3-methyl-1-butyl, or —C₁₀H₂₁.

144. The compound or pharmaceutically acceptable salt, solvate, or isotopolog of claim 142, wherein R³, R⁴ and R⁵ are the same unsubstituted alkyl.

145. The compound or pharmaceutically acceptable salt, solvate, or isotopolog of claim 142, wherein R³ and R⁴ are methyl, ethyl or isopropyl.

146. The compound or pharmaceutically acceptable salt, solvate, or isotopolog of claim 145, wherein R⁵ is ethyl, isopropyl, or tert-butyl.

147. The compound or pharmaceutically acceptable salt, solvate, or isotopolog of claim 142, wherein (i) R³ and R⁴ are methyl, R⁵ is ethyl; (ii) R³, R⁴ and R⁵ are isopropyl; or (iii) R³, R⁴ and R⁵ are ethyl.

148. The compound or pharmaceutically acceptable salt, solvate, or isotopolog of claim 89 or claim 138, wherein each of R³, R⁴ and R⁵ is independently heteroalkyl.

149. The compound or pharmaceutically acceptable salt, solvate, or isotopolog of claim 89 or claim 138, wherein each of R³, R⁴ and R⁵ is independently unsubstituted or substituted aryl (e.g., phenyl).

150. The compound or pharmaceutically acceptable salt, solvate, or isotopolog of claim 1, wherein the compound has the structure of Formula (Ij):

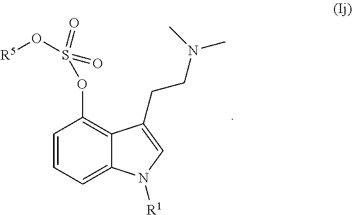

151. The compound or pharmaceutically acceptable salt, solvate, or isotopolog of claim 150, wherein R⁵ is unsubstituted or substituted alkyl, heteroalkyl, cycloalkyl, or heterocyclylalkyl.

152. The compound or pharmaceutically acceptable salt, solvate, or isotopolog of claim 150 or 151, wherein R⁵ is unsubstituted or substituted alkyl.

153. The compound or pharmaceutically acceptable salt, solvate, or isotopolog of claim 150 or 151, wherein R⁵ is alkyl substituted with one or more substituent R^{A}, and wherein each R^{A} is independently selected from alkyl, heteroalkyl, cycloalkyl, heterocyclylalkyl, aryl, heteroaryl, an amino acid side chain, —OR¹³, —N(R¹⁸)R¹⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹], —C(O)N(R¹⁸)R¹⁹, —OC(O)N(R¹⁸)R¹⁹, or —OP(O)OR²⁰(OR²¹).

154. The compound or pharmaceutically acceptable salt, solvate, or isotopolog of any one of claims 150 to 153, wherein R⁵ is unsubstituted alkyl.

155. The compound or pharmaceutically acceptable salt, solvate, or isotopolog of claim 154, wherein R⁵ is methyl, ethyl, n-propyl, isopropyl, n-butyl, tert-butyl, n-pentyl, 3-methyl-1-butyl, or —C₁₀H₂₁.

156. The compound or pharmaceutically acceptable salt, solvate, or isotopolog of any one of claims 150 to 153, wherein R⁵ is alkyl substituted with —C(O)OR¹³, wherein R¹³ is hydrogen or alkyl.

157. The compound or pharmaceutically acceptable salt, solvate, or isotopolog of claim 156, wherein R⁵ is hydrogen, methyl, ethyl, isopropyl, or tert-butyl.

158. The compound or pharmaceutically acceptable salt, solvate, or isotopolog of any one of claims 150 to 153, wherein R⁵ is alkyl substituted with —N(R¹⁸)R¹⁹, wherein each of R¹⁸ and R¹⁹ is independently hydrogen or methyl.

159. The compound or pharmaceutically acceptable salt, solvate, or isotopolog of any one of claims 150 to 153, wherein R⁵ is alkyl substituted with —N(R¹³)C(O)R¹⁴, wherein each of R¹³ and R¹⁴ is independently hydrogen or methyl.

160. The compound or pharmaceutically acceptable salt, solvate, or isotopolog of any one of claims 150 to 153, wherein R⁵ is alkyl substituted with —N(R¹³)C(O)R¹⁴, wherein each of R¹³ is hydrogen or methyl, and R¹⁴ is hydrogen, alkyl, alkenyl, heteroalkyl, cycloalkyl, heterocyclylalkyl, aryl, or heteroaryl, wherein alkyl, heteroalkyl, cycloalkyl, heterocyclylalkyl, aryl, and heteroaryl is unsubstituted or further substituted with one or more halogen, amino, cyano, hydroxyl, alkyl, acetyl, or benzoyl.

161. The compound or pharmaceutically acceptable salt, solvate, or isotopolog of any one of claims 150 to 153, wherein R⁵ is alkyl substituted with —N(R¹³)C(O)OR¹⁴, wherein each of R¹³ and R¹⁴ is independently hydrogen, methyl, or ethyl.

162. The compound or pharmaceutically acceptable salt, solvate, or isotopolog of claim 150 or 151, wherein R⁵ is heterocyclylalkyl.

163. The compound or pharmaceutically acceptable salt, solvate, or isotopolog of claim 162, wherein R⁵ is selected from aziridinyl, oxetanyl, azetidinyl, tetrahydrofuranyl, pyrrolidinyl, piperidinyl, piperazinyl, morpholinyl, thiomorpholinyl, thiomorpholinyl dioxide, diazinanyl, 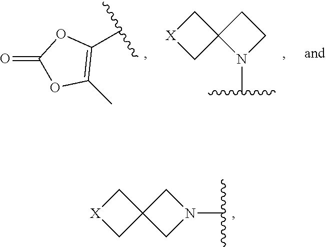
wherein X is —CH₂—, —O—, —S—, —SO₂, —NH—, or —NMe.

164. The compound or pharmaceutically acceptable salt, solvate, or isotopolog of any one of claims 1 to 163, wherein R¹ is hydrogen.

165. A compound of Formula (II), or a pharmaceutically acceptable salt, solvate, or isotopolog thereof: 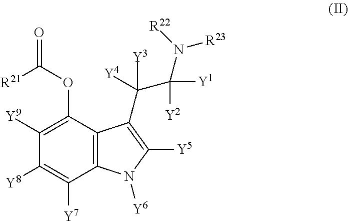
wherein:
R²¹ is CH₃, CH₂D, CHD₂, or CD₃;
each of R²² and R²³ is independently hydrogen or alkyl, wherein one or more of the hydrogens in the alkyl is optionally substituted with deuterium;
each of Y¹, Y², Y³, Y⁴, Y⁵, Y⁶, Y⁷, Y⁸, and Y⁹ is independently hydrogen or deuterium; and
wherein when R²¹ is CH₃, and R²² and R²³ do not comprise deuterium, at least one of Y¹, Y², Y³, Y⁴, Y⁵, Y⁶, Y⁷, Y⁸, and Y⁹ is deuterium.

166. The compound or pharmaceutically acceptable salt, solvate, or isotopolog of claim 111, wherein R²¹ is —CH₃.

167. The compound or pharmaceutically acceptable salt, solvate, or isotopolog of claim 111, wherein R²¹ is —CD₃.

168. The compound or pharmaceutically acceptable salt, solvate, or isotopolog of any one of claims 111 to 113, wherein R²² and R²³ are each independently —CH₃, —CH₂D, —CHD₂, or —CD₃.

169. The compound or pharmaceutically acceptable salt, solvate, or isotopolog of any one of claims 111 to 113, wherein at least one of R²² and R²³ comprises deuterium.

170. The compound or pharmaceutically acceptable salt, solvate, or isotopolog of any one of claims 111 to 113, wherein one of R²² and R²³ is —CD₃.

171. The compound or pharmaceutically acceptable salt, solvate, or isotopolog of any one of claims 111 to 113, wherein both R²² and R²³ are —CD₃.

172. The compound or pharmaceutically acceptable salt, solvate, or isotopolog of any one of claims 111 to 117, wherein Y¹ is D.

173. The compound or pharmaceutically acceptable salt, solvate, or isotopolog of any one of claims 111 to 117, wherein Y³ is D.

174. The compound or pharmaceutically acceptable salt, solvate, or isotopolog of any one of claims 111 to 117, wherein Y¹ and Y² are each D.

175. The compound or pharmaceutically acceptable salt, solvate, or isotopolog of any one of claims 111 to 117, wherein Y³ and Y⁴ are each D.

176. The compound or pharmaceutically acceptable salt, solvate, or isotopolog of any one of claims 111 to 117, wherein Y¹, Y², Y³, and Y⁴ are each D.

177. The compound or pharmaceutically acceptable salt, solvate, or isotopolog of any one of claims 111 to 117, wherein Y⁶ is H.

178. The compound or pharmaceutically acceptable salt, solvate, or isotopolog of claim 111, wherein the compound is selected from the group consisting of:

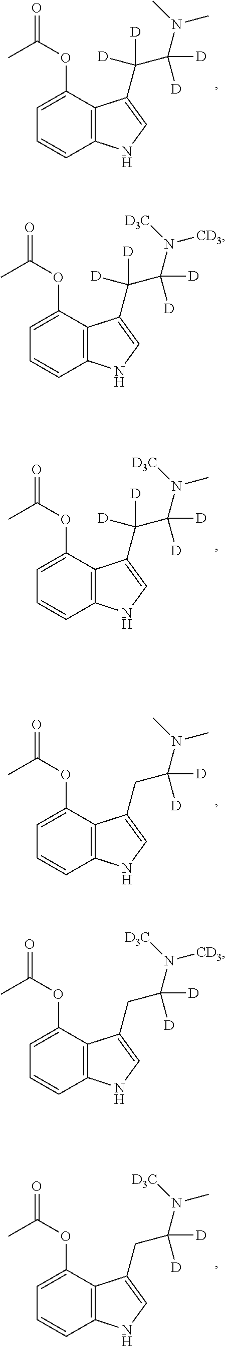

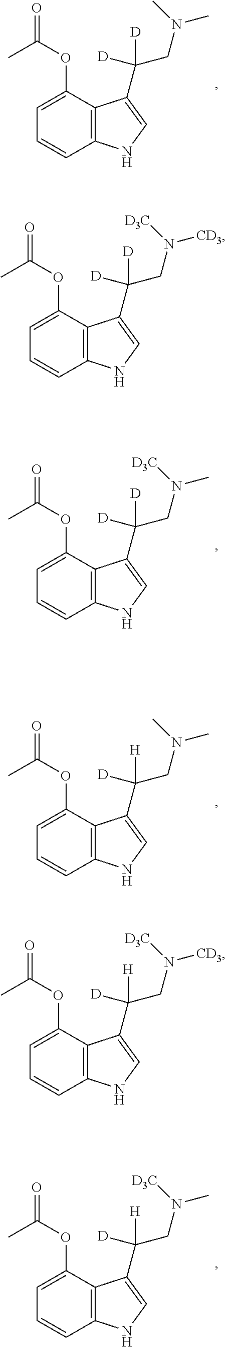

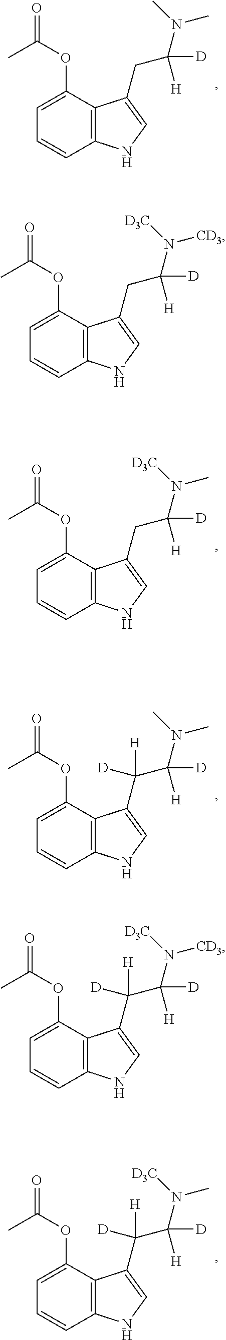

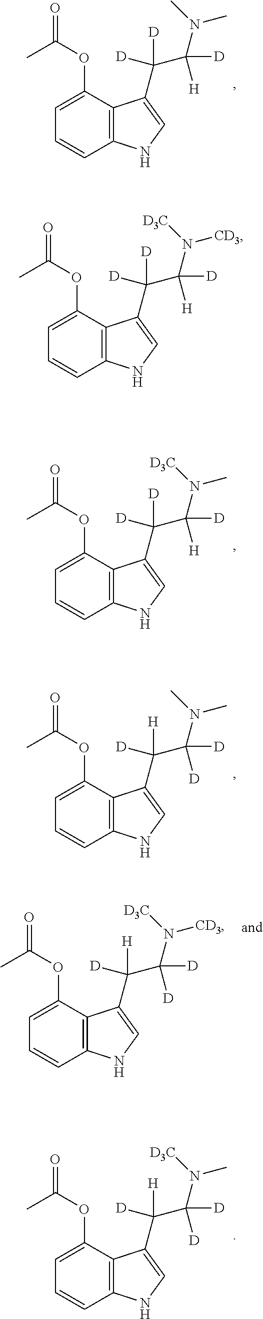

179. The compound or pharmaceutically acceptable salt, solvate, or isotopolog of claim 1, wherein the compound is a compound described in Table 1.

180. The compound or pharmaceutically acceptable salt, solvate, or isotopolog of claim 1, wherein the compound is selected from the group consisting of:

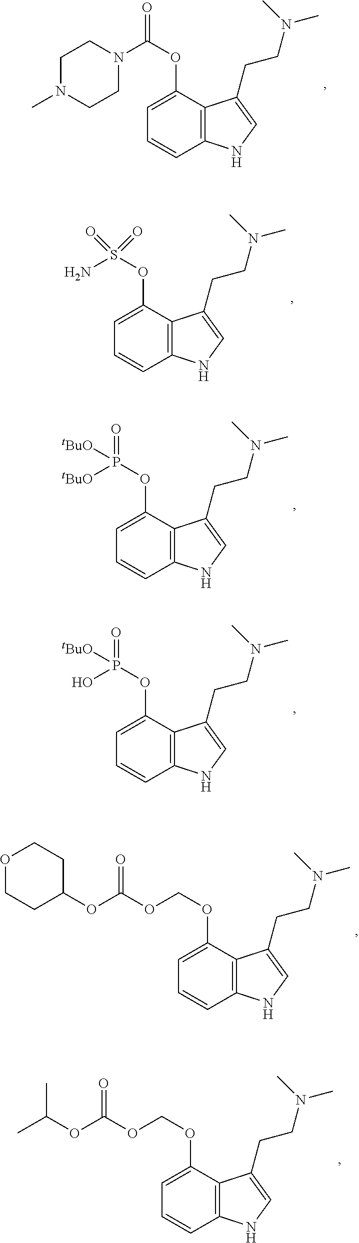

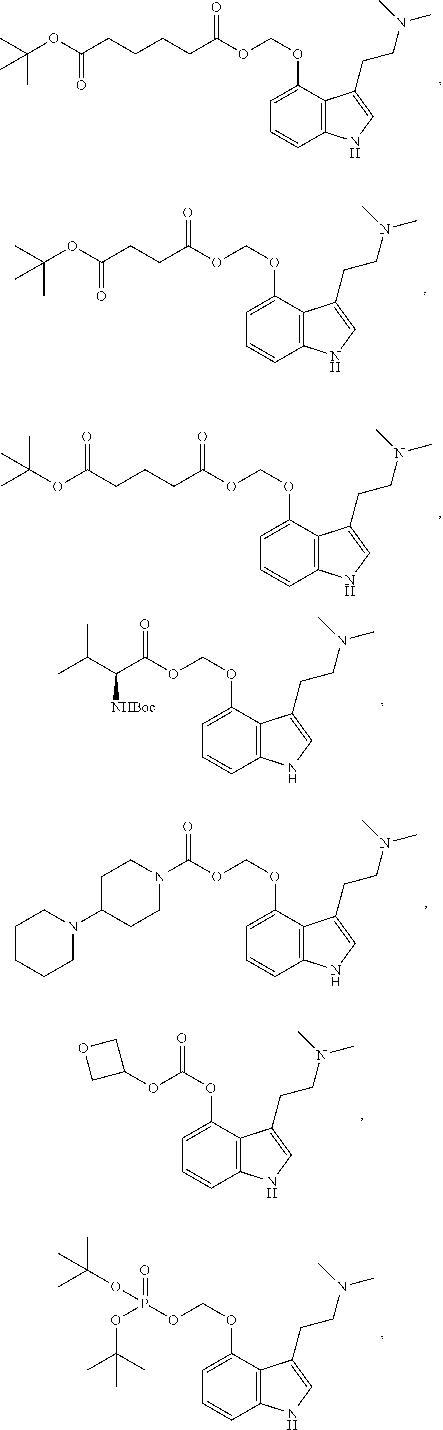

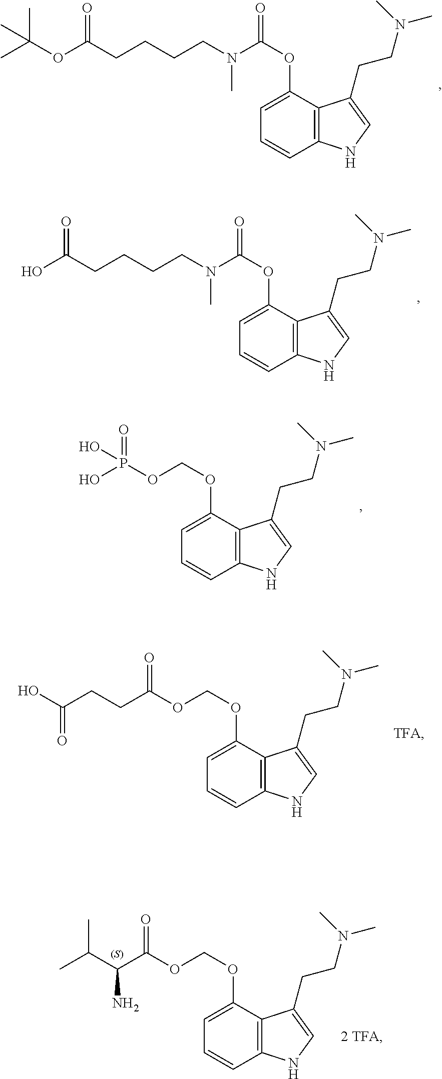

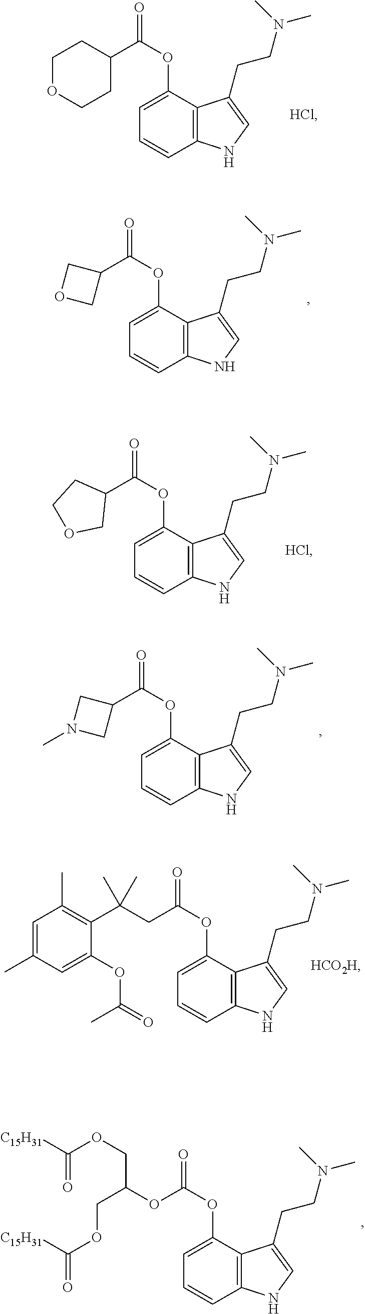

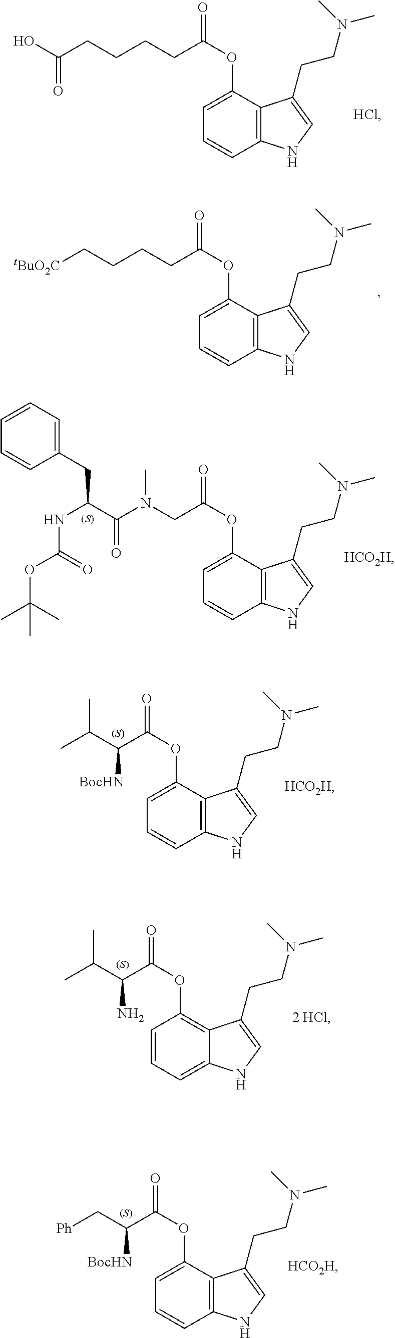

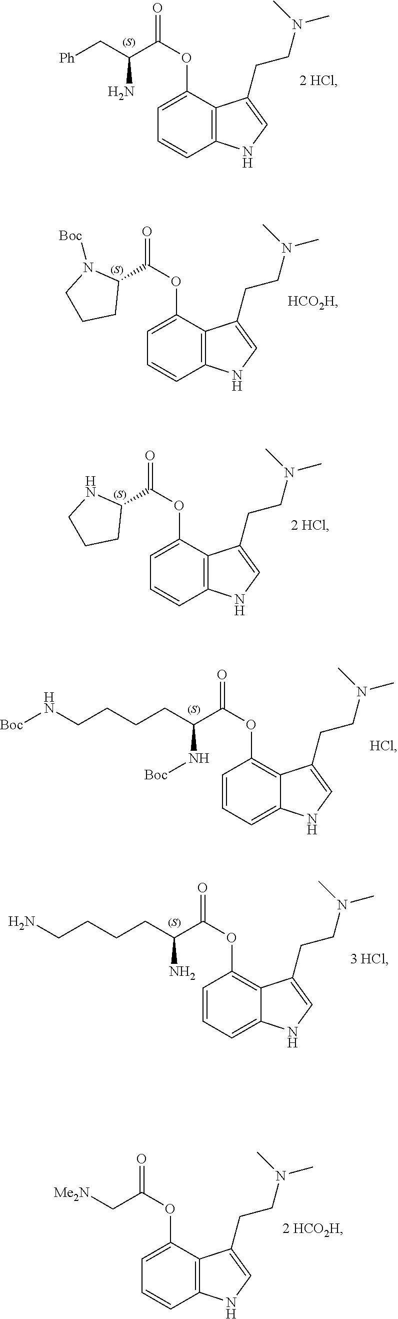

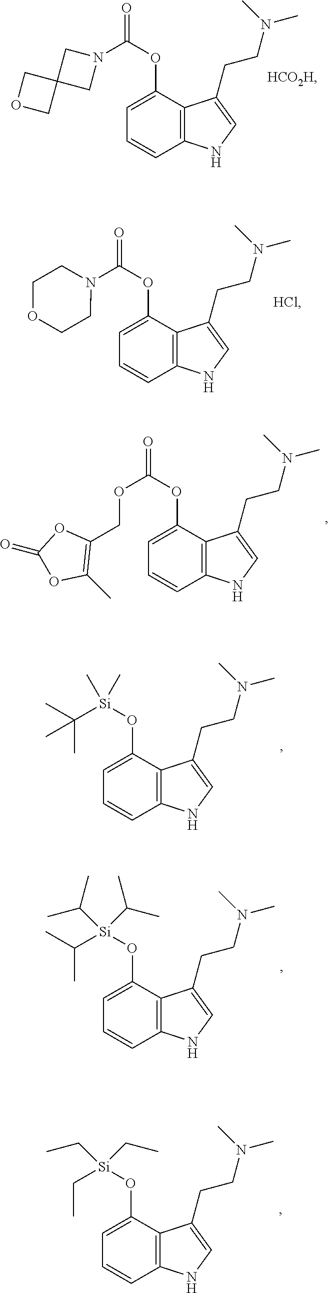

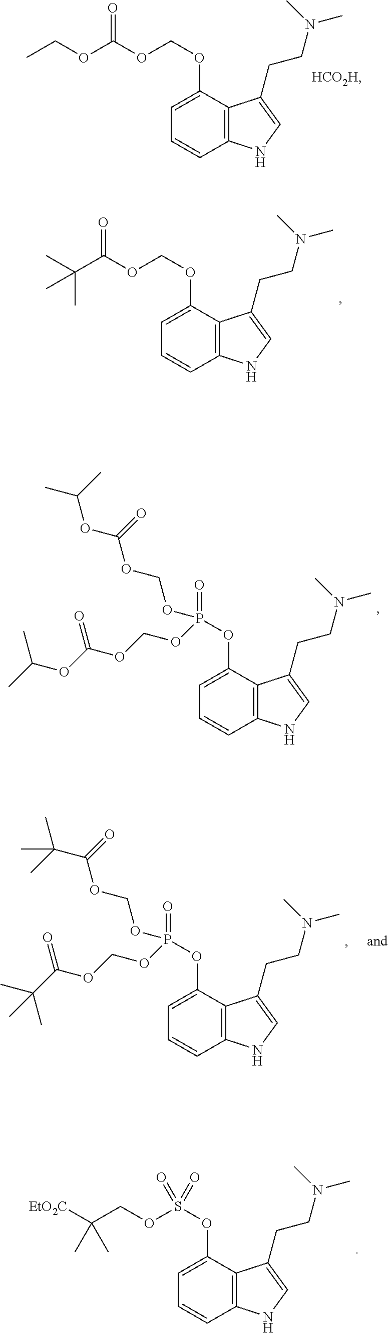

181. A pharmaceutically composition comprising a compound according to any one of claims 1-180, or a pharmaceutically acceptable salt thereof, and a pharmaceutically acceptable excipient or carrier.

182. A method of treating or preventing a disease, disorder, or condition in which an increased level of psilocin is beneficial, comprising administering to a subject in need thereof an effective amount of the compound or pharmaceutically acceptable salt, solvate, or isotopolog according to any one of claims 1-180 or the pharmaceutical composition of claim 181.

183. The method of claim 182, wherein the disease, disorder, or condition is selected from post-traumatic stress disorder, major depression, schizophrenia, Alzheimer's disease, frontotemporal dementia, Parkinson's disease, Parkinson's dementia, dementia, Lewy body dementia, multiple system atrophy, and substance abuse.
